(19) United States
(12) Patent Application Publication
(10) Pub. No.: US 2026/0176353 A1
(43) Pub. Date: Jun. 25, 2026

(54) ANTI-CACFD1 (CALCIUM CHANNEL FLOWER DOMAIN CONTAINING 1) ANTIBODIES AND USES THEREOF

(71) Applicant: FITNESS FINGERPRINT THERAPEUTICS, Henrico, VA (US)

(72) Inventor: Rajan GOGNA, Henrico, VA (US)

(73) Assignee: FITNESS FINGERPRINT THERAPEUTICS, Henrico, VA (US)

(21) Appl. No.: 19/431,026

(22) Filed: Dec. 23, 2025

Related U.S. Application Data
(63) Continuation of application No. PCT/US25/10520, filed on Jan. 7, 2025.
(60) Provisional application No. 63/618,539, filed on Jan. 8, 2024.
(60) Provisional application No. 63/701,726, filed on Oct. 1, 2024.

(51) Int. Cl. C07K 16/28; A61K 39/00; A61P 35/00

(52) U.S. Cl. CPC C07K 16/28; A61P 35/00; A61K 2039/505; C07K 2317/24; C07K 2317/565; C07K 2317/92

(57) ABSTRACT

The present disclosure provides antibodies that bind to calcium channel flower homolog proteins such as hFwe1, hFwe2, hFwe3 and hFwe4. It is known that these calcium channel flower homolog proteins and their downstream players are a common, tunable read-out of cellular fitness likely functional in many tissue systems as means to continuously survey cellular status and rid suboptimal cells. The antibodies provided herein are capable of increasing the cellular fitness of various cell types, and thus would be useful in methods of treating cancer and other diseases or conditions.

### CROSS-REFERENCE TO RELATED APPLICATIONS

This Application is a Continuation Application of PCT International Application No. PCT/US25/10520, International Filing Date Jan. 7, 2025 which published at Publication No. WO 2025/151389 on Jul. 17, 2025, which claims the benefit of priority of U.S. Provisional Application No. 63/618,539 filed Jan. 8, 2024 and U.S. Provisional Application No. 63/701,726 filed Oct. 1, 2024, which are all hereby incorporated by reference in their entirety.

### SEQUENCE LISTING STATEMENT

The instant application contains a Sequence Listing which has been submitted electronically in XML file format and is hereby incorporated by reference in its entirety. Said XML copy, created on Dec. 10, 2024, is named P-634137-PC_SL.xml and is 360,600 bytes in size.

### FIELD OF THE INVENTION

The present disclosure relates in general to the field of cancer therapy. In one embodiment, there is provided anti-CACFD1 (calcium channel Flower domain containing 1) antibodies and their uses in increasing the cellular fitness of cells or tissues.

### BACKGROUND

Accumulation of damage and genetic aberrations within a cell may render the cell suboptimal, or less fit, but still viable, Over time, retention of suboptimal cells likely results in tissue abnormalities, disease, or loss of function. Cell competition (CC) is a highly conserved biological mechanism among multicellular organisms that results in the removal of less fit, suboptimal cells by their more fit neighbors. CC is active during tissue development to ensure the contribution of robust cell populations and throughout life as a homeostatic surveillance mechanism to maintain tissue function and health.

Mechanistic detection of cellular fitness remains unclear. Thus far, the only fitness-sensing mechanism identified is the Flower (Fwe) “fitness fingerprints”, which are highly conserved transmembrane proteins that directly communicate fitness status and drive competition. In Drosophila, there are three alternately spliced Flower isoforms: Fwe^{ubi}, Fwe^{Lose A} and Fwe^{Lose B} The ubiquitous form, Fwe^{ubi}, is constantly expressed. Under competitive stress, Fwe^{ubi} is downregulated and Fwe^{LoseA} and Fwe^{LoseB} are upregulated in loser cells that undergo apoptosis. Importantly, membrane Flower^{Lose} presence does not autonomously induce apoptosis and Flower^{Lose} cells are not eliminated when surrounded by Flower^{Lose} neighbors. It is clear that in Drosophila, Flower proteins act as direct molecular determinants of cell fitness that are used to detect and eliminate many viable but relatively less healthy cells. It is believed that the Flower code of fitness fingerprints and its downstream players are a common, tunable read-out of cellular fitness likely functional in many tissue systems as means to continuously survey cellular status and rid suboptimal cells.

Flower is a highly conserved gene, and this fitness-sensing program also occurs in mammals. In humans, the Fwe locus generates four isoforms: two hFwe^{Win} and two hFwe^{Lose}. Two Flower isoforms (hFwe2 and hFwe4) behave as Flower-Win proteins, whereas the other isoforms (hFwe1 and hFwe3) behave as Flower-Lose proteins. Like in Drosophila, hFwe-Lose-expressing cells are viable in a homotypic environment. However, when confronted by hFwe-Win-expressing cells, Lose cells are outcompeted and undergo apoptosis. Human Flower-mediated competition requires contact between Win and Lose cells and was not dependent on soluble signal exchange.

The tumor microenvironment (TME), particularly the tumor-adjacent stroma, plays a crucial role in cancer initiation and progression. The TME can both impede and facilitate tumor development and growth. The tumor-stroma interface is a highly dynamic zone that directly mediates tumor outgrowth and local invasion. One mechanism of tumor-host cell competition (CC) fueling oncogenesis is via direct fitness sensing through Flower transmembrane proteins, which were considered to be fitness fingerprints that allow cells to directly communicate the fitness status of their neighbors. Distinct membrane isoform expression marks cells as fit (Flower-Win) or as suboptimal (Flower-Lose) for neighboring cells to recognize. When Flower-Win cells are cocultured in direct contact with Flower-Lose cells, Flower-Win cells eliminate via apoptosis Flower-Lose cells and undergo compensatory proliferation. Strikingly, several types of human cancers express high levels of Flower-Win, in contrast to the adjacent stroma, which expresses high levels of Flower-Lose, concomitant to increased expression of several proapoptotic genes. These results suggest that in human cancers, Flower-Win expressing cancer cells outcompete and eliminate Flower-Lose expressing stromal cells, resulting in tissue attrition, and enabling tumor outgrowth. In addition, in vivo mouse models showed that xenografts of Flower-Win expressing cancer cells against a Flower-Lose tissue background resulted in aggressive tumor growth and metastasis, which is in stark contrast to the negligible tumor growth observed from xenografts of Flower-Lose cells against a Flower-Win background. These results indicate that tumor growth is not the outcome solely of the cancer genome but is mostly a function of fitness inequalities and the elicited CC between the tumor and the host microenvironment.

Because the expression of Flower-Win in tumor and Lose in stroma appears to be a general striking feature of solid tumors and an unrecognized prerequisite for tumor growth, it is of interest to investigate the mechanisms that promote the generation of Flower-Lose isoforms in the TME. High expression of Lose could be a tumor-induced effect or a pre-existing phenotype of the host tissue. Understanding the mechanism of cell competition and fitness comparison between cancer and the tumor microenvironment would provide new insight for cancer therapy and prognosis.

### SUMMARY OF THE DISCLOSURE

In one embodiment, the present disclosure provides antibodies that bind to calcium channel flower homolog protein, the antibodies comprise three heavy chain complementarity determining regions (HCDR1, HCDR2, HCDR3) and three light chain CDRs (LCDR1, LCDR2, LCDR3), wherein
(i) the HCDR1, HCDR2, and HCDR3 have the sequences of SEQ ID NOs:326-328 respectively, and the LCDR1, LCDR2, and LCDR3 have the sequences of SEQ ID NOs:329-331 respectively; or
(ii) the HCDR1, HCDR2, and HCDR3 have the sequences of SEQ ID NOs:266-268 respectively, and the LCDR1, LCDR2, and LCDR3 have the sequences of SEQ ID NOs:269-271 respectively; or
(iii) the HCDR1, HCDR2, and HCDR3 have the sequences of SEQ ID NOs:272-274 respectively, and the LCDR1, LCDR2, and LCDR3 have the sequences of SEQ ID NOs:275-277 respectively; or
(iv) the HCDR1, HCDR2, and HCDR3 have the sequences of SEQ ID NOs:278-280 respectively, and the LCDR1, LCDR2, and LCDR3 have the sequences of SEQ ID NOs:281-283 respectively; or
(v) the HCDR1, HCDR2, and HCDR3 have the sequences of SEQ ID NOs:284-286 respectively, and the LCDR1, LCDR2, and LCDR3 have the sequences of SEQ ID NOs:287-289 respectively; or
(vi) the HCDR1, HCDR2, and HCDR3 have the sequences of SEQ ID NOs:290-292 respectively, and the LCDR1, LCDR2, and LCDR3 have the sequences of SEQ ID NOs:293-295 respectively; or
(vii) the HCDR1, HCDR2, and HCDR3 have the sequences of SEQ ID NOs:296-298 respectively, and the LCDR1, LCDR2, and LCDR3 have the sequences of SEQ ID NOs:299-301 respectively; or
(viii) the HCDR1, HCDR2, and HCDR3 have the sequences of SEQ ID NOs:302-304 respectively, and the LCDR1, LCDR2, and LCDR3 have the sequences of SEQ ID NOs:305-307 respectively; or
(ix) the HCDR1, HCDR2, and HCDR3 have the sequences of SEQ ID NOs:308-310 respectively, and the LCDR1, LCDR2, and LCDR3 have the sequences of SEQ ID NOs:311-313, respectively.

In one embodiment, the present disclosure provides antibodies comprising
(i) a heavy chain variable region having an amino acid sequence at least 80% identical to one of SEQ ID NOs:314, 316, 318, 320, 322, or 324, and a light chain variable region having an amino acid sequence at least 80% identical to one of SEQ ID NOs:315, 317, 319, 321, 323, or 325; or
(ii) a heavy chain variable region having an amino acid sequence at least 80% identical to SEQ ID NO:235, and a light chain variable region having an amino acid sequence at least 80% identical to SEQ ID NO:236; or
(iii) a heavy chain variable region having an amino acid sequence at least 80% identical to SEQ ID NO:237, and a light chain variable region having an amino acid sequence at least 80% identical to SEQ ID NO:238; or
(iv) a heavy chain variable region having an amino acid sequence at least 80% identical to SEQ ID NO:239, and a light chain variable region having an amino acid sequence at least 80% identical to SEQ ID NO:240; or
(v) a heavy chain variable region having an amino acid sequence at least 80% identical to SEQ ID NO:241, and a light chain variable region having an amino acid sequence at least 80% identical to SEQ ID NO:242; or
(vi) a heavy chain variable region having an amino acid sequence at least 80% identical to SEQ ID NO:243, and a light chain variable region having an amino acid sequence at least 80% identical to SEQ ID NO:244; or
(vii) a heavy chain variable region having an amino acid sequence at least 80% identical to SEQ ID NO:245, and a light chain variable region having an amino acid sequence at least 80% identical to SEQ ID NO:246; or
(viii) a heavy chain variable region having an amino acid sequence at least 80% identical to SEQ ID NO:247, and a light chain variable region having an amino acid sequence at least 80% identical to SEQ ID NO:248; or
(ix) a heavy chain variable region having an amino acid sequence at least 80% identical to SEQ ID NO:249, and a light chain variable region having an amino acid sequence at least 80% identical to SEQ ID NO:250.

In one embodiment, the present disclosure provides antibodies comprising
(i) a heavy chain variable region having the amino acid sequence of one of SEQ ID NOs:314, 316, 318, 320, 322, or 324, and a light chain variable region having the amino acid sequence of one of SEQ ID NOs:315, 317, 319, 321, 323, or 325; or
(ii) a heavy chain variable region having the amino acid sequence of SEQ ID NO:235, and a light chain variable region having the amino acid sequence of SEQ ID NO:236; or
(iii) a heavy chain variable region having the amino acid sequence of SEQ ID NO:237, and a light chain variable region having the amino acid sequence of SEQ ID NO:238; or
(iv) a heavy chain variable region having the amino acid sequence of SEQ ID NO:239, and a light chain variable region having the amino acid sequence of SEQ ID NO:240; or
(v) a heavy chain variable region having the amino acid sequence of SEQ ID NO:241, and a light chain variable region having the amino acid sequence of SEQ ID NO:242; or
(vi) a heavy chain variable region having the amino acid sequence of SEQ ID NO:243, and a light chain variable region having the amino acid sequence of SEQ ID NO:244; or
(vii) a heavy chain variable region having the amino acid sequence of SEQ ID NO:245, and a light chain variable region having the amino acid sequence of SEQ ID NO:246; or
(viii) a heavy chain variable region having the amino acid sequence of SEQ ID NO:247, and a light chain variable region having the amino acid sequence of SEQ ID NO:248; or
(ix) a heavy chain variable region having the amino acid sequence of SEQ ID NO:249, and a light chain variable region having the amino acid sequence of SEQ ID NO:250.

In one embodiment, the present disclosure provides antibodies that binds to a target having the amino acid sequence of one of SEQ ID NOs:251-258. In some embodiments, the antibodies comprise the CDR sequences as described above. In some embodiments, the antibodies comprise heavy and light chain variable regions as described above.

In one embodiment, the present disclosure provides pharmaceutical composition comprising the antibodies disclosed herein and a pharmaceutically acceptable carrier.

In one embodiment, the present disclosure provides small molecules that bind to a target having the amino acid sequence of one of SEQ ID NOs:251-258. In one embodiment, the present disclosure provides pharmaceutical composition comprising the small molecules disclosed herein and a pharmaceutically acceptable carrier.

In other embodiments, the present disclosure provides isolated polynucleotides encoding the antibodies disclosed herein; expression vectors comprising such polynucleotides; and host cells comprising such expression vectors. In view of the amino acid sequences disclosed herein, one of ordinary skill in the art would readily construct the corresponding polynucleotides, expression vectors, and host cells according to methods and techniques generally known in the art.

In another embodiment, the present disclosure provides a method of increasing cellular fitness of a population of cells, comprising contacting the population of cells with an agent that binds to a target having the amino acid sequence of one of SEQ ID NOs:251-258. In one embodiment, the agent comprises one or more of the antibodies disclosed herein. In one embodiment, the agent comprises the small molecules disclosed herein. In some embodiments, an agent is an antibody or a small molecule.

In another embodiment, the present disclosure provides a method of preventing apoptosis of a population of cells, comprising contacting the population of cells with an agent that binds to a target having the amino acid sequence of one of SEQ ID NOs:251-258. In one embodiment, the agent comprises one or more of the antibodies disclosed herein. In one embodiment, the agent comprises the small molecules disclosed herein.

In another embodiment, the present disclosure provides a method of treating a cancer, or a disease or condition in a subject in need thereof, comprising administering to the subject an agent that binds to a target having the amino acid sequence of one of SEQ ID NOs:251-258. In one embodiment, the agent comprises one or more of the antibodies disclosed herein. In one embodiment, the agent comprises the small molecules disclosed herein.

These and other aspects of the invention will be appreciated from the ensuing descriptions of the figures and detailed description of the invention.

### BRIEF DESCRIPTION OF THE DRAWINGS

The patent or patent application file contains at least one drawing executed in color. Copies of this patent or patent application publication with color drawing(s) will be provided by the Office upon request and payment of the necessary fee.

Some embodiments of the antibodies targeting calcium channel flower homolog protein and uses thereof, are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the antibodies targeting calcium channel flower homolog protein and uses thereof may be practiced.

FIGS. 1A-1H show stromal Flower-Lose expression is associated with hFwe Exon3 methylation and LINCO1914 expression.

FIG. 1A: A schematic representation of the six exons in the Flower gene locus is shown. The Flower Lose isoforms (hFwe1 and hFwe3) do not include Exon 3 in the final transcript, whereas Flower Win isoforms (hFwe2 and hFwe4) specifically include Exon 3.

FIG. 1B: A model depicting the occupancy of amino acid chains corresponding to different exons in the transmembrane. hFwe1 and hFwe3 lack Exon 3 and encode Lose isoforms. In the Flower Lose isoforms, the N-terminus region is extracellular.

FIG. 1C: Multiplex immunohistochemistry was used to define the location of Flower Win and Lose expression in stromal and cancer components of HGSC tissue (scale 50 μm). Staining with the Flower N-terminus Ab (detects both Win and Lose isoforms) shows staining in both stromal and tumor regions. Flower Win Ab (against Exon 3; detects only Win isoforms) demonstrates staining in only the tumor compartment. Multiplexing of Vimentin and Cytokeratin Ab with Flower N-terminus Ab and Flower Win Ab shows that the Flower Lose isoform is specifically expressed in a majority of fibroblasts.

FIG. 1D: A model derived from PCA exploratory analysis and ChIP experiments explains how Exon 3 is included in the HGSC tumor and excluded in the stromal tissue.

FIG. 1E: RNA capping of LINC01914 is analyzed by CIP-TAP linker ligation. Polyadenylation status of LINC01914 is analyzed. LINC01914 amplification corresponding to the presence of Poly-A (pA+) tail relative to control (pA) is shown.

FIG. 1F: The gel shows the 5′ RACE and 3′ RACE amplifications of LINC01914 transcript from normal, tumor and stroma cells. For 3′ RACE, 330 bp nucleotide fragment was obtained, for 5′ RACE, a 245-bp nucleotide fragment was obtained.

FIG. 1G: ChIP on Exon 3 and 100 bp flanking regions in WT stromal cells and LINC01914 overexpressing stromal cells shows significantly increased association of DNA methylation, DNMT3A, G9a, GLP, H3K9Me3, HP1, and SRSF3 after overexpression.

FIG. 1H: The model depicts RT-PCR of hwFwe isoforms based on the amplification of PCR products spanning exons 2 to 4 and Exon 3 to identify hFwe Exon 3 skipping. Since hFwe-Win isoforms include the Exon 3, it is expected to obtain a 330 bp RT-PCR product for primers spanning exons 2 to 4 and a 126 bp amplicon for the Exon 3-specific primers. Since hFwe-Lose isoforms lack Exon 3, RT-PCR with the primers spanning exons 2 to 4 will result in amplification of a 204 bp product.

FIGS. 2A-2N show tumor cells synthesize and export Tu-Stroma to stromal cells via DDX3X-assisted exosomal packaging.

FIG. 2A: Expression of hFwe-Win and Lose isoforms in stromal cells after co-culture with OVCAR8 cells. The expression of hFwe isoforms was also observed upon silencing of LINC01914 expression mediated by Poly-A insertion at LINC01914 locus.

FIG. 2B: ChOP of hFwe Exon 3 in stromal cells co-cultured with OVCAR8 WT cells shows LINC01914 enrichment at hFwe Exon 3 loci. The association of LINC01914 with the hFwe Exon 3 region is poorly observed when stromal cells are co-cultured with OVCAR8 with LINC01914 KO (p=5.9×10⁻⁹). The ChOP pull-down analyzed by qPCR is calculated as the percentage of input (n=5, horizontal bars indicate mean values).

FIG. 2C: The model shows that the hFwe Exon 3 can establish a DNA-RNA adduct via Hoogsteen base pairing. Near palindromic association and complementarity within the GA-enriched motif sequences between the hFwe Exon 3 and LINC01914 is depicted in orange color.

FIG. 2D: EMSA assay is performed to evaluate the level of DNA-RNA adduct formation between full-length LINC01914 (490 bp) and full-length Exon 3 DNA (126 bp) in vitro.

FIG. 2E: Nascent RNA transcript analysis from normal, HGSC, and stromal tissue derived from FFPE samples of distinct patient tumors shows that LINC01914 is expressed 12-fold in the HGSC tumor tissue compared to the matched normal ovarian and stromal tissue (p=1.5×10⁻⁵), (n=3; technical replicates, horizontal bars indicate mean values).

FIG. 2F: Exosomes collected from the blood of HGSC individuals (n=5) and normal non-HGSC individuals (n=5 biological replicates) show high expression of LINC01914 in exosomes of HGSC individuals relative to exosomes from non-cancer subjects (p=3.6×10⁻⁴), horizontal bars indicate mean values).

FIG. 2G: Model depicting the synthesis and overexpression of barcoded LINC01914 in OVCAR8 WT, OVCAR8 LINC01914 KO, and OVCAR8 Rab27a/Rab27b/LINC01914 triple KO cells.

FIG. 2H: RT-qPCR products of cultures with OVCAR8 LINC01914 KO cells (up-right). The gel shows the expression of endogenous LINC01914 in monocultures of OVCAR8 WT cells (lane 2). A second agarose gel electrophoresis shows the RT-qPCR products of cultures with OVCAR8 Rab27a Rab27b/LINC01914 triple KO cells (bottom-right). The * signals the overexpression of WT LINC01914 in stromal cells.

FIG. 21: PCR products from the RACE assay using RNA collected the HGSC tissue samples and exosomal fractions from the HGSC tissue, full-length Tu-stroma of 490 bp in observed in the exosomal fractions (lane 3).

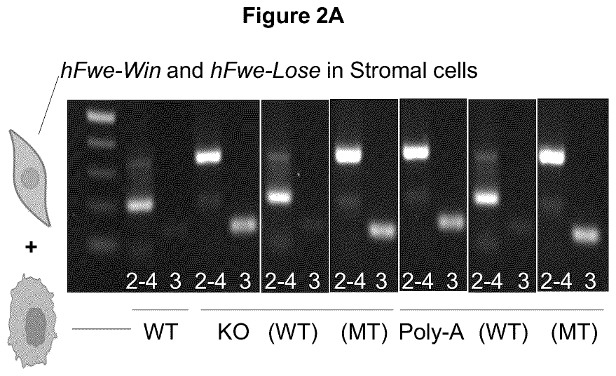

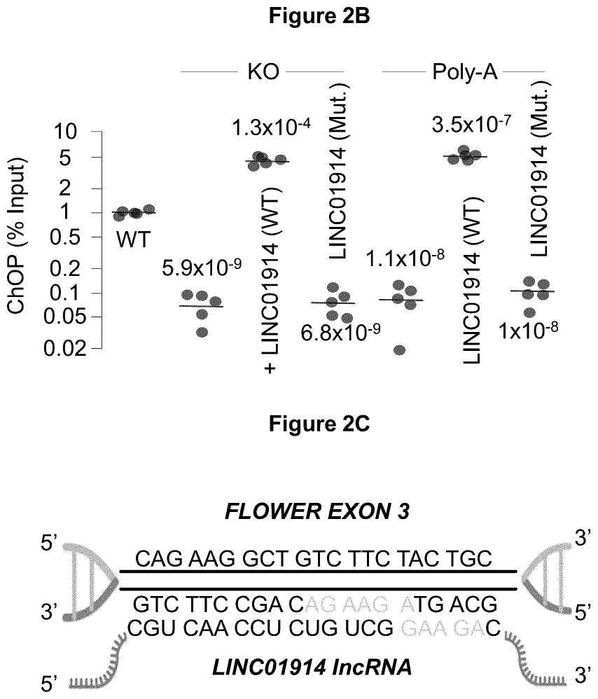

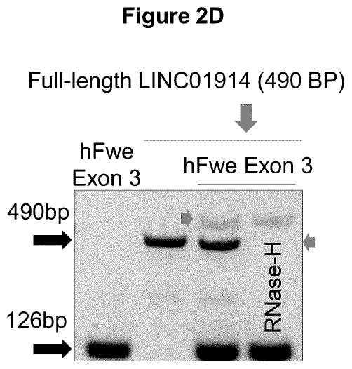

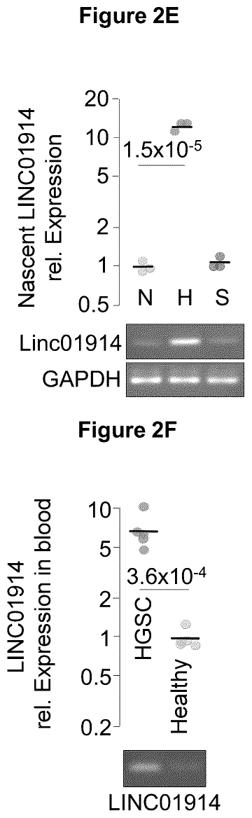

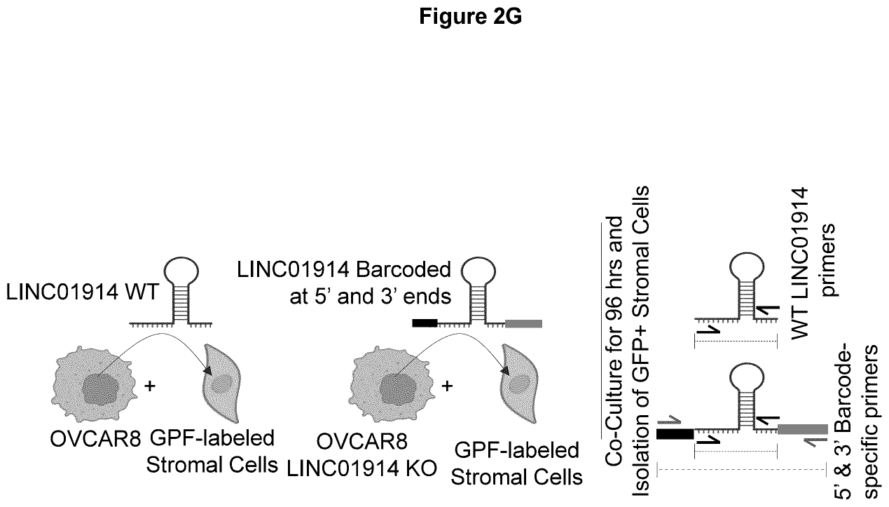

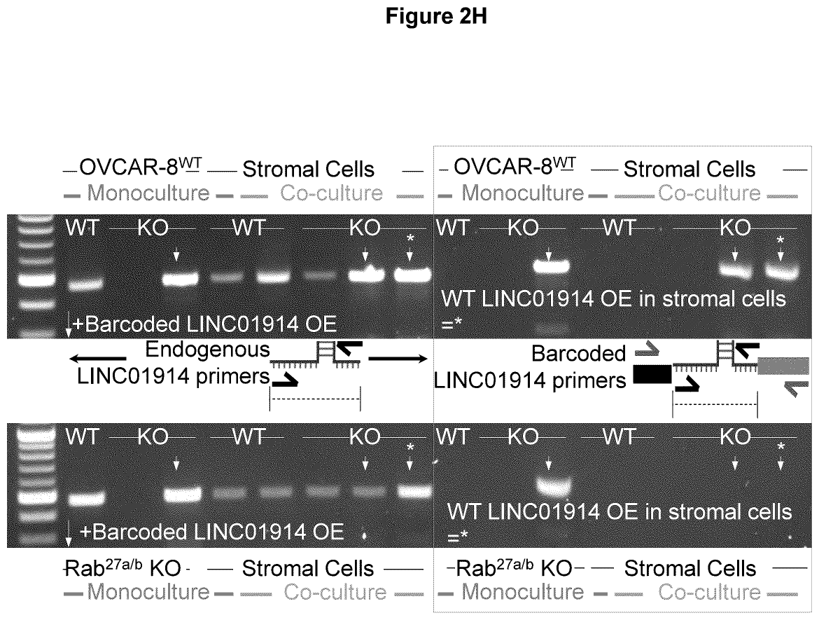

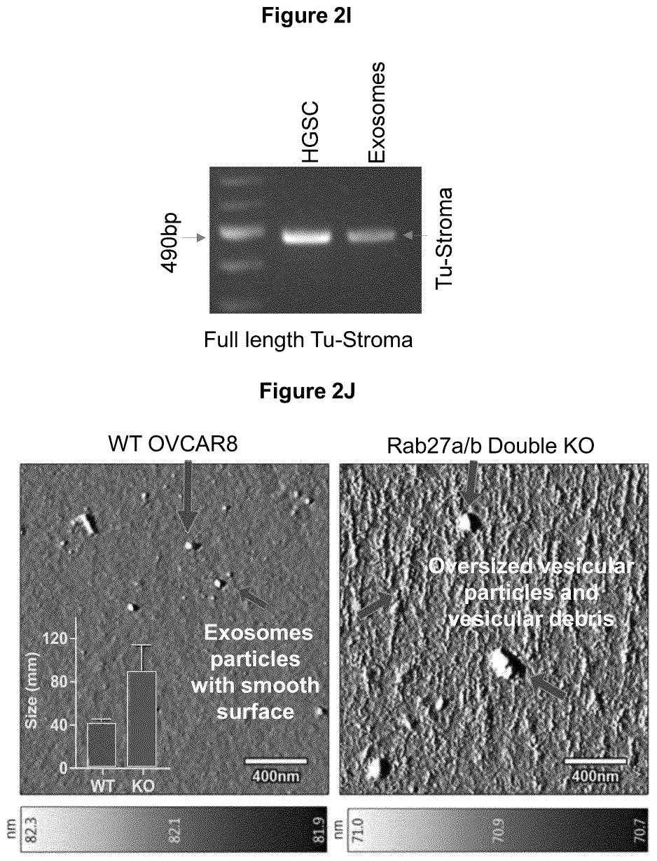

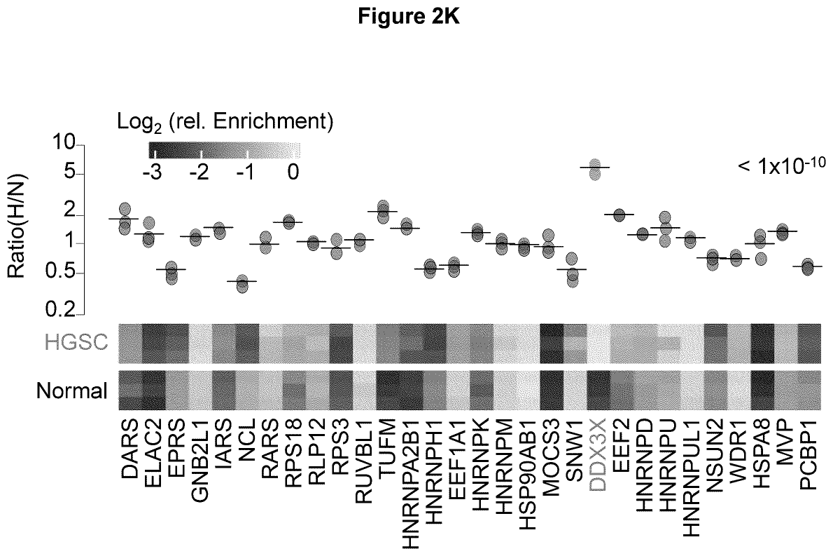

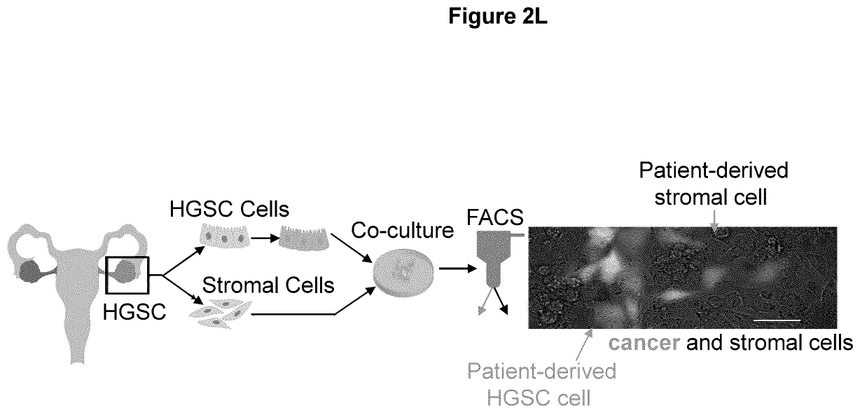

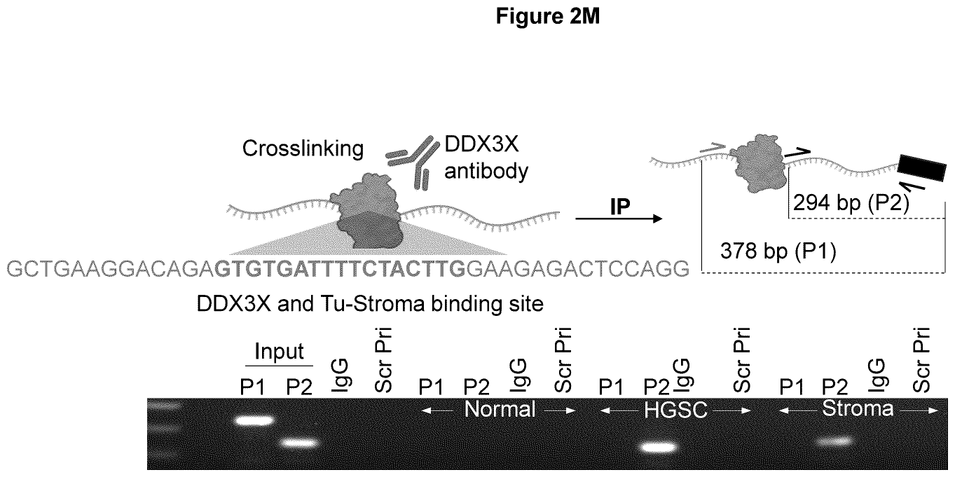

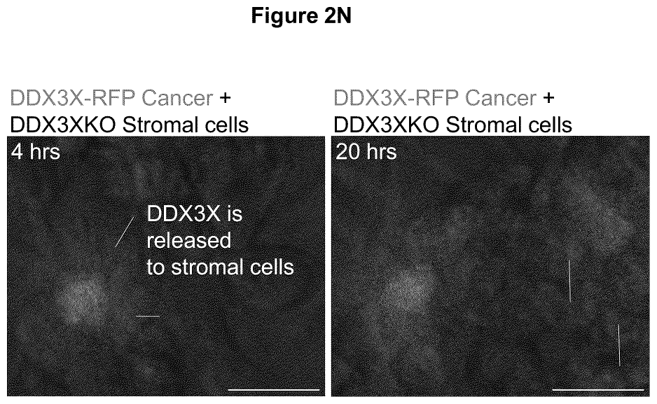

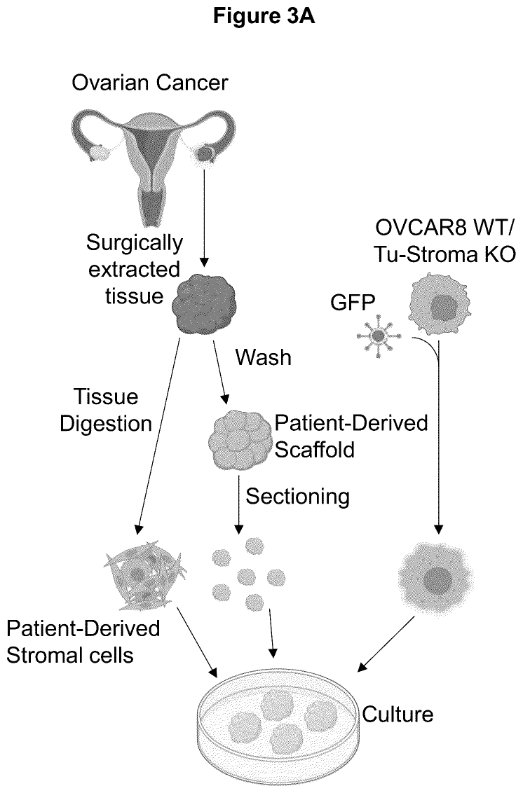

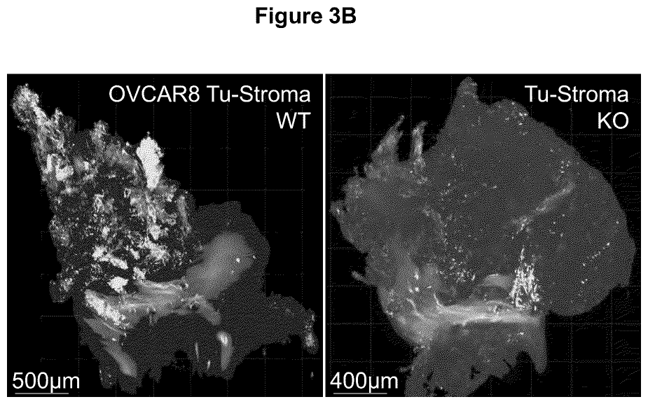

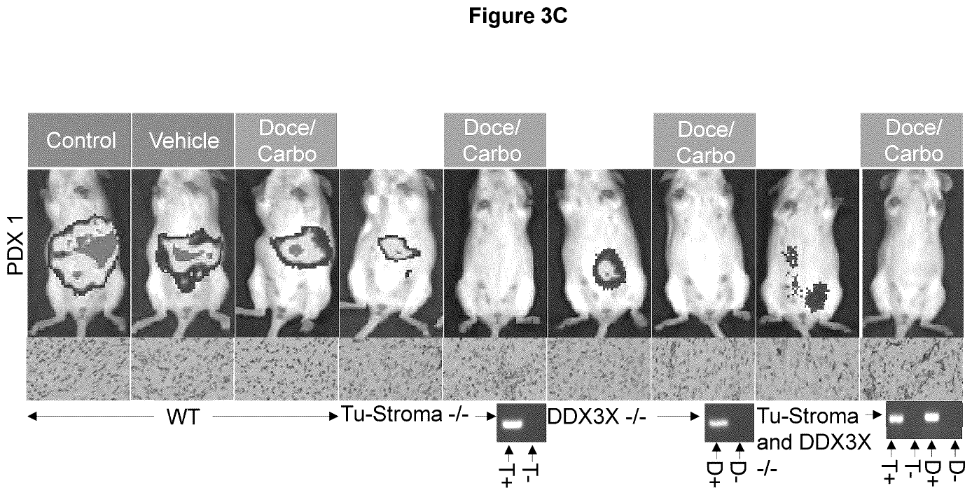

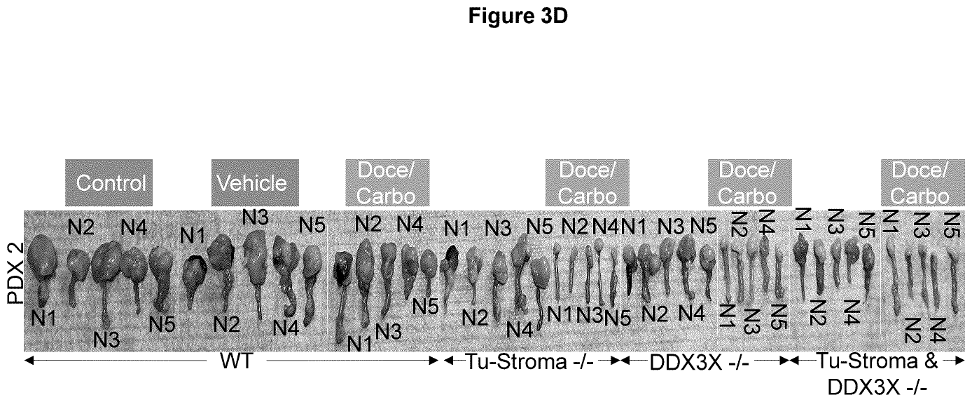

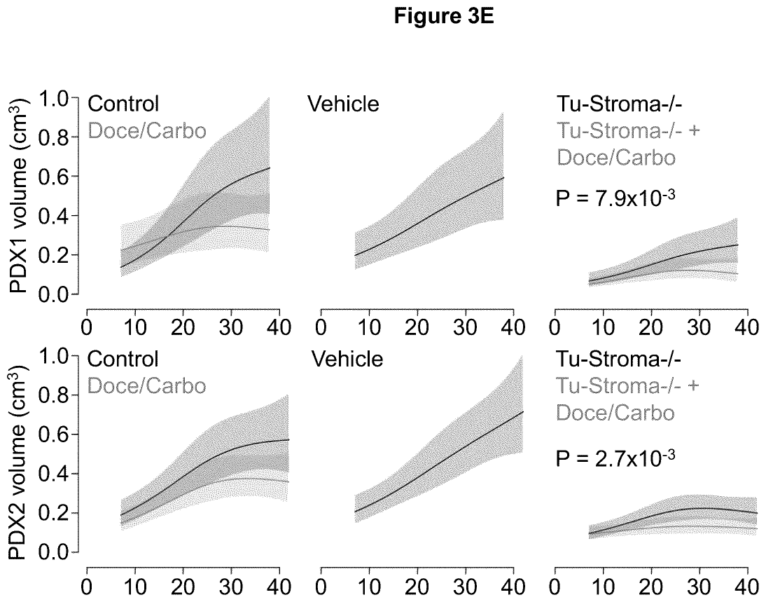

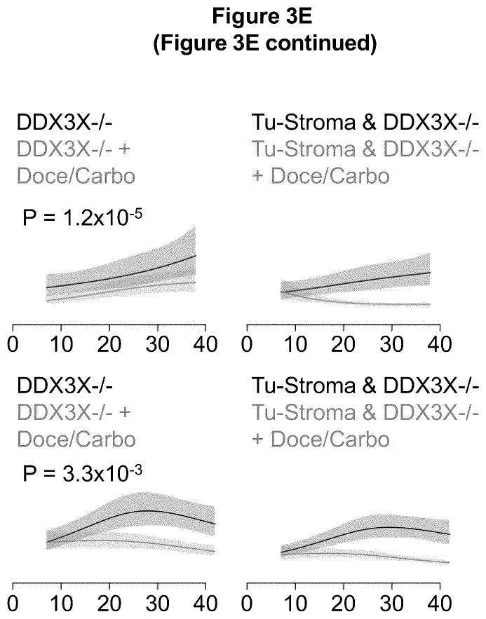

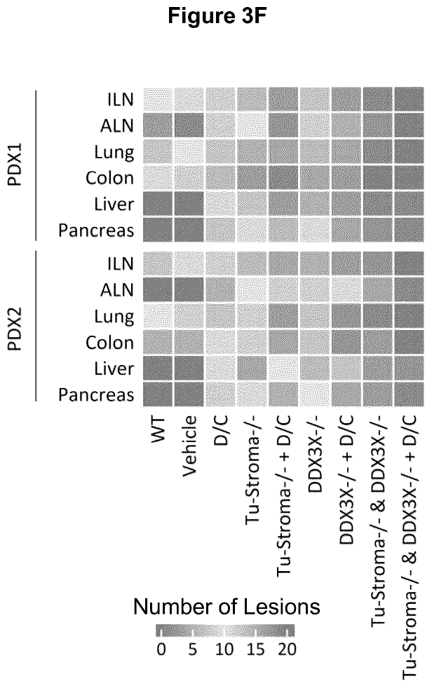

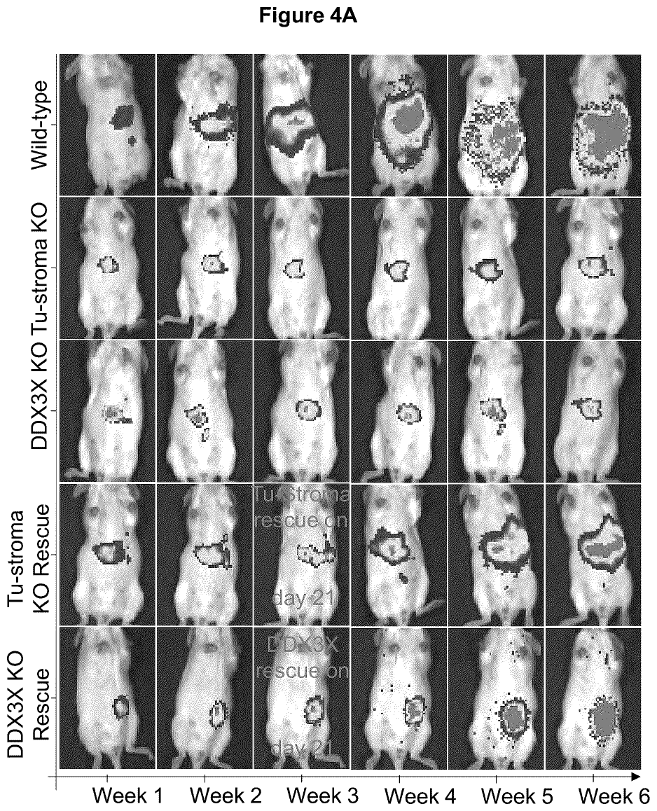

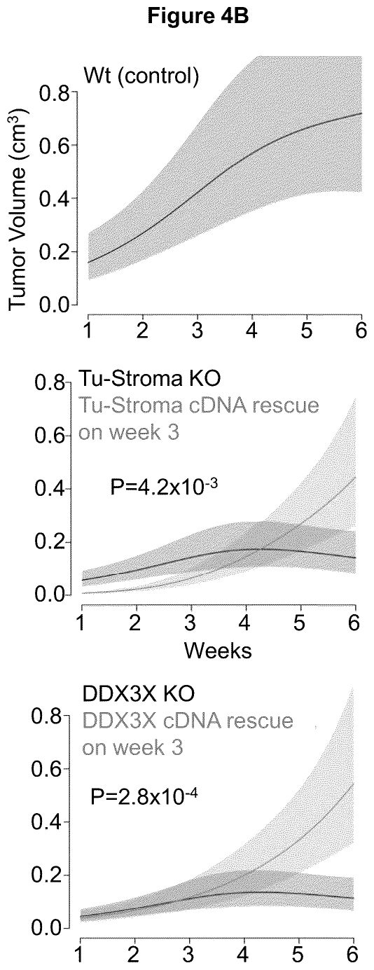

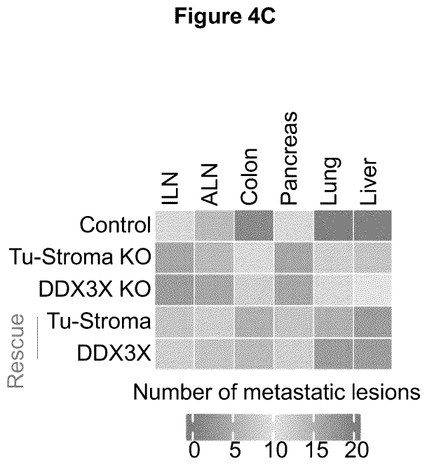

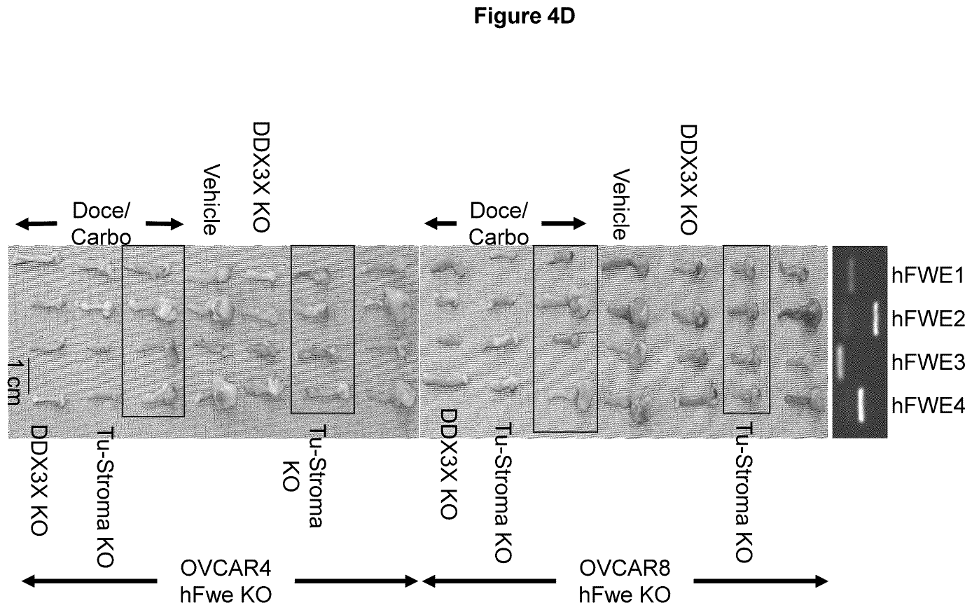

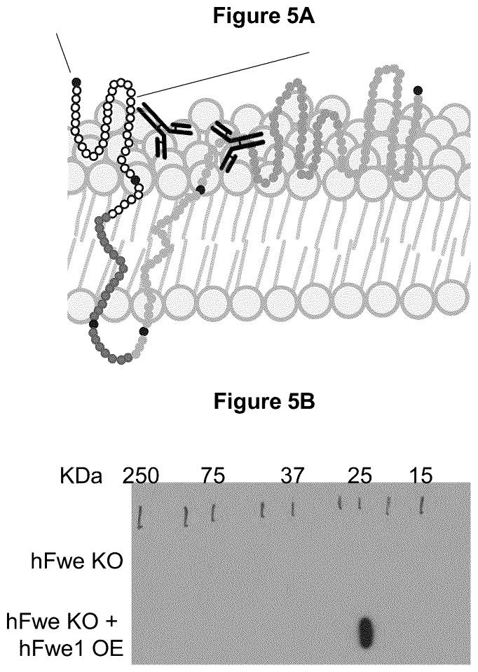

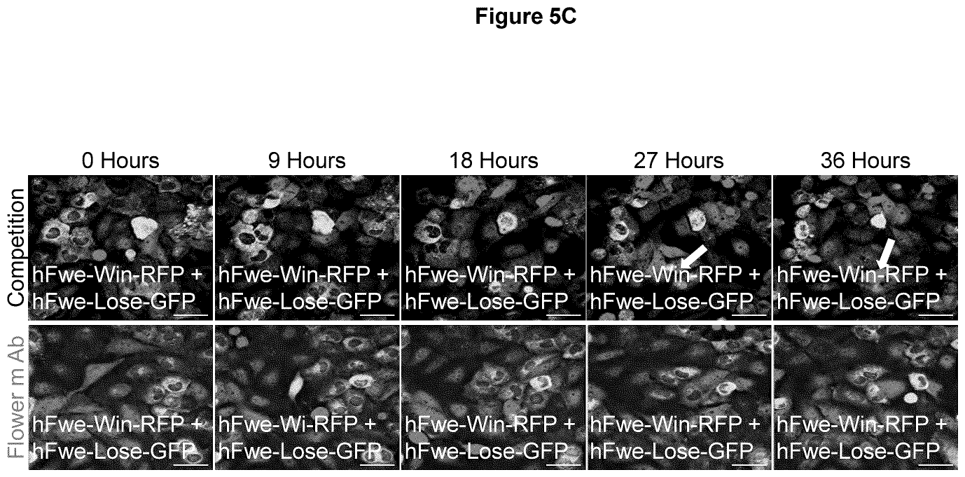

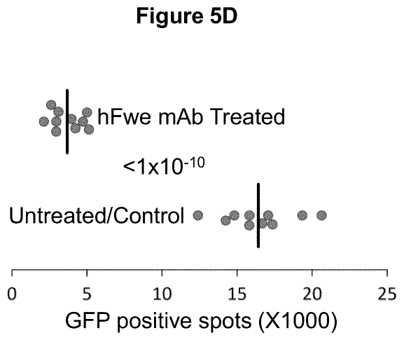

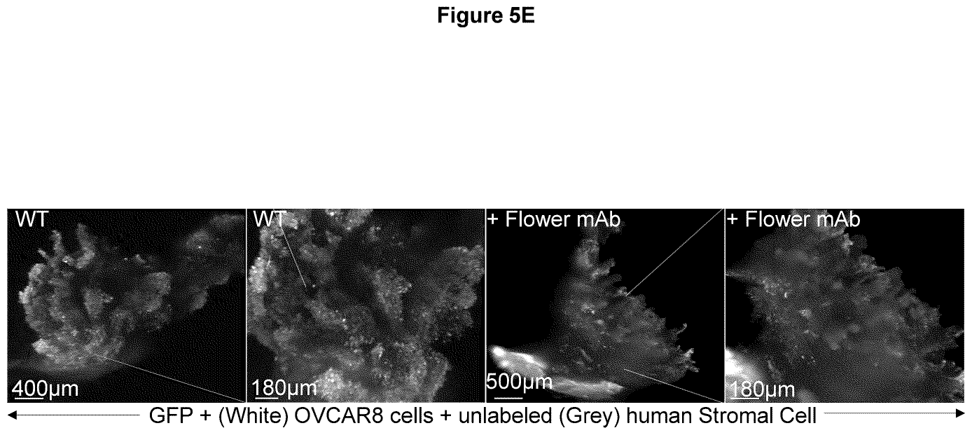

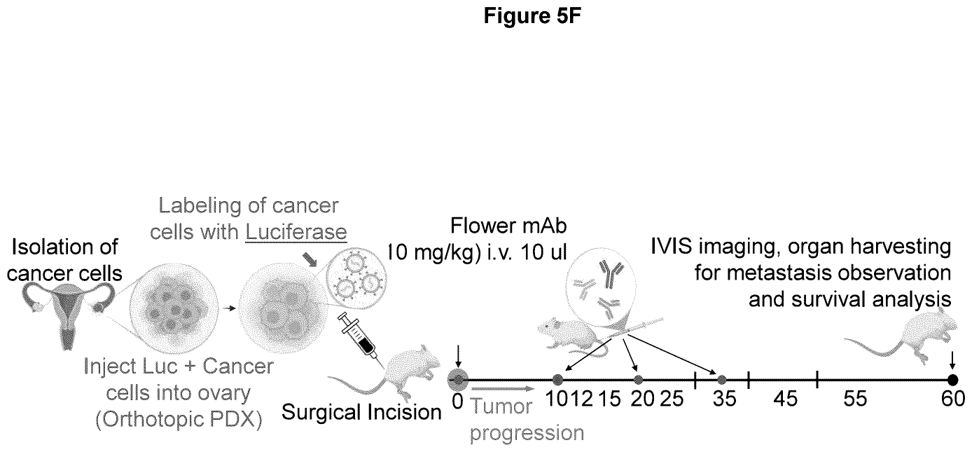

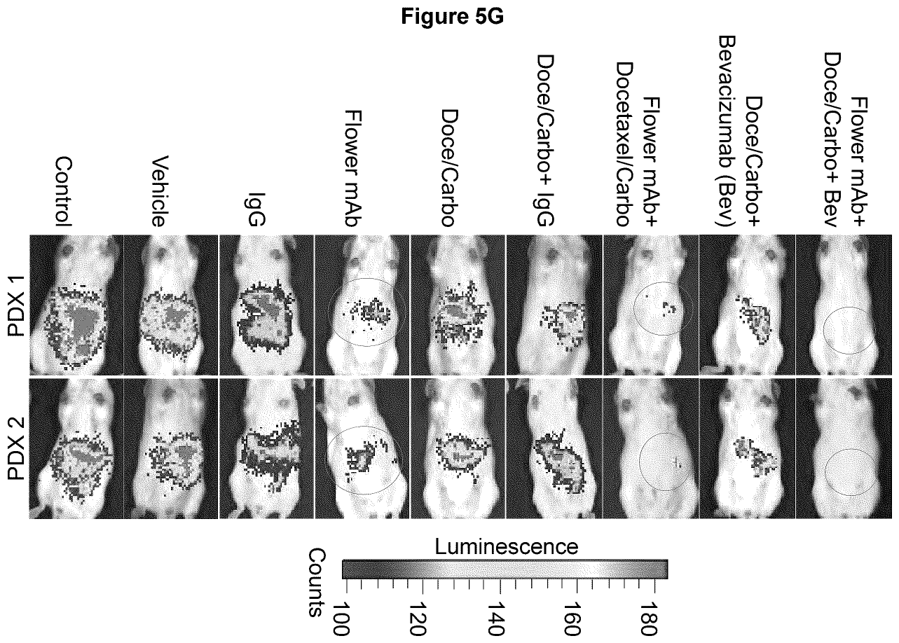

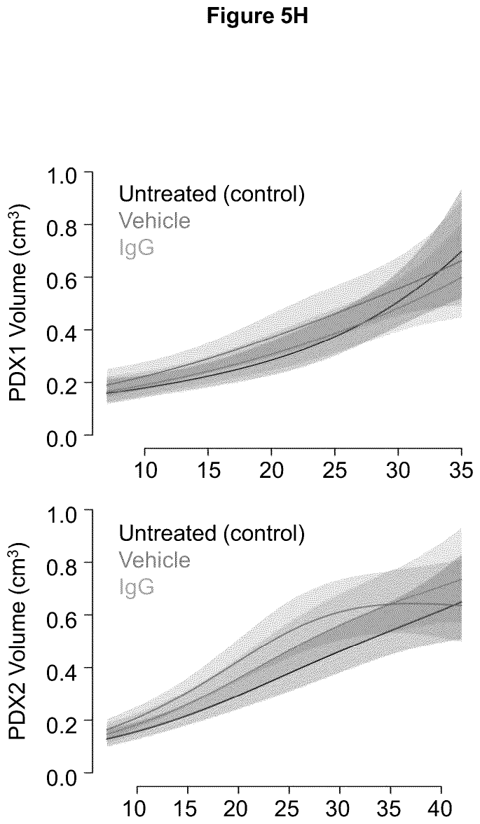

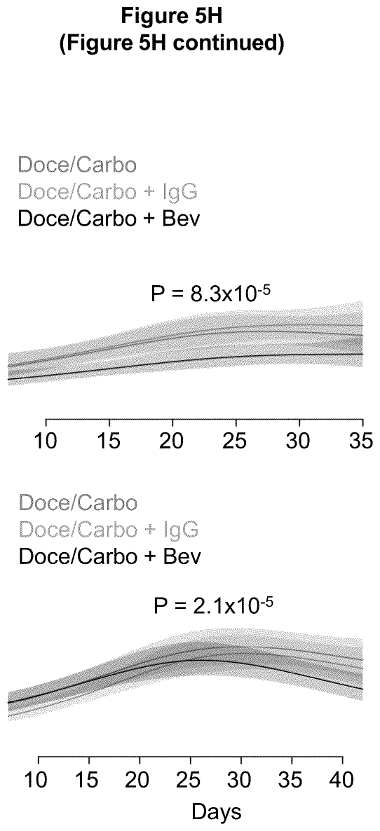

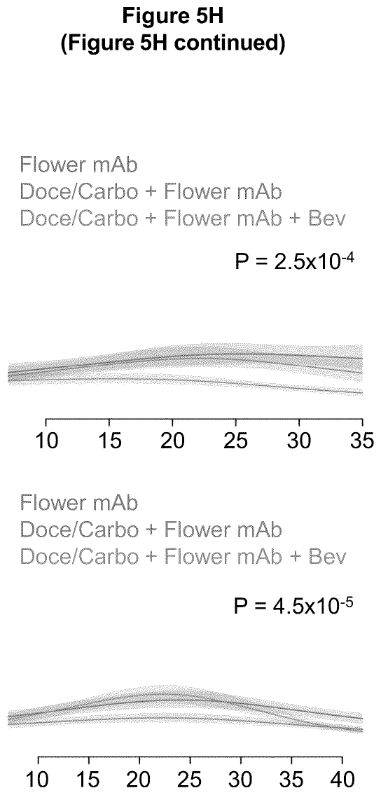

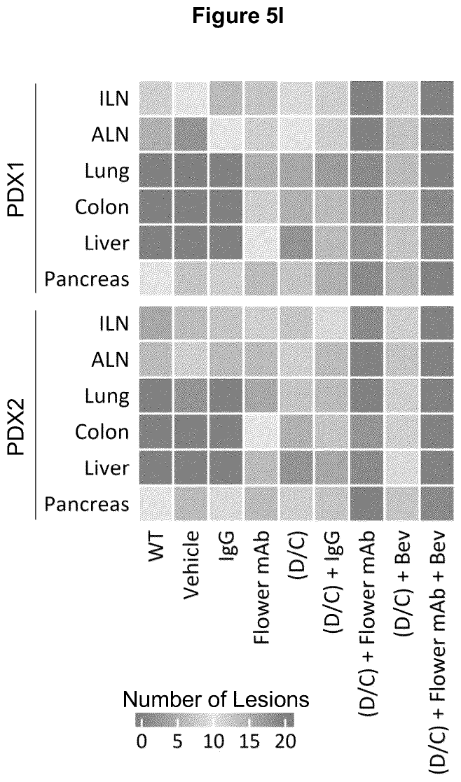

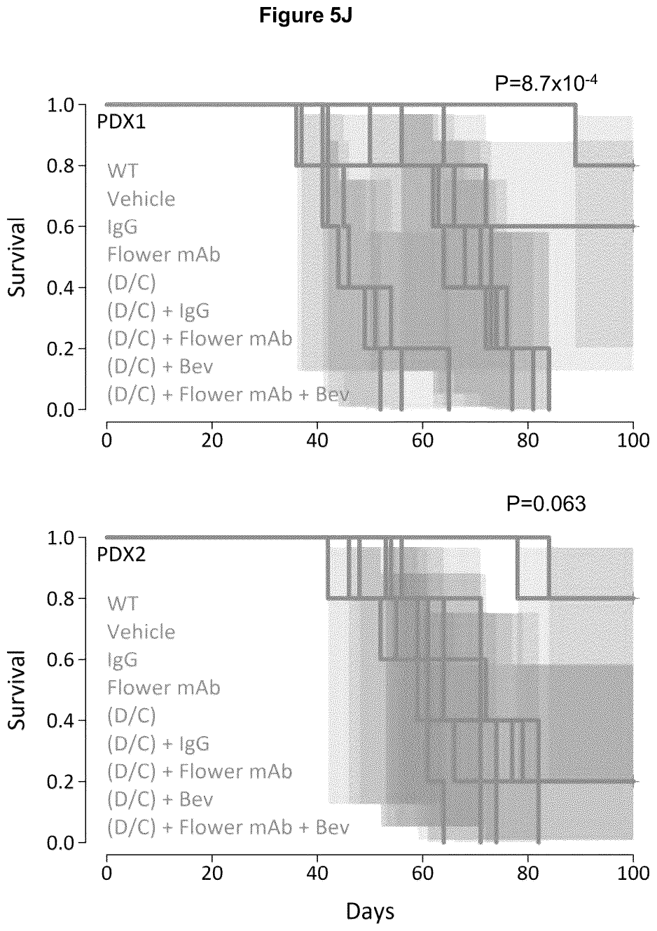

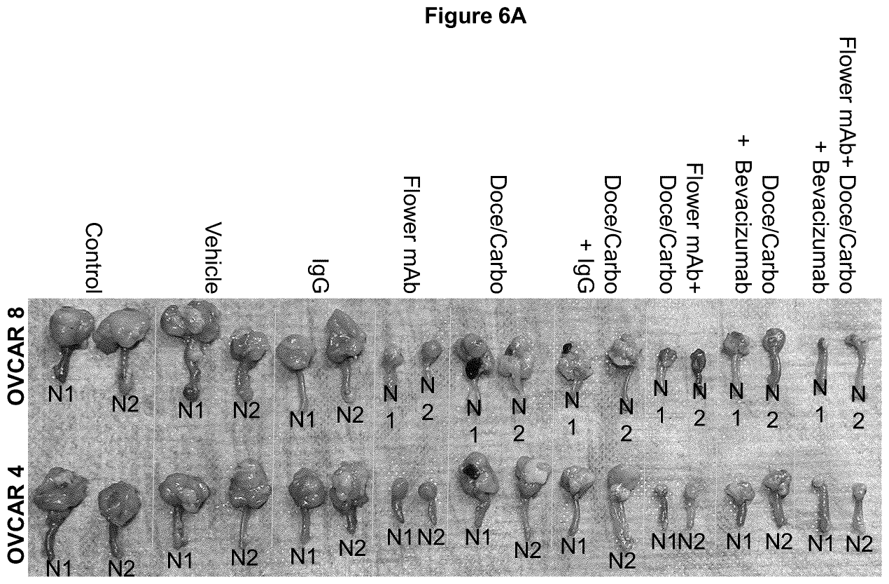

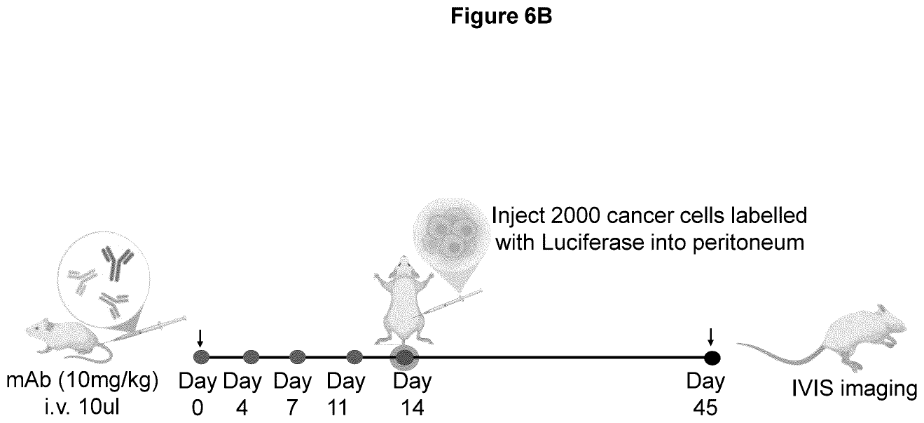

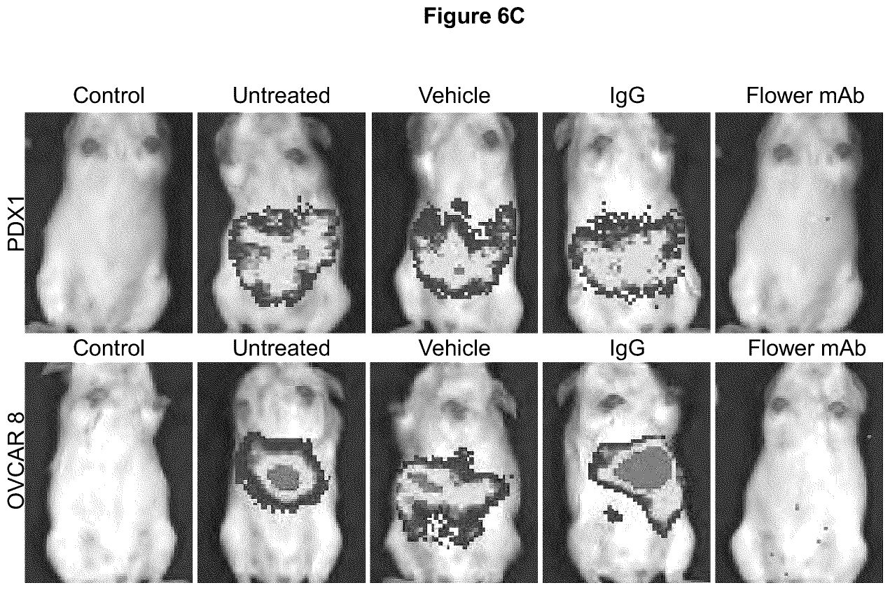

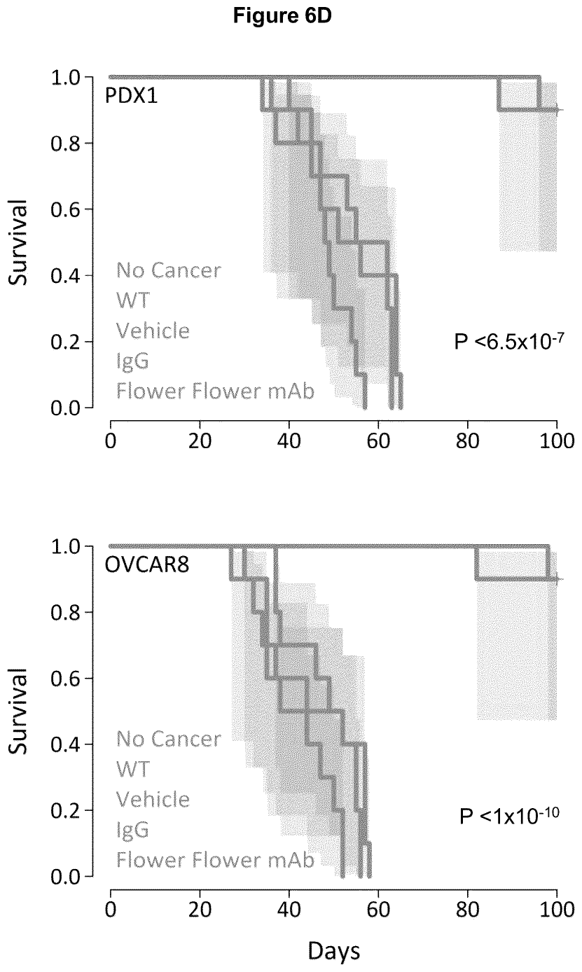

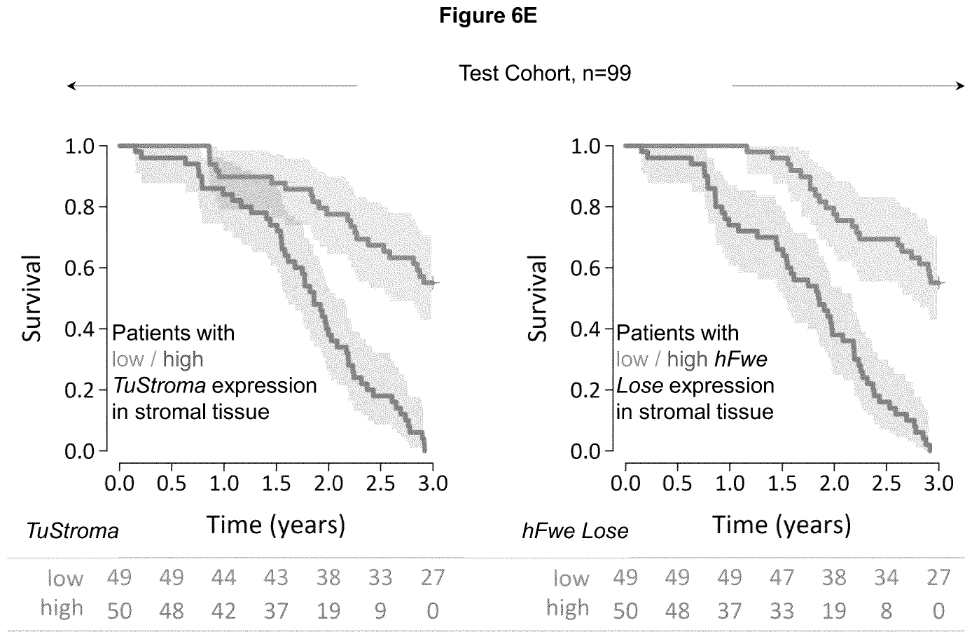

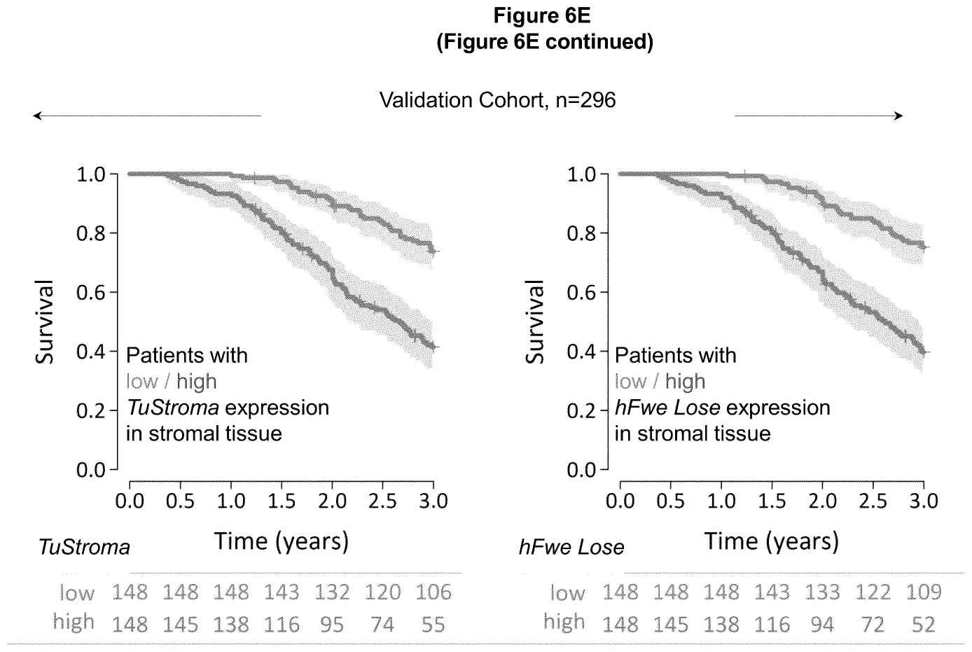

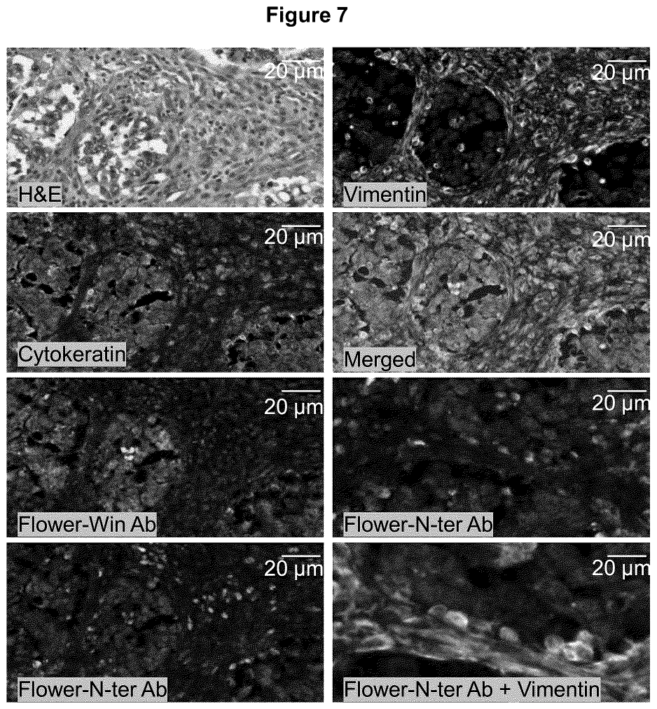

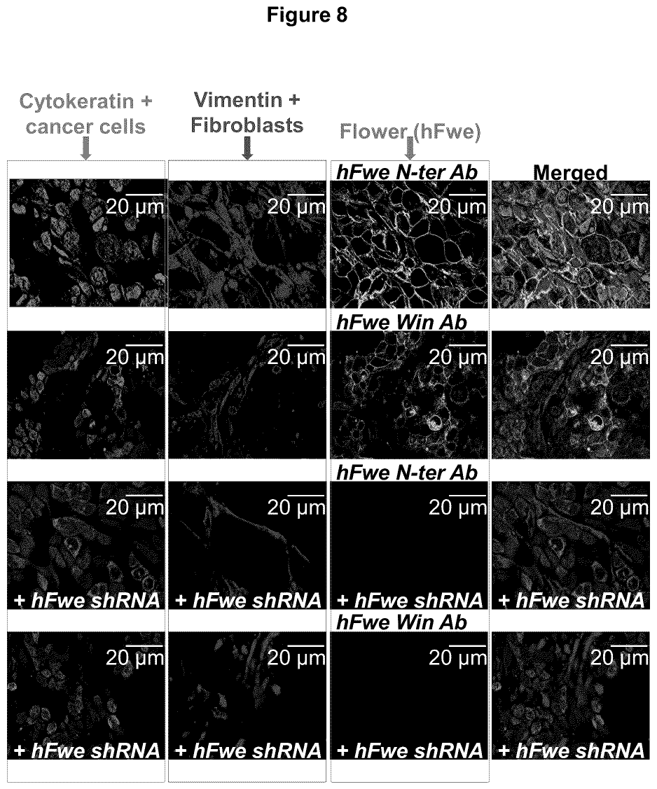

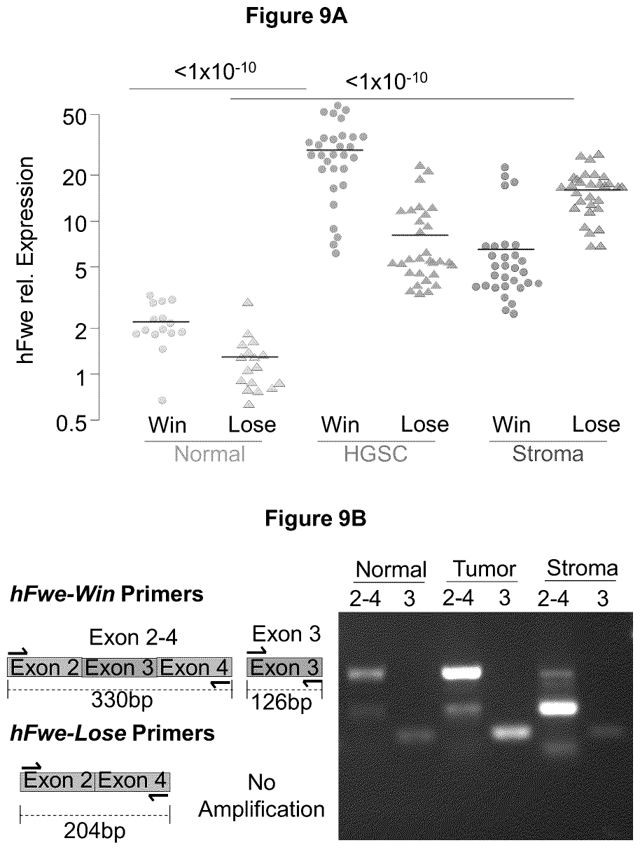

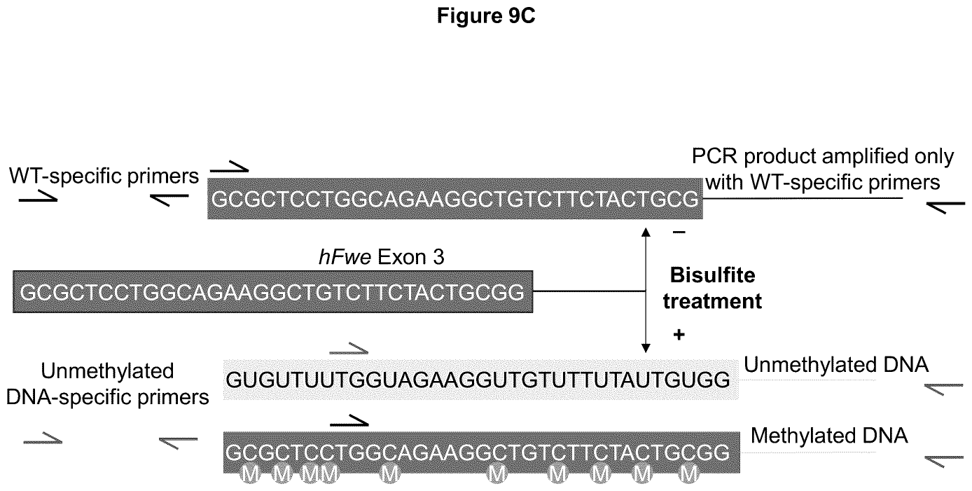

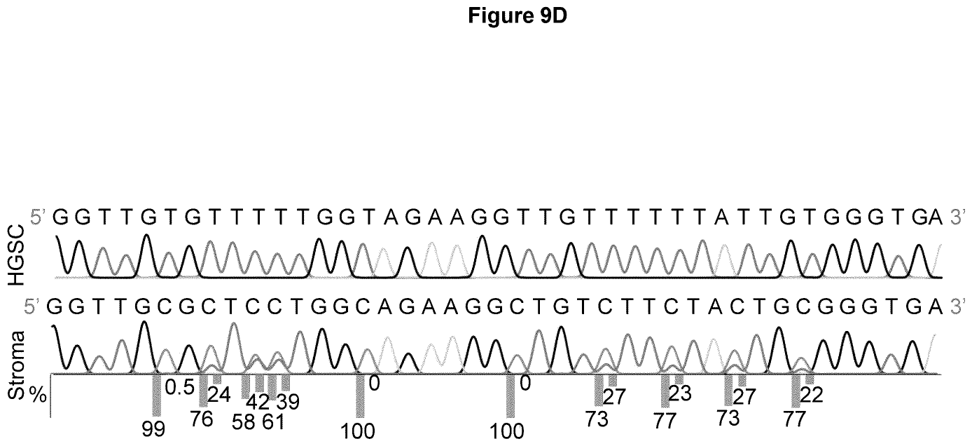

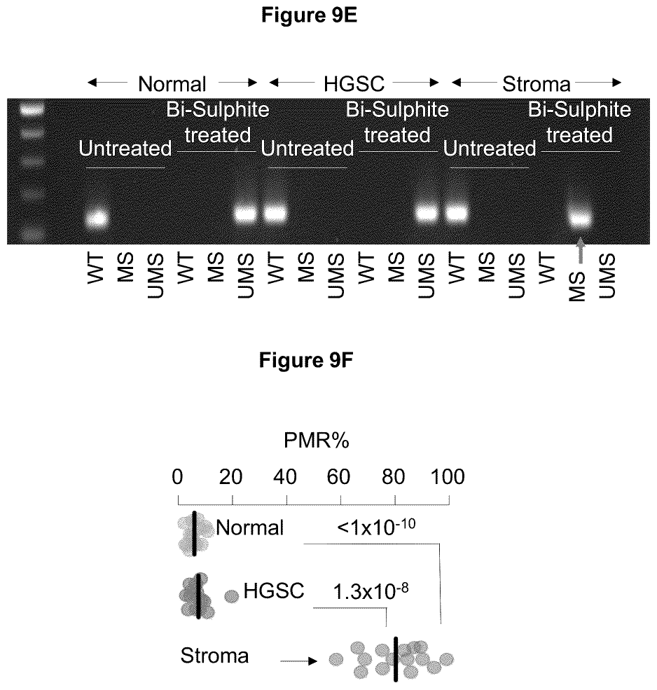

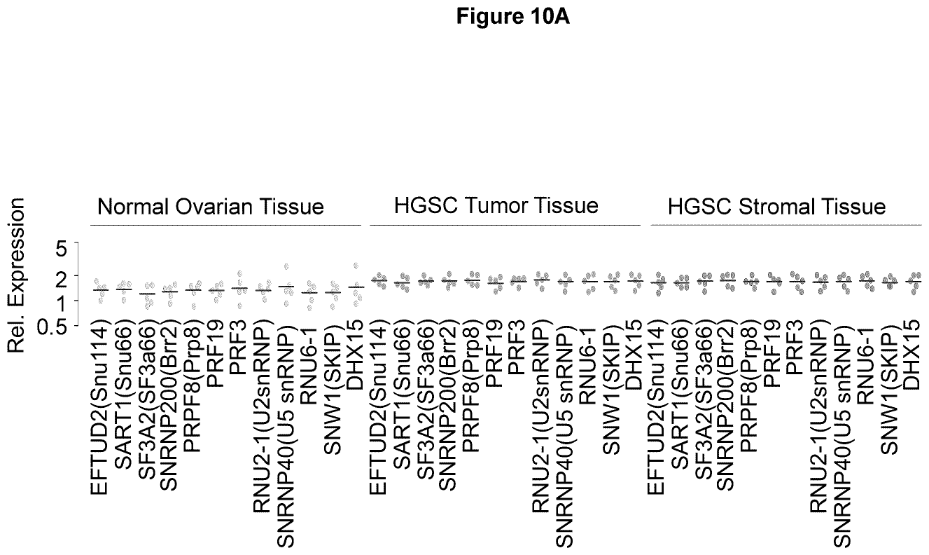

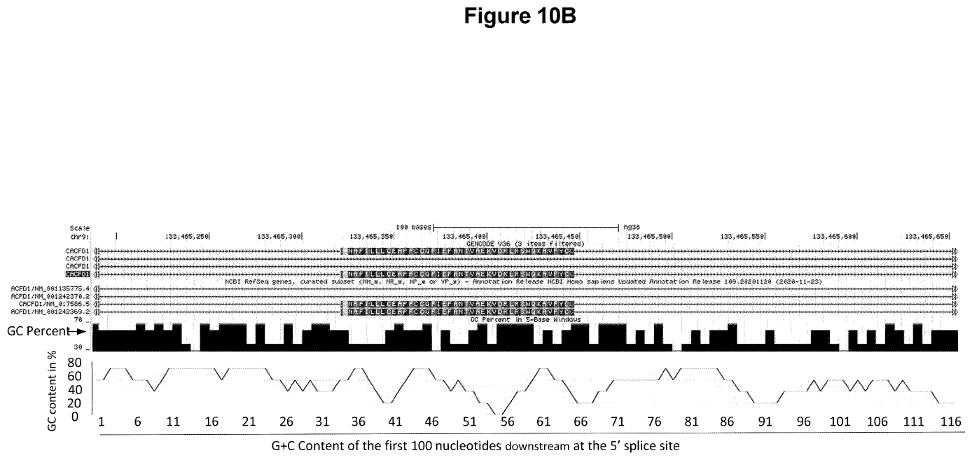

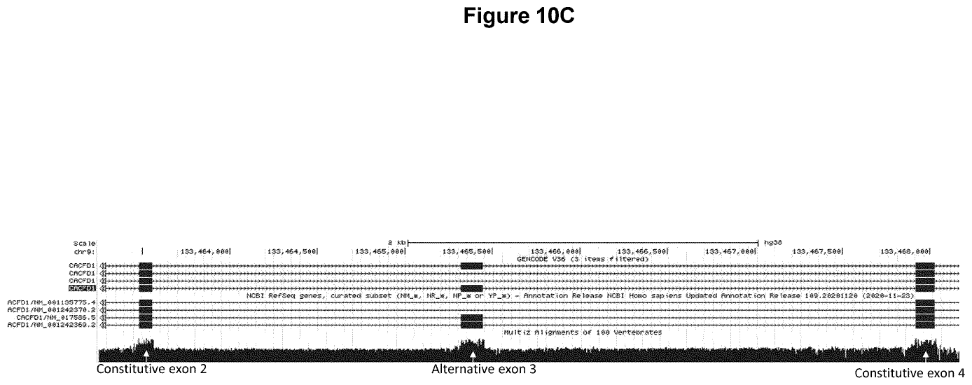

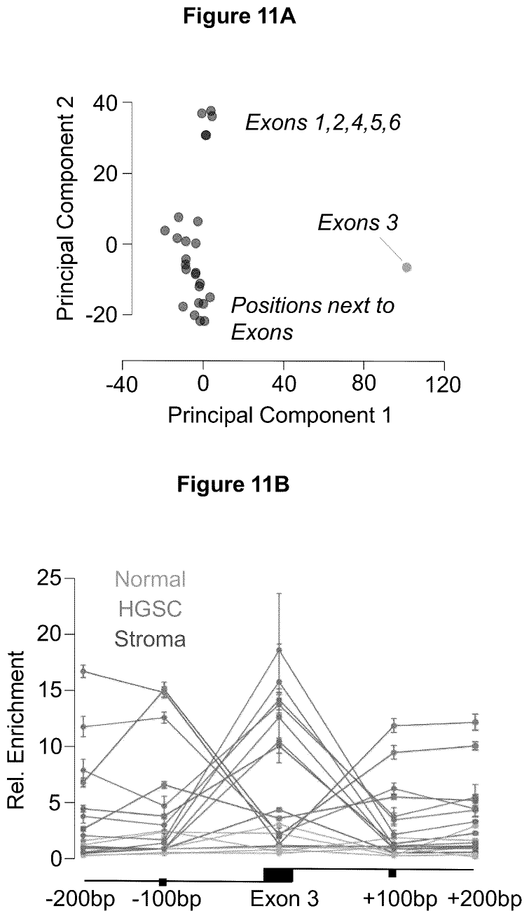

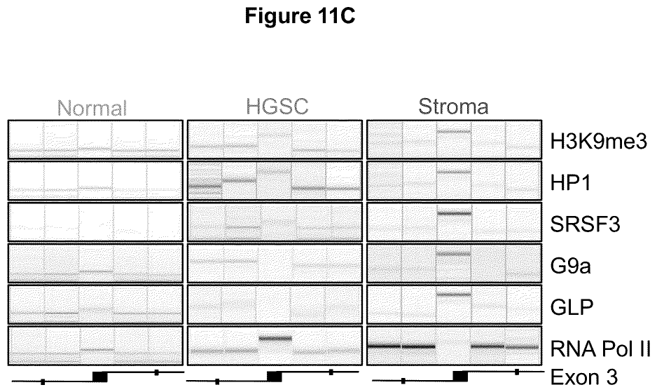

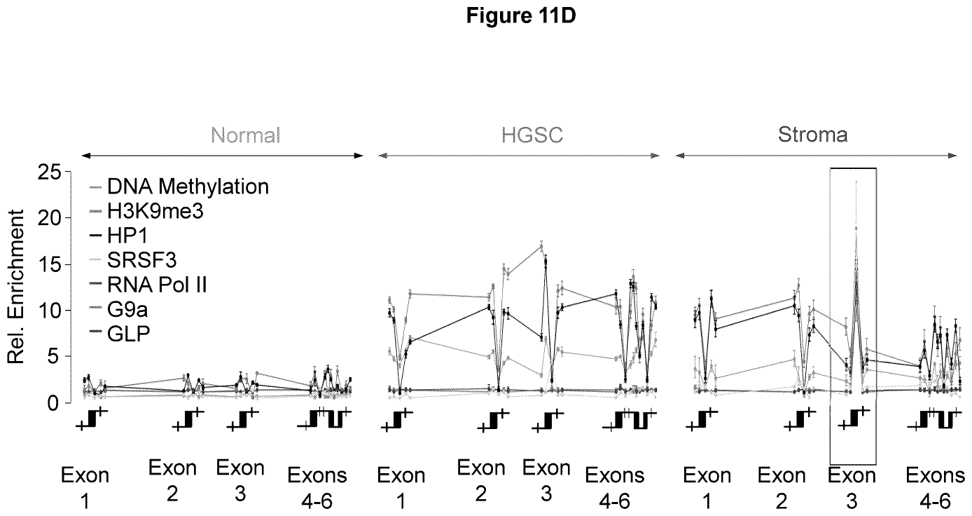

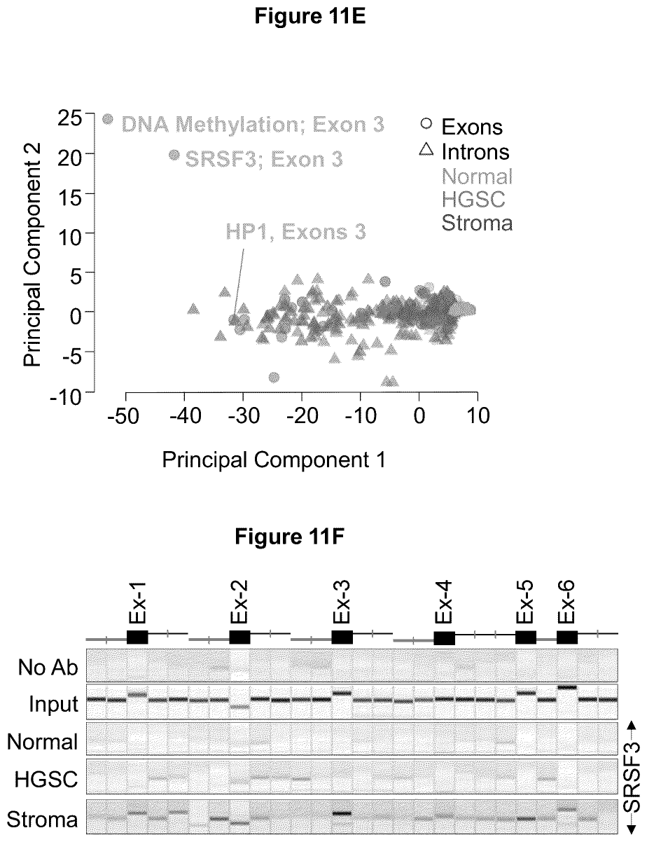

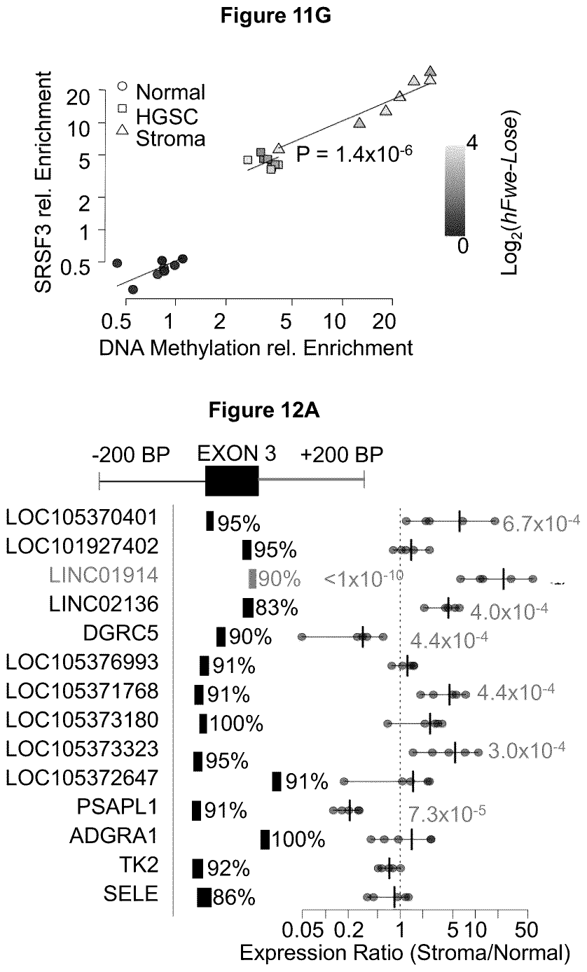

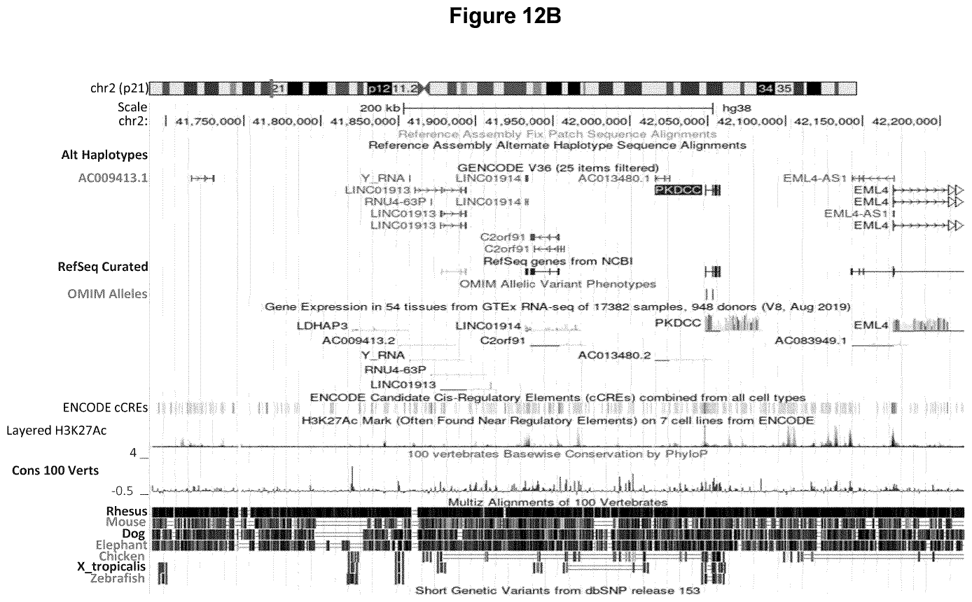

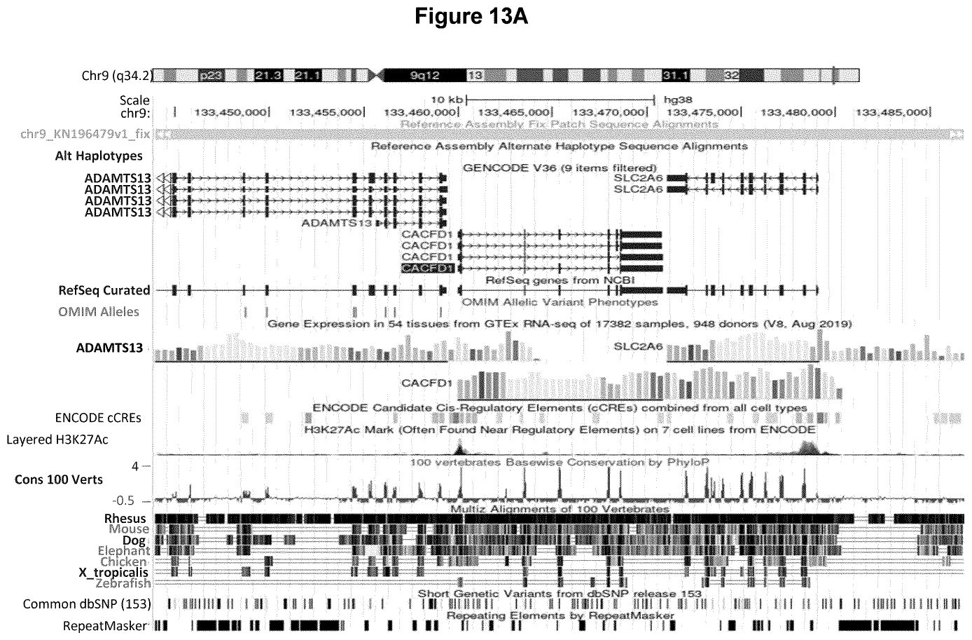

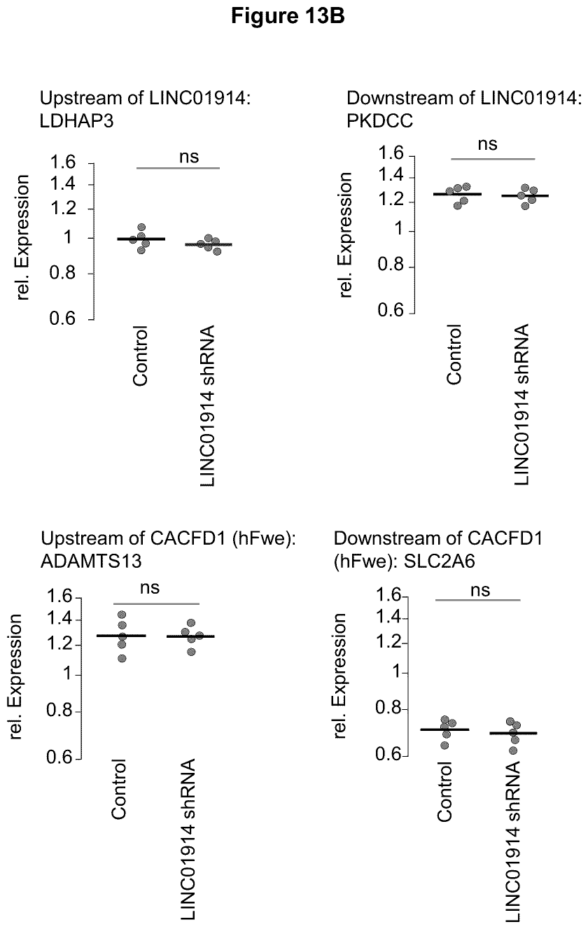

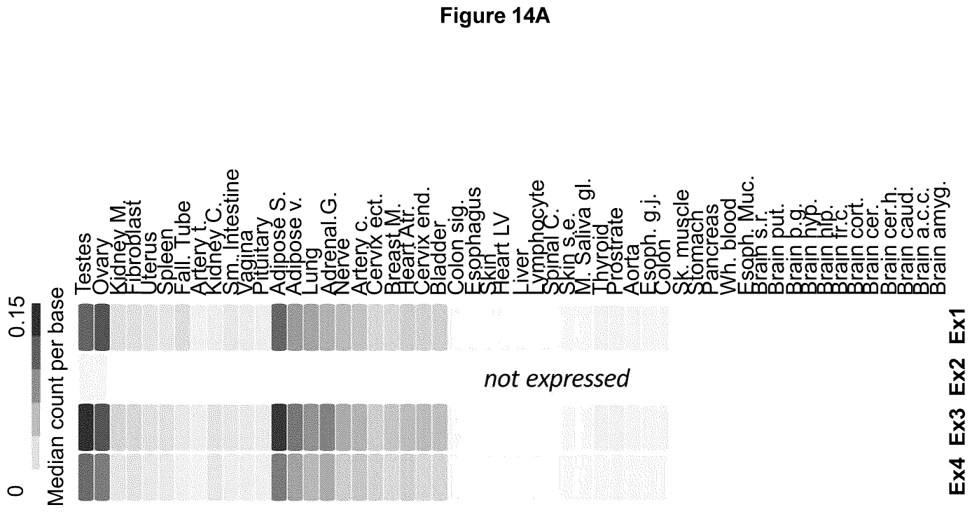

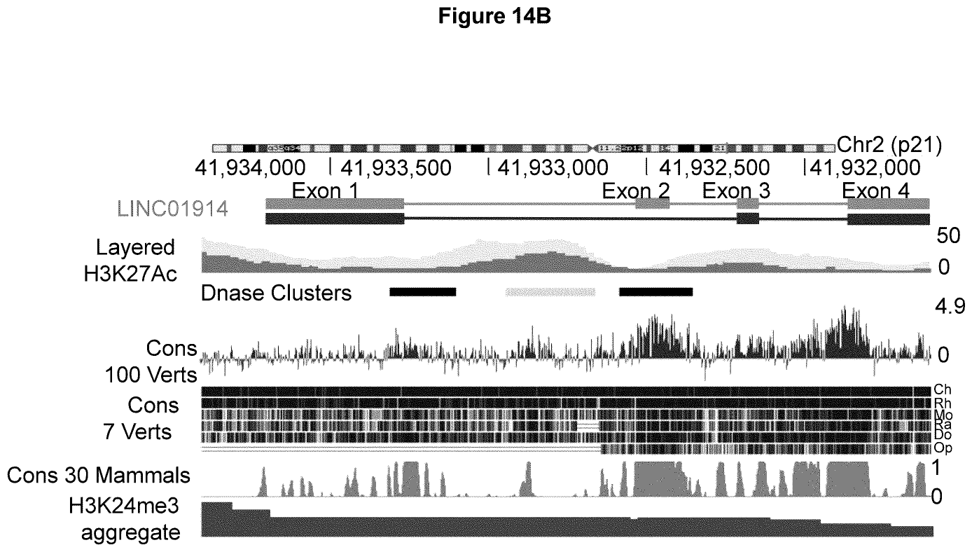

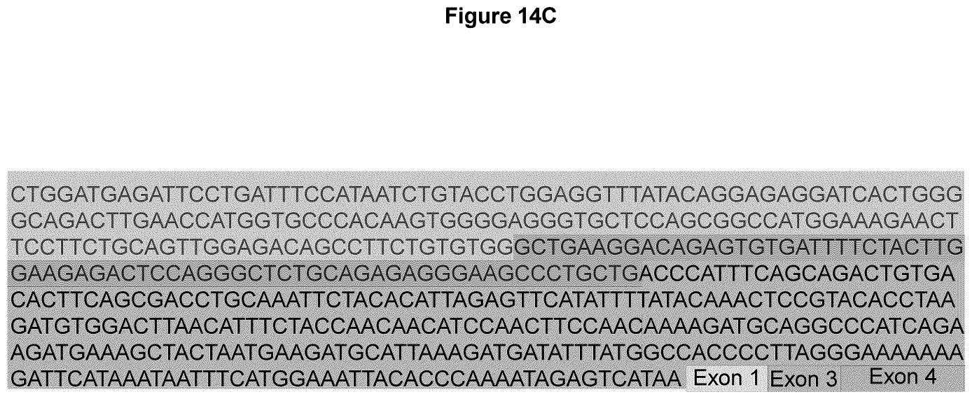

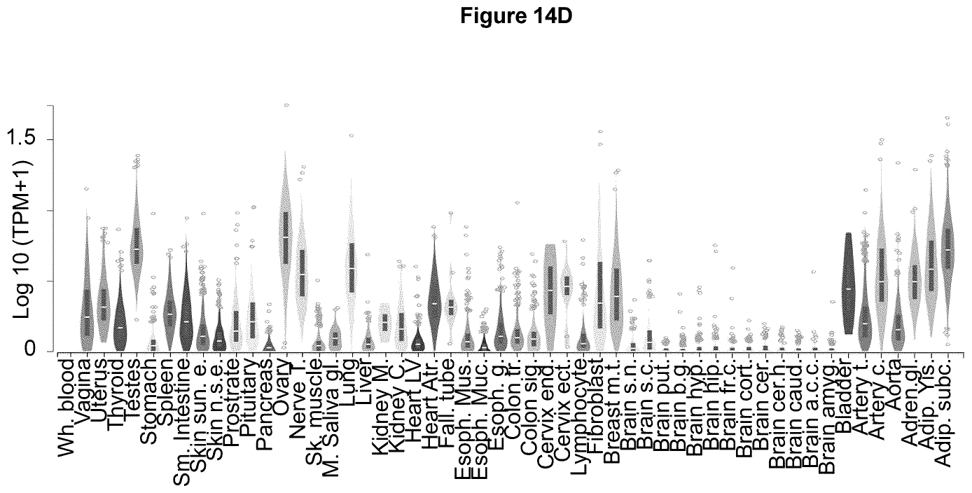

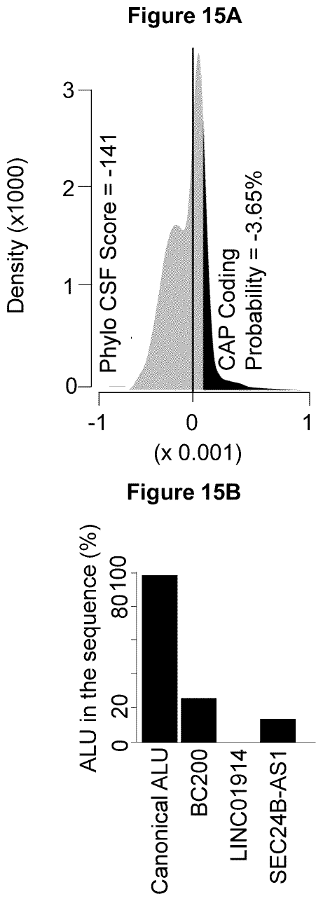

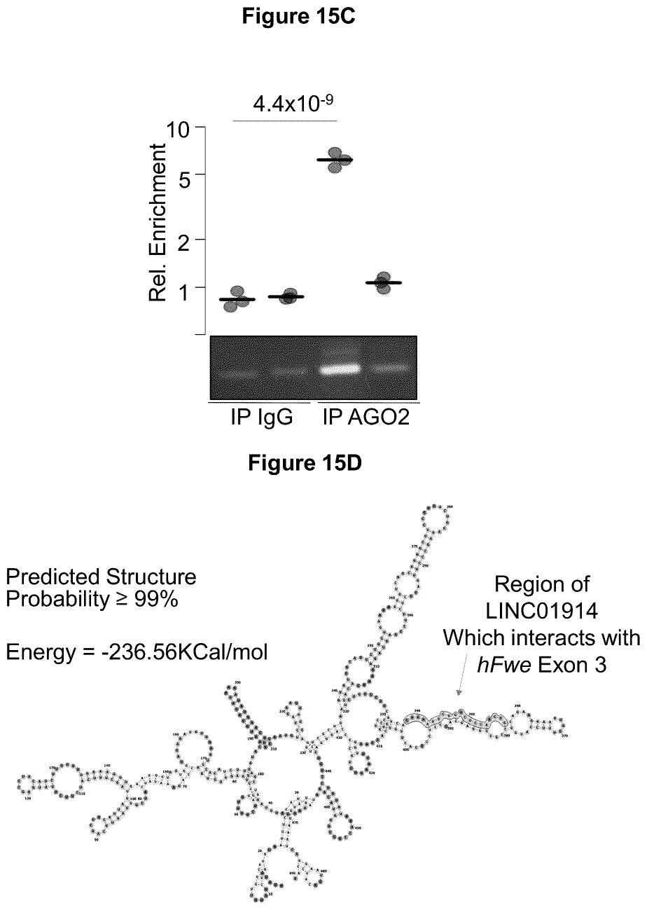

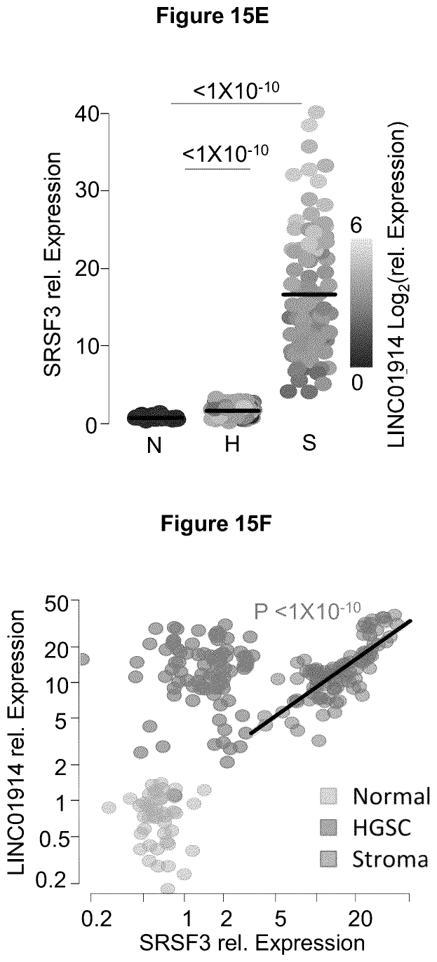

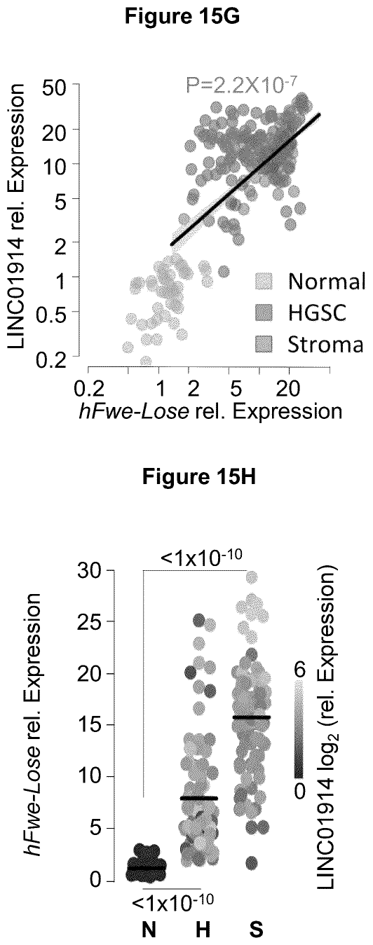

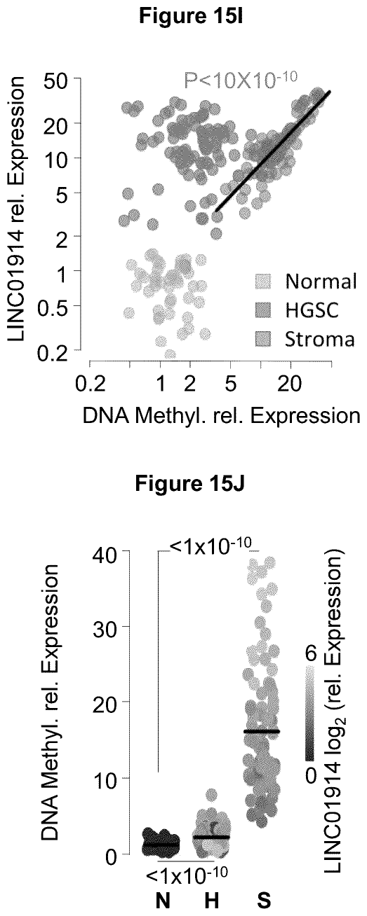

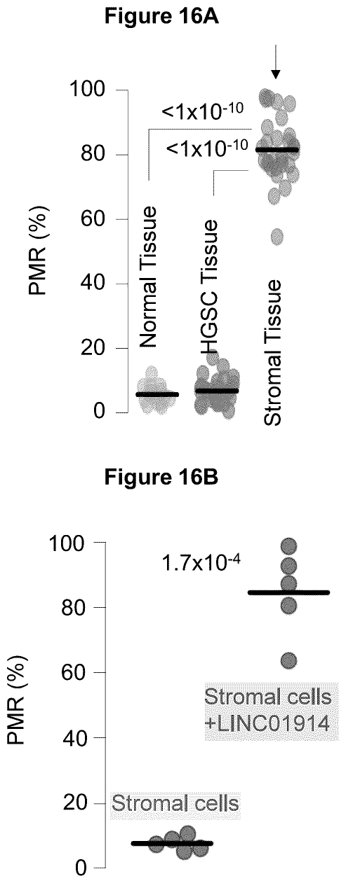

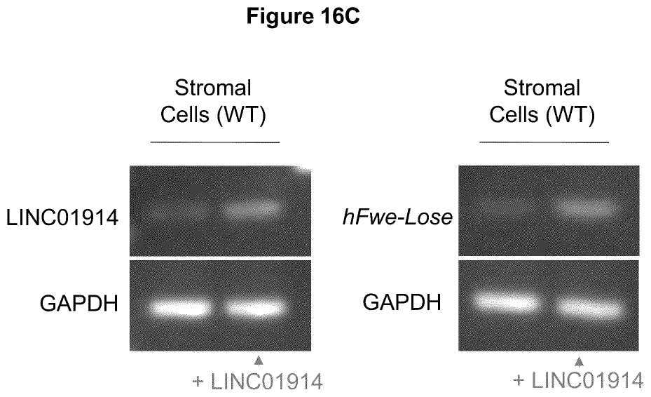

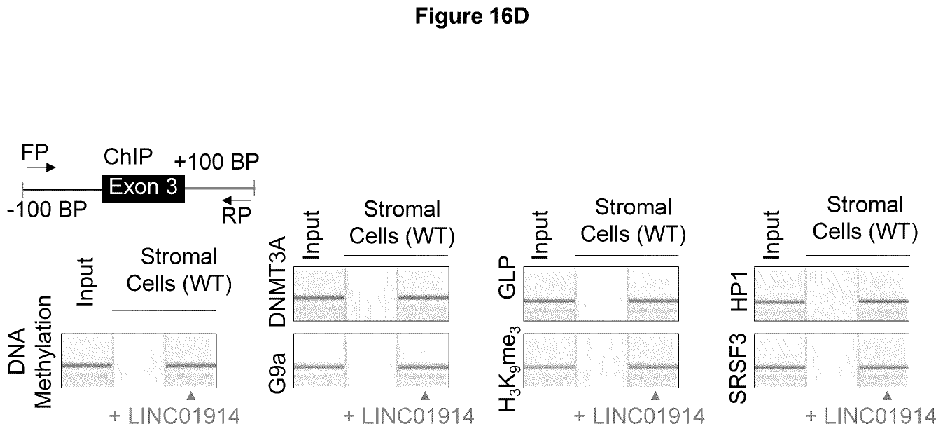

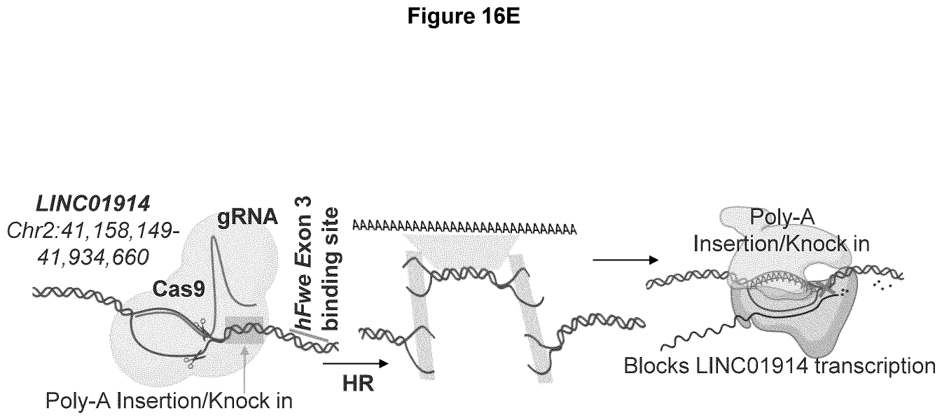

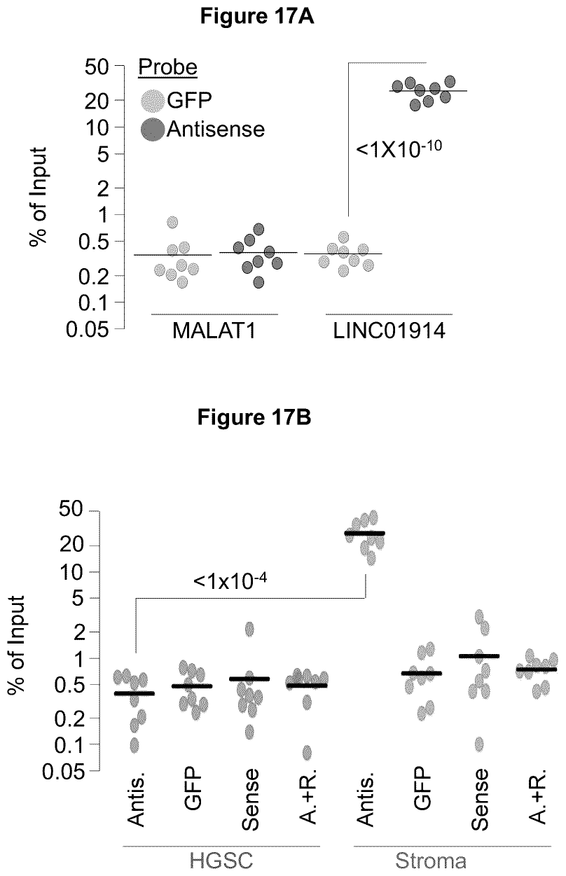

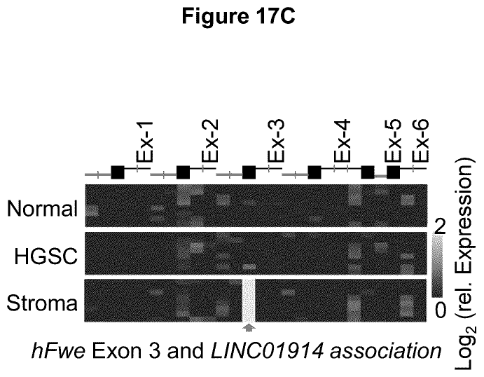

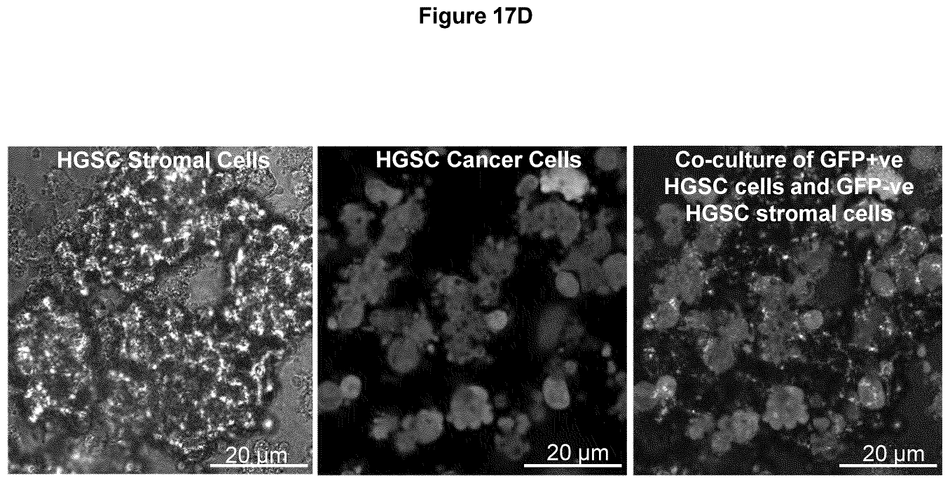

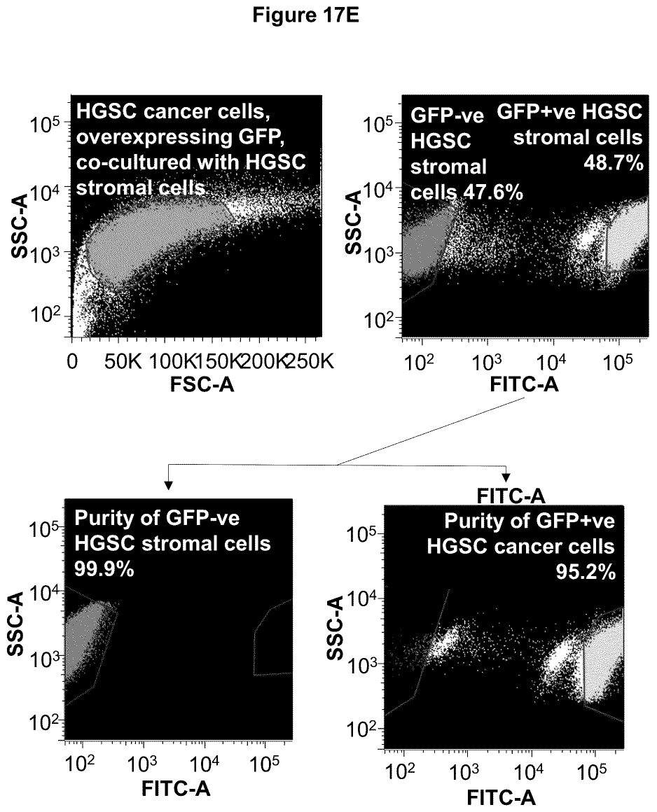

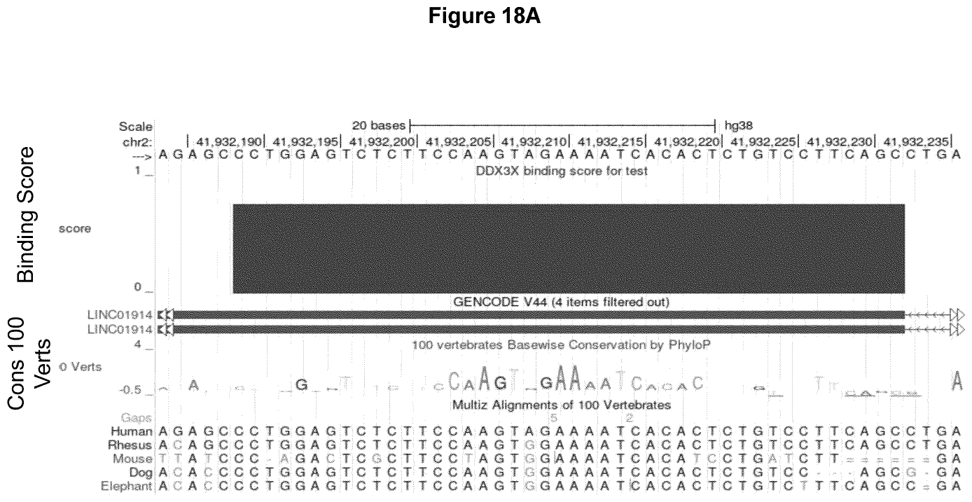

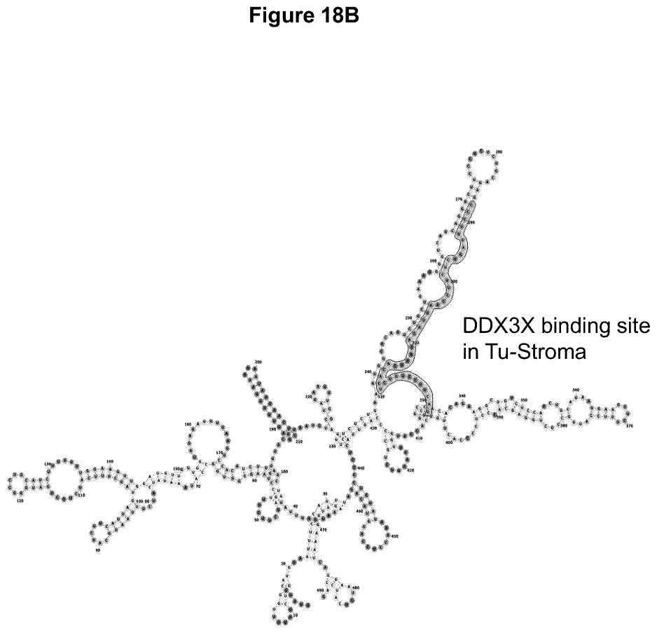

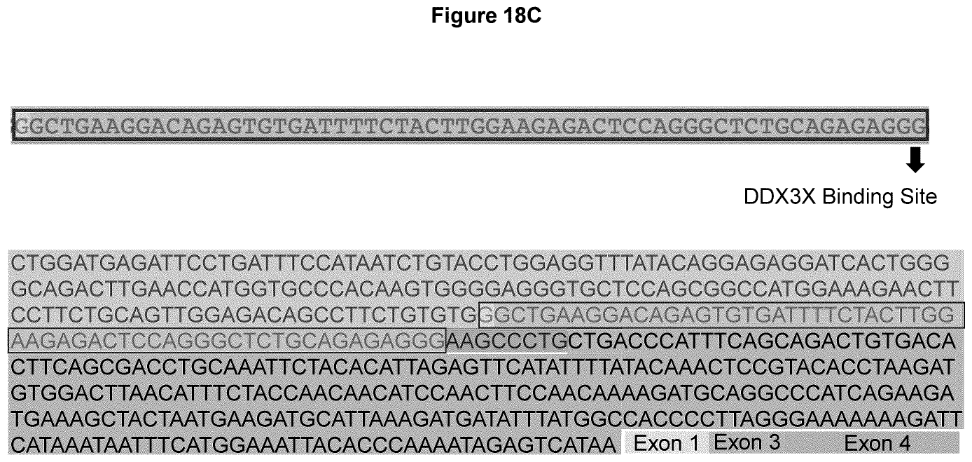

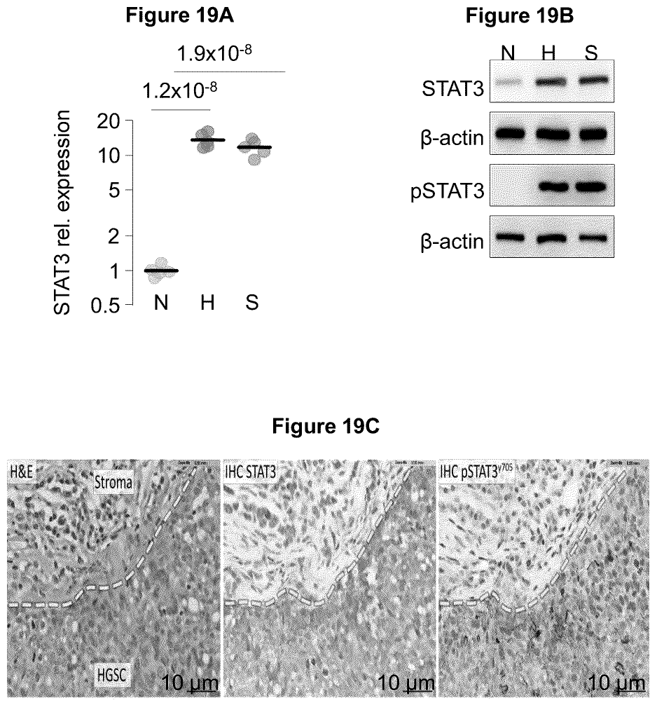

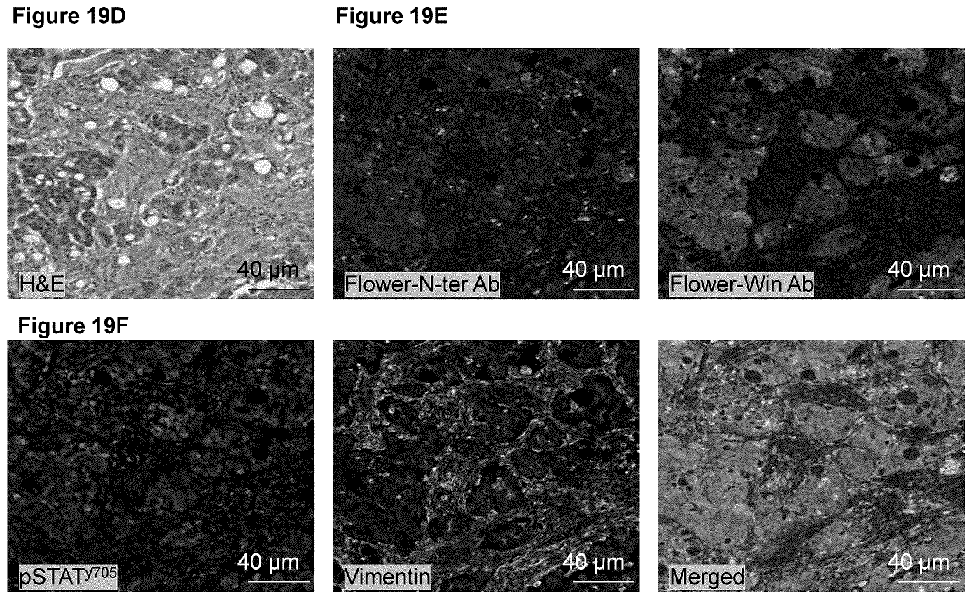

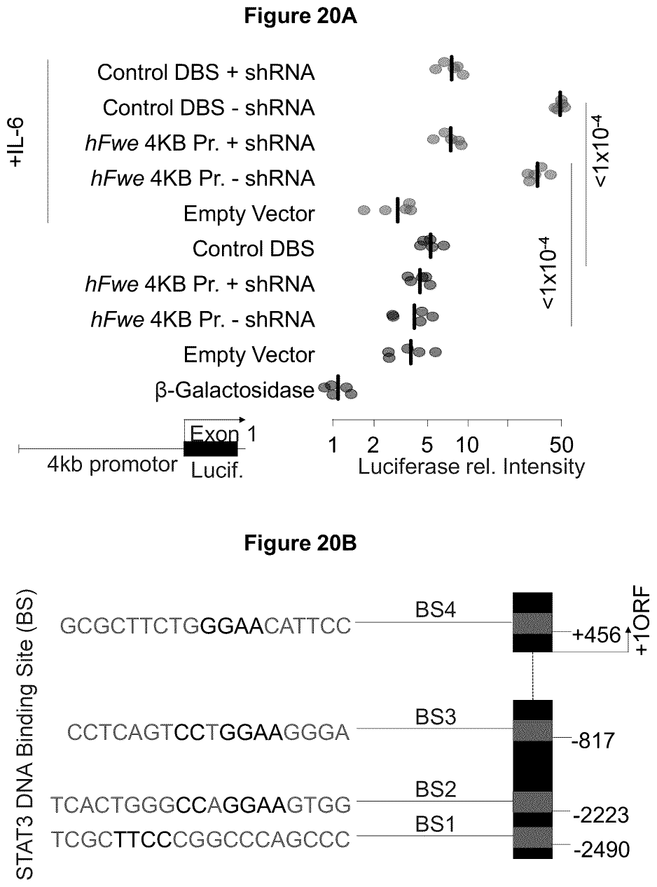

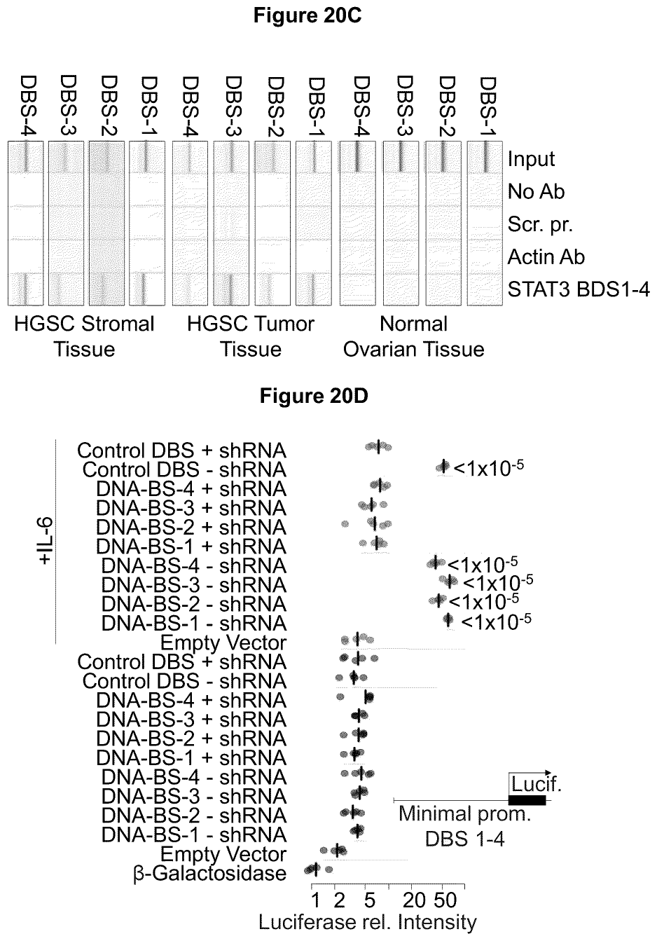

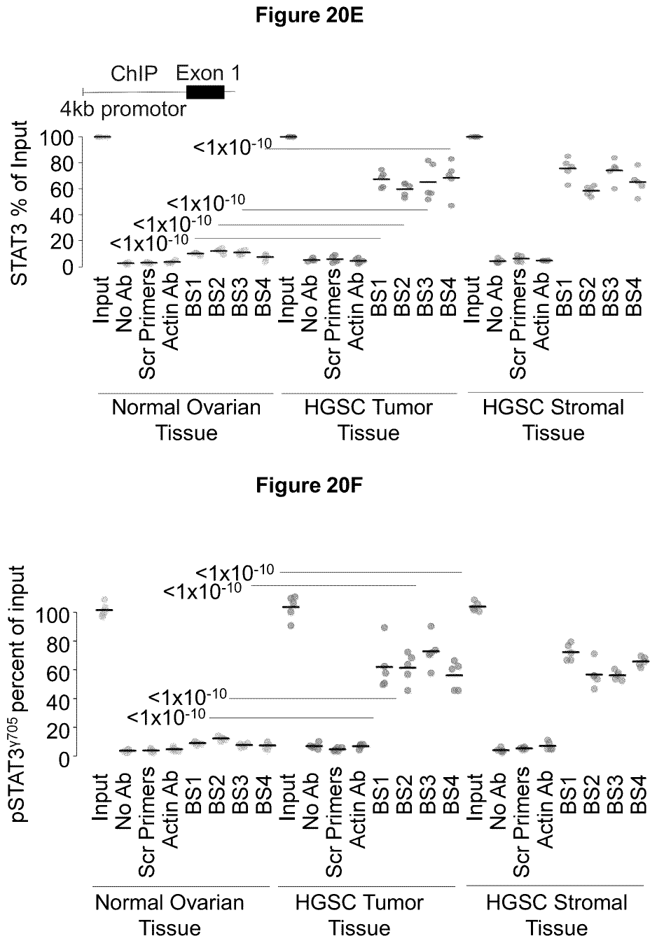

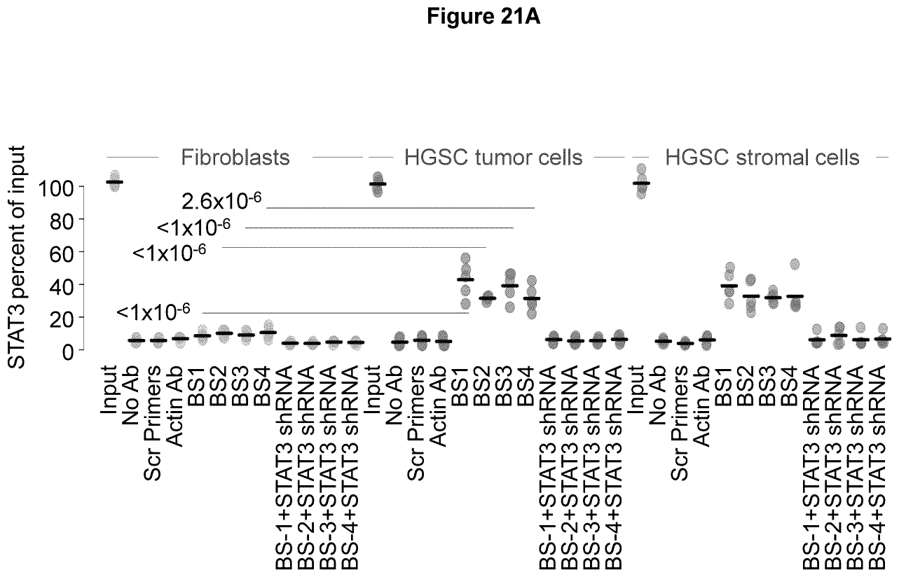

FIG. 2J: AFM shows that WT-exosomes (40.7±5.13 nm) are smaller than the Rab27 a b KO-exosomes (90.0 nm±25.0 nm), (scale 400 nm).

FIG. 2K: ELISA show DDX3X protein is differentially expressed between these samples (highlighted in red, p=2.1X10⁻³⁰; p-value is Bonferroni-Holm corrected for multiple testing) (n=3, horizontal bars indicate mean values)

FIG. 2L: A model depicting the protocol for isolation and primary cell culture/co-culture of HGSC cancerous (GFP-expressing) and HGSC stromal cells (scale 15 μm).

FIG. 2M: HGSC and stroma shows amplification of P2 after UV crosslinked cDNA and absence of P1 amplicon confirms the binding of DDX3X to Tu-stroma (bottom panel).

FIG. 2N: Tumor cells expressing DDX3X tagged with RFP and unlabeled stromal cells (observed as gray cells) were co-cultured to observe the movement of RFP labeled DDX3X from tumor cells to stromal cells (scale 10 μm).

FIGS. 3A-3F show Tu-Stroma and DDX3X knockdown synergize with chemotherapy to reduce tumor growth and metastasis.

FIG. 3A: Schematic outline of 3D co-culture of OVCAR8 cells along with primary stromal cells.

FIG. 3B: Light sheet microscopy images showing the growth of GFP-labeled WT and KO OVCAR8 cells in patient derived 3D scaffolds.

FIG. 3C: Luciferase expressing patient-derived xenografts (PDX1) were transduced with shRNA against Tu-Stroma, DDX3X or both using lentivirus. The WT (lane 1-3) and KD (Tu-Stroma KD in lanes 4 and 5, DDX3X KD in lanes 6 and 7, and double KD in lanes 8 and 9) PDXs were then orthotopically implanted in NSG mice. Once tumors were established, these mice were randomly divided into different treatment groups (n=5) based on similar IVIS images. Each group is either treated with vehicle (lane 2, 4, 6 and 8) or standard-of-care chemotherapy (Docetaxel/Carboplatin, lane 3, 5, 7 and 9). Representative IVIS images were shown at the experimental end point (top panel). The efficiency of the Tu-Stroma and DDX3X KD was confirmed by agarose gel electrophoresis of the RT-qPCR products of Tu-Stroma and DDX3X (middle panel). IHC staining for active caspase-3 was performed in tissue sections collected from the ovaries of each mouse to observe caspase-dependent cell death within the tissue (lower panel). The images shown are representative of each group.

FIG. 3D: A second set of PDX (PDX2) was used to perform an experiment similar to the one described in FIG. 3C. At the end of the experiment, all 5 ovaries from each group were collected as shown.

FIG. 3E: Growth curves with 95% confidence intervals of tumors from the PDXs described in FIG. 3C (top) and FIG. 3D (bottom) demonstrate the efficiency of Tu-Stroma KD, DDX3X KD, and Tu-Stroma and DDX3X double KD in restraining tumor growth (gray lines of the top and bottom plots 1, 3, 4, and 5). Combining the KDs with chemotherapy improves the efficiency of treatment (red lines of the top and bottom plots 1, 3, 4, and 5). The p-values compare the tumor volume at the endpoint of Tu-Stroma KD in combination with chemotherapy (PDX1: p=7.9×10⁻³; PDX2: 2.7×10⁻³) and DDX3X KD in combination with chemotherapy (PDX1: p=1.2×10⁻⁵; PDX2: 3.3×10⁻³) with the control. (n=5, curves were predicted from a generalized additive mixed model).

FIG. 3F: The heatmap shows the number of metastatic lesions detected in the liver, axillary lymph nodes, colon, pancreas, lung, and inguinal lymph nodes of PDX1 and PDX2 at the endpoint of the experiment (left). A colored and numbered label coding key is shown to identify the groups used in the experiment (right). In PDX1, Tu-Stroma KD with chemotherapy (p=5.2×10⁻⁵) and DDX3X KD with chemotherapy (p=7.1×10⁻⁵) have significantly fewer lesions compared to the untreated WT group. In PDX2, Tu-Stroma KD with chemotherapy (p=5.8×10⁻³) and DDX3X KD with chemotherapy (p=4.0×10⁻³) have significantly fewer lesions compared to the untreated WT group.

FIGS. 4A-4D show Tu-Stroma and DDX3X rescue after initial knockdown promotes tumor growth and metastasis.

FIG. 4A: Luciferase expressing WT (lane 1), Tu-Stroma KO (lane 2 and 4) or DDX3X KO (lane 3 and 5) OVCAR8 cells were orthotopically implanted in NSG mice. These mice were divided randomly into two groups (Tu-Stroma KO and DDX3X KO, lanes 2-5, n=5). Three weeks after tumor implantation, these mice were injected with lentiviruses to overexpress Tu-Stroma (lane 4) and DDX3X (lane 5). Representative IVIS images were shown from each group for 6 weeks.

FIG. 4B: Growth curves with 95% confidence intervals of tumors from the orthotopic NSG mice described in FIG. 4A. Gray lines of plots 2 and 3 show Tu-Stroma and DDX3X KO tumor growth and red lines in plots 2 and 3 show the tumor growth in rescue of Tu-Stroma and DDX3X expression. The p-values shown compare tumor volumes at the endpoint of the experiment of Tu-Stroma KO with Tu-Stroma KO recued with Tu-Stroma cDNA at week 3 (p=4.2×10⁻³), and DDX3X KO with DDX3X KO recued with DDX3X cDNA at week 3 (p=2.8×10⁻⁴). (n=5, curves were predicted from a generalized additive mixed model)

FIG. 4C: The heatmap shows the tumor volumes of each group at the endpoint of the experiment described in FIG. 4B.

FIG. 4D: Agarose gel electrophoresis of hFwe isoforms using primers spanning Exons 2 to 4 from RT-PCR products of orthotopic mice injected with OVCAR4 hFwe KO and OVCAR8 hFwe KO cells expressing the different isoforms of hFwe (column 1—hFwe1; column 2—hFwe2; column 3—hFwe3; column 4—hFwe4) (Top panel). Representative pictures of the ovaries from mice injected with OVCAR8 hFwe KO (lane 1), OVCAR8 hFwe/Tu-Stroma double KO (lane 2), OVCAR8 hFwe/DDX3X double KO (lane 3), OVCAR8 hFwe KO treated with vehicle (lane 4), OVCAR8 hFwe KO treated with Docetaxel/Carboplatin (lane 5), OVCAR8 hFwe/Tu-Stroma double KO treated with Docetaxel/Carboplatin (lane 6), OVCAR8 hFwe/DDX3X double KO treated with Docetaxel/Carboplatin (lane 7), OVCAR4 hFwe KO (lane 8), OVCAR4 hFwe/Tu-Stroma double KO (lane 9), OVCAR4 hFwe/DDX3X double KO (lane 10), OVCAR4 hFwe KO treated with vehicle (lane 11), OVCAR4 hFwe KO treated with Docetaxel/Carboplatin (lane 12), OVCAR4 hFwe/Tu-Stroma double KO treated with Docetaxel/Carboplatin (lane 13), OVCAR4 hFwe/DDX3X double KO treated with Docetaxel/Carboplatin (lane 14) (Middle panel).

FIGS. 5A-5J show Flower monoclonal antibody works synergistically with chemotherapy to mitigate tumor growth and metastasis and improving overall survival in HGSC.

FIGS. 5A: A diagram showing the exact amino-acid sequence and Flower protein region targeted by the Flower monoclonal antibody.

FIGS. 5B: Western blot analysis of the Flower mAb was performed using hFwe KO OVCAR8 cells and KO cells specifically expressing hFwe1 isoform. The Flower mAb specifically recognizes exogenously expressed hFwe protein at 21 kDa.

FIGS. 5C: Live-cell imaging (scale 20 μm) demonstrates the efficacy of the Flower mAb to protect hFwe-Lose expressing cells from competition-induced apoptosis. Top row shows a co-culture of GFP and hFwe-Lose expressing cells and RFP and hFwe-Win expressing epithelial cancer cells at 0, 9, 18,27, and 36 hours, as demonstrated previously. The bottom row shows the same co-culture with application of the Flower mAb, demonstrating the rescue of hFwe-Lose expressing cells from competition-induced apoptosis.

FIGS. 5D: The number of GFP+ spots was measured in patient-derived 3D scaffolds populated with OVCAR8 cells and human stromal cells (p=2.6×10⁻¹¹).

FIGS. 5E: GFP+ (white) OVCAR8 cells were co-cultured with unlabeled (grey) human stromal cells in a patient-derived 3D scaffold obtained from decellularized patient tissue.

FIGS. 5F: Schematic outline of antibody experiment in an orthotopic murine model. Human derived HGSC cells were tagged with luciferase and orthotopically injected into ovaries of NSG mice.

FIGS. 5G: Two patient derived xenografts (PDX1 and 2) with luciferase tags were orthotopically implanted into NSG mice, subjected to various treatments, and tracked via IVIS.

FIGS. 5H: Tumor volumes of the PDX1 and PDX2 cohorts tracked over time with 95% confidence intervals demonstrate the ability of Flower mAb to decrease tumor growth as a single agent and in combination with standard chemotherapy. The p-values shown compare tumor volumes at the endpoint of the experiment of mice untreated mice with Docetaxel/Carboplatin and Bevacizumab treated (PDX1: p=8.3×10⁻⁵; PDX2: p=2.1×10⁻⁵), and Flower mAb (PDX1: p=2.5.×10⁻⁴; PDX2: p=4.5×10⁻⁵). (n=5, curves were predicted from a generalized additive mixed model)

FIG. 5I: Heatmap demonstrating impact of Flower mAb on tumor metastasis, indicated by the number of lesions.

FIGS. 5J: Survival curves of the PDX1 and PDX2 cohorts demonstrate the impact of Flower mAb treatment on survival (p=8.7×10⁻⁴).

FIGS. 6A-6E show Flower and Tu-Stroma shape the microenvironment to favor tumor growth and impact overall survival.

FIGS. 6A: NSG mice received orthotopic injections of PDX1 and OVCAR8 cells and were subjected to the same treatment as shown in FIG. 5F. Representative tumors from each cohort are shown. Tumors treated with standard chemotherapy had smaller tumors compared to control and vehicle-treated tumors. The addition of Flower mAb further diminished tumor size.

FIGS. 6B: Schematic outline of antibody experiment in a peritoneal murine model to determine prophylactic efficacy of Flower mAb. Mice were divided into untreated, vehicle-treated, IgG-treated, and Flower mAb-treated groups and received treatments at days 0, 4, 7, and 11. On day 14, all mice received an intraperitoneal injection of 2000 luciferase labelled human derived HGSC cells (PDX1 and OVCAR8). The mice were monitored until day 45, at which point they were imaged.

FIGS. 6C: IVIS imaging of mice that received intraperitoneal injection of Luciferase labeled HGSOC cells (PDX1 on top row and OVCAR8 on bottom row) is shown. As demonstrated, there is a substantial degree of peritoneal spread in the untreated, vehicle-treated, and IgG-treated mice. In contrast, there is a very minimal tumor present in the Flower antibody-treated group.

FIGS. 6D: Survival curves of the mice that received intraperitoneal injection of Luciferase labeled HGSC cells is shown. Curves for PDX1 and OVCAR8 are shown on the top and bottom panel, respectively. Compared to WT and vehicle treatment groups, mice pre-treated 5 with Flower mAb had significantly improved survival (PDX1: p=6.5×10⁻⁷; OVCAR8: p<1×10⁻¹⁰; log-rank tests).

FIGS. 6E: Survival curves stratified by hFwe and Tu-Stroma expression in the test cohort (n=99) and the validation cohort (n=296). The expression was dichotomized by a median split. The plots indicate a worse survival of patients with a high expression of either hFwe-Lose or Tu-Stroma. The association of expression and survival, adjusted for age and stage, was statistically significant (p=3.2×10⁻¹⁰ (test cohort TuStroma), 1.9×10⁻⁸ (test cohort Flower Lose), 6.0×10⁻⁸ (validation cohort TuStroma), 3.7×10⁻⁹ (validation cohort Flower Lose), Cox proportional hazards models).

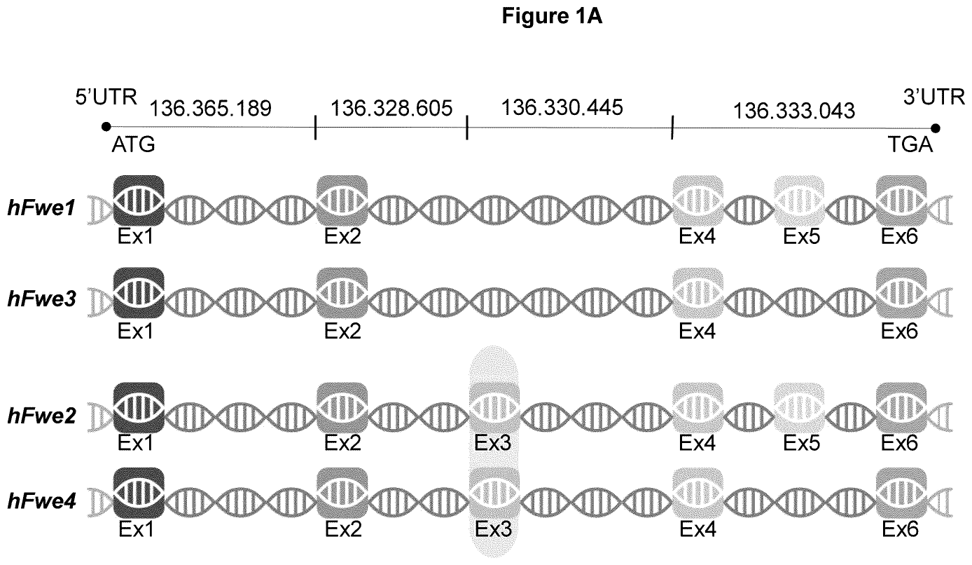

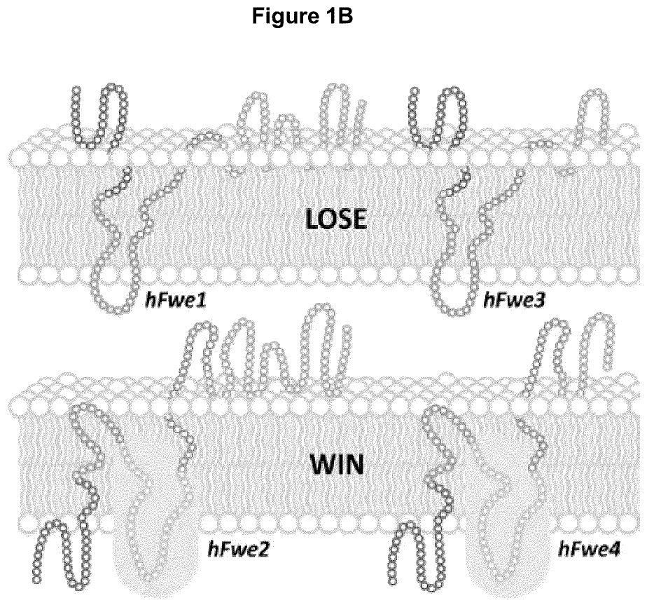

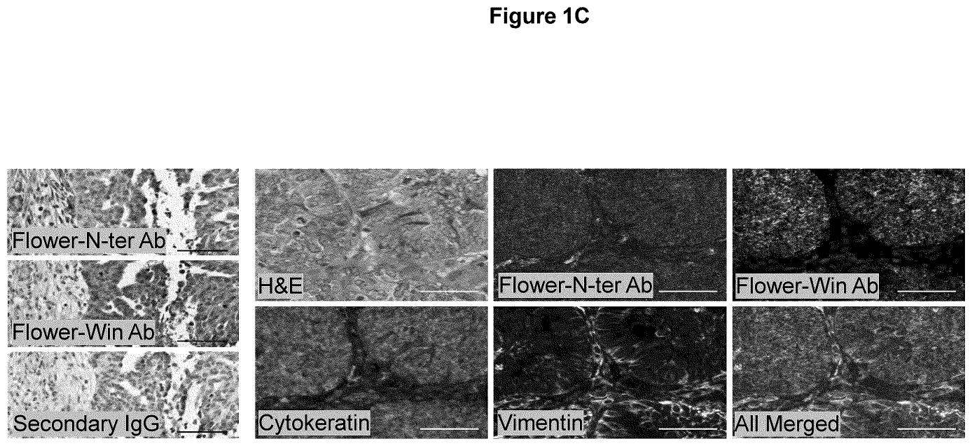

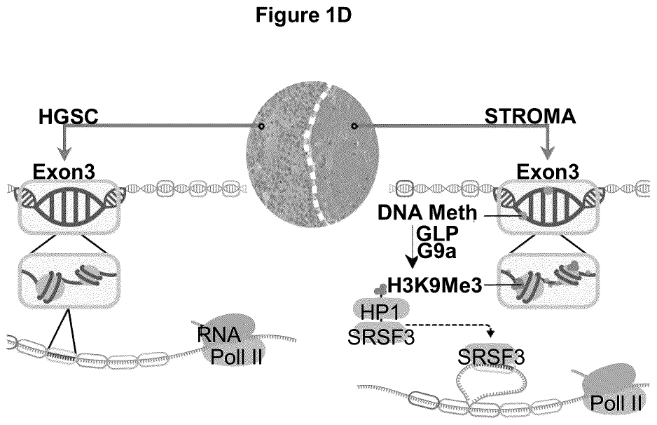

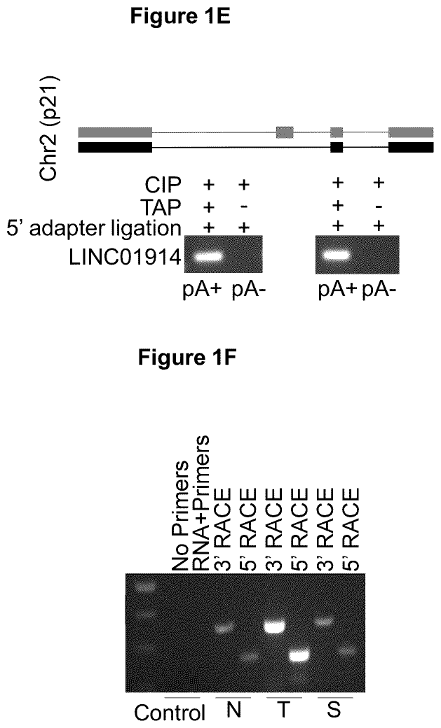

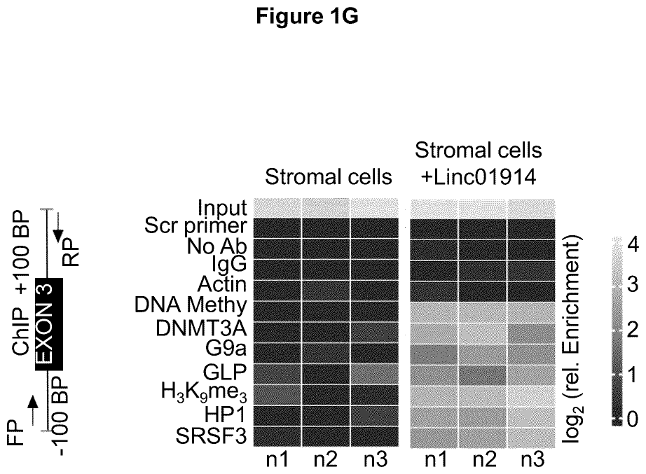

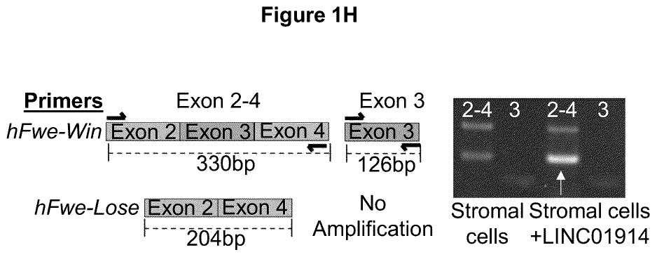

FIG. 7 shows multiplexed immunohistochemistry demonstrating location of Flower Win and Lose expression in HGSC tissue samples. Routine immunohistochemistry using Flower N-terminus and Win Ab and control with secondary IgG is shown in the left panel. Multiplex IHC (right panel) was used to define the precise location of Flower Win and Lose expression in stromal and cancer components of a second HGSC tissue sample. Similar to FIG. 1C, staining was performed with the Flower N-terminus Ab and Flower Win Ab. Staining with Vimentin Ab and Cytokeratin was again used to mark the presence of fibroblasts and epithelial cells in the HGSC samples, respectively.

FIG. 8 shows immunofluorescence experiment demonstrating location of Flower Win and Lose expression. Immunofluorescence from co-cultures of HGSC patient-derived cancer cells and HGSC patient-derived fibroblasts. HGSC patient-derived cancer cells are marked with Alexa Fluor® 594 anti-Cytokeratin-19, shown in the first column in red. HGSC patient-derived fibroblasts are marked Alexa Fluor® 647 anti-Vimentin, shown in the second column in blue. Flower was targeted with anti-Flower N-terminus (targeted in both hFwe Lose and Win) and anti-Flower Win, which was marked with goat anti-mouse IgG AlexaFluor 488, show in the third column in green. The three-color channels were merged to identify which cell type expresses Flower (fourth column). The first row represents a co-culture of HGSC patient-derived cancer cells and HGSC patient-derived fibroblasts incubated with anti-Flower N-terminus. Here, both cell types express hFwe since we can observe that the membrane staining specifically of the Flower N-terminus antibody (green) co-localizes with both the expression of cytokeratin (cancer cell marker in red) and vimentin (fibroblast marker in blue). The second row represents a co-culture of HGSC patient-derived cancer cells and HGSC patient-derived fibroblasts incubated with anti-Flower Win, which shows that Flower Win expression is specific for cancer cells since membrane staining only co-localizes with the expression of cytokeratin (red). As controls, co-cultures of HGSC patient-derived hFwe KD cancer cells and HGSC patient-derived hFwe KD fibroblasts were used and incubated either with anti-Flower N-terminus (third row), or anti-Flower Win (fourth row). The figure shows that the expression of hFwe is not observed in the membrane of both cancer cells and fibroblasts regardless of the hFwe antibody used.

FIGS. 9A-9F shows Flower Lose Expression and Exon 3 Methylation occurs in the stroma of HGSC tissue.

FIGS. 9A: hFwe-Win is expressed abundantly in HGSC tumor while hFwe-Lose isoforms are expressed in stromal tissue. hFwe-Win and hFwe-Lose expression was analyzed by qRT-PCR in 30 matched HGSC FFPE samples and 15 normal ovarian FFPE tissue samples. hFwe-Win were significantly abundant in HGSC tumor versus their adjacent normal ovarian tissues (unmatched and matched t-test p<1×10⁻¹⁰). While hFwe-Lose were abundant in stromal tissue (unmatched and matched t-test p<1×10⁻⁰).

FIGS. 9B: RT-PCR was used to demonstrate Exon 3 skipping and synthesis of hFwe-Lose expression. The locations of the four PCR primers used for amplifications are represented schematically on the structure of the human hFwe gene. Total RNA was extracted from normal, HGSC tumor and stromal tissue and amplified by nested RT-PCR using primers annealing to exons 2 and 4. The 330-bp and shorter product of 204 bp (corresponding to the removal of Exon 3) transcript was detected together in all samples with higher expression in tumor. A shorter product of 126 bp, shows higher expression in stromal tissue.

FIGS. 9C: Model depicting methylation-specific PCR of hFwe Exon 3 is shown. Briefly, we designed two sets of primers targeting the same genomic region of hFwe Exon 3: one for WT DNA and another for unmethylated DNA. Without bisulfite treatment, only the WT primers can generate a PCR product. Upon bisulfite treatment, all unmethylated cytosines are converted to uracil. In that case, if hFwe Exon 3 is fully unmethylated only the unmethylated DNA-specific primers show amplification. If hFwe Exon 3 is methylated, bisulfite treatment cannot convert cytosine to uracil, thus maintaining the same sequence of the WT DNA. Since the potential methylation region of hFwe Exon 3 is small, there will only be amplification if we combine the forward WT-specific primer targeting hFwe Exon 3 with the reverse unmethylated DNA-specific primer targeting hFwe Intron3.

FIGS. 9D: DNA was extracted from 15 matched normal, stromal, and tumor tissue samples and subjected to bisulfite treatment. Sequencing results of bisulfite treated HGSC (top) and stroma (bottom) DNA are shown. The PCR products amplified from bisulfite-treated tissue DNA were cloned into the pUC57 vector via the restriction site and then sequenced. The plot under the sequencing results for bi-sulfite-treated stromal samples identifies the methylated cytosines and their percentage of occurrence.

FIGS. 9E: Methylation-specific PCR (MSP) was performed on both the control and the bisulfite-treated DNA (pooled from 15 matched normal, stromal, and tumor tissue samples). Results of the MSP for normal (lanes 1-6), tumor (lanes 7-12), and stroma (lanes 13-18) tissue are shown. Each tissue type is further subdivided into untreated (first 3 lanes) and bisulfite treated (last 3 lanes). The distribution shown further validates the methylation pattern in the stromal tissue. In all three tissue types, the WT band is present in the untreated group. For the normal and HGSC tissue, the band in the treated group appears only in the presence of unmethylation primers. However, in the bisulfite treated DNA from stromal tissue, amplification occurred only with methylation-specific primers.

FIGS. 9F: The methylation levels (percentage of methylated reference, or PMR) observed by qPCR were significantly different between Normal, HGSC and Stroma. Significantly higher methylation level was observed in the case of the Stroma compared to the normal (p<1×10⁻¹⁰) and HGSC (unmatched t-test p<1.3×10⁻⁸; matched t-test p<1×10⁻⁰).

FIGS. 10A-10C shows characterization of all Flower exons.

FIG. 10A: qPCR experiment is performed to study the expression of spliceosome components (12) in normal ovarian (white bar plot), HGSC tumor (red bar plot), and stromal (blue bar plot) tissue samples (5 HGSC patients). Results show no significant difference in the expression profile of the spliceosome proteins in all the three tissue samples (all p-values>0.05).

FIG. 10B: The G+C content (as a percentage) signal 100 nucleotides downstream of the 5′splice site at Exon 3 of hFwe gene locus is depicted. This Figure discloses amino acid sequence “MNAFILLLCEAPFCCQFIEFANTVAEKVDRLRSWQKAVFYCG” as SEQ ID NO: 398.

FIG. 10C: The PhastCons score of the hFwe gene at Exons 2, 3 and 4, which represents gene conservation, is depicted. Conservation peaks at Exons 2, 3, and 4 are highlighted with white arrows.

FIGS. 11A-11G shows HGSC stromal tissue shows a specific enrichment of splicing machinery components at Flower Exon 3.

FIG. 11A: The principal component analysis (PCA), focusing on the genomic positions, shows clusters of exons and positions next to exons (+/−200 bp), as well as the remote position of the Exon 3 in the dimensional reduction space. The red-highlighted dot implies the distinctiveness of the Exon 3 body of the hFwe from all other positions on the hFwe gene, explaining ˜90% of the first principal component variance.

FIG. 11B: The line plot summarizes the hFwe Exon 3 body and the adjacent 5′ & 3′ flanking 200 bp regions from matched normal, tumor, and stromal tissue. The colors green, red, and blue represent the tissue origin (representing pooled normal, tumor, and stroma tissue samples respectively). The plot shows increased DNA methylation and H3K9Me3, and binding of the SRSF3, GLP, G9a, and HP1, with decreased RNA Poll II occupancy at the hFwe Exon 3 in the stromal samples (all data points have p<0.05; the error bars represent standard errors centered around the mean, biological replicates, n=8).

FIG. 11C: ChIP is performed in matched normal ovarian, HGSC tumor, and stromal tissue for H3K9Me3, HP1, SRSF3, G9a, GLP, and RNA Poll II occupancy on the hFwe Exon 3 and the adjacent 5′ & 3′ 200 bp flanking regions. Amplicons are analyzed by electrophoresis using the QIAxcel instrument. Results show a poor association of all the epigenetic modifiers across all Exons and the flanking regions. The HGSC tumor and stromal tissue show a reverse binding pattern showing high binding exclusively on Exon 3 and low association at the 5′ & 3′ flanking regions in the stromal tissue samples and vice versa for H3K9Me3, HP1, SRSF3, G9a, GLP, and RNA Pol II. RNA Pol II occupancy on contrary is found to be high on Exon 3 on the HGSC tumor tissue compared with the stromal tissue.

FIG. 11D: The line plot shows aggregated traces of DNA methylation and H3K9Me3, as well as binding of HP1, SRSF3, RNA Pol II, G9a, and GLP across all the six Exons and flanking regions of the hFwe gene locus in the matched normal ovarian, HGSC tumor, and stromal tissue samples from 8 HGSC patients. Normal ovarian tissue shows a relatively poor association of the epigenetic modifiers across all the six exons and the flanking regions in comparison to the HGSC tumor tissue. A unique association of epigenetic modifiers with the exception of RNA Pol II binding on the Exon 3 body in the stromal tissue is outlined within the black box. Each error bar represents the standard error, centered around the mean (Biological replicates, n=8).

FIG. 11E: The principal component analysis, using all available ChIP-qPCR data as input (genomic location+all modifications/binding), shows the distinctiveness of the DNA Methylation (˜50% and ˜25% of the first and second principle components variances explained, respectively) and SRSF3 binding (˜40% and ˜20% of the first and second principle components variances explained, respectively) exclusively on the hFwe Exon 3 (blue dots outlined within the red box corresponding to the stromal tissue samples). Their remote position on the PCA dimensional reduction suggests their crucial role in making the hFwe Exon 3 genomic region epigenetically distinct. Colors green, red, and blue correspond to the matched normal ovarian, HGSC tumor, and stromal tissue samples from 8 HGSC patients.

FIG. 11F: Exon walking is performed on the hFwe gene and 200 bp flanking regions upstream (E1-6−200 bp, −100 bp) and downstream (Ex1-6+100 bp, +200 bp) of each Exon for SRF3 binding by ChIP analyzed by electrophoresis using QIAxcel instrument. Image displays no antibody (negative control, row1); Input (positive control, row 2); Normal ovarian (row 3); HGSC tumor (row 4); and Stromal (row 5) matched tissue samples from 8 HGSC patients, depicting a site-specific association of SRSF3 exclusively to the Exon 3 body in the stromal tissue with no binding observed on any Exons in the normal ovarian or the HGSC tumor tissue.

FIG. 11G: The scatter plot shows a clear separation of the normal (green), tumor (red), and stromal (blue) sample clusters based on DNA Methylation and SRSF3 enrichment (the black line represents the linear regression with (p=1.4×10⁻⁶) between the tumor and stroma). The size of the colored dots represents the corresponding increase in the expression or the hFwe-Lose (fold change ranging from 5 to 15) with respect to the DNA Methylation and SRSF3 fold enrichment.

FIGS. 12A-12B show characterization of LINC01914 locus.

FIG. 12A: An analysis derived from sequence-specific BLAST between Exon 3 and transcripts with potential complementarity with hFwe Exon 3 and flanking regions shows a list of potential candidates which may influence DNA methylation in a site-specific manner. The percent complementarity and location of complementarity relative to Exon 3 is shown (left panel). Box plot (right panel) shows expression of these transcripts in matched stromal and normal tissue. LINC01914 shows 90% sequence homology with the core element of hFwe Exon 3 and has high expression in the stromal tissue (p<1×10⁻¹⁰) (the error bars represent standard errors centered around the mean, biological replicates, n=5).

FIG. 12B: UCSC Genome Browser maps the human LINC01914 gene on chromosome 2. The map provides annotations of genes upstream and downstream of the LINC01914 gene marked as LDHAP3 and PKDCC, respectively.

FIGS. 13A-13B show the knockdown of LINC01914 does not impact the expression of the genes upstream and downstream of LINC01914 and CACFD1.

FIG. 13A: UCSC Genome Browser maps the human CACFD1 gene on chromosome 9. The map provides annotations of genes upstream and downstream of the CACFD1 gene marked as ADAMTS13 and SLC2A6, respectively.

FIG. 13B: RT-qPCR experiment is conducted to assess the cis- or trans-regulatory role of LINC01914 ncRNA. The experiment is performed in the control primary cultured stromal cells and experimental stromal cells upon silencing of LINC01914 lncRNA. Results show no effect on the expression of the nearby genes on chromosome 2 (LINC01914) and chromosome 9 (CACFD1), showing it works in trans (all p-values>0.05) (Biological replicates, n=5).

FIGS. 14A-14D show characterization of LINC01914 exons and expression of LINC01914 in different tissue types.

FIG. 14A: Heatmap representation of the expression of LINC01914 exons 1-4 (Ex1-4) in indicated tissues calculated by median count per base. Among other tissues, ovaries express large amounts of LINC01914.

FIG. 14B: ENCODE data mining for gene characterization of LINC01914. Two isoforms of LINC01914 on Chromosome 2 are depicted. The isoform with 4 exons (top; grey color) is not synthesized as Exon 2 is not included in the principal transcript. The isoform (bottom; black color) with 3 Exons is the primary transcript that is expressed in HGSC stromal tissue. H3K27Ac ChIP shows active gene promoter, DNase I activity also shows active transcription at this gene. Gene conservation mapping shows that this lncRNA is conserved between vertebrates.

FIG. 14C: The sequence and the color-coded exon structure of the spliced, functional variant of LINC01914 (SEQ ID NO: 399) is shown.

FIG. 14D: Violin plots show the expression of LINC01914 across indicated tissue types. Among other tissues, LINC01914 is highly expressed in ovaries.

FIGS. 15A-15J show characterization of LINC01914 structure and correlation of LINC01914 expression with markers of Flower Lose generation.

FIG. 15A: LINC01914 was analyzed for conserved protein-coding probability using Phylo CSF. A Phylo CSF score of −141.44 was obtained showing low protein-coding probability.

FIG. 15B: LINC01914 sequence (lane 3) was analyzed for the presence of Alu elements relative to other lncRNAs (BC200 and SEC24B-ASI) and canonical Alu sequence (lane 1).

FIG. 15C: Immunoprecipitation was performed using IgG or AGO2 antibodies and fold enrichments of SOCS1 (a protein-coding gene) and Tu-Stroma (non-protein coding) RNA were analyzed by qPCR. The fold enrichment is depicted as a bar plot and the gel image is presented below. The error bar represents the standard error (p=4 0.4×10⁻⁹) (Technical replicates, n=3).

FIG. 15D: The RNA structure of LINC01914 was predicted using RNAWebSuite, ViennaRNA Web Services and minimum free energy structure is depicted. Base colors indicate probability of folding. The 21 bp complementary sequence to hFwe Exon 3 is highlighted in red. The sequence presented in this figure is set forth in SEQ ID NO: 397.

FIG. 15E: The box plot shows increased SRSF3 binding to the hFwe Exon 3 in the stromal samples (unmatched p<1×10⁻¹⁰ and matched p=7.2×10⁻⁷ normal vs. HGSC; unmatched and matched p<1×10⁻¹⁰ HGSC vs. stroma). The color of the dot represents the log 2 fold change of the LINC01914 expression.

FIG. 15F: The scatter plot shows a positive correlation of LINC01914 expression and SRSF3 binding to the hFwe Exon 3 (the black line represents linear regression with R²=0.67 in the stromal samples; p<1×10⁻⁰).

FIG. 15G: The scatter plot shows positive correlation of LINC01914 expression and hFwe-Lose (the black line represents linear regression in the stromal samples; p=2.2×10⁻⁷).

FIG. 15H: The box plot shows increased hFwe-Lose expression in the stromal samples (unmatched and matched p<1×10⁻¹⁰ normal vs. HGSC; unmatched p<1×10⁻¹⁰ and matched p=9.6×10⁻⁷ HGSC vs. stroma). The color of the dot represents the log 2 fold change of the LINC01914 expression.

FIG. 151: The scatter plot shows positive correlation of LINC01914 expression and hFwe Exon 3 DNA Methylation (the black line represents linear regression with R²=0.82 in the stromal samples; p<1×10⁻⁰).

FIG. 15J: The box plot shows increased DNA Methylation of the hFwe Exon 3 in the Stromal samples (unmatched p<1×10⁻¹⁰ and matched p=1.1×10⁻³ normal vs. HGSC; matched and unmatched p<1×10⁻¹⁰ HGSC vs. stroma). The color of the dot represents the log 2 fold change of the LINC01914 expression.

FIGS. 16A-16E show elevated LINC01914 expression in stroma leads to increased enrichment of splicing machinery at Flower Exon 3.

FIG. 16A: The distribution of PMR values demonstrating hFwe Exon 3 methylation in a new cohort of HGSC tumor, stromal, and normal ovarian tissue is shown. Significantly higher methylation level was observed in the case of the Stroma compared to the normal (p<1×10⁻⁰) and HGSC (unmatched and matched p<1×10⁻¹⁰) (Biological replicates for normal tissue n=15, biological replicates for tumor and stroma tissue n=30).

FIG. 16B: MethyLight assay shows Flower Exon 3 DNA methylation in WT (row 1) and LINC01914 overexpressing (row 2) stromal cells. Data significantly higher methylation in the presence of LINC01914 overexpression (p=1.7×10⁻⁴) (Technical replicates, n=5).

FIG. 16C: RT-PCR confirmed overexpression of LINC01914 upon transfection compared to control in WT stromal cells and increase in hFwe-Lose transcript upon LINC01914 overexpression in in WT stromal cells.

FIG. 16D: ChIP on Exon 3 and 100 bp flanking regions in WT stromal cells and LINC01914 overexpressing stromal cells shows significantly increased association of DNA methylation, DNMT3A, G9a, GLP, H3K9Me3, HP1, and SRSF3 after LINC01914 overexpression.

FIG. 16E: The model depicts the method used to silence LJNC01914 expression. A Poly-A sequence was inserted in the LINC019/4 locus, specifically before the region that binds to hFwe Exon 3. Briefly, CRISPR-Cas9 homology repair directs the insertion of a Poly-A sequence prior to the binding site sequence of LINC01914 to hFwe Exon 3, thus blocking LINC01914 transcription by RNA polymerase.

FIGS. 17A-17E show LINC01914 binds to Flower at Exon 3.

FIG. 17A: ChOP pull-down assay is performed in the pooled set of 8 stromal tissue fractions collected from the HGSC patients to determine the physical association of LINC01914 on the Exon 3 body on the hFwe gene locus. The assay includes the use of biotin-labeled antisense DNA oligos spanning across the region complementary to the Exon 3 body to allow capture of LINC01914 RNA-associated Exon 3 body of the hFwe gene loci with streptavidin beads. qPCR analysis shows the enrichment of LINC01914 RNA on the Exon 3 body using the LINC01914 anti-sense oligos (LINC01914 Pr) (red and black dots indicates the experiment repeated 8 times) but not for the positive control, MALAT1(nuclear-enriched LnC-RNA) using MALAT1 anti-sense oligos (MALAT1Pr) (represented as a percentage of input). Pull-down with biotin probes against the green fluorescent protein (GFP) (used for both MALAT1 and LINC01914) RNA is used as a negative control, as it shows no enrichment on the Exon 3 body. All the data shows the mean values±SE (error bars) from eight replicates. P-value is shown in the figure (p<1×10⁻⁰).

FIG. 17B: ChOP pull-down assay is performed using sense and antisense oligos to determine the physical association of LINC01914 on the Exon 3 body on the hFwe gene locus in the pooled set of HSGC tumor (red) and stromal (blue) tissue fractions collected from 8 HGSC patients. Data shows no enrichment of LINC01914 RNA on the Exon 3 body using the LINC01914 anti-sense and sense oligos in the HGSC tumor fractions. No enrichment is observed with the LINC01914 anti-sense oligos upon pretreating the chromatin with RNase A (R). On the other hand, enrichment of LINC01914 RNA on the Exon 3 body is observed exclusively upon using the LINC01914 anti-sense oligos. Pull-down with biotin probes against the GFP (used for both MALAT1 and LINC01914) RNA is used as a negative control. All the data shows the mean values±SE (error bars) from eight replicates. P-value is shown in the figure (p<1×10⁻⁴).

FIG. 17C: ChOP analysis is performed on each Exon (1-6) and 200 bp flanking regions upstream (5′) and downstream (3′) from each exon of the hFwe gene locus in 8 normal ovarian, HGSC tumor, and stromal tissue samples. Results reveal the physical association of LINC01914 exclusively to the Exon 3 body and not to any other exons or their adjacent 5′ or 3′ flanking regions in the stromal tissue samples (highlighted blue color). Normal ovarian and HGSC tumor tissue show no physical interaction on either exons or the adjacent flanking regions. The heatmap depicts individual data points showing color deviation from low to high expression (yellow-black-blue).

FIG. 17D: The efficiency of GFP-lentivirus infection is confirmed by detecting GFP expression using fluorescence microscopy in primary cultured HGSC cancer cells. Images represent the primary co-culture of non-GFP labeled stromal cells and GFP-labeled cancer cells extracted from the HGSC patient sample.

FIG. 17E: HGSC GFP-positive cancer cells and HGSC GFP-negative stromal cells are co-cultured for 8 days, and then isolated by BD FACSAria™. HGSC cancer cells were isolated as GFP positive cells with a purity of 99.9%, while HGSC stromal cells were sorted out as GFP negative cells with a purity of 95.2%.

FIGS. 18A-18C show identification of DDX3X binding site within LINC01914.

FIG. 18A: A visual representation is presented to evaluate the binding affinity of DDX3X in the LINC01914 context (SEQ ID NO: 400). The figure includes a genome browser depiction featuring conservation and base-wise conservation tracks at the LINC01914 locus. The “Cons 100 Verts” track illustrates conservation across 100 vertebrate species, measured by PhastCons. Additionally, conservation comparisons between humans (SEQ ID NO: 400) and other species are demonstrated in tracks labeled with the respective species names.

FIG. 18B: A visual representation highlighting the significance of the LINC01914 site when in a double-stranded conformation is shown. This configuration is predicted to be a binding site for the DDX3X protein (shown in red). The map offers insights into the regions or features that are most influential or critical for the predicted interaction between DDX3X and the LINC01914 site in its double-stranded conformation.

FIG. 18C: The figure visually represents the sequence of the DDX3X binding site (nucleotides 158-217 of SEQ ID NO: 399), highlighted in brown, within the genomic context of LINC01914 (SEQ ID NO: 399). In this illustration, the exonic regions of LINC01914 are distinguished: Exon 1 is depicted in green, Exon 3 in purple, and Exon 4 in gray. The purpose of this representation is to provide a clear and detailed view of the spatial arrangement of the DDX3X binding site in relation to the specific exonic regions of the LINC01914.

FIGS. 19A-19F show STAT3 and phospho-STAT3 expression is elevated in the HGSC compared to normal ovarian tissue.

FIG. 19A: STAT3 mRNA expression was analyzed in pooled normal ovarian, HGSC cancer and stromal matched tissue from 8 patients using RT-qPCR and relative fold expression ±SE is depicted (unmatched p=1.2×10⁻⁸ and matched p<1×10⁻¹⁰ normal vs. HGSC; unmatched p=1.9×10⁻⁸ and matched p HGSC vs. stroma).

FIG. 19B: STAT3 and STAT3^{Y705} protein levels were analyzed in pooled normal ovarian, HGSC cancer and stromal matched tissue from 8 patients using western blot analysis and a representative blot is depicted.

FIG. 19C: STAT3 and STAT3^{Y705} levels were analyzed stroma and HGSC cancer tissue sections. H&E staining shows stroma-tumor boundary. IHC staining using STAT3 and pSTAT3^{Y705} antibodies shows high expression both in the stroma and tumor tissue.

FIG. 19D: Hematoxylin and eosin staining of a HGSC sample with both tumor and stromal regions is shown.

FIG. 19E: Multiplexed IHC of the same HGSC sample from FIG. 19D using Flower Win Ab (panel 2), and Flower N-terminus Ab (panel 1) is shown.

FIG. 19F: Multiplexed IHC of the same HGSC sample (FIG. 19D) using pSTAT3^{Y705}Ab (left panel), Vimentin Ab (middle panel) and a merge (right panel) of all antibodies used in FIGS. 19E-19F are shown.

FIGS. 20A-20F show STAT3 controls transcription at the hFwe promoter in both cancer and stromal cells.

FIG. 20A: Luciferase assay is performed on the 4kb promoter of hFwe gene cloned in pGL4 vector transfected in OVCAR8 cells in the presence or absence of IL-6. c-Fos promoter (control STAT3 DBS) is used as positive control whereas empty vector and P-galactosidase are used as negative controls. The specificity of IL-6 mediated STAT3-dependent activation of hFwe is examined by shRNA mediated silencing of STAT3 in IL-6 treated cells (STAT3 shRNA; p<1×10⁻⁴) (Technical replicates, n=5).

FIG. 20B: Schematic representation of the four DNA binding sites (DBS 1-4) on the hFwe promoter region is presented along with their location relative to the ORF. Notably, site 4 lies downstream of the hFwe ORF.

FIG. 20C: The occupancy of STAT3 on hFwe DBS is measured by ChIP using indicated antibodies in pooled normal ovarian, HGSC cancer and stromal matched tissue. A band in STAT3 DBS1-4 confirms the presence of STAT3 on all four DBS in HGSC cancer and HGSC stroma but absent in normal ovarian tissue.

FIG. 20D: Luciferase assay is performed on minimal hFwe promoter with each of the four putative DBS sites (DBS 1-4) in the presence or absence of IL-6. c-Fos promoter (control STAT3 DBS) is used as positive control whereas empty vector and β-galactosidase are negative controls. The specificity of IL-6 mediated STAT3-dependent activation of hFwe is examined by shRNA mediated silencing of STAT3 in IL-6 treated cells (STAT3 shRNA; p<1×10⁻⁵) (Technical replicates, n=5).

FIG. 20E: The occupancy of STAT3 on each of the four putative DBS sites on hFwe promoter is measured by ChIP using indicated antibodies in pooled normal ovarian, HGSC cancer and stromal matched tissue. The result of fold enrichment ±SE relative to input is depicted p<1×10⁻¹⁰) (Biological replicates, n=5).

FIG. 20F: The occupancy of pSTAT3^{Y705} on each of the four putative DBS sites on hFwe promoter is measured by ChIP using indicated antibodies in pooled normal ovarian, HGSC cancer and stromal matched tissue. Results show that pSTAT3^{Y705} binds to all the four STAT3 DBS (STAT3 DNA-BS-1-4) in the HGSC tumor and stroma tissue samples. Data from qPCR-based analysis is depicted as fold enrichment relative to input. The respective p-values are shown on the plot (p<1×10⁻¹⁰) (Biological replicates, n=5).

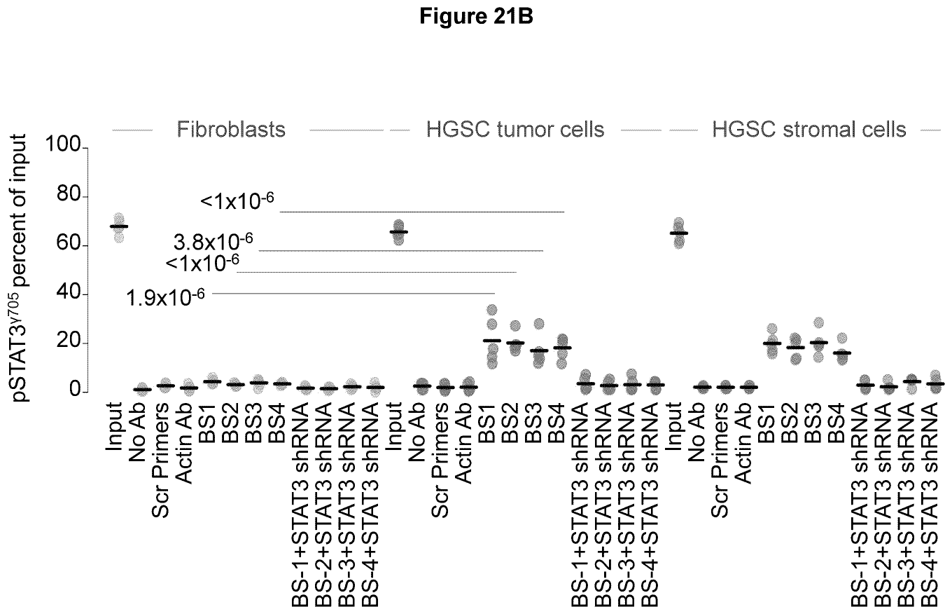

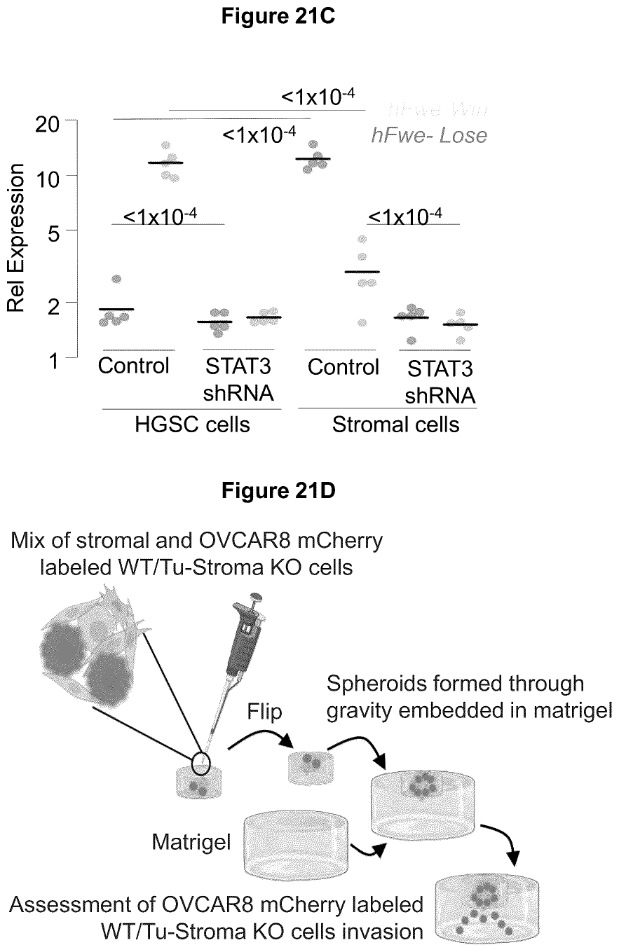

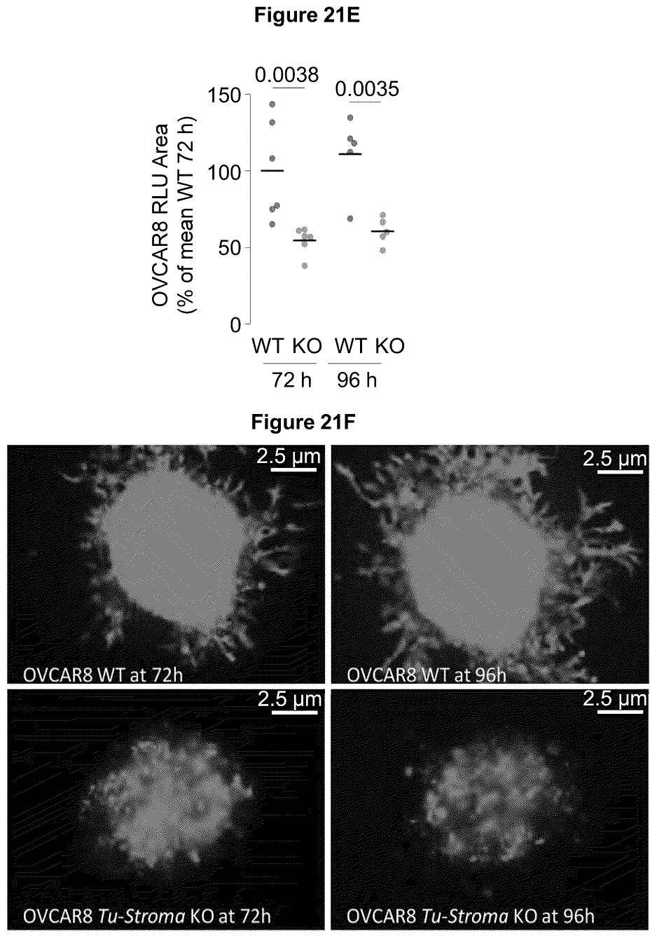

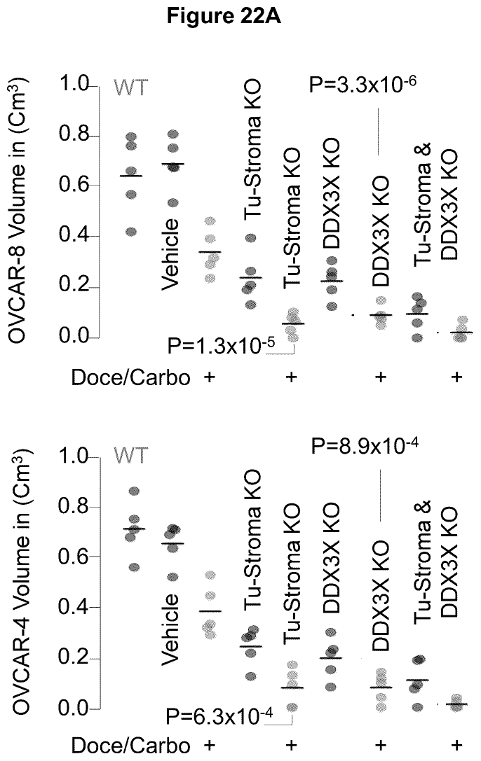

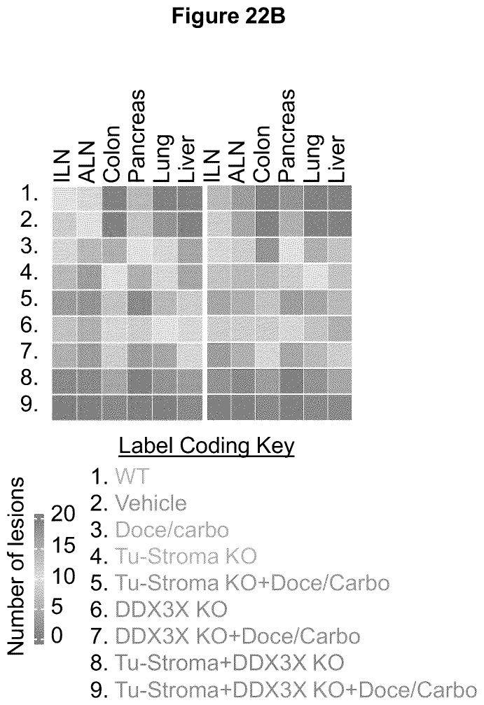

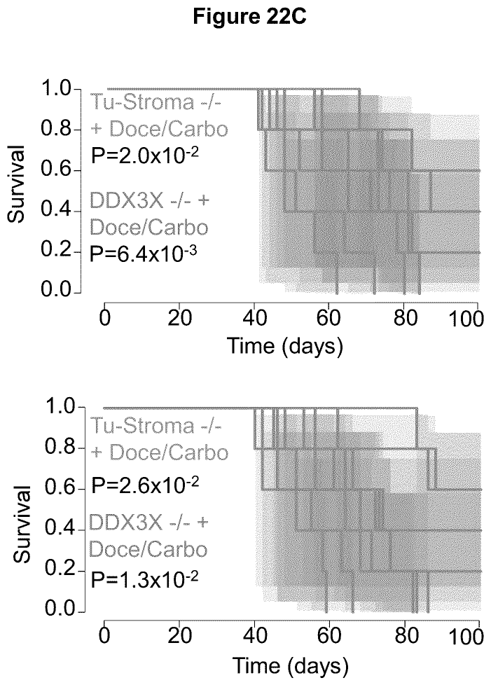

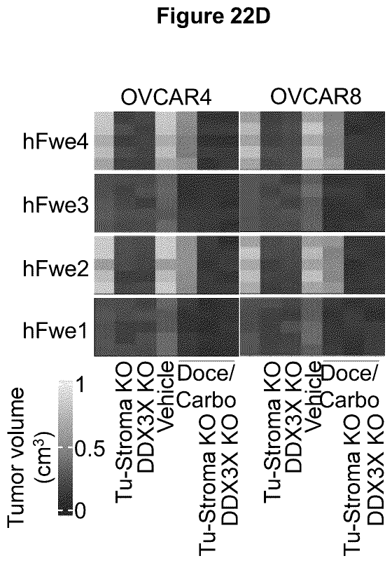

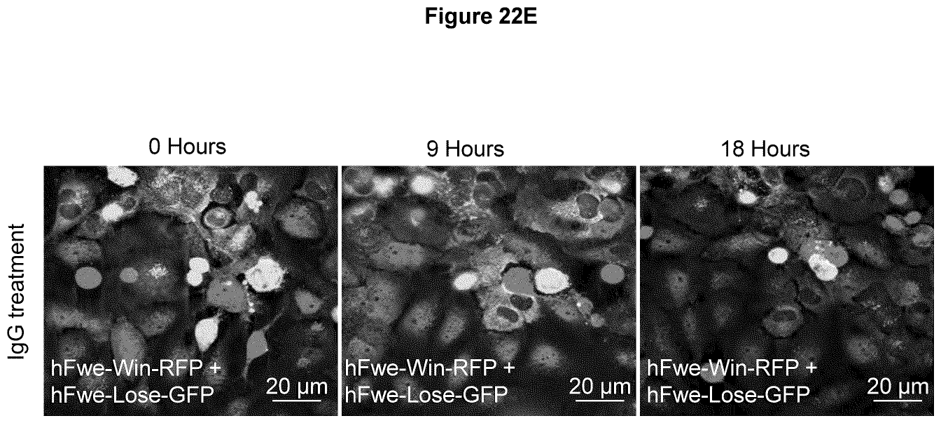

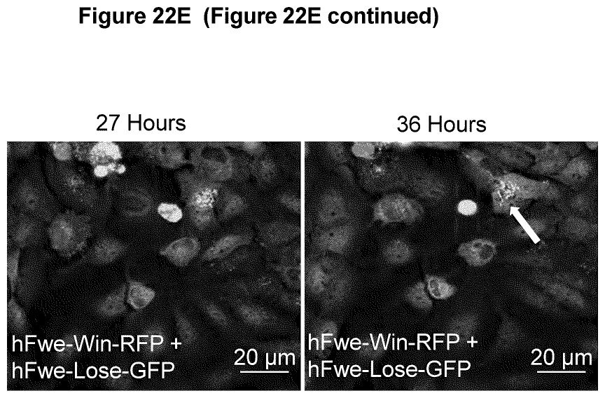

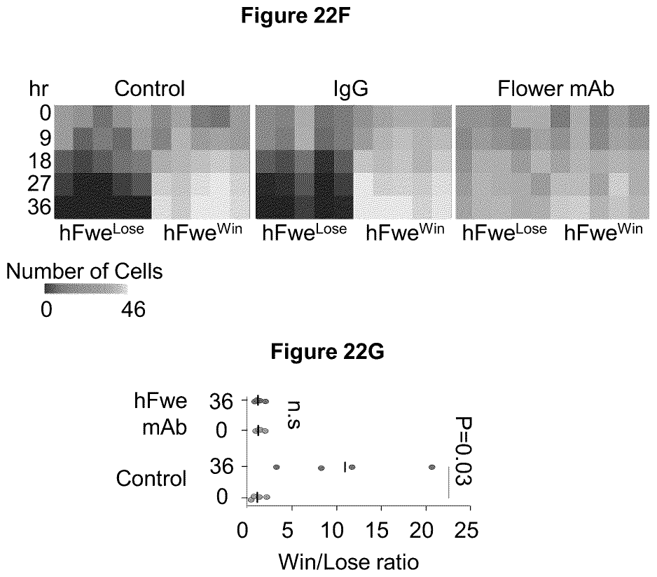

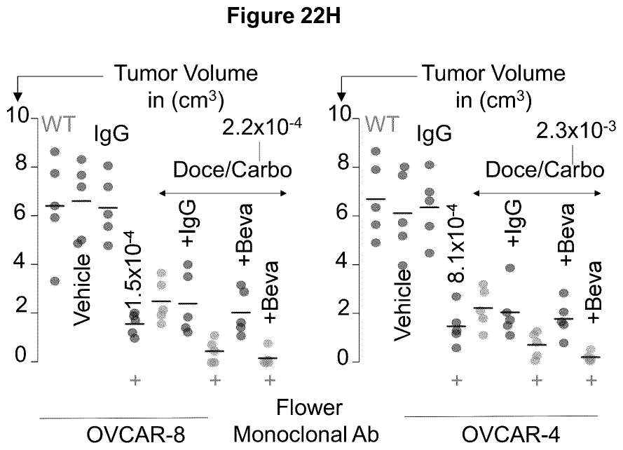

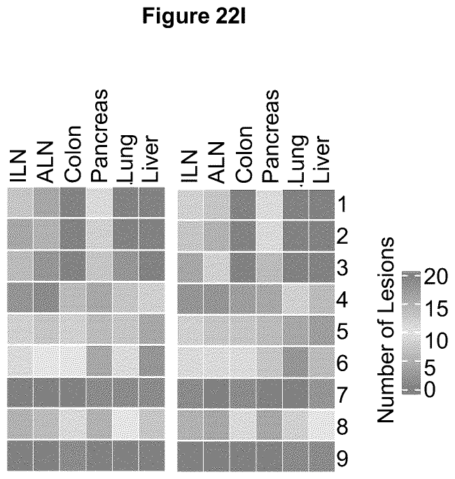

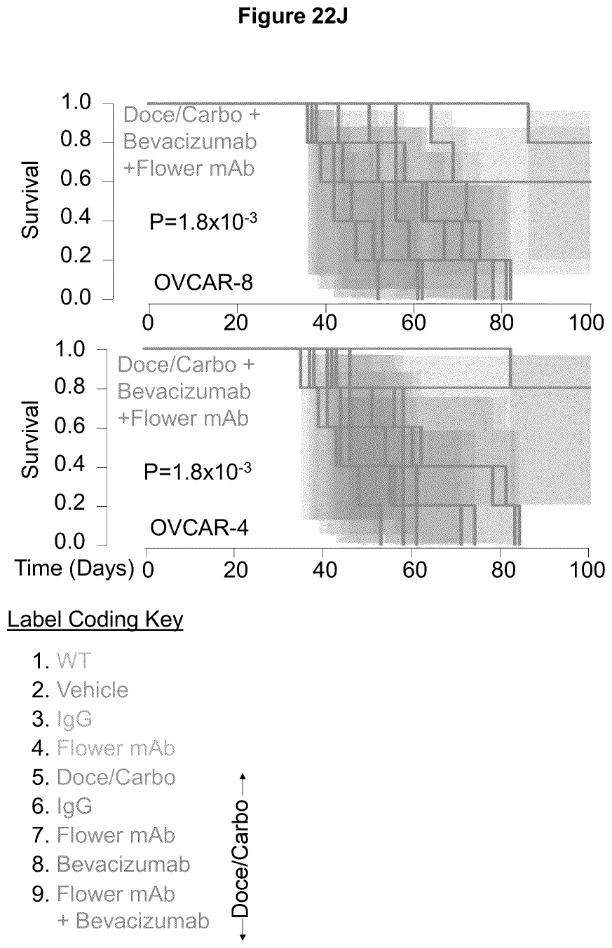

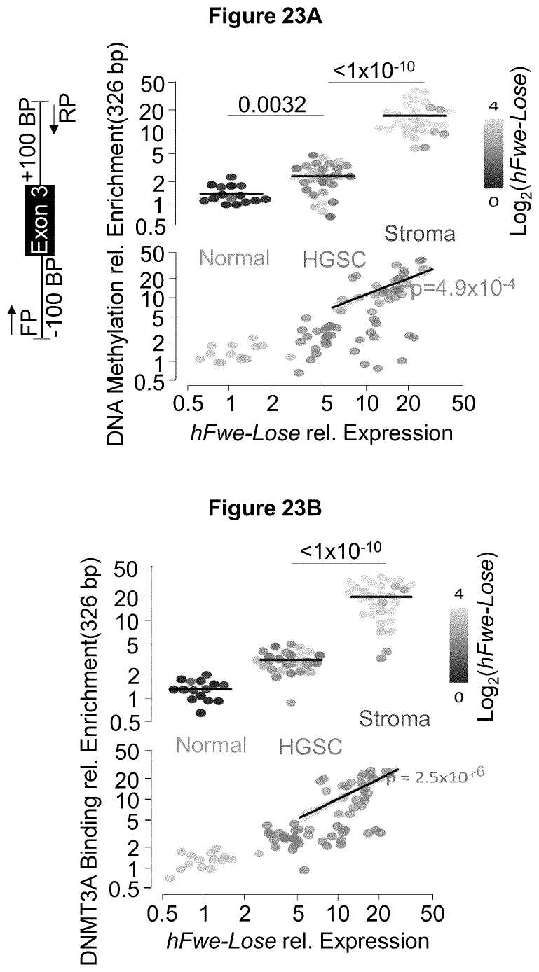

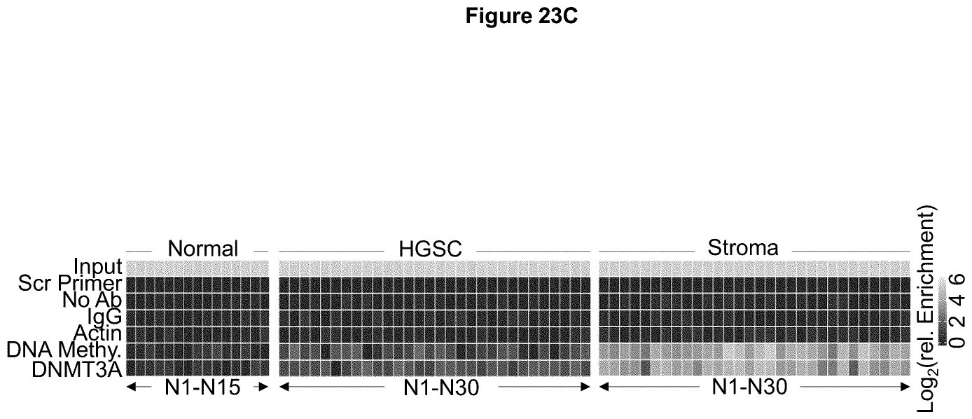

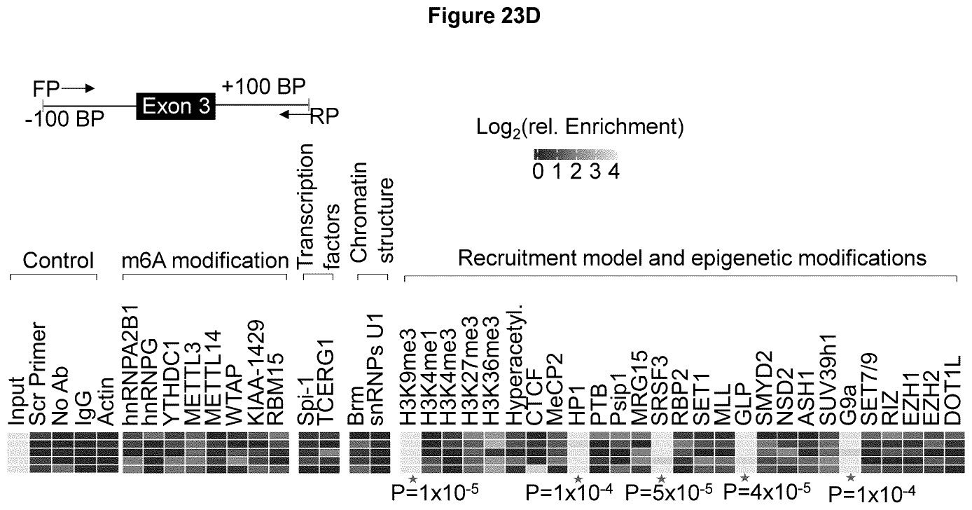

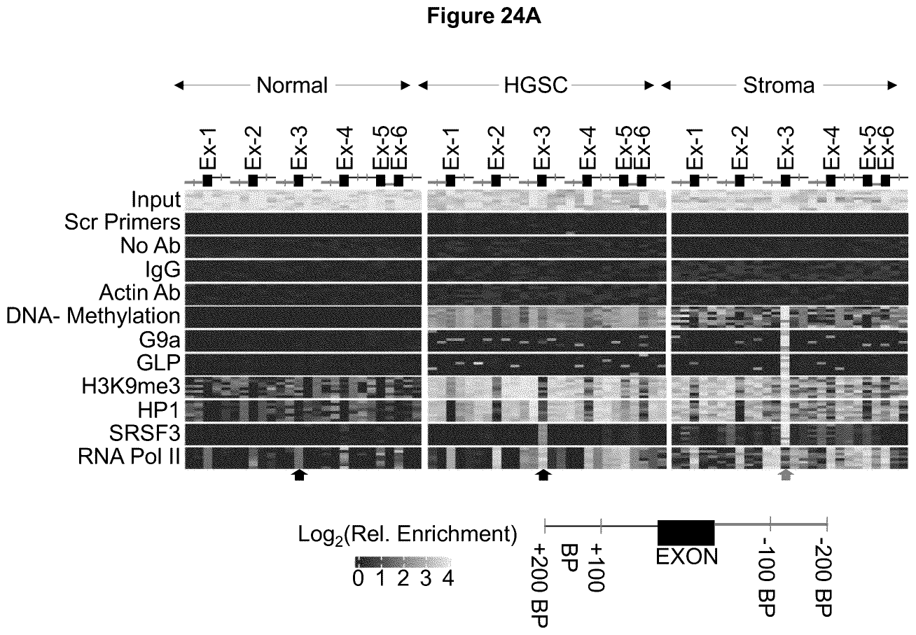

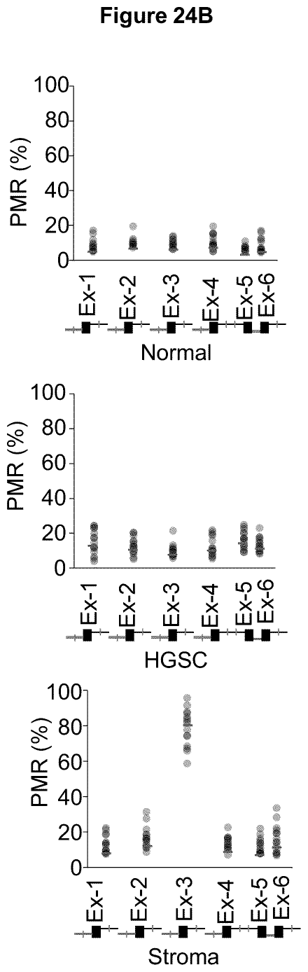

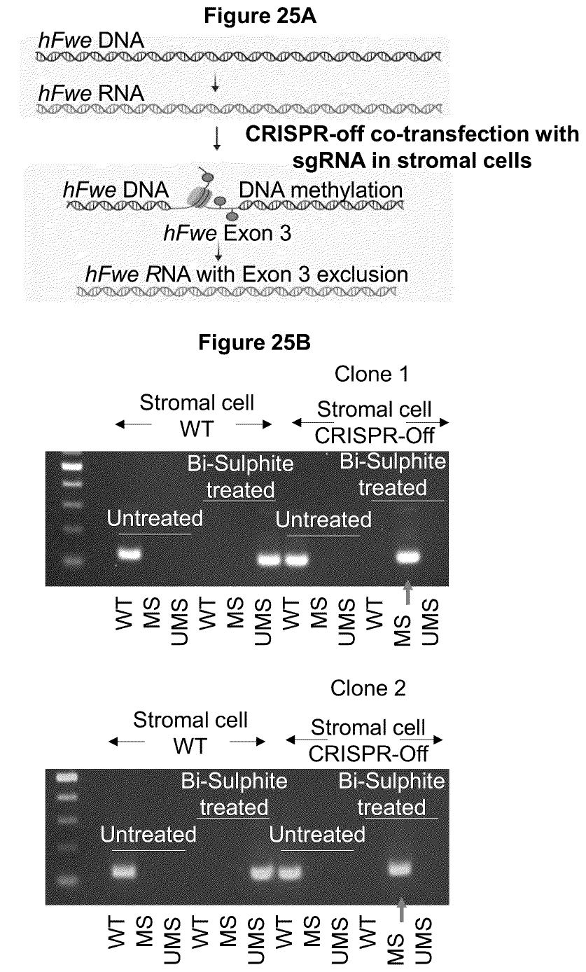

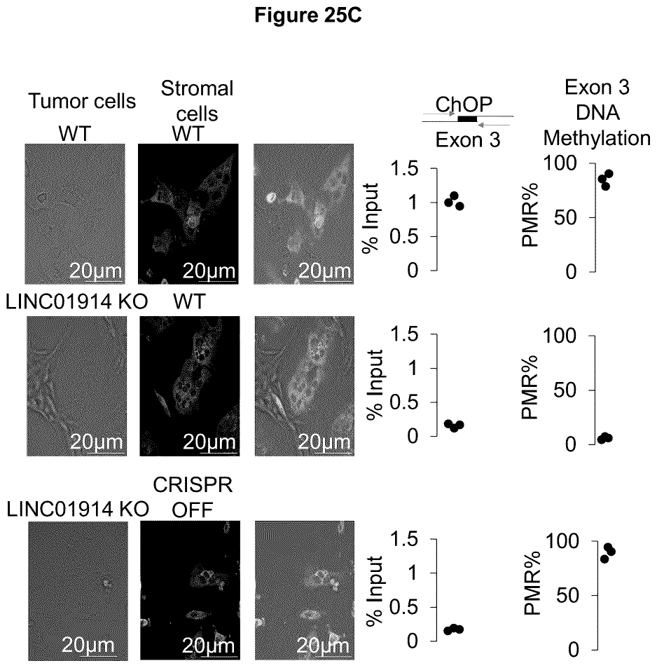

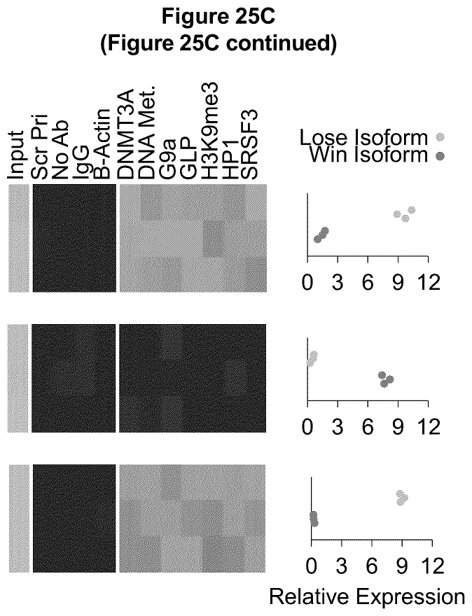

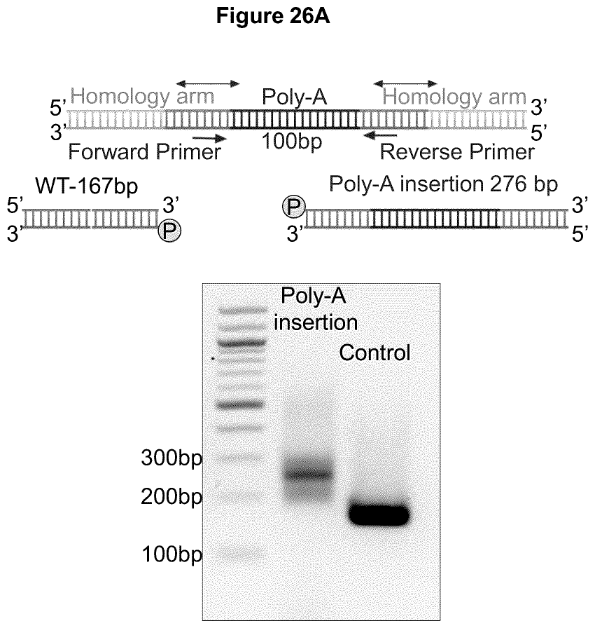

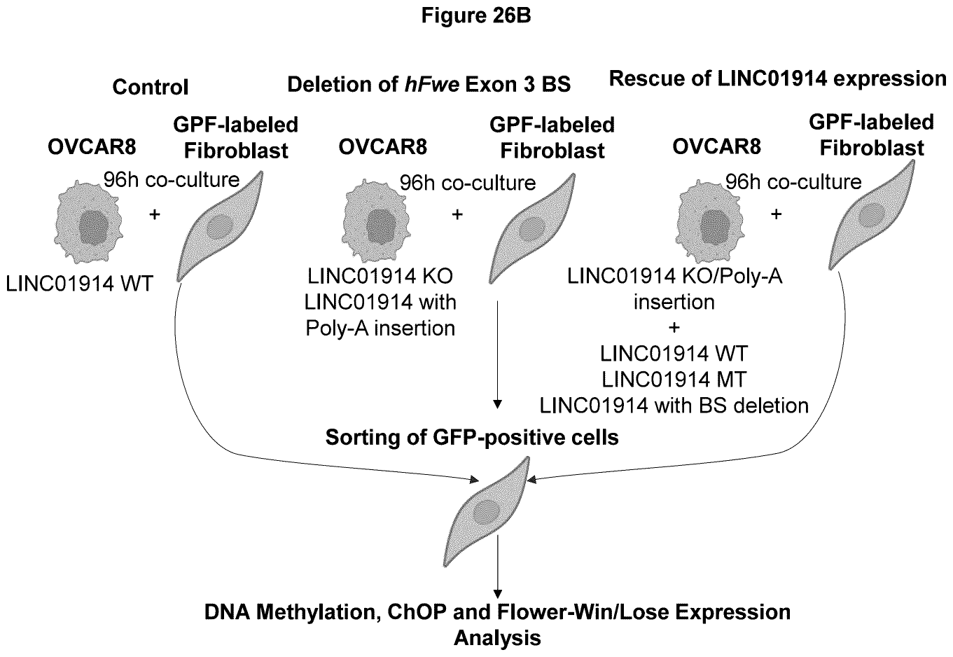

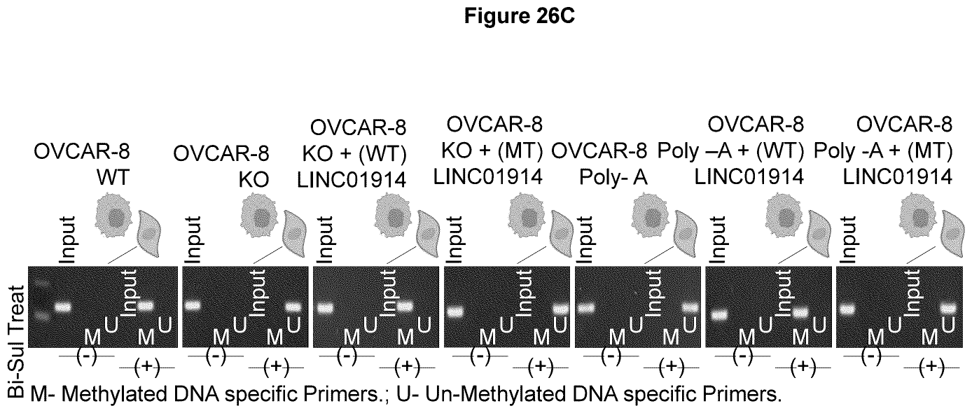

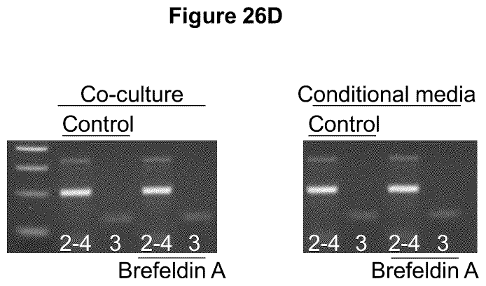

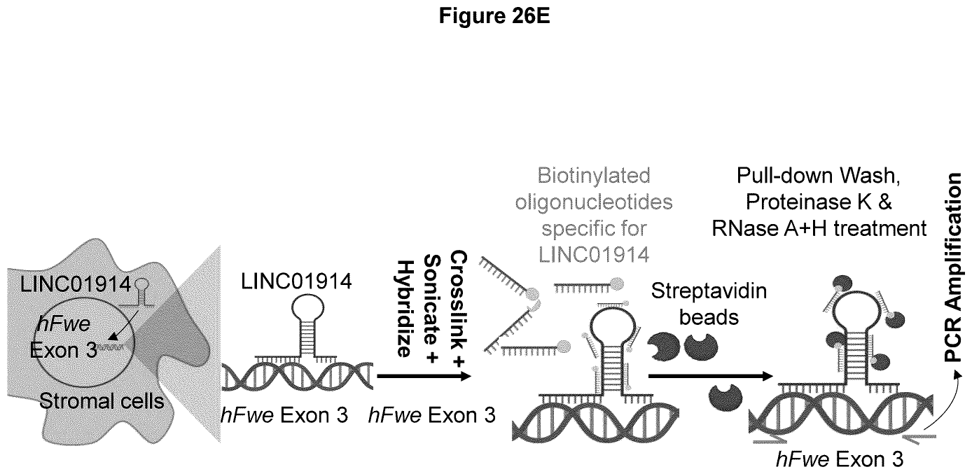

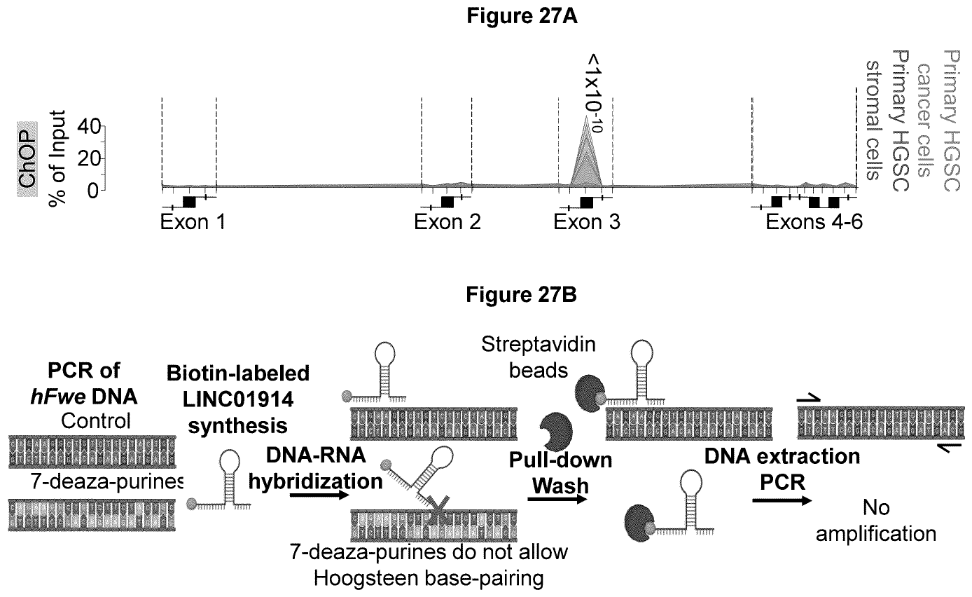

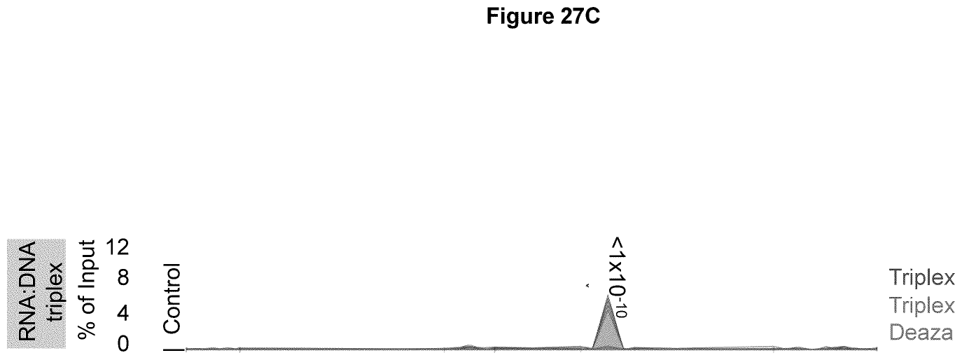

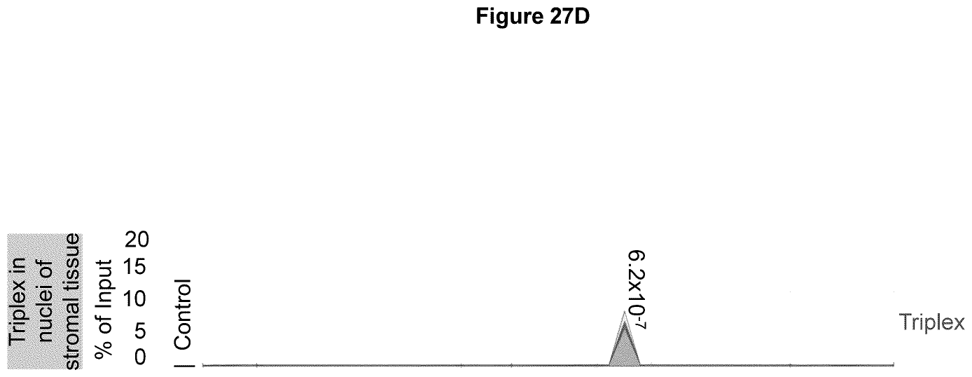

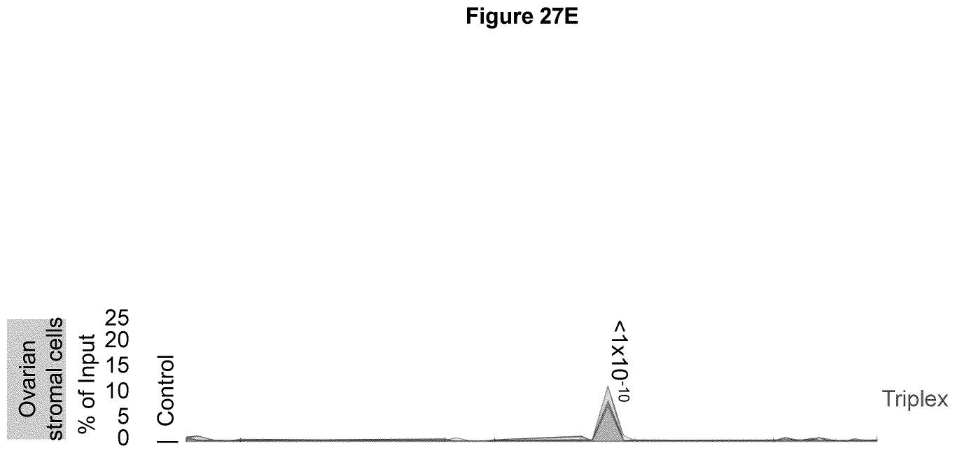

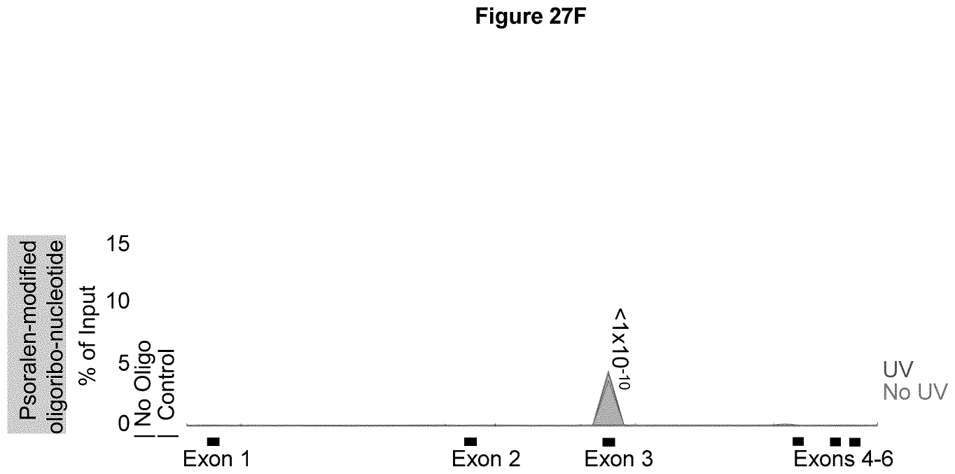

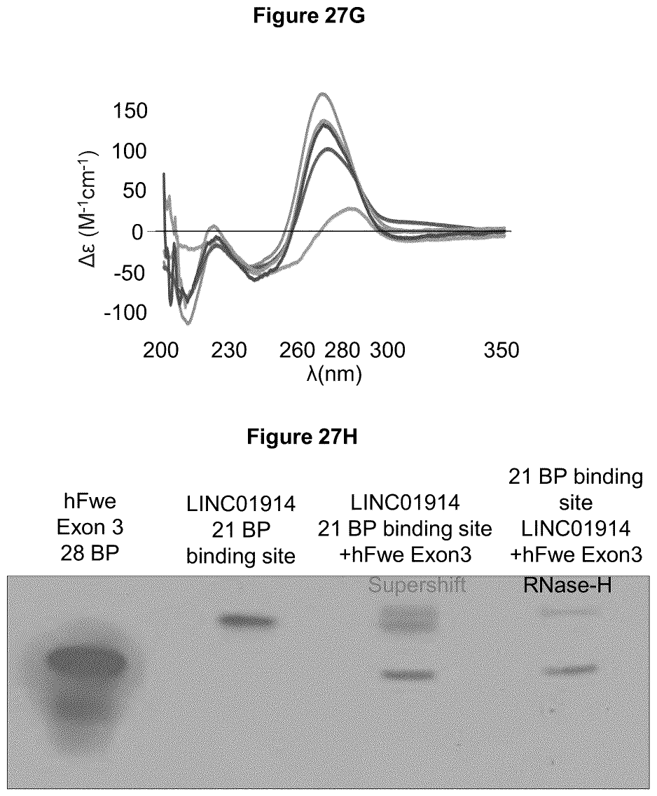

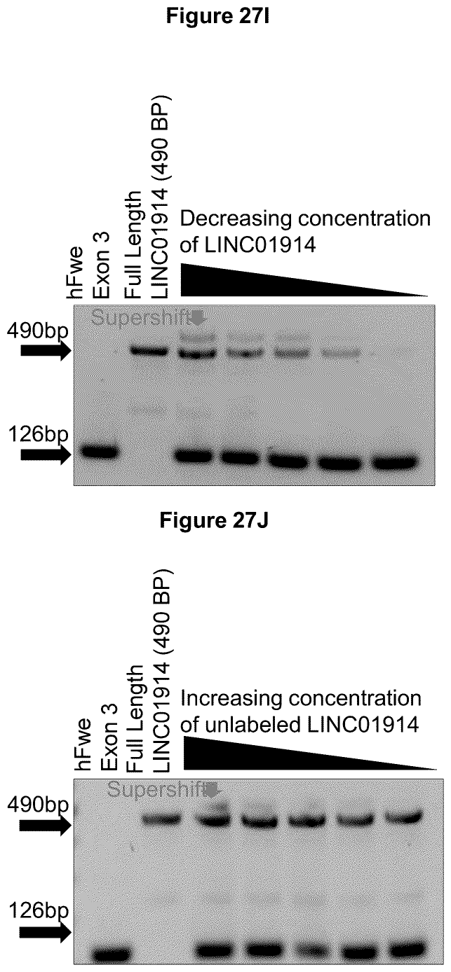

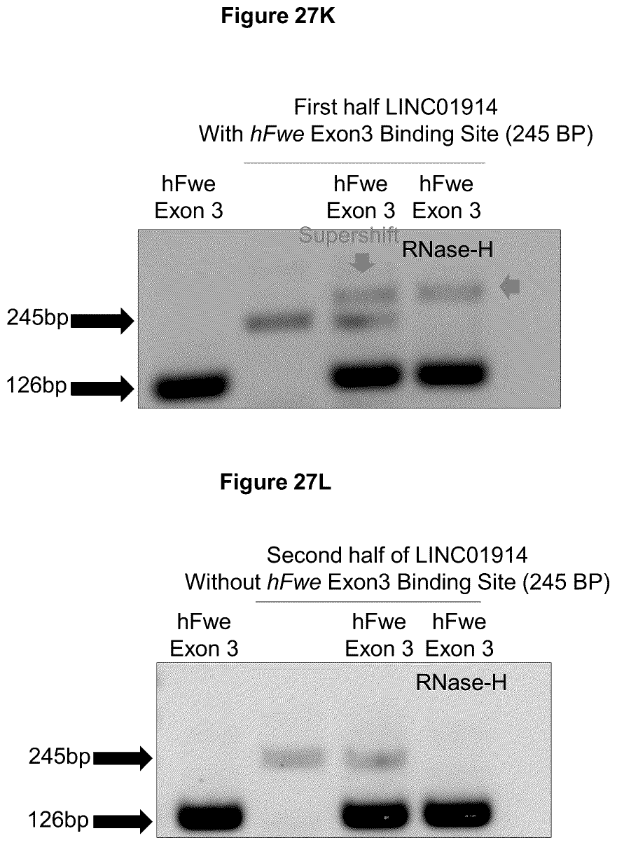

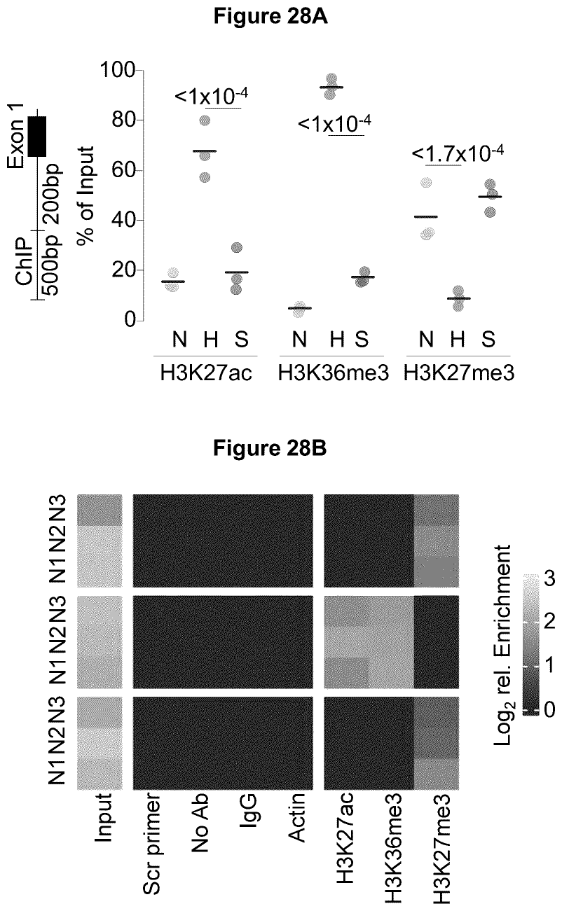

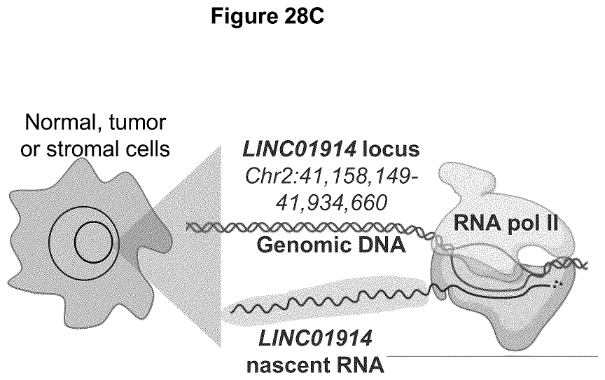

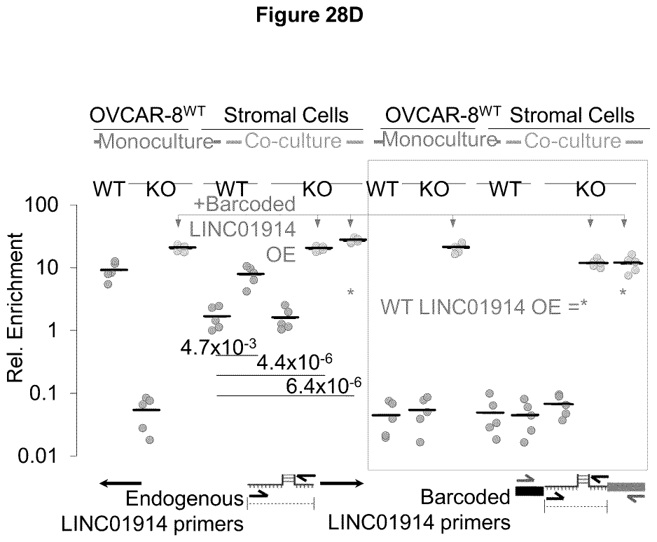

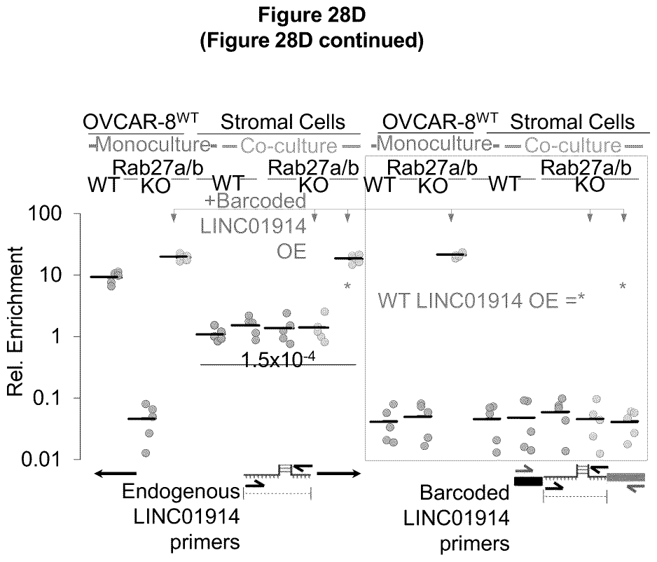

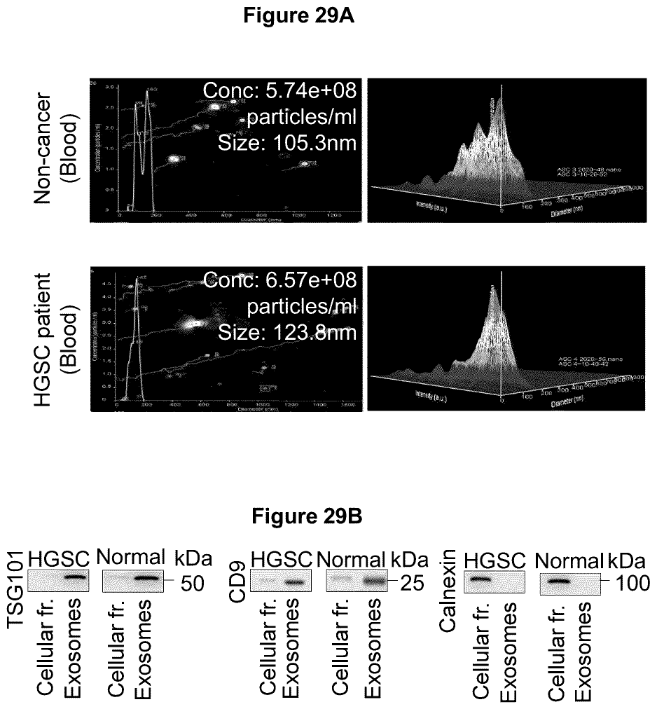

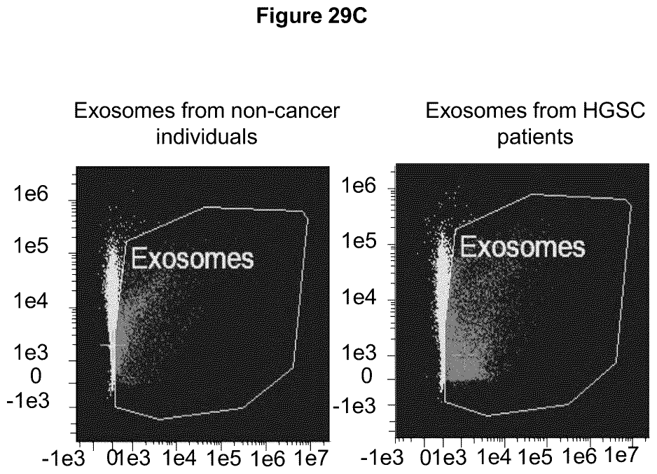

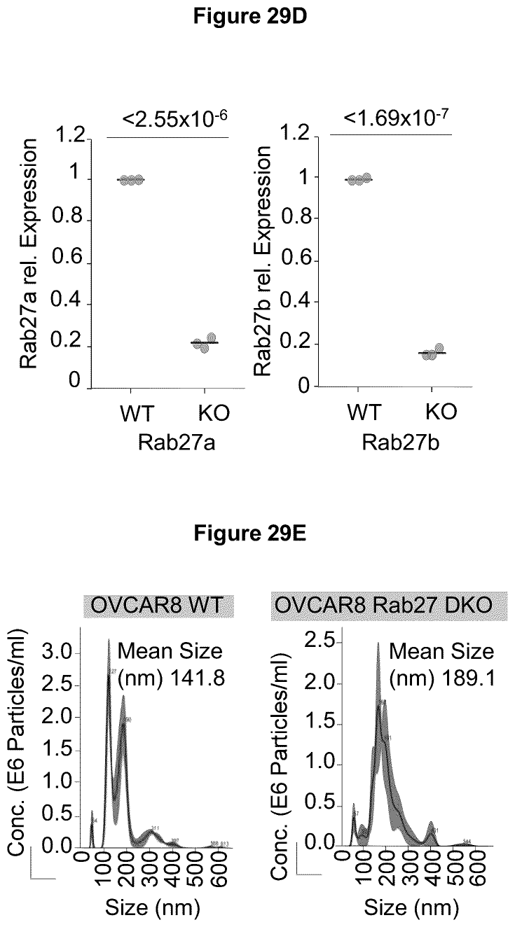

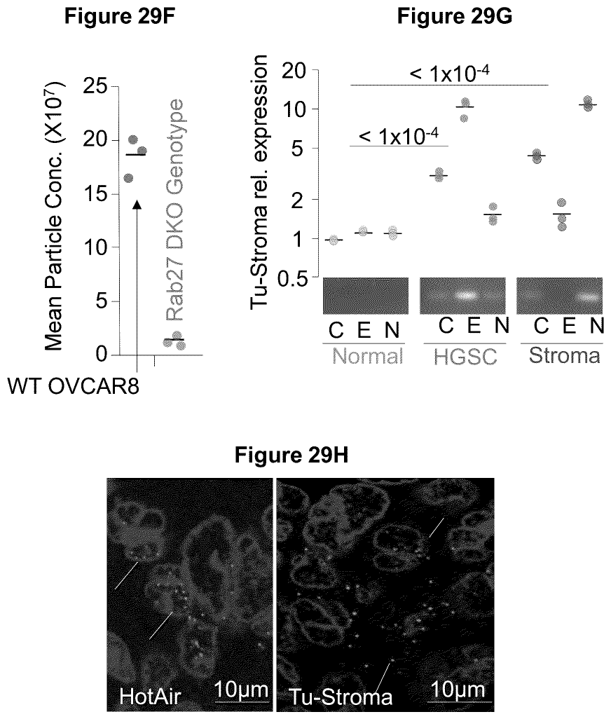

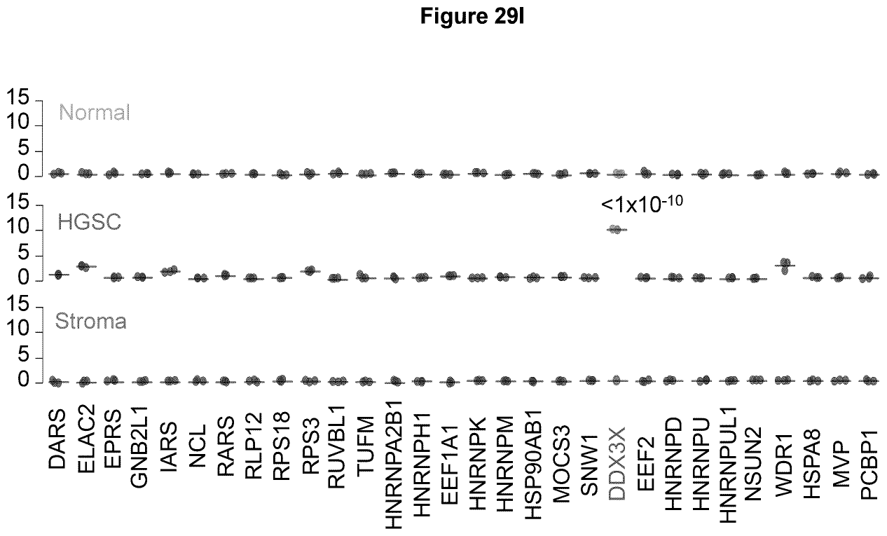

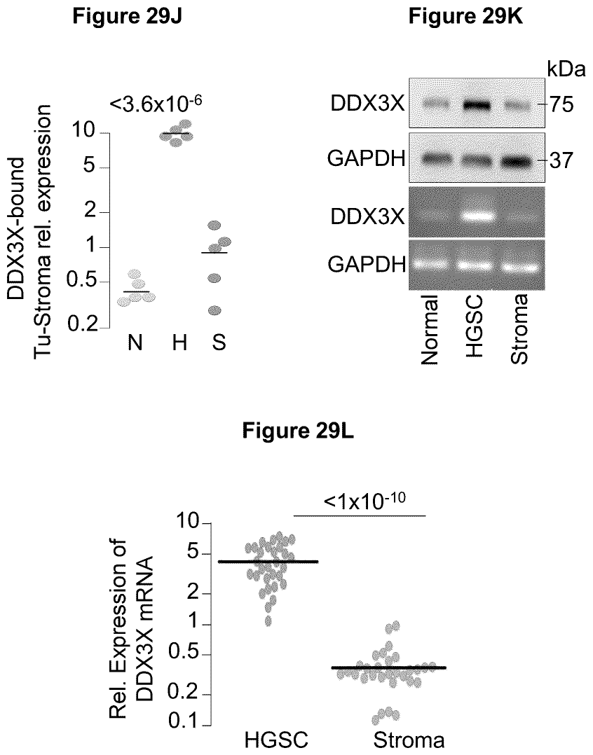

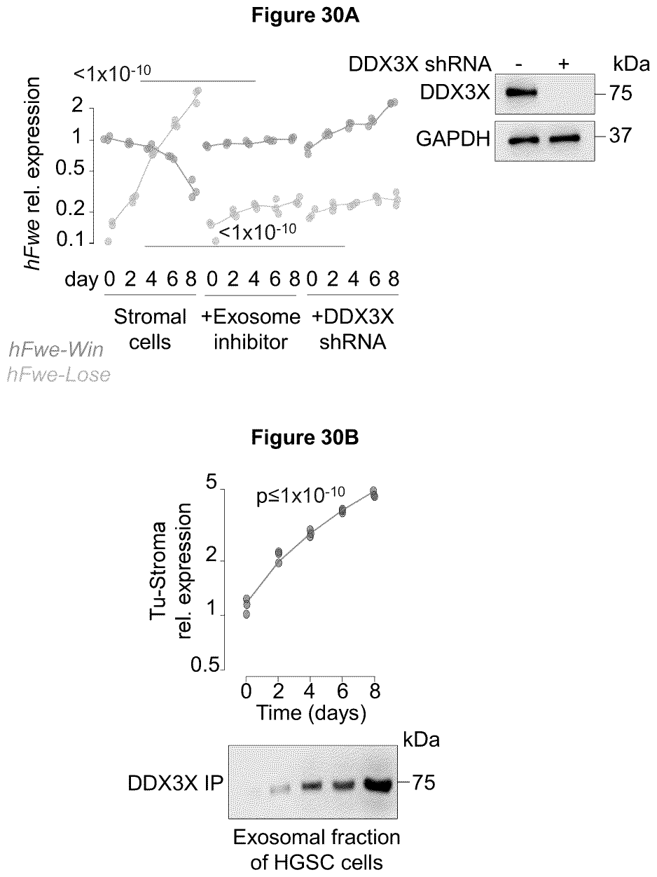

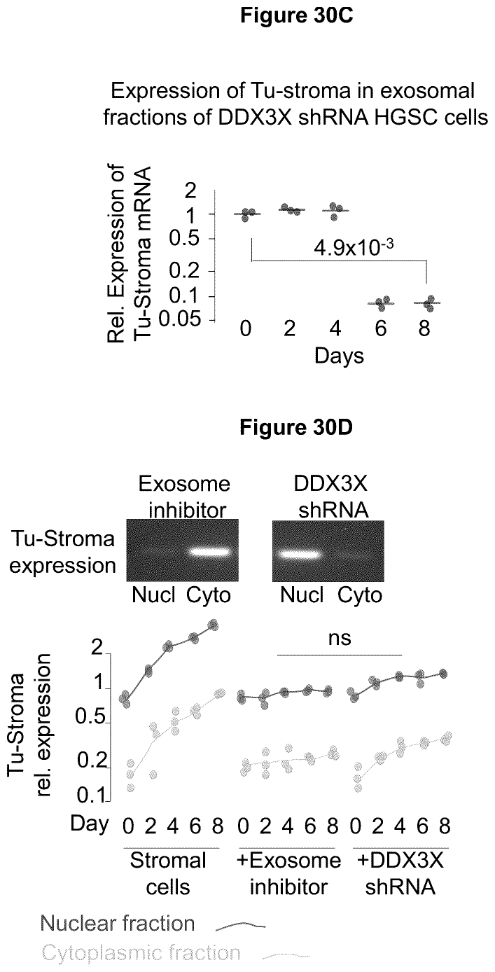

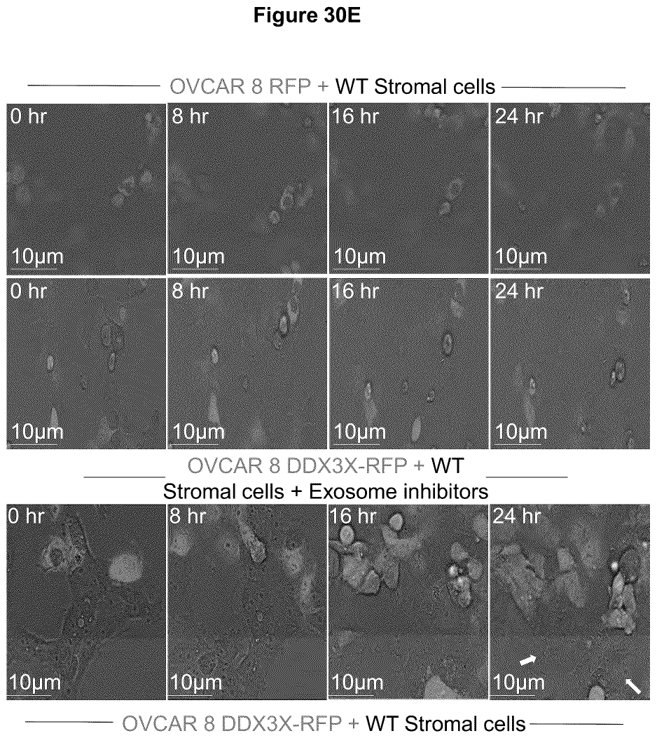

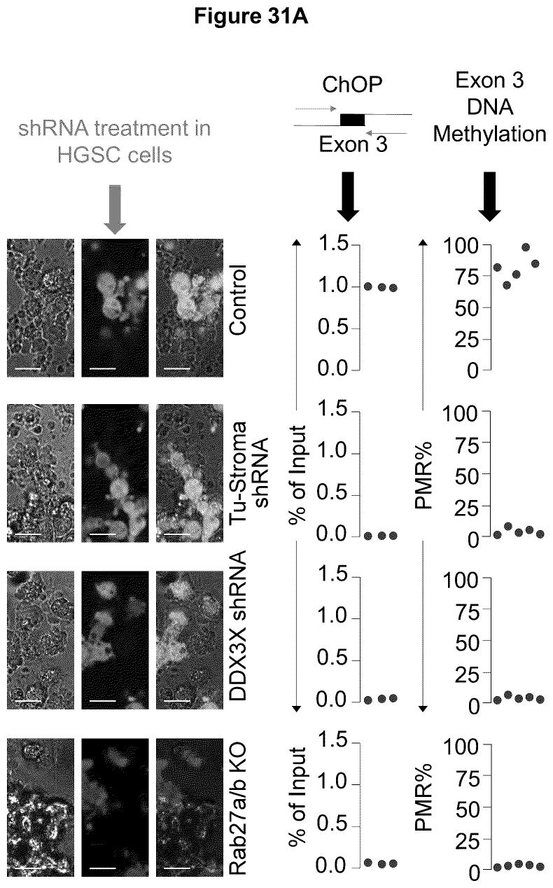

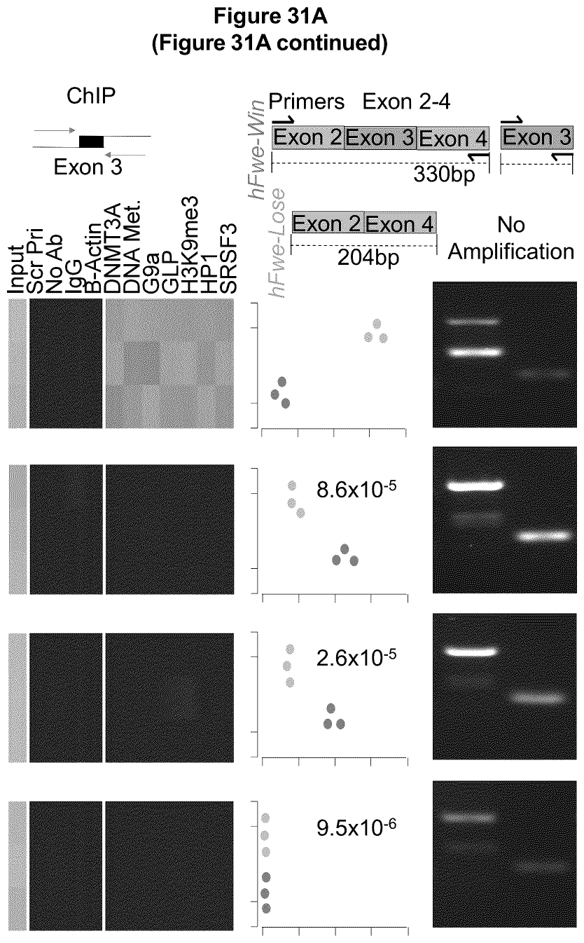

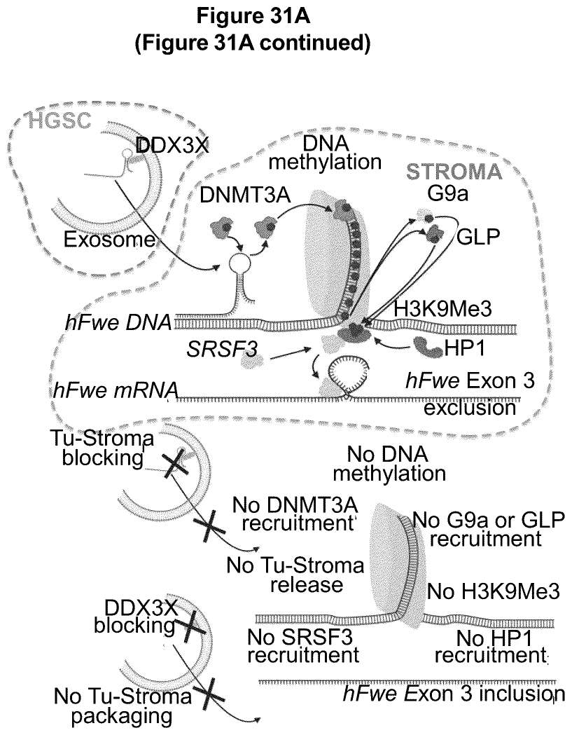

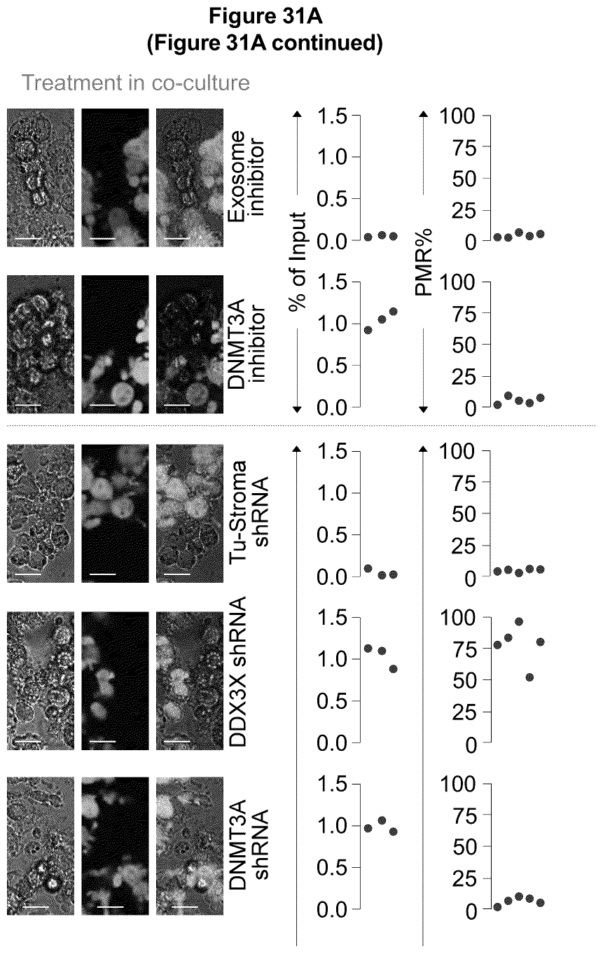

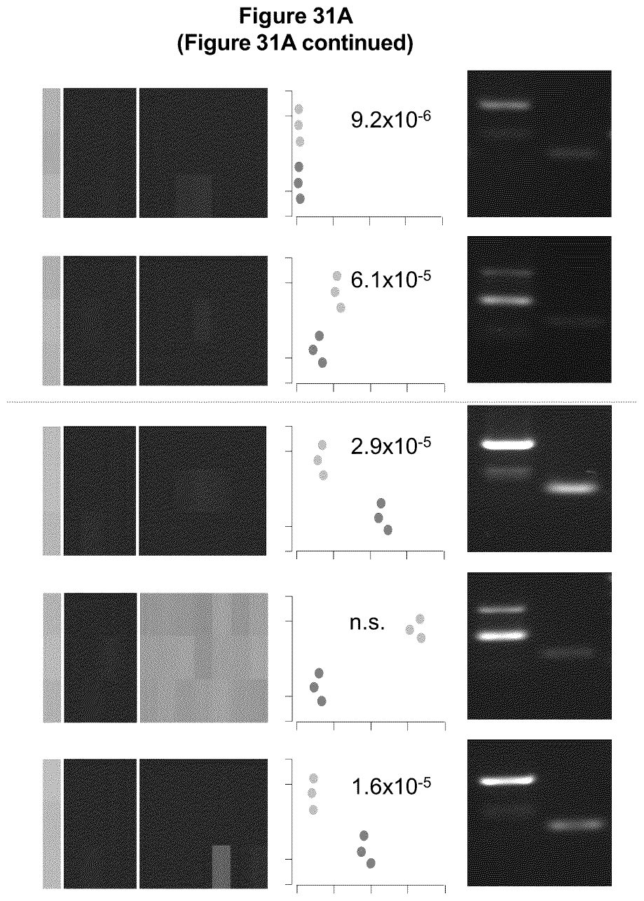

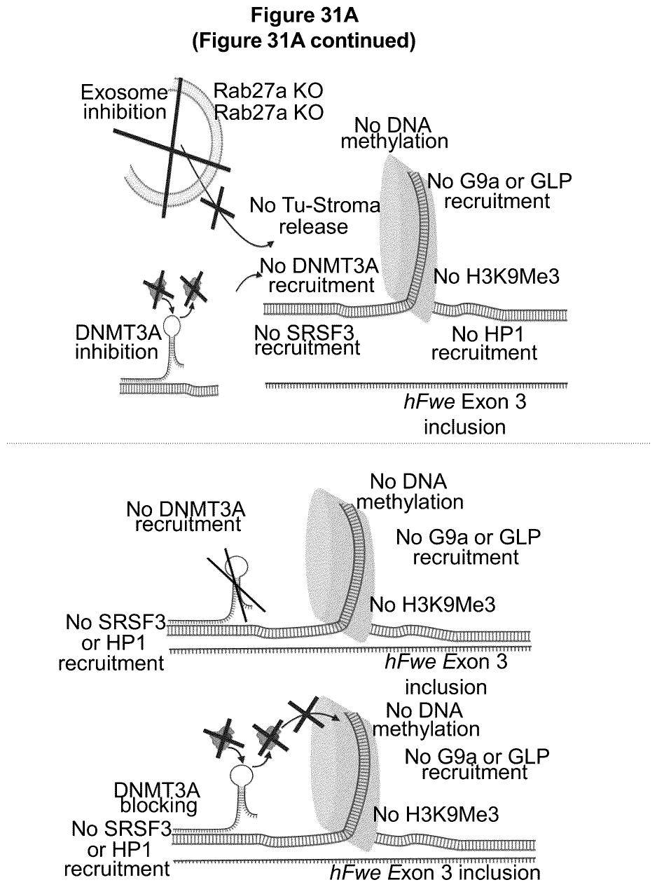

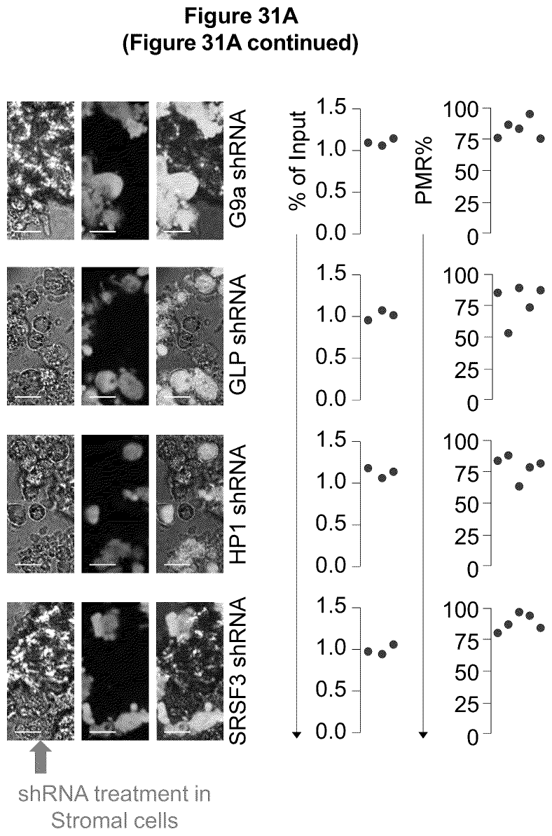

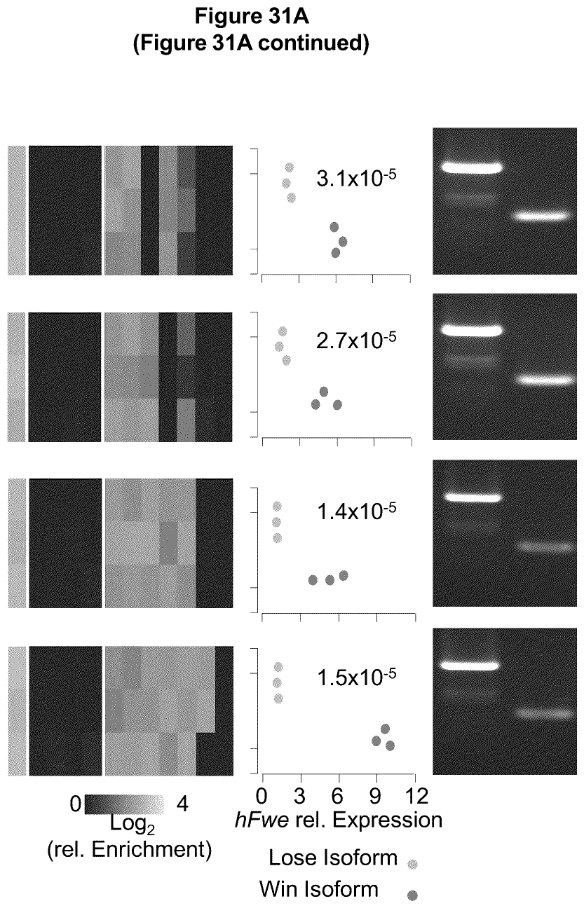

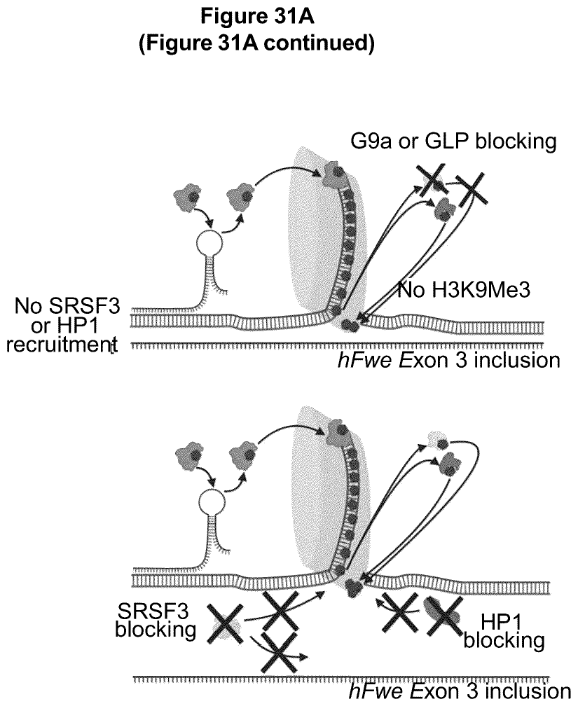

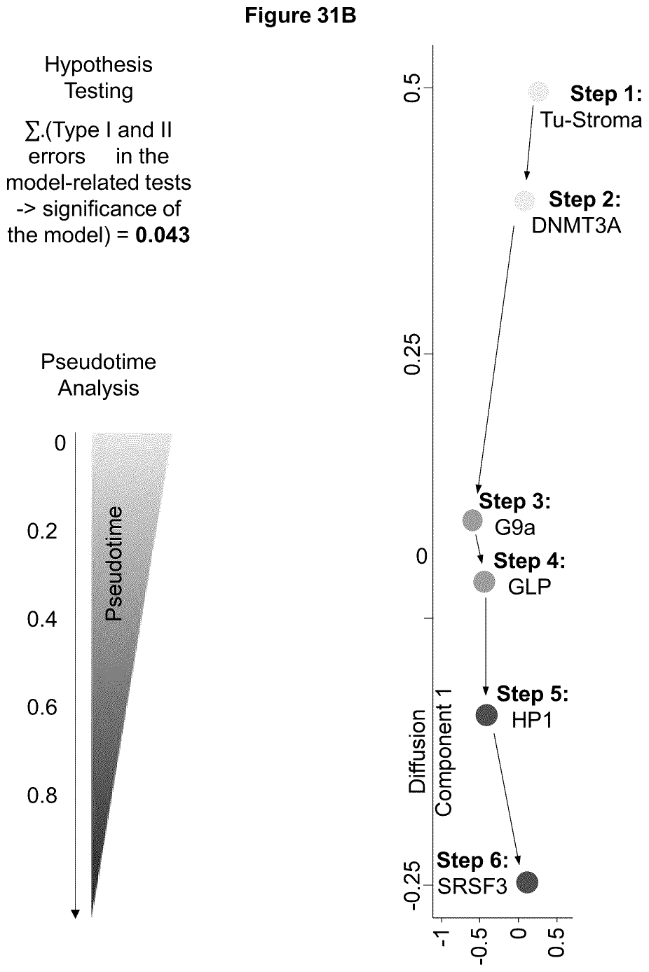

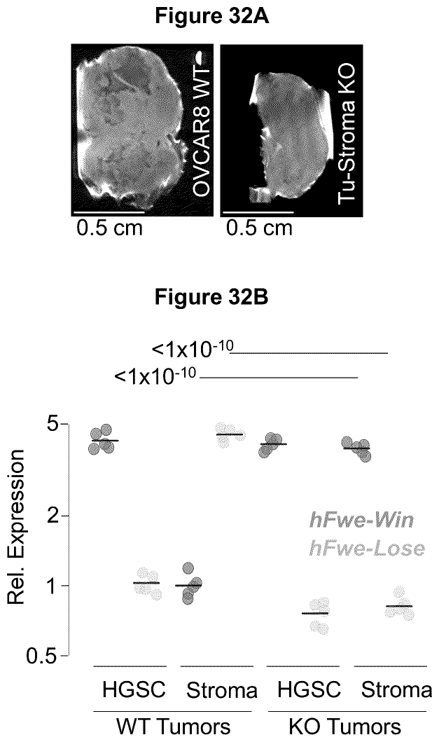

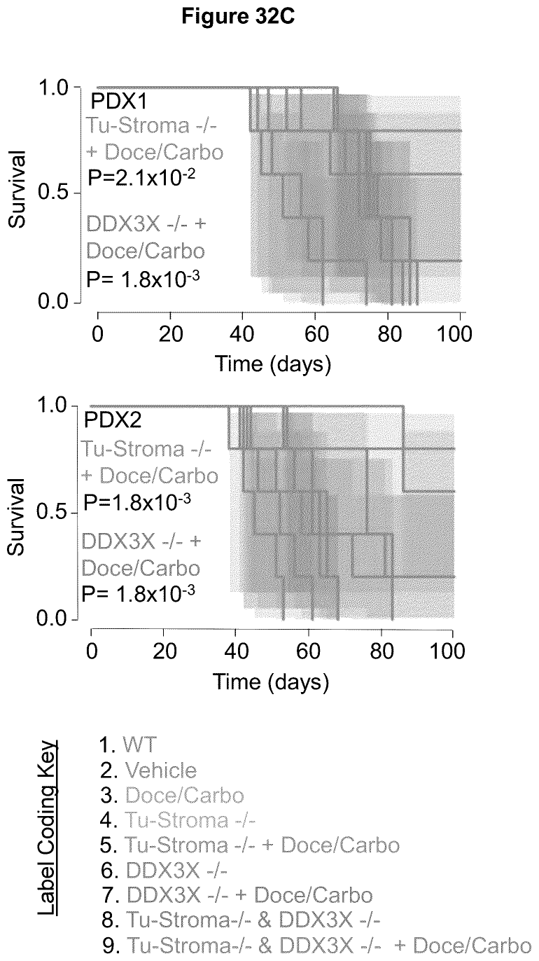

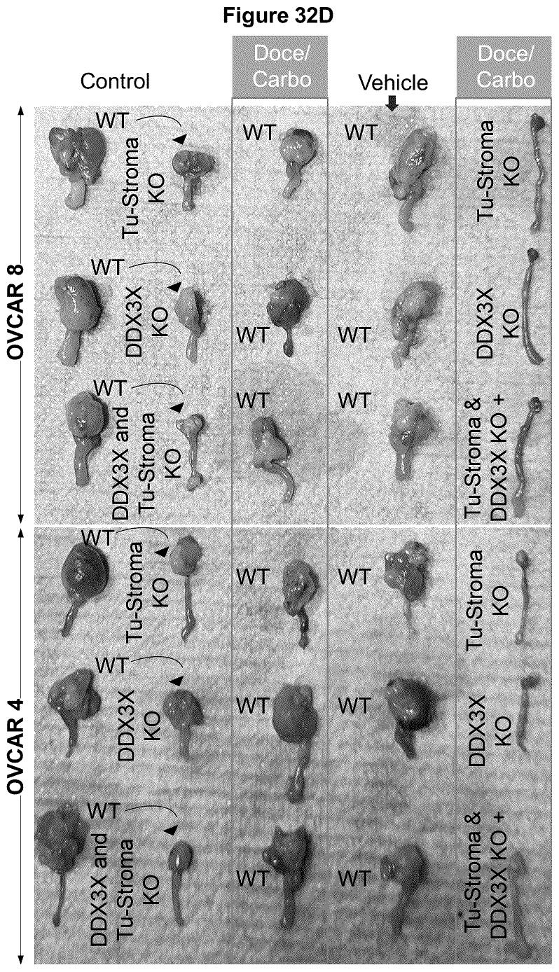

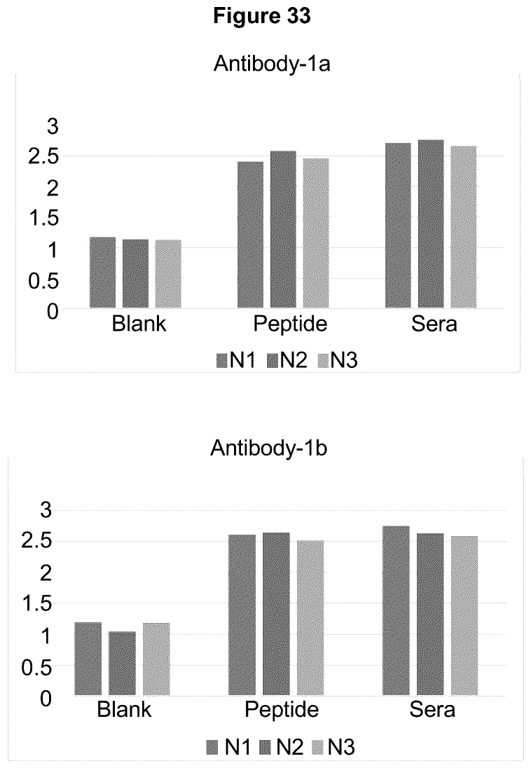

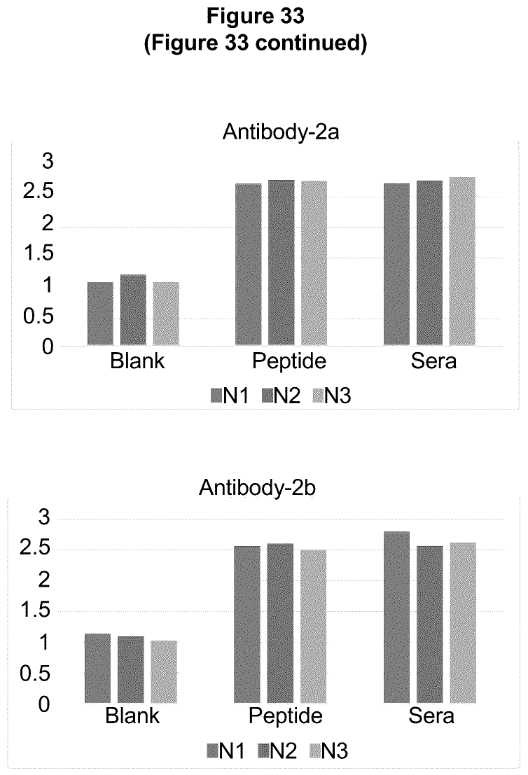

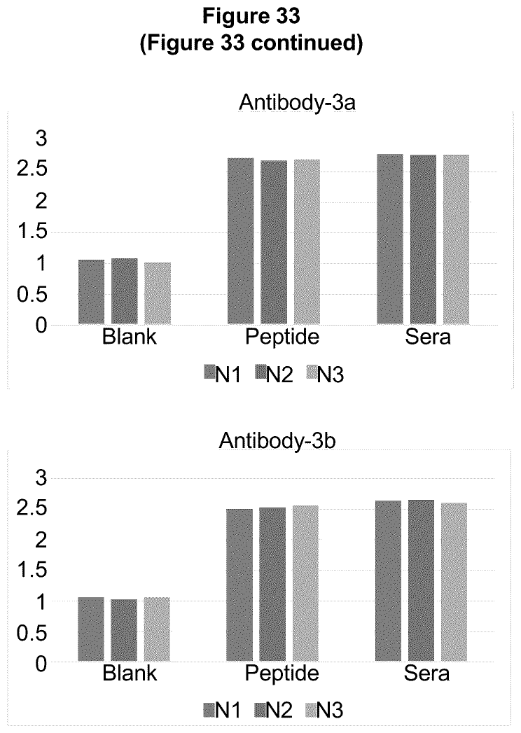

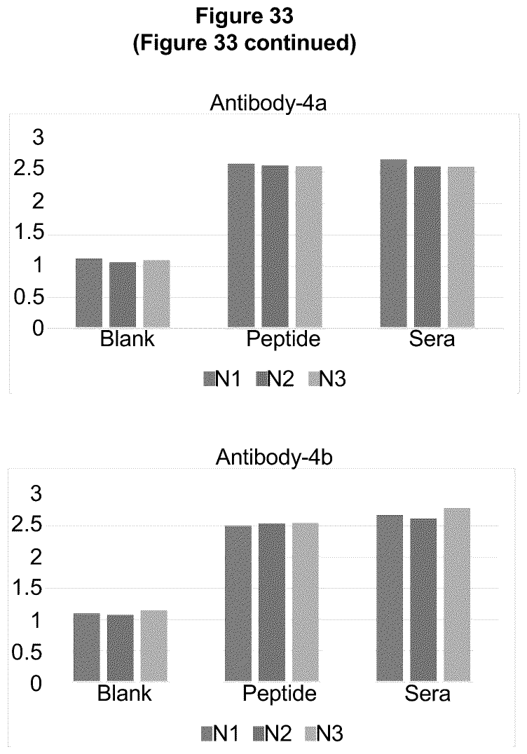

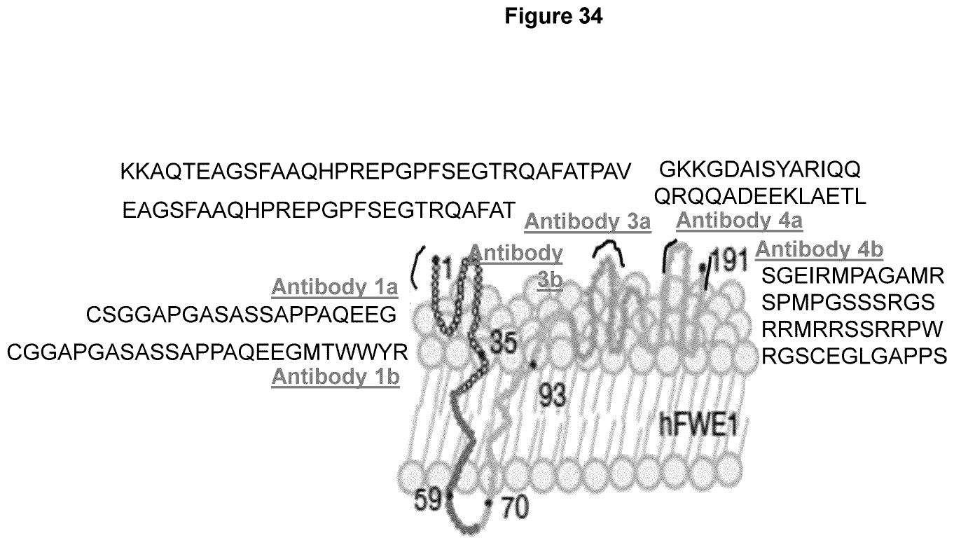

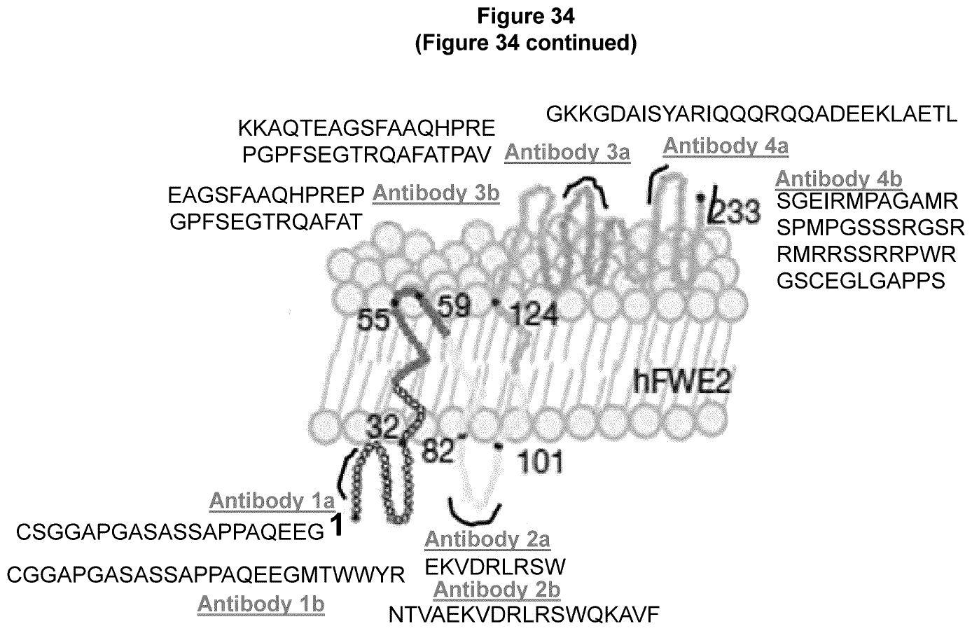

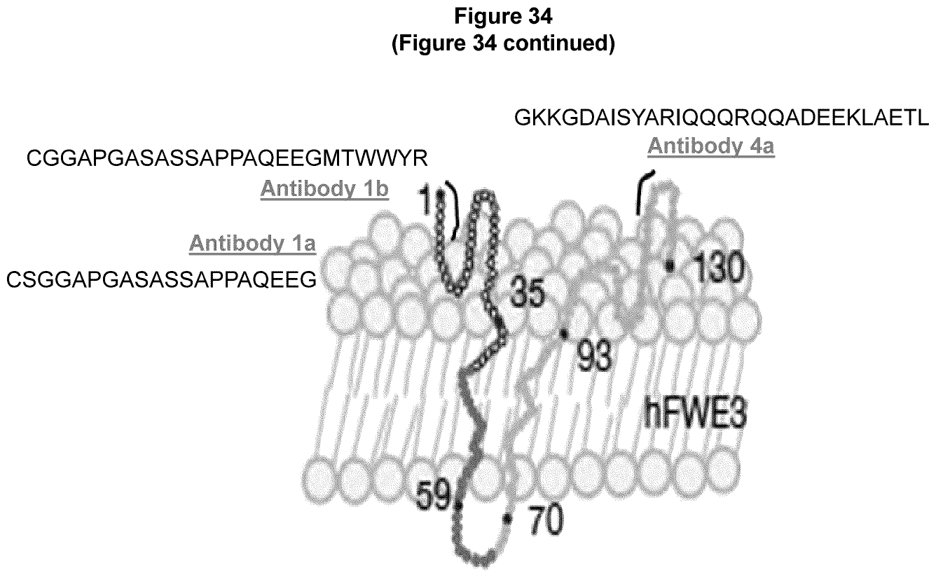

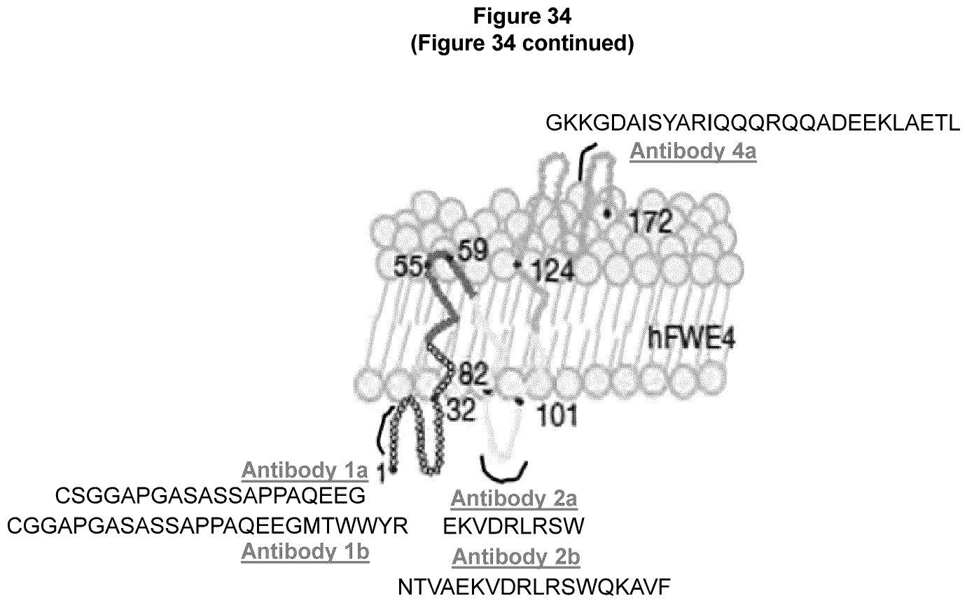

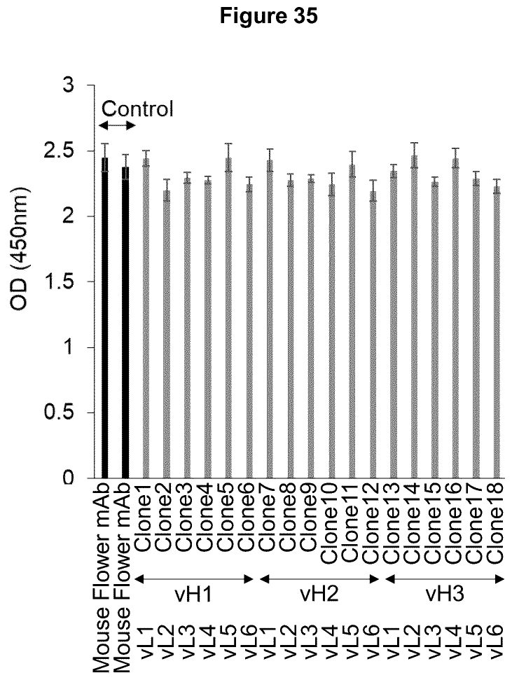

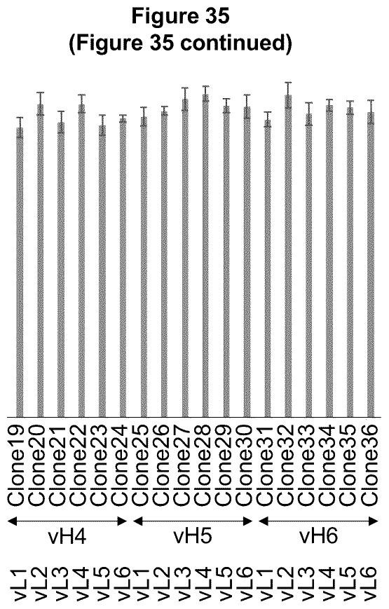

FIGS. 21A-21F show transcriptional and epigenetic regulation of Flower isoform expression promotes tumor growth.

FIGS. 21A-21B: Relative occupancy of STAT3 (FIG. 21A) and pSTAT3^{Y705} (FIG. 21B) is measured by qChIP for the indicated antibodies on DBS1-4 sites in primary cultured fibroblasts, HGSC tumor cells and HGSC stromal cells in the presence or absence of STAT3 shRNA. Results show a high fold enrichment on all the four STAT3-DBS in co-cultured and separated HGSC tumor and HGSC stromal cells, however, silencing of STAT3 using STAT3 shRNA, abrogates the effect. No antibody (no Ab), scrambled primer (scr. prim.) and actin antibody are used as negative controls. Data from qPCR-based analysis is depicted as fold enrichment relative to input. The respective p-values are shown on the plot (Technical replicates, n=5).

FIG. 21C: HGSC and stromal cells are co-cultured for 8 days in the presence or absence of STAT3 shRNA. HGSC and stromal cells are then sorted. hFwe-Win and hFwe-Lose expression is analyzed using RT-qPCR and the relative fold change ±SE is depicted p<1×10⁻⁴) (Technical replicates, n=5).

FIG. 21D: Schematic outline depicting the OVCAR8 growth and invasion assay. Briefly, spheroids containing Tu-Stroma WT or KO OVCAR8 cells labeled with mCherry were grown in hanging drops and subsequently embedded in Matrigel.

FIG. 21E: The capacity of OVCAR8 WT and Tu-Stroma KO cells to invade the surrounding Matrigel is assessed (n=5). OVCAR8 growth and invasion is measured at 72 h and 96 h and is expressed as relative light units (RLU) as percent WT cells at 72 h and 96h. Tu-Stroma KO significantly redSuces the invasiveness of OVCAR8 after 72 (p=3.8×10⁻³) and 96 hours (p=3.5×10⁻³).

FIG. 21F: Representative images of data in FIG. 21E are shown.

FIGS. 22A-22J show Tu-Stroma KO, DDX3X KO, and Flower monoclonal antibody therapy reduce tumor size and improve survival in orthotopic HGSC models.

FIG. 22A: Tumor volumes of each of the cohorts in FIG. 32D are shown for both OVCAR8 (top) and OVCAR4 (bottom) groups. Red dots identify the groups treated with Docetaxel/Carboplatin therapy. The p-values compare the tumor volume at the endpoint of Tu-Stroma KO in combination with chemotherapy (OVCAR8: p=1.3×10⁻⁵; OVCAR4: 6.3×10⁻⁴) and DDX3X KO in combination with chemotherapy (OVCAR8: p=3.3×10⁻⁶; OVCAR4: 8.9×10⁻⁴) with the control (Biological replicates, n=5).

FIG. 22B: The heatmaps show the number of lesions detected in the liver, axillary lymph nodes, colon, pancreas, lung, and inguinal lymph nodes at the endpoint of the experiment described in FIG. 32D and FIG. 22A (top). A colored and numbered label coding key is shown to identify the groups used in the experiment (bottom).

FIG. 22C: Kaplan-Meier curves of each group used in OVCAR8 and OVCAR4 orthotopic experiment shown in FIG. 32D. Each group line is colored as per the label coding key shown in FIG. 22B. The p-values show the comparison in survival between Tu-Stroma KO in combination with chemotherapy (OVCAR8: p=2.0×10⁻²; OVCAR4: 6.4×10⁻³) and DDX3X KO in combination with chemotherapy (OVCAR8: p=2.6×10⁻²; OVCAR4: 1.3×10⁻²) with the WT. The number of mice used in each group was equal to 5.

FIG. 22D: The heatmap shows the tumor volumes of each group at the endpoint of the experiment described in FIG. 4D.

FIG. 22E: Live-cell imaging demonstrates the efficacy of the Flower mAb to protect hFwe-Lose expressing cells from competition-induced apoptosis. Top row shows a co-culture of GFP and hFwe-Lose expressing cells and RFP and hFwe-Win expressing epithelial cancer cells at 0, 9, 18,27, and 36 hours, as demonstrated previously”. The bottom row shows the same co-culture with application of the Flower mAb, demonstrating the rescue of hFwe-Lose expressing cells from competition-induced apoptosis.

FIG. 22F: Heatmap showing a time-dependent elimination of hFwe-Lose expressing cells in the cell-competition assay in the presence and absence of Flower monoclonal antibody.

FIG. 22G: The impact of Flower mAb in protecting hFwe-Lose cells from apoptosis during cell-competition is highlighted by the change in ratio of hFwe-Win and hFwe-Lose expressing cells in presence and absence of Flower mAb. The ratio of Win/Lose isoforms changes significantly over 36 hours in the absence of Flower mAb (p=3×10⁻²) but remains unaffected with Flower mAb (Biological replicates, n=5).

FIG. 22H: Tumor volumes of each of the cohorts in FIG. 6A are shown for both OVCAR4 (left) and OVCAR8 (right) groups. Groups that received the Flower mAb are designated with a red plus under the corresponding data points. As shown, addition of Flower mAb significantly reduces tumor volume compared to the control groups and groups that did not receive Flower mAb. The p-values shown compare tumor volumes at the endpoint of the experiment of mice untreated mice with Docetaxel/Carboplatin and Bevacizumab treated (OVCAR8: p=2.2×10⁻⁴; OVCAR4: p=2.3×10⁻³), and Flower mAb (OVCAR8: p=1.5×10⁻⁴; OVCAR4: p=8.1×10⁻⁴).

The number of mice used in each group was equal to 5.

FIG. 221: Heatmaps demonstrate the number of metastatic lesions in each of the treatment groups that received OVCAR4 (left) and OVCAR8 (right) implants. Each row represents a treatment group that is designated on the right of the heatmaps. These results further demonstrate the ability of Flower mAb to attenuate metastatic spread both as a monotherapy and in combination with standard chemotherapy.

FIG. 22J: Kaplan-Meier analysis of each of the OVCAR4 and OVCAR8 cohorts demonstrates the impact of Flower mAb treatment on survival. The color of each line represents a treatment cohort corresponding to the labeling key. Mice that received treatment with Docetaxel/Carboplatin/Bevacizumab/Flower mAb had the highest survival rate, with 80% survival at the end of the experiment (OVCAR8: p=1.8×10⁻³; OVCAR4: p=1.8×10⁻³).

FIGS. 23A-23D show Flower Exon 3 demonstrates increased enrichment of alternative splicing factors and epigenetic modifications.

FIGS. 23A-23B: Box plots show increased DNA methylation of the hFwe Exon 3 (unmatched p=3.2×10⁻³ and matched p=1.1×10⁻³ normal vs. HGSC; unmatched and matched p <1×10⁻¹⁰ HGSC vs. stroma), as well as an increased DNMT3A binding to the hFwe Exon 3 (unmatched p=5.8×10⁻⁷ and matched p=1.9×10⁻⁴ normal vs. HGSC; p<1×10⁻¹⁰ HGSC vs. stroma) in the stroma vs normal and HGSC tissue of patient derived samples. Scatter plots show a significant correlation between DNA Methylation of the hFwe Exon 3 and hFwe-Lose expression (p=4.9×10⁻⁴), as well as between DNMT3A binding to the hFwe Exon 3 and hFwe-Lose expression (p=2.5×10⁻⁶) in the stroma. In the box plots, the color scale represents log 2 fold change of hFwe-Lose expression, while in the correlation analysis, the color delineates normal, tumor and stromal samples. (Biological replicates for normal tissue n=15, biological replicates for tumor and stroma tissue n=30)

FIG. 23C: The control for qChIP experiment demonstrated in FIG. 23A & 23B are presented.

FIG. 23D: Heatmap of qChIP in stromal tissue from patient derived HGSC samples on Flower Exon 3 shows association of 39 proteins which participate in DNA methylation induced RNA splicing via DNA methylation changes. H3K9Me3 (p=1×10⁻⁵), HP1 (p=1×10⁻⁴), SRSF3 (p=5×10⁻⁵), GLP (p=4×10⁻⁵) and G9a (p=1×10⁻⁴) are the most abundant, (Biological replicates, n=5).

FIGS. 24A-24B show the enrichment of splicing machinery at Flower Exon 3 is specific to HGSC stromal tissue.

FIG. 24A: A heatmap depicting relative enrichment from ChIP influenced gene walking experiment showing association of DNA methylation, G9a, GLP, H3K9Me3, HP1, SRSF3, and RNA Pol II, on each Exon (1-6) and 200 bp flanking regions upstream (5′) and downstream (3′) from each Exon of the hFwe gene locus. The bar representing the value ranges 0.3 and 4 (color yellow-green-blue) illustrates the relative binding/association of the epigenetic markers (left column) on the respective Exons 1-6 and 200 bp 5′ & 3′ flanking regions (Biological replicates, n=8).

FIG. 24B: Methylight assay performed in all hFwe Exon bodies in normal, HGSC tumor, and stromal samples showing specific and exclusive methylation of hFwe Exon 3 in HGSC stromal tissue. Data are presented as mean values +/−SEM, (Biological replicates, n=15).

FIGS. 25A-25C show biological assay using CRISPR-off at Flower Exon 3 impacts Flower isoform expression.

FIG. 25A: A model depicts CRISPR-off strategy for hFwe Exon 3.

FIG. 25B: Agarose gel electrophoresis of Bi-sulfite-treated and untreated samples derived from WT stromal cells and two clones of CRISPR-off engineered stromal cells. Stromal cells of Clone 1 were co-transfected with CRISPR-off plasmids dCas9-KRAB, dCAS9-D3A-3L, sgRNA targeting hFwe Exon 3. Stromal cells of Clone 2 were co-transfected with CRISPR-off plasmids dCas9-DNMT3A, dCAS9—DNMT3B, sgRNA targeting hFwe Exon 3. To confirm DNA methylation, we performed Methylation-specific PCR in samples treated or untreated with bi-sulfite and ran the products in agarose gels.

FIG. 25C: Co-culture of WT tumor and stromal cells shows binding of LINC01914 to hFwe Exon 3 DNA, high hFwe Exon 3 DNA methylation, enrichment of splicing machinery and high expression of hFwe-Lose isoforms, in stromal cells. Co-culture of LINC01914KO tumor cells and hFwe Exon-3 CRISPR-off stromal cells show no binding of LINC01914 to hFwe Exon 3, high DNA methylation, enrichment of splicing machinery at hFwe Exon 3, and high expression of hFwe-Lose.

FIGS. 26A-26E show silencing of LINC01914 expression prevents Flower Exon 3 methylation in stromal cells.

FIG. 26A: Model depicting the insertion of the Poly-A sequence within LINC01914 locus and respective homology arms,

FIG. 26B: Model depicting the experiments performed in stromal cells after co-culture with OVCAR8 cells. Experiments related to LINCO1914 deletion and rescue are performed in co-culture. For the control experiment, OVCAR8 LIN(C01914 WT and GFP-labeled fibroblasts were cultured for 96h. For the deletion of hFwe Exon 3 binding site, we co-cultured GFP-labeled fibroblasts either with OVCAR8 LINC01914 KO or with OVCAR8 LLNC01914 with Poly-A insertion. For the rescue experiments, we co-cultured GFP-labeled fibroblasts either with OVCAR8 LIVC01914 KO overexpressing a WT LINC01914, a MT (mutated hFwe Exon 3 binding site) LINC01914, or a LINC01914 deletion construct (missing Exon 3 binding site).

FIG. 26C: Methylation-specific PCR (MSP) of hFwe Exon 3 in stromal cells co-cultured with OVCAR8 cells is shown. Each panel represents a specific co-culture experiment with and without bisulfite treatment (indicated at bottom of gel with +/−signs). Bi sulfite treatment of stromal DNA shows that stromal cells have hFwe Exon 3 methylation after co-culture with WT OVCAR8 cells (panel 1, lane 6). The methylation of hFwe Exon 3 cannot be observed in stromal cells co-cultured with LINC01914 KO OVCAR8 cells since only un-methylated DNA specific primers show amplification of hFwe Exon 3 after bisulfite treatment (panel 2, lane 6). The methylation of hFwe Exon 3 in stromal cells is rescued when these cells are co-cultured with LINC(101914 KO OVCAR8 cells overexpressing the WT LINC01914 (panel 3, lane 5). Stromal cells co-cultured with LLNC01914 KO OVCAR8 cells overexpressing the MT LINC01914 are unable to rescue the WT methylation pattern.

FIG. 26D: Analysis of hFwe isoform expression pattern upon treatment with the cytokine inhibitor Brefeldin A. OVCAR8 WT and GFP-labeled fibroblasts were co-cultured in media containing Brefeldin A for 96h (left). GFP-labeled fibroblasts were also cultured alone and treated with conditional media containing Brefeldin A (right). The gels show that treatment with Brefeldin A does not affect Exon 3 skipping and hFwe-Lose expression in stromal cells.

FIG. 26E: Model depicting ChOP to observe the DNA-RNA adduct between LINC01914 and hFwe Exon 3.

FIGS. 27A-27L show LINC01914 binds to Flower at Exon 3 through Hoogsteen base-pairing.

FIG. 27A: ChOP shows an aggregate association of LINC01914 to the Exon 3 body of the hFwe gene loci. The experiment is conducted in primary cancer (labeled with GFP) and stromal cells extracted and co-cultured (for 8 days) from 8 HGSC patient samples. Sorted primary stromal cell fraction upon incubation with LINC01914 anti-sense oligos show association exclusively on the Exon 3 body (blue peak) and not on any other exon body or the 5′ & 3′ flanking regions. The ChOP pull-down analyzed by qPCR is calculated as the percentage of input (p<1×10⁻¹⁰).

FIG. 27B: Model depicting the experimental confirmation of the formation of a DNA-RNA adduct between hFwe and Tu-Stroma mediated by Hoogsteen base-pairing.

FIGS. 27C-27D: Validation of the physical association of LINC01914 and Exon 3 DNA body (containing complementary GA-rich sequences) via Hoogsteen base-pairing is performed by in-vitro capture assay using biotinylated LINC01914. Data shows a peak (red) generated specifically on the Exon 3 body, while incubation with 7-deaza-purine nucleotides (blue) abolishes the Hoogsteen base-pairing (p<1×10⁻¹⁰). The interaction is further validated in the nuclei isolated from HGSC stromal cells (8 HGSC patients). A consistent and exclusive enrichment of peak (red) is primarily observed on the Exon 3 body upon incubation of the stromal nuclei with the ectopic biotinylated LINC01914 by qPCR analysis (p=6.2×10⁻⁷).

FIGS. 27E-27F: The in-vivo capture assay shows the physical association and DNA-RNA adduct formation between LINC01914 and Exon 3 DNA body in the primary stromal cells extracted from the HGSC patients. Results show an exclusive enrichment of peak (red) on the Exon 3 DNA body and not on other Exons or the 5′ & 3′ flanking regions of hFwe gene, upon transfection of stromal cells with biotinylated LINC01914 (p<1×10⁻¹⁰). Results show a significant adduct enrichment on Exon 3 body (red) in the photo-activated nuclear extracts of the stromal transfected cells in comparison to the non-photo-activated nuclear extracts (blue) (p<1×10⁻⁰).

FIG. 27G: Exon 3 dsDNA and LINC01914 ssRNA shown in blue and orange spectra respectively correspond as the controls. Theoretical mean spectra [(LINC01914+ Exon 3)/2]shown in red is the sum of the individual CD spectra for Exon 3 dsDNA and LINC01914 ssRNA.

FIG. 27H: Data shows the location of the Exon 3 DNA body (lane 1), and the location of single-stranded LINC01914 (lane 2). Lane 3 shows the formation of a DNA-RNA complex upon mixing; the bottom shows DNA, the middle shows RNA, and a DNA-RNA complex is observed as a supershift (top band marked with red arrow). In lane 4, the unbound single-stranded RNA is destroyed upon the addition of RNase H and only the DNA-RNA adduct band is visible at the top.

FIG. 271: Data shows the location of the Exon 3 DNA body (lane 1) and the location of single-stranded LINC01914 (lane 2). Lanes 3 to 7 show the formation of a DNA-RNA complex (top band marked with red arrow) upon mixing with decreasing concentrations of LINC01914 (middle band), which leads to a decrease in the intensity of the supershift (top band marked with red arrow).

FIG. 27J: Data shows the location of the Exon 3 DNA body (lane 1), and the location of single-stranded LINC01914 (lane 2). Lanes 3 to 7 show the formation of a DNA-RNA complex (top band marked with red arrow) upon mixing with labeled LINC01914 (middle band) and increasing concentrations of unlabeled LINC01914, which leads to a decrease in the intensity of the supershift (top band marked with red arrow).

FIG. 27K: Data shows the location of the Exon 3 DNA body (lane 1) and the location of single-stranded LINC01914 (lane 2). Lane 3 shows the formation of a DNA-RNA complex (top band marked with red arrow) upon mixing with the first half of the LINC01914 sequence (middle band). In lane 4, the unbound single-stranded RNA is destroyed upon the addition of RNase H and only the DNA-RNA adduct band is visible at the top.

FIG. 27L: Data shows the location of the Exon 3 DNA body (lane 1), and the location of single-stranded LINC01914 (lane 2). Lane 3 does not show the formation of a DNA-RNA complex upon mixing with the second half of the LINC01914 sequence (top band). In lane 4, the unbound single-stranded RNA is destroyed upon the addition of RNase H and only the DNA band is visible.

FIGS. 28A-28D show that increased levels of mature LINC01914 in stroma is secondary to elevated expression in tumor tissue.

FIG. 28A: qChIP is performed on the promoter region of LINC01914 (Exon1 and 700 bp upstream of Exon 1) in normal ovarian (N—grey color), HGSC tumor (H—red color) and stromal (S—blue color) tissue samples (from 8 HGSC patients) using anti-H3K27Ac (p<1×10⁻⁴), anti-H3K36Me3 (p<1×10⁻⁴), and anti-H3K27Me3 antibodies (p=1.7×10⁻⁴). The results show repressed LINC01914 promoter activity in the normal ovarian, and HGSC stromal tissue whereas an active LINC01914 promoter status in the HGSC tumor tissue. Data is expressed as the percent of pre-IP input for each sample ±SE and is representative of at least 3 independent IPs.

FIG. 28B: A comprehensive set of internal controls such as scramble primer, IgG antibody, Actin antibody, and no antibody were included in the ChIP assay along with several antibodies H3K27ac, H3K36me3, H3K27me3.

FIG. 28C: Model depicting the analysis of the expression of LINC01914 nascent RNA (left). Nascent RNA transcript analysis from normal, HGSC and stromal tissue shows that LINC01914 is expressed 12-fold in the HGSC tumor tissue compared to the normal ovarian and the stromal tissue. Mature RNA signal found in the stromal tissue is most likely a result of RNA transport from the tumor tissue (p=1.5×10⁻⁵) (right).

FIG. 28D: Analysis of RT-qPCR of LINC01914 using WT LINC01914 primers and 5′ & 3′ barcoded-specific primers. (Top) The plot shows the expression of endogenous LINC01914 in monocultures of OVCAR8 WT cells (lane 2), OVCAR8 LINC01914 KO cells (lane 3), OVCAR8 LINC01914 KO cells with overexpression of barcoded LINC01914 (lane 4), and stromal cells (lane 5, p=4,7×10⁻³). The expression of endogenous LINC01914 in stromal cells is also shown upon co-culture with OVCAR8 WT cells (lane 6), OVCAR8 LINC01914 KO cells (lane 7, p=4.4×10⁻⁶), OVCAR8 LINC01914 KO cells with overexpression of barcoded LINC01914 (lane 8, p=6.4×10⁻⁶), and OVCAR8 LINC01914 KO cells with overexpression of barcoded LINC01914 with simultaneous overexpression of WT LINC01914 in stromal cells (lane 9). The expression of endogenous LINC01914 in stromal cells is also shown upon co-culture with OVCAR8 WT cells (lane 6), OVCAR8 Rab27a/b, LINC01914 triple KO cells (lane 7), OVCAR8 Rab27a/b, LINC01914 triple KO cells with overexpression of barcoded LINC01914 (lane 8, p=1.5×10⁻⁴), and Rab27a/b, LINC01914 triple KO cells with overexpression of barcoded LINC01914 with simultaneous overexpression of WT LINC01914 in stromal cells (lane 9). The green arrows mark the lanes in which the barcoded LINC01914 was overexpressed by cancer cells. The * signals the overexpression of WT LINC01914 in stromal cells. A model of the location of WT LINC01914 primers and 5′ & 3′ barcoded-specific primers is shown at the bottom of each plot. LINC01914 enrichment was quantified with N=5 and presented on a logarithmic scale.

FIGS. 29A-29L show HGSC tumor cells pack Tu-Stroma in exosomes via DDX3X binding.

FIG. 29A: Representative nanoparticle tracking analysis showing a snapshot of the video recordings of the particles recorded and the graph showing the particle size and concentration in control and HGSC serum samples.

FIG. 29B: Western blot analysis confirms the purity of the exosomes isolated from the blood from the HGSC patients and normal non-HGSC individuals, as indicated by the expression of calnexin, CD9 and TSG101 (markers of exosomes).

FIG. 29C: Exosome particles confirmed by image stream flow cytometry (the bar surrounding color indicated exosome count) in control, & HGSOC serum samples.

FIG. 29D: Relative expression of Rab27a and Rab27b are higher in WT OVCAR8 cells compared to OVCAR8 cells with Rab27a and Rab27b double knockout (Technical replicates, n=3).

FIG. 29E: Data shows nanoparticle tracking (NTA) with exosomes extracted from KO cells having a larger size compared to exosomes from WT cells (189.1±12.4 nm vs. 141.8+5.13 nm).

FIG. 29F: Mean particle concentration with 95% confidence intervals, determined by NTA, demonstrated a significantly lower concentration of exosomes from Rab27 KO cells compared to WT cells.

FIG. 29G: RT-qPCR experiment is performed to understand the cellular localization of LINC01914 (Tu-Stroma) in matched normal, tumor, and stromal tissue from distinct patient samples. In HGSC tissue it is highly expressed in exosomal fraction, followed by the cytoplasmic fraction (p<1X10⁻⁴). In the stromal tissue, maximum expression of LINC01914 is observed in the nuclear fraction (p<1X10⁻⁴), (Technical replicates, n=3).

FIG. 29H: The visualization of LINC01914 transcripts expression in FFPE HGSC sections was achieved through ViewRNA™ in situ hybridization analysis. Large cut sections of selected high-grade ovarian tumor tissue samples were used for staining HOTAIR and LINC01914. Counterstaining was performed using DAPI. Positive LINC01914 staining was observed in HGSC (red spots).

FIG. 291: To determine the binding of LINC01914 exclusively to DDX3X, RIP experiment is performed in matched normal ovarian tissue, HGSC cancer, and HGSC stromal tissue. All the 30 RBPs are immunoprecipitated from each of the three samples and analyzed for the enrichment of LINC01914 by qPCR analysis. The results show that DDX3X is significantly and exclusively upregulated in the cancer tissue (p<1×10⁻¹⁰), (Biological replicates, n=3).

FIG. 29J: RT-qPCR analysis is conducted exosomal fractions isolated from matched normal ovarian tissue, HGSC cancer, and HGSC stromal tissue. RT-qPCR results show that DDX3X exclusively binds to the LINC01914 in exosomal fraction isolated from the HGSC tumor tissue by a 9-fold high expression compared to the stromal exosomal fraction (unmatched p=3.6×10⁻⁶ and matched p=3.9×10⁻⁶). The western blot results confirm that high expression of DDX3X exclusively in the exosomes from HGSC tumor tissue, (Biological replicates, n=5).

FIG. 29K: Western blot analysis is conducted in exosomal fractions isolated from normal ovarian tissue, HGSC cancer, and HGSC stromal tissue (15 HGSC patients). The western blot results show high expression of DDX3X in the exosomes from HGSC tumor tissue.

FIG. 29L: RT-qPCR measurement is conducted to study the fold change expression of DDX3X mRNA in pooled set of laser-captured. Results show a significant increase in the DDX3X mRNA expression in HGSC tumor tissue compared to the stroma (unmatched and matched p<1×10⁻¹⁰), (Biological replicates for tumor and stroma tissue, n=30).

FIGS. 30A-30E show uptake of tumor-derived exosomes changes the expression of Flower isoforms in stromal cells.

FIG. 30A: qPCR experiment is conducted to study the expression of hFwe-Win and hFwe-Lose isoforms in the primary HGSC stromal cells co-cultured with GFP-infected primary HGSC cancer cells on day 0, day 2, day 4, day 6, and day 8. Exogenous addition of exosome inhibitor (GW4869) in the co-cultured cells abrogates the increase in total hFwe mRNA and hFwe-Lose expression (p<1×10⁻¹⁰). Knockdown of DDX3X by lentivirus-mediated shRNA in the co-culture mix results in a comparatively higher expression of total hFwe mRNA with similar expression levels of hFwe-Lose isoform as observed in exosome inhibitor-treated (p<1×10⁻⁰).

FIG. 30B: qPCR and Immunoprecipitation experiments are conducted to validate the role of DDX3X in the exosomal packaging of Tu-Stroma, in the exosomal fractions of HGSC cells on day 0, day 2, day 4, day, and day 8. IP results show a steady and an increasing DDX3X expression in the exosomal fractions from day 0 to day 8. A coherent and significant increase in the Tu-Stroma expression is observed in the same exosomal fractions, as analyzed by qPCR analysis (p<1×10⁻⁰).

FIG. 30C: RT-qPCR measurement is performed to analyze the expression of Tu-Stroma mRNA in the exosomal fractions of primary HGSC cancer cells post DDX3X shRNA transfection (from day 0 to day 8), (Technical replicates, n=3).

FIG. 30D: qPCR is conducted to study the effect of exosome inhibitors and DDX3X knockdown on Tu-Stroma localization and expression in the nuclear and cytoplasmic fractions of primary HGSC cancer cells. Results show no change in the nuclear to the cytoplasmic distribution of Tu-Stroma expression in the exosome inhibitor treated HGSC cancer cells compared to untreated cells. However, knockdown of DDX3X shows an increase in the Tu-Stroma expression in nucleus compared to cytoplasm. Tu-Stroma expression is studied in the nuclear and cytoplasmic fraction of the HGSC stromal cells post co-culture with HGSC cancer cells from day 0-day 8 by qPCR analysis. β-values are indicated in the figure.

FIG. 30E: Live-cell imaging of co-cultures experiments for 24 h. The top panel shows the co-culture of OVCAR8 DDX3X KO cells expressing RFP-blank with WT stromal cells. The images show that both RFP-tagged OVCAR8 DDX3X KO cells and stromal cells proliferate and survive over time. The middle panel shows the co-culture of OVCAR8 DDX3X KO cells expressing RFP-fused DDX3X with WT stromal cells. The images show that WT stromal cells start expressing RFP within 24 hours. The bottom panel shows the co-culture of OVCAR8 DDX3X KO cells expressing RFP-fused DDX3X with WT stromal cells in the presence of exosome inhibitors.

FIGS. 31A-31B show epistatic pathway of Tu-Stroma mediated Exon 3 methylation and Flower Lose generation in stromal cells.

FIG. 31A: A functional genetic assay is performed on HGSC and Stromal cells cultured individually (row 1) or in co-culture using a series of loss-of-function experiments using Tu-Stroma (row 2) and DDX3X (row 3) shRNA (HGSC), double KO of Rab27a and Rab27b (row 4), Exosome (row 5) and DNMT3A (row 6) inhibitors (co-culture) or shRNA against Tu-Stroma (row 7), DDX3X (row 8), DNMT3A (row 9), G9a (row 10), GLP (row 11), HP1 (row 12) and SRSF3 (row 13). We used five sets of molecular experiments to determine the steps in this molecular mechanism. These experiments were a) ChOP on Exon 3 region of HGSC stromal cells to identify the association of Tu-Stroma at this gene locus, b) MethyLight Assay to evaluate level of DNA methylation on this region, c) a series of ChIP experiments (with input, no Ab, Actin Ab, IgG Ab, and Scramble primers as controls) to identify DNA methylation status, and association of DNMT3A, G9a, GLP, H3K9Me3, HP1, and SRSF3 on hFwe Exon 3 gene locus in HGSC stromal cells, d) qPCR-based analysis of hFwe-Lose isoform, hFwe-Win isoform, and Exon 3 expression in HGSC stromal cells, and e) RT-PCR based validation of Exon 3 cassette deletion. Using the results from this assay, a series of hypotheses are generated to construct the epistatic relationship between individual components in the pathway and depicted as a model. The p-values shown in d) compare the expression of hFwe-Lose in each group with the expression in the control. Microscopy images of each experiment are shown in the first column (scale 10 μm).

FIG. 31B: Pseudotime algorithm model to determine the sequence of epigenetic events at the Exon 3 hFwe locus. The current model represents the most plausible series of steps (significance of the model (p) is 0.043, which is calculated as a sum of the type I and type II errors across all steps). This pseudo time analysis combined with the known biological function of individual components reveals the mechanism of Tu-Stroma mediated exclusion of Exon 3 in stromal cells.

FIGS. 32A-32D show knockout of Tu-Stroma and DDX3X expression leads to reduced tumor growth and improves overall survival.

FIG. 32A: Tu-Stroma WT or KO OVCAR8 were orthotopically implanted in female NOD.Cg-Prkdc^{scid} Il2rg^{tmlWjl}/SzJ (NSG) mice and tumor spread in ovaries was determined using MRI.

FIG. 32B: RT-qPCR data shows high expression of hFwe-Win in tumor tissue and hFwe-Lose in stromal tissue in WT OVCAR8 xenografts. In Tu-Stroma KO OVCAR8 xenografts, hFwe-Win expression is high in both tumor and stromal tissue and hFwe-Lose expression in stromal tissue was diminished, (Biological replicates, n=5).

FIG. 32C: Kaplan-Meier curves are colored as per the label coding key shown in the figure. The p-values show the comparison in survival between Tu-Stroma KD in combination with chemotherapy (PDX1: p=2.1×10⁻²; PDX2: 1.8×10⁻³) and DDX3X KD in combination with chemotherapy (PDX1: p=1.8×10⁻³; PDX2: 1.3×10⁻³) with the WT. The number of mice used in each group was equal to 5.

FIG. 32D: After orthotopic implantation, mice with each knockout line (DDX3X KO, a Tu-Stroma KO, and a combined DDX3X/Tu-Stroma KO) were either untreated or received Docetaxel/Carboplatin therapy. Representative tumors from each group (n=5) are shown with OVCAR8 tumors in the top half and OVCAR4 tumors in the bottom half Tumors treated with Docetaxel/Carboplatin are indicated with an open box.

FIG. 33 shows affinity binding data for the eight antibodies disclosed herein (antibody 1a, antibody 1b, antibody 2a, antibody 2b, antibody 3a, antibody 3b, antibody 4a, antibody 4b).

FIG. 34 shows schematic and sequences where the eight antibodies disclosed herein (antibody 1a, antibody 1b, antibody 2a, antibody 2b, antibody 3a, antibody 3b, antibody 4a, antibody 4b) bind to the Flower proteins hFwe1, hFwe2, hFwe3, and hFwe4, respectively:

(SEQ ID NO: 251)
CSGGAPGASASSAPPAQEEG,
(SEQ ID NO: 252)
CGGAPGASASSAPPAQEEGMTWWYR,
(SEQ ID NO: 253)
EKVDRLRSW,
(SEQ ID NO: 254)
NTVAEKVDRLRSWQKAVF,
(SEQ ID NO: 255)
KKAQTEAGSFAAQHPREPGPFSEGTRQAFATPAV,
(SEQ ID NO: 256)
EAGSFAAQHPREPGPFSEGTRQAFAT,
(SEQ ID NO: 257)
GKKGDAISYARIQQQRQQADEEKLAETL,
(SEQ ID NO: 258)
SGEIRMPAGAMRSPMPGSSSRGSRRMRRSSRRPWRGSCEGLGAPPS.

FIG. 35 shows ELISA binding results of various combinations of six VH sequences (VH1, VH2, VH3, VH4, VH5, VH6) with six VL sequences (VL1, VL2, VL3, VL4, VL5, VL6).

### DETAILED DESCRIPTION OF THE DISCLOSURE

In the current study, we set to investigate the mechanisms that promote the generation of Flower-Lose isoforms in the tumor microenvironment (TME). High expression of Lose could be a tumor-induced effect or a pre-existing phenotype of the host tissue. We focused our attention on high grade serous ovarian carcinoma (HGSC) given its high expression of the Flower isoforms and its aggressive course. Here, we find that cancer cells are programmed to promote the expression of Lose in the TME. We have discovered that human Flower isoforms in both the tumor and stroma are under STAT3 transcriptional regulation, which explains the unusually high levels of expression in the tumor and the stroma. Further, we found that tumor cells manipulate the splicing pattern of the Flower gene non-cell autonomously by targeting Exon 3 through exosome-mediated secretion of a long non-coding RNA (lncRNA), which specifically binds to Flower Exon3 locus, leading to downstream epigenetic changes. We demonstrate the role of this lncRNA, in regulating cancer growth, metastasis and host survival in patient-derived 3D scaffold models, orthotopic PDX models and cancer patients. Finally, we developed an antibody against the Flower protein that has a significant impact on in-vivo tumor growth and metastatic spread both as monotherapy and in combination with standard of care chemotherapy regimens. In this study, we demonstrate a concept by which cancer cells actively manipulate the fitness of TME cells via a very specific non-cell autonomous signal to achieve evolutionary growth advantages.

In the following detailed description, numerous specific details are set forth in order to provide a thorough understanding of the anti-CACFD1 (calcium channel Flower domain containing 1) antibodies or CACFD1 binding antibodies (Abs) and uses thereof. In some instances, well-known methods, procedures, and components have not been described in detail so as not to obscure the Abs disclosed herein and methods of use thereof.

The present disclosure and Examples provide anti-CACFD1 Abs that bind to certain epitopes on the human CACFD1 proteins such as hFwe1, hFwe2, hFwe3 and hFwe4. Some of the present anti-CACFD1 antibodies bind to calcium channel flower homolog proteins that behave as Flower-Lose proteins (e.g., hFwe1 and hFwe3), whereas some of the other anti-CACFD1 antibodies disclosed herein bind to calcium channel flower homolog proteins that behave as Flower-Win proteins (e.g., hFwe2 and hFwe4). The antibodies disclosed herein are useful for increasing the cellular fitness of a group of cells or tissues.

As used herein, the singular form “a”, “an” and “the” include plural references unless the context clearly dictates otherwise. For example, the term “a protein” or “at least one protein” may include a plurality of proteins, including mixtures thereof.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting. Each literature reference or other citation referred to herein is incorporated herein by reference in its entirety.

In the description presented herein, elements of the antibodies targeting calcium channel flower homolog protein and uses thereof, and variations thereof are described. This description is not intended to be limiting and changes in the components, sequence of steps, and other variations would be understood to be within the scope of the present invention.

It is appreciated that certain features of the antibodies targeting calcium channel flower homolog protein and uses thereof, which are, for clarity, described in the context of separate embodiments, may also be provided in combination in a single embodiment. Conversely, various features of the anti-calcium channel flower homolog protein antibodies and uses thereof which are, for brevity, described in the context of a single embodiment, may also be provided separately or in any suitable sub combination or as suitable in any other described embodiment of the invention. Certain features described in the context of various embodiments are not to be considered essential features of those embodiments, unless the embodiment is inoperative without those elements.

As used herein, the term “antibody” may be used interchangeably with the term “immunoglobulin”, having all the same qualities and meanings. An antibody binding domain or an antigen binding site can be a fragment of an antibody or a genetically engineered product of one or more fragments of the antibody, which fragment is involved in specifically binding with a target antigen. By “specifically binding” is meant that the binding is selective for the antigen of interest and can be discriminated from unwanted or nonspecific interactions. For example, an antibody is said to specifically bind to a target antigen when the equilibrium dissociation constant is <10⁻⁵, 10⁻⁶, or 10⁻⁷ M. In some embodiments, the equilibrium dissociation constant may be <10⁻⁸ M or 10⁻⁹ M. In some further embodiments, the equilibrium dissociation constant may be <10⁻¹⁰ M, 10⁻¹¹ M, or 10⁻¹²M. In some embodiments, the equilibrium dissociation constant may be in the range of <10⁻⁵ M to 10⁻¹²M.

As used herein, the term “antibody” encompasses an antibody fragment or fragments that retain binding specificity including, but not limited to, IgG, heavy chain variable region (VH), light chain variable region (VL), Fab fragments, F(ab′)2 fragments, scFv fragments, Fv fragments, a nanobody, minibodies, diabodies, triabodies, tetrabodies, and single domain antibodies as they are generally known in the art (see, e.g., Hudson and Souriau, Nature Med. 9: 129-134 (2003)). Also encompassed are humanized, primatized, and chimeric antibodies as these terms are generally understood in the art. The term “antibody” also encompasses bispecific and multi-specific antibodies.

As used herein, the term “heavy chain variable region” may be used interchangeably with the term “VH domain” or the term “VH”, having all the same meanings and qualities. As used herein, the term “light chain variable region” may be used interchangeably with the term “VL domain” or the term “VL”, having all the same meanings and qualities. A skilled artisan would recognize that a “heavy chain variable region” or “VH” with regard to an antibody encompasses the fragment of the heavy chain that contains three complementarity determining regions (CDRs) interposed between flanking stretches known as framework regions. The framework regions are more highly conserved than the CDRs and form a scaffold to support the CDRs. Similarly, a skilled artisan would also recognize that a “light chain variable region” or “VL” with regard to an antibody encompasses the fragment of the light chain that contains three CDRs interposed between framework regions.

As used herein, the term “complementarity determining region” or “CDR” refers to the hypervariable region(s) of a heavy or light chain variable region. Proceeding from the N-terminus, each of a heavy or light chain polypeptide has three CDRs denoted as “CDR1,” “CDR2,” and “CDR3”. Crystallographic analysis of a number of antigen-antibody complexes has demonstrated that the amino acid residues of CDRs form extensive contact with a bound antigen, wherein the most extensive antigen contact is with the heavy chain CDR3. Thus, the CDR regions are primarily responsible for the specificity of an antigen-binding site. In one embodiment, an antigen-binding site includes six CDRs, comprising the CDRs from each of a heavy and a light chain variable region. One of ordinary skill in the art would readily determine the amino acid residues of CDRs by methods and techniques generally known in the art.

As used herein, the term “epitope” refers to an immunogenic amino acid sequence. An epitope may refer to a minimum amino acid sequence of, for example, 6-8 amino acids which are immunogenic, when removed from its natural context. An epitope also may refer, in other embodiments, to that portion of a natural polypeptide which is immunogenic, where the natural polypeptide containing the epitope is referred to as an antigen. In some embodiments, a polypeptide or antigen may contain one or more distinct epitopes. An epitope may refer, in some embodiments, to an immunogenic portion of a multichain polypeptide, e.g., which is encoded by distinct open reading frames. The terms epitope, peptide, and polypeptide all refer to a series of amino acids connected one to the other by peptide bonds between the alpha-amino and alpha-carboxy groups of adjacent amino acids, and may contain or be free of modifications such as glycosylation, side chain oxidation, or phosphorylation, provided such modifications, or lack thereof, do not destroy immunogenicity. As used herein, the term “peptide” is meant to refer to both a peptide and a polypeptide or protein.

As used herein, the terms “tumor microenvironment”, “cancer microenvironment”, “TME”, and “tumor milieu” may be used interchangeably having the same qualities and meanings and encompassing the microenvironment to tumor development. While the normal cellular microenvironment can inhibit malignant cell growth, the modifications that occur in the tumor microenvironment may synergistically support cell proliferation.

It is known that tumors shape their microenvironment and support the development of both tumor cells and non-malignant cells. The tumor microenvironment affects angiogenesis by interfering with the signaling pathways required for cell recruitment and vascular construction. In addition, proteins secreted by the tumor modify the microenvironment by contributing growth factors and proteases that degrade the extracellular matrix and affect cell motility and adhesion. Stromal cells secrete extracellular matrix proteins, cytokines, growth factors, proteases, protease inhibitors, and endoglycosidases such as heparinase.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible sub ranges as well as individual numerical values within that range. For example, description of a range such as from 1 to 6 should be considered to have specifically disclosed sub ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between.

### Antibodies

A skilled artisan would appreciate that an “anti-CACFD1 (calcium channel Flower domain containing 1) antibody” or “CACFD1 binding antibody” encompasses in its broadest sense an antibody that specifically binds an antigenic determinant of a CACFD1 protein or a Flower protein. The skilled artisan would appreciate that specificity for binding to CACFD1 protein reflects that the binding is selective for the CACFD1 protein and can be discriminated from unwanted or nonspecific interactions. In certain embodiments, a CACFD1 protein binding antibody comprises an antibody fragment or fragments. In one embodiment, the antibodies bind to hFwe1, hFwe2, hFwe3 or hFwe4.

A skilled artisan would appreciate that the term “homology”, and grammatical forms thereof, encompasses the degree of similarity between two or more structures. The term “homologous sequences” refers to regions in macromolecules that have a similar order of monomers. When used in relation to polypeptide (or protein) sequences, the term “homology” refers to the degree of similarity between two or more polypeptide (or protein) sequences or fragments thereof. Typically, the degree of similarity between two or more polypeptide (or protein) sequences refers to the degree of similarity of the composition, order, or arrangement of two or more amino acid of the two or more polypeptides (or proteins). The two or more polypeptides (or proteins) may be of the same or different species or group. The term “percent homology” when used in relation to polypeptide (or protein) sequences, refers generally to a percent degree of similarity between the amino acid sequences of two or more polypeptide (or protein) sequences. The term “homologous polypeptides” or “homologous proteins” generally refers to polypeptides or proteins that have amino acid sequences and functions that are similar. Such homologous polypeptides or proteins may be related by having amino acid sequences and functions that are similar but are derived or evolved from different or the same species using techniques generally known in the art.

In some embodiments, homologues comprise polypeptides which are at least 50%, at least 55%, at least 60%, at least 65%, at least 70%, at least 75%, at least 80%, at least 85%, at least 87%, at least 89%, at least 91%, at least 93%, at least 95%, at least 96%, at least 97%, at least 98%, or at least 99% homologous to a polypeptide or a portion thereof disclosed herein, as determined using BlastP® software of the National Center of Biotechnology Information (NCBI) using default parameters.

In some embodiments, homology also encompasses deletion, insertion, or substitution variants, including an amino acid substitution thereof and biologically active polypeptide fragments thereof. In one embodiment, the variant comprises conservative substitutions, or deletions, insertions, or substitutions that do not significantly alter the three-dimensional structure of the polypeptide of interest described herein. In some embodiments, the deletion, insertion, or substitution does not alter the function of the polypeptide of interest disclosed herein.

In some embodiments, the anti-CACFD1 antibodies disclosed herein comprise three heavy chain complementarity determining regions (HCDR1, HCDR2, HCDR3) and three light chain CDRs (LCDR1, LCDR2, LCDR3), wherein the HCDR1, HCDR2, and HCDR3 have the sequences of SEQ ID NOs:326-328 respectively, and the LCDR1, LCDR2, and LCDR3 have the sequences of SEQ ID NOs:329-331 respectively, the HCDR1, HCDR2, and HCDR3 have the sequences of SEQ ID NOs:266-268 respectively, and the LCDR1, LCDR2, and LCDR3 have the sequences of SEQ ID NOs:269-271 respectively; or the HCDR1, HCDR2, and HCDR3 have the sequences of SEQ ID NOs:272-274 respectively, and the LCDR1, LCDR2, and LCDR3 have the sequences of SEQ ID NOs:275-277 respectively; or the HCDR1, HCDR2, and HCDR3 have the sequences of SEQ ID NOs:278-280 respectively, and the LCDR1, LCDR2, and LCDR3 have the sequences of SEQ ID NOs:281-283 respectively; or the HCDR1, HCDR2, and HCDR3 have the sequences of SEQ ID NOs:284-286 respectively, and the LCDR1, LCDR2, and LCDR3 have the sequences of SEQ ID NOs:287-289 respectively; or the HCDR1, HCDR2, and HCDR3 have the sequences of SEQ ID NOs:290-292 respectively, and the LCDR1, LCDR2, and LCDR3 have the sequences of SEQ ID NOs:293-295 respectively; or the HCDR1, HCDR2, and HCDR3 have the sequences of SEQ ID NOs:296-298 respectively, and the LCDR1, LCDR2, and LCDR3 have the sequences of SEQ ID NOs:299-301 respectively; or the HCDR1, HCDR2, and HCDR3 have the sequences of SEQ ID NOs:302-304 respectively, and the LCDR1, LCDR2, and LCDR3 have the sequences of SEQ ID NOs:305-307 respectively; or the HCDR1, HCDR2, and HCDR3 have the sequences of SEQ ID NOs:308-310 respectively, and the LCDR1, LCDR2, and LCDR3 have the sequences of SEQ ID NOs:311-313 respectively.

In some embodiments, the anti-CACFD1 antibodies disclosed herein comprise heavy chain and light chain CDR sequences that are at least 80% (e.g., at least 85%, 90%, 95%, 96%, 97%, 98%, or 99%) identical to the CDR sequences disclosed herein. One skilled in the art would appreciate that the anti-CACFD1 antibodies comprising CDR sequences with at least 80% identity to the CDR sequences disclosed herein, maintain their specific binding to any of SEQ ID NOs: 251-258.

In some embodiments, the antibodies comprise VH and VL sequences that are at least 80% (e.g., at least 85%, 90%, 95%, 96%, 97%, 98%, or 99%) identical to the VH and VL sequences set forth herein. One skilled in the art would appreciate that the anti-CACFD1 antibodies comprising VH and VL sequences with at least 80% identity to the VH and VL sequences disclosed herein, maintain their specific binding to any of SEQ ID NOs: 251-258. Further, one skilled in the art would appreciate that percent sequence identity may be determined using any of a number of publicly available software application, for example but not limited to BlastP® software of the National Center of Biotechnology Information (NCBI) using default parameters. In one embodiment, the anti-CACFD1 antibodies disclosed herein comprise a heavy chain variable region having an amino acid sequence at least 80% identical to SEQ ID NO:235, and a light chain variable region having an amino acid sequence at least 80% identical to SEQ ID NO:236. In one embodiment, the anti-CACFD1 antibodies disclosed herein comprise a heavy chain variable region having an amino acid sequence at least 80% identical to SEQ ID NO:237, and a light chain variable region having an amino acid sequence at least 80% identical to SEQ ID NO:238. In one embodiment, the anti-CACFD1 antibodies disclosed herein comprise a heavy chain variable region having an amino acid sequence at least 80% identical to SEQ ID NO:239, and a light chain variable region having an amino acid sequence at least 80% identical to SEQ ID NO:240. In one embodiment, the anti-CACFD1 antibodies disclosed herein comprise a heavy chain variable region having an amino acid sequence at least 80% identical to SEQ ID NO:241, and a light chain variable region having an amino acid sequence at least 80% identical to SEQ ID NO:242. In one embodiment, the anti-CACFD1 antibodies disclosed herein comprise a heavy chain variable region having an amino acid sequence at least 80% identical to SEQ ID NO:243, and a light chain variable region having an amino acid sequence at least 80% identical to SEQ ID NO:244. In one embodiment, the anti-CACFD1 antibodies disclosed herein comprise a heavy chain variable region having an amino acid sequence at least 80% identical to SEQ ID NO:245, and a light chain variable region having an amino acid sequence at least 80% identical to SEQ ID NO:246. In one embodiment, the anti-CACFD1 antibodies disclosed herein comprise a heavy chain variable region having an amino acid sequence at least 80% identical to SEQ ID NO:247, and a light chain variable region having an amino acid sequence at least 80% identical to SEQ ID NO:248. In one embodiment, the anti-CACFD1 antibodies disclosed herein comprise a heavy chain variable region having an amino acid sequence at least 80% identical to SEQ ID NO:249, and a light chain variable region having an amino acid sequence at least 80% identical to SEQ ID NO:250. In one embodiment, the anti-CACFD1 antibodies disclosed herein comprise a heavy chain variable region having an amino acid sequence at least 80% identical to one of SEQ ID NOs:314, 316, 318, 320, 322, or 324, and a light chain variable region having an amino acid sequence at least 80% identical to one of SEQ ID NOs:315, 317, 319, 321, 323, or 325.

In one embodiment, the anti-CACFD1 antibodies disclosed herein comprise a heavy chain variable region having the amino acid sequence of SEQ ID NO:235, and a light chain variable region having the amino acid sequence of SEQ ID NO:236. In one embodiment, the anti-CACFD1 antibodies disclosed herein comprise a heavy chain variable region having the amino acid sequence of SEQ ID NO:237, and a light chain variable region having the amino acid sequence of SEQ ID NO:238. In one embodiment, the anti-CACFD1 antibodies disclosed herein comprise a heavy chain variable region having the amino acid sequence of SEQ ID NO:239, and a light chain variable region having the amino acid sequence of SEQ ID NO:240. In one embodiment, the anti-CACFD1 antibodies disclosed herein comprise a heavy chain variable region having the amino acid sequence of SEQ ID NO:241, and a light chain variable region having the amino acid sequence of SEQ ID NO:242. In one embodiment, the anti-CACFD1 antibodies disclosed herein comprise a heavy chain variable region having the amino acid sequence of SEQ ID NO:243, and a light chain variable region having the amino acid sequence of SEQ ID NO:244. In one embodiment, the anti-CACFD1 antibodies disclosed herein comprise a heavy chain variable region having the amino acid sequence of SEQ ID NO:245, and a light chain variable region having the amino acid sequence of SEQ ID NO:246. In one embodiment, the anti-CACFD1 antibodies disclosed herein comprise a heavy chain variable region having the amino acid sequence of SEQ ID NO:247, and a light chain variable region having the amino acid sequence of SEQ ID NO:248. In one embodiment, the anti-CACFD1 antibodies disclosed herein comprise a heavy chain variable region having the amino acid sequence of SEQ ID NO:249, and a light chain variable region having the amino acid sequence of SEQ ID NO:250. In one embodiment, the anti-CACFD1 antibodies disclosed herein comprise a heavy chain variable region having the amino acid sequence of one of SEQ ID NOs:314, 316, 318, 320, 322, or 324, and a light chain variable region having the amino acid sequence of one of SEQ ID NOs:315, 317, 319, 321, 323, or 325.

The data in Example 3 (FIG. 35) demonstrates that in the case of anti-CACFD1 antibodies comprising a heavy chain variable region having the amino acid sequence of one of SEQ ID NOs:314, 316, 318, 320, 322, or 324, and a light chain variable region having the amino acid sequence of one of SEQ ID NOs:315, 317, 319, 321, 323, or 325, any of these heavy chains may be paired with any of the light chains and the binding affinity to CACFD1 is maintained.

In some embodiments, the anti-CACFD1 antibodies disclosed herein comprise a heavy chain variable region having the amino acid sequence of SEQ ID NO:314, and a light chain variable region having the amino acid sequence of any of SEQ ID NOs:315, 317, 319, 321, 323, and 325.

In some embodiments, the anti-CACFD1 antibodies disclosed herein comprise a heavy chain variable region having the amino acid sequence of SEQ ID NO:316, and a light chain variable region having the amino acid sequence of any of SEQ ID NOs:315, 317, 319, 321, 323, and 325. In some embodiments, the anti-CACFD1 antibodies disclosed herein comprise a heavy chain variable region having the amino acid sequence of SEQ ID NO:318, and a light chain variable region having the amino acid sequence of any of SEQ ID NOs:315, 317, 319, 321, 323, and 325.

In some embodiments, the anti-CACFD1 antibodies disclosed herein comprise a heavy chain variable region having the amino acid sequence of SEQ ID NO:320, and a light chain variable region having the amino acid sequence of any of SEQ ID NOs:315, 317, 319, 321, 323, and 325.

In some embodiments, the anti-CACFD1 antibodies disclosed herein comprise a heavy chain variable region having the amino acid sequence of SEQ ID NO:322, and a light chain variable region having the amino acid sequence of any of SEQ ID NOs:315, 317, 319, 321, 323, and 325.

In some embodiments, the anti-CACFD1 antibodies disclosed herein comprise a heavy chain variable region having the amino acid sequence of SEQ ID NO:324, and a light chain variable region having the amino acid sequence of any of SEQ ID NOs:315, 317, 319, 321, 323, and 325.

In some embodiments, the anti-CACFD1 antibodies disclosed herein comprise a heavy chain variable region having the amino acid sequence of any of SEQ ID NOs:314, 316, 318, 320, and 322, and a light chain variable region having the amino acid sequence of SEQ ID NO: 315. In some embodiments, the anti-CACFD1 antibodies disclosed herein comprise a heavy chain variable region having the amino acid sequence of any of SEQ ID NOs:314, 316, 318, 320, and 322, and a light chain variable region having the amino acid sequence of SEQ ID NO: 317. In some embodiments, the anti-CACFD1 antibodies disclosed herein comprise a heavy chain variable region having the amino acid sequence of any of SEQ ID NOs:314, 316, 318, 320, and 322, and a light chain variable region having the amino acid sequence of SEQ ID NO: 319. In some embodiments, the anti-CACFD1 antibodies disclosed herein comprise a heavy chain variable region having the amino acid sequence of any of SEQ ID NOs:314, 316, 318, 320, and 322, and a light chain variable region having the amino acid sequence of SEQ ID NO: 321. In some embodiments, the anti-CACFD1 antibodies disclosed herein comprise a heavy chain variable region having the amino acid sequence of any of SEQ ID NOs:314, 316, 318, 320, and 322, and a light chain variable region having the amino acid sequence of SEQ ID NO: 323. In some embodiments, the anti-CACFD1 antibodies disclosed herein comprise a heavy chain variable region having the amino acid sequence of any of SEQ ID NOs:314, 316, 318, 320, and 322, and a light chain variable region having the amino acid sequence of SEQ ID NO: 325.

In some embodiments, the anti-CACFD1 antibodies disclosed herein comprise a heavy chain variable region having the amino acid sequence of SEQ ID NO:314 and alight chain variable region having the amino acid sequence of SEQ ID NO: 315. In some embodiments, the anti-CACFD1 antibodies disclosed herein comprise a heavy chain variable region having the amino acid sequence of SEQ ID NO:314 and a light chain variable region having the amino acid sequence of SEQ ID NO: 317. In some embodiments, the anti-CACFD1 antibodies disclosed herein comprise a heavy chain variable region having the amino acid sequence of SEQ ID NO:314 and a light chain variable region having the amino acid sequence of SEQ ID NO: 319. In some embodiments, the anti-CACFD1 antibodies disclosed herein comprise a heavy chain variable region having the amino acid sequence of SEQ ID NO:314 and a light chain variable region having the amino acid sequence of SEQ ID NO: 321. In some embodiments, the anti-CACFD1 antibodies disclosed herein comprise a heavy chain variable region having the amino acid sequence of SEQ ID NO:314 and a light chain variable region having the amino acid sequence of SEQ ID NO: 323. In some embodiments, the anti-CACFD1 antibodies disclosed herein comprise a heavy chain variable region having the amino acid sequence of SEQ ID NO:314 and a light chain variable region having the amino acid sequence of SEQ ID NO: 325.

In some embodiments, the anti-CACFD1 antibodies disclosed herein comprise a heavy chain variable region having the amino acid sequence of SEQ ID NO:316 and a light chain variable region having the amino acid sequence of SEQ ID NO: 315. In some embodiments, the anti-CACFD1 antibodies disclosed herein comprise a heavy chain variable region having the amino acid sequence of SEQ ID NO:316 and a light chain variable region having the amino acid sequence of SEQ ID NO: 317. In some embodiments, the anti-CACFD1 antibodies disclosed herein comprise a heavy chain variable region having the amino acid sequence of SEQ ID NO:316 and a light chain variable region having the amino acid sequence of SEQ ID NO: 319. In some embodiments, the anti-CACFD1 antibodies disclosed herein comprise a heavy chain variable region having the amino acid sequence of SEQ ID NO:316 and a light chain variable region having the amino acid sequence of SEQ ID NO: 321. In some embodiments, the anti-CACFD1 antibodies disclosed herein comprise a heavy chain variable region having the amino acid sequence of SEQ ID NO:316 and a light chain variable region having the amino acid sequence of SEQ ID NO: 323. In some embodiments, the anti-CACFD1 antibodies disclosed herein comprise a heavy chain variable region having the amino acid sequence of SEQ ID NO:316 and a light chain variable region having the amino acid sequence of SEQ ID NO: 325.

In some embodiments, the anti-CACFD1 antibodies disclosed herein comprise a heavy chain variable region having the amino acid sequence of SEQ IDNO:318 and alight chain variable region having the amino acid sequence of SEQ ID NO: 315. In some embodiments, the anti-CACFD1 antibodies disclosed herein comprise a heavy chain variable region having the amino acid sequence of SEQ ID NO:318 and a light chain variable region having the amino acid sequence of SEQ ID NO: 317. In some embodiments, the anti-CACFD1 antibodies disclosed herein comprise a heavy chain variable region having the amino acid sequence of SEQ ID NO:318 and a light chain variable region having the amino acid sequence of SEQ ID NO: 319. In some embodiments, the anti-CACFD1 antibodies disclosed herein comprise a heavy chain variable region having the amino acid sequence of SEQ ID NO:318 and a light chain variable region having the amino acid sequence of SEQ ID NO: 321. In some embodiments, the anti-CACFD1 antibodies disclosed herein comprise a heavy chain variable region having the amino acid sequence of SEQ ID NO:318 and a light chain variable region having the amino acid sequence of SEQ ID NO: 323. In some embodiments, the anti-CACFD1 antibodies disclosed herein comprise a heavy chain variable region having the amino acid sequence of SEQ ID NO:318 and a light chain variable region having the amino acid sequence of SEQ ID NO: 325.

In some embodiments, the anti-CACFD1 antibodies disclosed herein comprise a heavy chain variable region having the amino acid sequence of SEQ ID NO:320 and a light chain variable region having the amino acid sequence of SEQ ID NO: 315. In some embodiments, the anti-CACFD1 antibodies disclosed herein comprise a heavy chain variable region having the amino acid sequence of SEQ ID NO:320 and a light chain variable region having the amino acid sequence of SEQ ID NO: 317. In some embodiments, the anti-CACFD1 antibodies disclosed herein comprise a heavy chain variable region having the amino acid sequence of SEQ ID NO:320 and a light chain variable region having the amino acid sequence of SEQ ID NO: 319. In some embodiments, the anti-CACFD1 antibodies disclosed herein comprise a heavy chain variable region having the amino acid sequence of SEQ ID NO:320 and a light chain variable region having the amino acid sequence of SEQ ID NO: 321. In some embodiments, the anti-CACFD1 antibodies disclosed herein comprise a heavy chain variable region having the amino acid sequence of SEQ ID NO:320 and a light chain variable region having the amino acid sequence of SEQ ID NO: 323. In some embodiments, the anti-CACFD1 antibodies disclosed herein comprise a heavy chain variable region having the amino acid sequence of SEQ ID NO:320 and a light chain variable region having the amino acid sequence of SEQ ID NO: 325.

In some embodiments, the anti-CACFD1 antibodies disclosed herein comprise a heavy chain variable region having the amino acid sequence of SEQ ID NO:322 and a light chain variable region having the amino acid sequence of SEQ ID NO: 315. In some embodiments, the anti-CACFD1 antibodies disclosed herein comprise a heavy chain variable region having the amino acid sequence of SEQ ID NO:322 and a light chain variable region having the amino acid sequence of SEQ ID NO: 317. In some embodiments, the anti-CACFD1 antibodies disclosed herein comprise a heavy chain variable region having the amino acid sequence of SEQ ID NO:322 and a light chain variable region having the amino acid sequence of SEQ ID NO: 319. In some embodiments, the anti-CACFD1 antibodies disclosed herein comprise a heavy chain variable region having the amino acid sequence of SEQ ID NO:322 and a light chain variable region having the amino acid sequence of SEQ ID NO: 321. In some embodiments, the anti-CACFD1 antibodies disclosed herein comprise a heavy chain variable region having the amino acid sequence of SEQ ID NO:322 and a light chain variable region having the amino acid sequence of SEQ ID NO: 323. In some embodiments, the anti-CACFD1 antibodies disclosed herein comprise a heavy chain variable region having the amino acid sequence of SEQ ID NO:322 and a light chain variable region having the amino acid sequence of SEQ ID NO: 325.

In some embodiments, the anti-CACFD1 antibodies disclosed herein comprise a heavy chain variable region having the amino acid sequence of SEQ ID NO:324 and a light chain variable region having the amino acid sequence of SEQ ID NO: 315. In some embodiments, the anti-CACFD1 antibodies disclosed herein comprise a heavy chain variable region having the amino acid sequence of SEQ ID NO:324 and a light chain variable region having the amino acid sequence of SEQ ID NO: 317. In some embodiments, the anti-CACFD1 antibodies disclosed herein comprise a heavy chain variable region having the amino acid sequence of SEQ ID NO:324 and a light chain variable region having the amino acid sequence of SEQ ID NO: 319. In some embodiments, the anti-CACFD1 antibodies disclosed herein comprise a heavy chain variable region having the amino acid sequence of SEQ ID NO:324 and a light chain variable region having the amino acid sequence of SEQ ID NO: 321. In some embodiments, the anti-CACFD1 antibodies disclosed herein comprise a heavy chain variable region having the amino acid sequence of SEQ ID NO:324 and a light chain variable region having the amino acid sequence of SEQ ID NO: 323. In some embodiments, the anti-CACFD1 antibodies disclosed herein comprise a heavy chain variable region having the amino acid sequence of SEQ ID NO:324 and a light chain variable region having the amino acid sequence of SEQ ID NO: 325.

In some embodiments, the antibody disclosed herein comprises an IgG, IgA, IgM, IgE, IgD, a Fv, a scFv, a Fab, a F(ab′)2, a minibody, a diabody, a triabody, a multibody, a bispecific antibody, or a multi-specific antibody. In some embodiments, the IgG comprises an IgG1, IgG2, IgG3, or IgG4.

In other embodiments, the present disclosure provides an isolated antibody that binds to a target having the amino acid sequence of any one of SEQ ID NOs:251-258. In some embodiments, the antibody comprises the CDR sequences as described above. In some embodiments, the antibody comprises heavy and light chain variable regions that are at least 80% identical to the heavy and light chain variable regions described above. In some embodiments, the antibody comprises heavy and light chain variable regions as described above. In some embodiments, the antibody comprises an IgG, IgA, IgM, IgE, IgD, a Fv, a scFv, a Fab, a F(ab′)2, a minibody, a diabody, a triabody, a multibody, a bispecific antibody, or a multi-specific antibody. In some embodiments, the IgG comprises an IgG1, IgG2, IgG3, or IgG4.

### Small Molecules

In addition to the antibodies disclosed herein, the present disclosure also encompasses small molecules that target the CACFD1 proteins or Flower proteins described herein. In some embodiments, a “small molecule” may encompass a substantially non-peptidic, non-oligomeric organic molecule either prepared in the laboratory or found in nature. Small molecules, as used herein, may in certain embodiments encompass small molecules that are “natural product-like,” however, the term “small molecule” is not limited to “natural product-like” compounds. Rather, a small molecule is typically characterized in that it contains several carbon-carbon bonds and has a molecular weight of less than 1500 g/mol, less than 1250 g/mol, less than 1000 g/mol, less than 750 g/mol, less than 500 g/mol, or less than 250 g/mol, although this characterization is not intended to be limiting for the purposes of the small molecules disclosed herein.

In some embodiments, a small molecule specifically binds a ligand. It will be understood by those skilled in the art that the term “ligand” generally refers to a substance that forms a complex with another biomolecule. In certain embodiments, a small molecule binds to a polypeptide ligand. In certain embodiments, the ligand comprises a CACFD1 polypeptide. In certain embodiments, the ligand comprises a cell-surface CACFD1 polypeptide. In certain embodiments, the ligand comprises a CACFD1 polypeptide set forth in any one of SEQ ID NOs:231-234. In some embodiments, the small molecule binds to a region having the sequence set forth in any one of SEQ ID NOs:251-258. In one embodiment, the small molecule provided herein binds to a target having the amino acid sequence of SEQ ID NO:251. In one embodiment, the small molecule provided herein binds to a target having the amino acid sequence of SEQ ID NO:252. In one embodiment, the small molecule provided herein binds to a target having the amino acid sequence of SEQ ID NO:253. In one embodiment, the small molecule provided herein binds to a target having the amino acid sequence of SEQ ID NO:254. In one embodiment, the small molecule provided herein binds to a target having the amino acid sequence of SEQ ID NO:255. In one embodiment, the small molecule provided herein binds to a target having the amino acid sequence of SEQ ID NO:256. In one embodiment, the small molecule provided herein binds to a target having the amino acid sequence of SEQ ID NO:257. In one embodiment, the small molecule provided herein binds to a target having the amino acid sequence of SEQ ID NO:258.

Similar to the antibodies disclosed herein, the small molecules described herein would be useful in increasing cellular fitness. In some embodiments, small molecules that bind to Flower-Lose isoforms on host cells would protect the cells from competition, whereas small molecules that bind to Flower-Win isoforms on cancer cells or other cells in diseased states would reduce the ability of those cells to be overly competitive.

### Pharmaceutical Compositions

In some embodiments, disclosed herein are compositions for therapeutic use. In some embodiments, a composition described herein comprises the antibodies as disclosed herein and a pharmaceutically acceptable carrier.

As used herein, the terms “composition” and pharmaceutical composition” may in some embodiments be used interchangeably having all the same qualities and meanings. In some embodiments, disclosed herein is a pharmaceutical composition for the treatment of a condition or disease as described herein.

In some embodiments, disclosed herein are pharmaceutical compositions for use in a combination therapy.

In some embodiments, disclosed herein are compositions for use in treating a disease or condition in a subject. In some embodiments, the disease comprises a cancer. In some embodiments, the disease or condition comprises aging, stroke, cardiovascular or liver disease.

The compositions comprising the VH and/or VL polypeptides disclosed herein can be administered to a subject (e.g., a human or an animal) alone, or in combination with a carrier, i.e., a pharmaceutically acceptable carrier. By pharmaceutically acceptable is meant a material that is not biologically or otherwise undesirable, i.e., the material can be administered to a subject without causing any undesirable biological effects or interacting in a deleterious manner with any of the other components of the pharmaceutical composition in which it is contained. As would be well-known to one of ordinary skill in the art, the carrier is selected to minimize any degradation of the polypeptides disclosed herein and to minimize any adverse side effects in the subject. The pharmaceutical compositions may be prepared by methodology well known in the pharmaceutical art.

The above pharmaceutical compositions comprising the polypeptides disclosed herein can be administered (e.g., to a mammal, a cell, or a tissue) in any suitable manner depending on whether local or systemic treatment is desired. For example, the composition can be administered topically (e.g., ophthalmically, vaginally, rectally, intranasally, transdermally, and the like), orally, by inhalation, or parenterally (including by intravenous drip or subcutaneous, intracavity, intraperitoneal, intradermal, or intramuscular injection). Topical intranasal administration refers to delivery of the compositions into the nose and nasal passages through one or both of the nares.

The composition can be delivered by a spraying mechanism or droplet mechanism, or through aerosolization. Delivery can also be directed to any area of the respiratory system (e.g., lungs) via intubation. Alternatively, administration can be intratumoral, e.g., local, or intravenous injection.

A skilled artisan would appreciate that the phrases “physiologically acceptable carrier”, “pharmaceutically acceptable carrier”, “physiologically acceptable excipient”, and “pharmaceutically acceptable excipient”, may be used interchangeably may encompass a carrier, excipient, or a diluent that does not cause significant irritation to an organism and does not abrogate the biological activity and properties of the administered active ingredient.

A skilled artisan would appreciate that an “excipient” may encompass an inert substance added to a pharmaceutical composition to further facilitate administration of an active ingredient. In some embodiments, excipients include calcium carbonate, calcium phosphate, various sugars and types of starch, cellulose derivatives, gelatin, vegetable oils, and polyethylene glycols.

Techniques for formulation and administration of drugs are found in “Remington's Pharmaceutical Sciences,” Mack Publishing Co., Easton, PA, latest edition, which is incorporated herein by reference.

In some embodiments, the composition as disclosed herein comprises a therapeutic composition. In some embodiments, the composition as disclosed herein comprises a therapeutic efficacy.

In some embodiments, the present disclosure provides compositions comprising the anti-CACFD1 antibodies disclosed herein. In some embodiments, the antibodies comprise the CDR sequences as described above. In some embodiments, the antibodies comprise heavy and light chain variable regions that are at least 80% identical to the heavy and light chain variable regions described above. In some embodiments, the antibodies comprise heavy and light chain variable regions as described above. In some embodiments, the antibody comprises an IgG, IgA, IgM, IgE, IgD, a Fv, a scFv, a Fab, a F(ab′)2, a minibody, a diabody, a triabody, a multibody, a bispecific antibody, or a multi-specific antibody. In some embodiments, the IgG comprises an IgG1, IgG2, IgG3, or IgG4.

In some embodiments, the present disclosure provides compositions comprising the small molecules described herein, i.e., small molecules that bind to a target having one of the amino acid sequence of SEQ ID NOs:251-258 as described above.

### Methods of Producing an Anti-Cacfd1 Antibody or Binding Fragment Thereof/Methods of Use

In one embodiment, the present disclosure provides a method of producing a heavy chain variable region of an anti-CACFD1 antibody, the method comprises the step of culturing host cells under conditions conducive to expressing a vector encoding the heavy chain variable region, thereby producing the heavy chain variable region of the anti-CACFD1 antibody disclosed herein.

In one embodiment, the present disclosure provides a method of producing a light chain variable region of an anti-CACFD1 antibody, the method comprises the step of culturing host cells under conditions conducive to expressing a vector encoding the light chain variable region, thereby producing the light chain variable region of the anti-CACFD1 antibody disclosed herein.

In one embodiment, the present disclosure provides a method of producing a VH and VL pair of an anti-CACFD1 antibody, the method comprises the step of culturing host cells under conditions conducive to expressing a vector encoding the heavy and light chain variable regions, thereby producing the VH and VL pair of an anti-CACFD1 antibody disclosed herein.

The exact amount of the present polypeptides or compositions thereof required to elicit the desired effects will vary from subject to subject, depending on the species, age, gender, weight, and general condition of the subject, the particular polypeptides, the route of administration, and whether other drugs are included in the regimen. Thus, it is not possible to specify an exact amount for every composition. However, an appropriate amount can be determined by one of ordinary skill in the art using routine experimentation. Dosages can vary, and the polypeptides can be administered in one or more (e.g., two or more, three or more, four or more, or five or more) doses daily, for one or more days. Guidance in selecting appropriate doses for antibodies can be readily found in the literature.

In one embodiment, the present disclosure provides a method of increasing cellular fitness of a population of cells, comprising contacting the population of cells with an agent that binds to a target having the amino acid sequence of one of SEQ ID NOs:251-258. In one embodiment, the agent is the small molecules described herein. In another embodiment, the agent is the antibodies described herein. In some embodiments, an agent comprises an antibody or a small molecule.

In some embodiments, disclosed herein are methods of increasing cellular fitness of a population of cell, comprising contacting the population of cells with an antibody, said antibody comprising three heavy chain complementarity determining regions (HCDR1, HCDR2, HCDR3) and three light chain CDRs (LCDR1, LCDR2, LCDR3), wherein
(a) the HCDR1, HCDR2, and HCDR3 have the sequences of SEQ ID NOs:266-268 respectively, and the LCDR1, LCDR2, and LCDR3 have the sequences of SEQ ID NOs:269-271 respectively; or
(b) the HCDR1, HCDR2, and HCDR3 have the sequences of SEQ ID NOs:272-274 respectively, and the LCDR1, LCDR2, and LCDR3 have the sequences of SEQ ID NOs:275-277 respectively; or
(c) the HCDR1, HCDR2, and HCDR3 have the sequences of SEQ ID NOs:278-280 respectively, and the LCDR1, LCDR2, and LCDR3 have the sequences of SEQ ID NOs:281-283 respectively; or
(d) the HCDR1, HCDR2, and HCDR3 have the sequences of SEQ ID NOs:284-286 respectively, and the LCDR1, LCDR2, and LCDR3 have the sequences of SEQ ID NOs:287-289 respectively; or
(e) the HCDR1, HCDR2, and HCDR3 have the sequences of SEQ ID NOs:290-292 respectively, and the LCDR1, LCDR2, and LCDR3 have the sequences of SEQ ID NOs:293-295 respectively; or
(f) the HCDR1, HCDR2, and HCDR3 have the sequences of SEQ ID NOs:296-298 respectively, and the LCDR1, LCDR2, and LCDR3 have the sequences of SEQ ID NOs:299-301 respectively; or
(g) the HCDR1, HCDR2, and HCDR3 have the sequences of SEQ ID NOs:302-304 respectively, and the LCDR1, LCDR2, and LCDR3 have the sequences of SEQ ID NOs:305-307 respectively; or
(h) the HCDR1, HCDR2, and HCDR3 have the sequences of SEQ ID NOs:308-310 respectively, and the LCDR1, LCDR2, and LCDR3 have the sequences of SEQ ID NOs:311-313 respectively; or
(i) the HCDR1, HCDR2, and HCDR3 have the sequences of SEQ ID NOs:326-328 respectively, and the LCDR1, LCDR2, and LCDR3 have the sequences of SEQ ID NOs:329-331, respectively.

In some embodiments, disclosed herein are methods of increasing cellular fitness of a population of cell, comprising contacting the population of cells with an antibody, said antibody comprises
(a) a heavy chain variable region having the amino acid sequence of SEQ ID NO:235 and a light chain variable region having the amino acid sequence of SEQ ID NO:236, or a heavy chain variable region having an amino acid sequence at least 80% identical to SEQ ID NO:235 and a light chain variable region having an amino acid sequence at least 80% identical to SEQ ID NO:236; or
(b) a heavy chain variable region having the amino acid sequence of SEQ ID NO:237 and a light chain variable region having the amino acid sequence of SEQ ID NO:238, or a heavy chain variable region having an amino acid sequence at least 80% identical to SEQ ID NO:237 and a light chain variable region having an amino acid sequence at least 80% identical to SEQ ID NO:238; or
(c) a heavy chain variable region having the amino acid sequence of SEQ ID NO:239 and a light chain variable region having the amino acid sequence of SEQ ID NO:240, or a heavy chain variable region having an amino acid sequence at least 80% identical to SEQ ID NO:239 and a light chain variable region having an amino acid sequence at least 80% identical to SEQ ID NO:240; or
(d) a heavy chain variable region having the amino acid sequence of SEQ ID NO:241 and a light chain variable region having the amino acid sequence of SEQ ID NO:242, or a heavy chain variable region having an amino acid sequence at least 80% identical to SEQ ID NO:241 and a light chain variable region having an amino acid sequence at least 80% identical to SEQ ID NO:242; or
(e) a heavy chain variable region having the amino acid sequence of SEQ ID NO:243 and a light chain variable region having the amino acid sequence of SEQ ID NO:244, or a heavy chain variable region having an amino acid sequence at least 80% identical to SEQ ID NO:243 and a light chain variable region having an amino acid sequence at least 80% identical to SEQ ID NO:244; or
(f) a heavy chain variable region having the amino acid sequence of SEQ ID NO:245 and a light chain variable region having the amino acid sequence of SEQ ID NO:246, or a heavy chain variable region having an amino acid sequence at least 80% identical to SEQ ID NO:245 and a light chain variable region having an amino acid sequence at least 80% identical to SEQ ID NO:246; or
(g) a heavy chain variable region having the amino acid sequence of SEQ ID NO:247 and a light chain variable region having the amino acid sequence of SEQ ID NO:248, or a heavy chain variable region having an amino acid sequence at least 80% identical to SEQ ID NO:247 and a light chain variable region having an amino acid sequence at least 80% identical to SEQ ID NO:248; or
(h) a heavy chain variable region having the amino acid sequence of SEQ ID NO:249 and a light chain variable region having the amino acid sequence of SEQ ID NO:250, or a heavy chain variable region having an amino acid sequence at least 80% identical to SEQ ID NO:249 and a light chain variable region having an amino acid sequence at least 80% identical to SEQ ID NO:250; or
(i) a heavy chain variable region having the amino acid sequence of one of SEQ ID NOs:314, 316, 318, 320, 322, or 324, and a light chain variable region having the amino acid sequence of one of SEQ ID NOs:315, 317, 319, 321, 323, or 325, or a heavy chain variable region having an amino acid sequence at least 80% identical to one of SEQ ID NOs:314, 316, 318, 320, 322, or 324, and a light chain variable region having an amino acid sequence at least 80% identical to one of SEQ ID NOs:315, 317, 319, 321, 323, or 325.

In one embodiment, the antibodies bind to one or more of hFwe1, hFwe2, hFwe3 and hFwe4. In one embodiment, the antibodies bind to calcium channel flower homolog protein that behaves as Flower-Win protein. In another embodiment, the antibodies bind to calcium channel flower homolog protein that behaves as Flower-Lose protein. In some embodiments, the antibodies comprise the CDR sequences as described above. In some embodiments, the antibodies comprise heavy and light chain variable regions that are at least 80% identical to the heavy and light chain variable regions described above. In some embodiments, the antibodies comprise heavy and light chain variable regions as described above. In some embodiments, the antibody comprises an IgG, IgA, IgM, IgE, IgD, a Fv, a scFv, a Fab, a F(ab′)2, a minibody, a diabody, a triabody, a multibody, a bispecific antibody, or a multi-specific antibody. In some embodiments, the IgG comprises an IgG1, IgG2, IgG3, or IgG4.

In another embodiment, the present disclosure provides a method of preventing apoptosis of a population of cells, comprising contacting the population of cells with an agent that binds to a target having the amino acid sequence of one of SEQ ID NOs:251-258. In one embodiment, the agent is the small molecules described herein. In another embodiment, the agent is the antibodies described herein. In one embodiment, the antibodies bind to one or more of hFwe1, hFwe2, hFwe3 and hFwe4. In one embodiment, the antibodies bind to calcium channel flower homolog protein that behaves as Flower-Win protein. In another embodiment, the antibodies bind to calcium channel flower homolog protein that behaves as Flower-Lose protein. In some embodiments, the antibodies comprise the CDR sequences as described above.

In some embodiments, the present disclosure provides a method of preventing apoptosis of a population of cells, comprising contacting the population of cells with an antibody, said antibody comprising three heavy chain complementarity determining regions (HCDR1, HCDR2, HCDR3) and three light chain CDRs (LCDR1, LCDR2, LCDR3), wherein
(a) the HCDR1, HCDR2, and HCDR3 have the sequences of SEQ ID NOs:266-268 respectively, and the LCDR1, LCDR2, and LCDR3 have the sequences of SEQ ID NOs:269-271 respectively; or
(b) the HCDR1, HCDR2, and HCDR3 have the sequences of SEQ ID NOs:272-274 respectively, and the LCDR1, LCDR2, and LCDR3 have the sequences of SEQ ID NOs:275-277 respectively; or
(c) the HCDR1, HCDR2, and HCDR3 have the sequences of SEQ ID NOs:278-280 respectively, and the LCDR1, LCDR2, and LCDR3 have the sequences of SEQ ID NOs:281-283 respectively; or
(d) the HCDR1, HCDR2, and HCDR3 have the sequences of SEQ ID NOs:284-286 respectively, and the LCDR1, LCDR2, and LCDR3 have the sequences of SEQ ID NOs:287-289 respectively; or
(e) the HCDR1, HCDR2, and HCDR3 have the sequences of SEQ ID NOs:290-292 respectively, and the LCDR1, LCDR2, and LCDR3 have the sequences of SEQ ID NOs:293-295 respectively; or
(f) the HCDR1, HCDR2, and HCDR3 have the sequences of SEQ ID NOs:296-298 respectively, and the LCDR1, LCDR2, and LCDR3 have the sequences of SEQ ID NOs:299-301 respectively; or
(g) the HCDR1, HCDR2, and HCDR3 have the sequences of SEQ ID NOs:302-304 respectively, and the LCDR1, LCDR2, and LCDR3 have the sequences of SEQ ID NOs:305-307 respectively; or
(h) the HCDR1, HCDR2, and HCDR3 have the sequences of SEQ ID NOs:308-310 respectively, and the LCDR1, LCDR2, and LCDR3 have the sequences of SEQ ID NOs:311-313 respectively; or
(i) the HCDR1, HCDR2, and HCDR3 have the sequences of SEQ ID NOs:326-328 respectively, and the LCDR1, LCDR2, and LCDR3 have the sequences of SEQ ID NOs:329-331, respectively.

In some embodiments, the present disclosure provides a method of preventing apoptosis of a population of cells, comprising contacting the population of cells with an antibody, said antibody comprising
(a) a heavy chain variable region having the amino acid sequence of SEQ ID NO:235 and a light chain variable region having the amino acid sequence of SEQ ID NO:236, or a heavy chain variable region having an amino acid sequence at least 80% identical to SEQ ID NO:235 and a light chain variable region having an amino acid sequence at least 80% identical to SEQ ID NO:236; or
(b) a heavy chain variable region having the amino acid sequence of SEQ ID NO:237 and a light chain variable region having the amino acid sequence of SEQ ID NO:238, or a heavy chain variable region having an amino acid sequence at least 80% identical to SEQ ID NO:237 and a light chain variable region having an amino acid sequence at least 80% identical to SEQ ID NO:238; or
(c) a heavy chain variable region having the amino acid sequence of SEQ ID NO:239 and a light chain variable region having the amino acid sequence of SEQ ID NO:240, or a heavy chain variable region having an amino acid sequence at least 80% identical to SEQ ID NO:239 and a light chain variable region having an amino acid sequence at least 80% identical to SEQ ID NO:240; or
(d) a heavy chain variable region having the amino acid sequence of SEQ ID NO:241 and a light chain variable region having the amino acid sequence of SEQ ID NO:242, or a heavy chain variable region having an amino acid sequence at least 80% identical to SEQ ID NO:241 and a light chain variable region having an amino acid sequence at least 80% identical to SEQ ID NO:242; or
(e) a heavy chain variable region having the amino acid sequence of SEQ ID NO:243 and a light chain variable region having the amino acid sequence of SEQ ID NO:244, or a heavy chain variable region having an amino acid sequence at least 80% identical to SEQ ID NO:243 and a light chain variable region having an amino acid sequence at least 80% identical to SEQ ID NO:244; or
(f) a heavy chain variable region having the amino acid sequence of SEQ ID NO:245 and a light chain variable region having the amino acid sequence of SEQ ID NO:246, or a heavy chain variable region having an amino acid sequence at least 80% identical to SEQ ID NO:245 and a light chain variable region having an amino acid sequence at least 80% identical to SEQ ID NO:246; or
(g) a heavy chain variable region having the amino acid sequence of SEQ ID NO:247 and a light chain variable region having the amino acid sequence of SEQ ID NO:248, or a heavy chain variable region having an amino acid sequence at least 80% identical to SEQ ID NO:247 and a light chain variable region having an amino acid sequence at least 80% identical to SEQ ID NO:248; or
(h) a heavy chain variable region having the amino acid sequence of SEQ ID NO:249 and a light chain variable region having the amino acid sequence of SEQ ID NO:250, or a heavy chain variable region having an amino acid sequence at least 80% identical to SEQ ID NO:249 and a light chain variable region having an amino acid sequence at least 80% identical to SEQ ID NO:250; or
(i) a heavy chain variable region having the amino acid sequence of one of SEQ ID NOs:314, 316, 318, 320, 322, or 324, and a light chain variable region having the amino acid sequence of one of SEQ ID NOs:315, 317, 319, 321, 323, or 325, or a heavy chain variable region having an amino acid sequence at least 80% identical to one of SEQ ID NOs:314, 316, 318, 320, 322, or 324, and a light chain variable region having an amino acid sequence at least 80% identical to one of SEQ ID NOs:315, 317, 319, 321, 323, or 325.

In some embodiments, the antibodies comprise heavy and light chain variable regions that are at least 80% identical to the heavy and light chain variable regions described above. In some embodiments, the antibodies comprise heavy and light chain variable regions as described above.

In some embodiments, the antibody comprises an IgG, IgA, IgM, IgE, IgD, a Fv, a scFv, a Fab, a F(ab′)2, a minibody, a diabody, a triabody, a multibody, a bispecific antibody, or a multi-specific antibody. In some embodiments, the IgG comprises an IgG1, IgG2, IgG3, or IgG4.

As discussed herein, it is believed that the cancerous mass behaves as a supercompetitive partner and carries the potential to outcompete the surrounding healthy cells. Thus, for cell competition at the tumor-stroma interface, the tumoral tissue might activate genetic and transcriptional changes (e.g., expression of Flower-Win isoforms), which help cancer tissue achieve supercompetitive status. The host tissue adjacent to the cancer might result in changes that transform the host cells into loser cells (e.g., upregulation of Flower-Lose isoforms), thus promoting their removal. Results presented herein show that Flower Win isoforms on cancer cells are activators of Flower Lose-dependent death of TME cells. The ability of cancer cells to outcompete stromal cells is drastically reduced in the presence of anti-CACFD1 antibodies disclosed herein. Anti-CACFD1 antibodies that bind to Flower-Lose isoforms on host cells would protect the stromal cells from competition, whereas anti-CACFD1 antibodies that bind to Flower-Win isoforms on cancer cells would reduce the ability of cancer cells to be overly competitive.

Similar to cell competition between cancer cells and host cells, cell competition also occurs in other diseases or conditions. For example, diseases such as stroke, cardiovascular disease (e.g., myocardial infarction), neurodegenerative diseases (e.g., Alzheimer's, Parkinson's, multiple sclerosis, Huntington's diseases), liver cirrhosis and even aging all involve cell competition with the expression of Flower-Win and Flower-Lose isoforms. Just like cancer cells clear the tumor micro-environment to expand, competitive cells in the neighborhood of infarct during myocardial infarction or stroke eliminate cells in regions surrounding the infarct. This leads to massive loss of tissue which is undesirable and eventually leads to death or ageing. In all these scenarios, Flower Lose isoforms are expressed in the cells that are being eliminated or at a competitive disadvantage. Targeting Flower-Lose isoforms in these conditions (e.g., with small molecules or the anti-CACFD1 antibodies disclosed herein) would protect these cells from being eliminated or out-competed by the surrounding cells. In other embodiments, in diseases like stroke and Alzheimer's, it may be more beneficial to target Flower-Win instead of Flower-Lose isoforms.

Accordingly, the present disclosure also provides a method of treating a cancer, a disease or a condition in a subject in need thereof, comprising administering to the subject an agent that binds to a target having the amino acid sequence of one of SEQ ID NOs:251-258. In some embodiments, the disease or condition comprises stroke, cardiovascular disease, neurodegenerative diseases, or aging. In one embodiment, the agent is the small molecules described herein. In another embodiment, the agent is the antibodies described herein. In one embodiment, the antibodies bind to one or more of hFwe1, hFwe2, hFwe3 and hFwe4. In one embodiment, the antibodies bind to calcium channel flower homolog protein that behaves as Flower-Win protein. In another embodiment, the antibodies bind to calcium channel flower homolog protein that behaves as Flower-Lose protein. In some embodiments, the antibodies comprise the CDR sequences as described above. In some embodiments, the antibodies comprise heavy and light chain variable regions that are at least 80% identical to the heavy and light chain variable regions described above. In some embodiments, the antibodies comprise heavy and light chain variable regions as described above.

In some embodiments, the present disclosure provides a method of treating a cancer, a disease or a condition in a subject in need thereof comprising administering to the subject an antibody, said antibody comprising three heavy chain complementarity determining regions (HCDR1, HCDR2, HCDR3) and three light chain CDRs (LCDR1, LCDR2, LCDR3), wherein
(a) the HCDR1, HCDR2, and HCDR3 have the sequences of SEQ ID NOs:266-268 respectively, and the LCDR1, LCDR2, and LCDR3 have the sequences of SEQ ID NOs:269-271 respectively; or
(b) the HCDR1, HCDR2, and HCDR3 have the sequences of SEQ ID NOs:272-274 respectively, and the LCDR1, LCDR2, and LCDR3 have the sequences of SEQ ID NOs:275-277 respectively; or
(c) the HCDR1, HCDR2, and HCDR3 have the sequences of SEQ ID NOs:278-280 respectively, and the LCDR1, LCDR2, and LCDR3 have the sequences of SEQ ID NOs:281-283 respectively; or
(d) the HCDR1, HCDR2, and HCDR3 have the sequences of SEQ ID NOs:284-286 respectively, and the LCDR1, LCDR2, and LCDR3 have the sequences of SEQ ID NOs:287-289 respectively; or
(e) the HCDR1, HCDR2, and HCDR3 have the sequences of SEQ ID NOs:290-292 respectively, and the LCDR1, LCDR2, and LCDR3 have the sequences of SEQ ID NOs:293-295 respectively; or
(f) the HCDR1, HCDR2, and HCDR3 have the sequences of SEQ ID NOs:296-298 respectively, and the LCDR1, LCDR2, and LCDR3 have the sequences of SEQ ID NOs:299-301 respectively; or
(g) the HCDR1, HCDR2, and HCDR3 have the sequences of SEQ ID NOs:302-304 respectively, and the LCDR1, LCDR2, and LCDR3 have the sequences of SEQ ID NOs:305-307 respectively; or
(h) the HCDR1, HCDR2, and HCDR3 have the sequences of SEQ ID NOs:308-310 respectively, and the LCDR1, LCDR2, and LCDR3 have the sequences of SEQ ID NOs:311-313 respectively; or
(i) the HCDR1, HCDR2, and HCDR3 have the sequences of SEQ ID NOs:326-328 respectively, and the LCDR1, LCDR2, and LCDR3 have the sequences of SEQ ID NOs:329-331, respectively.

In some embodiments, the present disclosure provides a method of treating a cancer, a disease or a condition in a subject in need thereof comprising administering to the subject an 25 antibody, said antibody comprising
(a) a heavy chain variable region having the amino acid sequence of SEQ ID NO:235 and a light chain variable region having the amino acid sequence of SEQ ID NO:236, or a heavy chain variable region having an amino acid sequence at least 80% identical to SEQ ID NO:235 and a light chain variable region having an amino acid sequence at least 80% identical to SEQ ID NO:236; or
(b) a heavy chain variable region having the amino acid sequence of SEQ ID NO:237 and a light chain variable region having the amino acid sequence of SEQ ID NO:238, or a heavy chain variable region having an amino acid sequence at least 80% identical to SEQ ID NO:237 and a light chain variable region having an amino acid sequence at least 80% identical to SEQ ID NO:238; or
(c) a heavy chain variable region having the amino acid sequence of SEQ ID NO:239 and a light chain variable region having the amino acid sequence of SEQ ID NO:240, or a heavy chain variable region having an amino acid sequence at least 80% identical to SEQ ID NO:239 and a light chain variable region having an amino acid sequence at least 80% identical to SEQ ID NO:240; or
(d) a heavy chain variable region having the amino acid sequence of SEQ ID NO:241 and a light chain variable region having the amino acid sequence of SEQ ID NO:242, or a heavy chain variable region having an amino acid sequence at least 80% identical to SEQ ID NO:241 and a light chain variable region having an amino acid sequence at least 80% identical to SEQ ID NO:242; or
(e) a heavy chain variable region having the amino acid sequence of SEQ ID NO:243 and a light chain variable region having the amino acid sequence of SEQ ID NO:244, or a heavy chain variable region having an amino acid sequence at least 80% identical to SEQ ID NO:243 and a light chain variable region having an amino acid sequence at least 80% identical to SEQ ID NO:244; or
(f) a heavy chain variable region having the amino acid sequence of SEQ ID NO:245 and a light chain variable region having the amino acid sequence of SEQ ID NO:246, or a heavy chain variable region having an amino acid sequence at least 80% identical to SEQ ID NO:245 and a light chain variable region having an amino acid sequence at least 80% identical to SEQ ID NO:246; or
(g) a heavy chain variable region having the amino acid sequence of SEQ ID NO:247 and a light chain variable region having the amino acid sequence of SEQ ID NO:248, or a heavy chain variable region having an amino acid sequence at least 80% identical to SEQ ID NO:247 and a light chain variable region having an amino acid sequence at least 80% identical to SEQ ID NO:248; or
(h) a heavy chain variable region having the amino acid sequence of SEQ ID NO:249 and a light chain variable region having the amino acid sequence of SEQ ID NO:250, or a heavy chain variable region having an amino acid sequence at least 80% identical to SEQ ID NO:249 and a light chain variable region having an amino acid sequence at least 80% identical to SEQ ID NO:250; or
(i) a heavy chain variable region having the amino acid sequence of one of SEQ ID NOs:314, 316, 318, 320, 322, or 324, and a light chain variable region having the amino acid sequence of one of SEQ ID NOs:315, 317, 319, 321, 323, or 325, or a heavy chain variable region having an amino acid sequence at least 80% identical to one of SEQ ID NOs:314, 316, 318, 320, 322, or 324, and a light chain variable region having an amino acid sequence at least 80% identical to one of SEQ ID NOs:315, 317, 319, 321, 323, or 325.

In some embodiments, the antibody comprises an IgG, IgA, IgM, IgE, IgD, a Fv, a scFv, a Fab, a F(ab′)2, a minibody, a diabody, a triabody, a multibody, a bispecific antibody, or a multi-specific antibody. In some embodiments, the IgG comprises an IgG1, IgG2, IgG3, or IgG4.

A skilled artisan would appreciate that the term “treating” and grammatical forms thereof, may in some embodiments encompass both therapeutic treatment and prophylactic or preventative measures with respect to a tumor or cancer, wherein the object is to prevent or lessen the targeted tumor or cancer. Thus, in some embodiments of methods disclosed herein, treating may include directly affecting or curing, suppressing, inhibiting, preventing, reducing the severity of, delaying the onset of, reducing symptoms associated with the disease, disorder or condition, or a combination thereof, for example, when said disease or disorder comprises a cancer or tumor. Thus, in some embodiments, “treating” encompasses preventing, delaying progression, inhibiting the growth of, delaying disease progression, reducing tumor load, reducing the incidence of, expediting remission, inducing remission, augmenting remission, speeding recovery, increasing efficacy of, or decreasing resistance to alternative therapeutics, or a combination thereof. In some embodiments, “preventing” encompasses delaying the onset of symptoms, preventing relapse to a disease, decreasing the number or frequency of relapse episodes, increasing latency between symptomatic episodes, or a combination thereof. In some embodiments, “suppressing” or “inhibiting”, encompass reducing the severity of symptoms, reducing the severity of an acute episode, reducing the number of symptoms, reducing the incidence of disease-related symptoms, reducing the latency of symptoms, ameliorating symptoms, reducing secondary symptoms, reducing secondary infections, prolonging patient survival, or a combination thereof.

In some embodiments, the size of a cancer or tumor is reduced. In some embodiments, the growth rate of a cancer or tumor is reduced. In some embodiments, the size or the growth rate or a combination thereof, of a cancer or tumor is reduced. In some embodiments, the survival of the subject in need is increased. In some embodiments, the size or the growth rate or a combination thereof, of a cancer or tumor is reduced, or wherein the survival of the subject in need is increased or a combination thereof.

In some embodiments, the subject in need is a human subject. In some embodiments, the subject in need is a human child. In some embodiments, the subject in need is an adult human. In some embodiments, the subject in need is a human infant.

In certain embodiments, the amount administered is sufficient to result in tumor regression, as indicated by a statistically significant decrease in the amount of viable tumor, for example, at least a 50% decrease in tumor mass, or by altered (e.g., decreased with statistical significance) scan dimensions. In other embodiments, the amount administered is sufficient to result in clinically relevant reduction in disease symptoms as would be known to the skilled clinician.

The precise dosage and duration of treatment is a function of the disease being treated and may be determined empirically using known testing protocols or by testing the compositions in model systems known in the art and extrapolating therefrom. Controlled clinical trials may also be performed. Dosages may also vary with the severity of the condition to be alleviated. A pharmaceutical composition is generally formulated and administered to exert a therapeutically useful effect while minimizing undesirable side effects. The composition may be administered one time or may be divided into a number of smaller doses to be administered at intervals of time. For any particular subject, specific dosage regimens may be adjusted over time according to the individual need.

In some embodiments, the cancer or tumor is a solid tumor. Solid tumors may be benign (not cancer), or malignant (cancer). Different types of solid tumors are named after the type of cells that form them. Examples of solid tumors are sarcomas, carcinomas, and lymphomas. In some embodiments, solid tumors are neoplasms (new growth of cells), or lesions (damage of anatomic structures or disturbance of physiological functions) formed by an abnormal growth of body tissue cells other than blood, bone marrow, or lymphatic cells. In some embodiments, a solid tumor consists of an abnormal mass of cells which may stem from different tissue types such as liver, colon, breast, or lung, and which initially grows in the organ of its cellular origin. However, such cancers may spread to other organs through metastatic tumor growth in advanced stages of the disease.

In some embodiments, the solid tumor comprises an ovarian cancer or tumor for example but not limited to a high grade serous ovarian carcinoma (HGSG), a colon carcinoma, a pancreatic cancer or tumor, a breast cancer or tumor, a prostate cancer or tumor, a squamous cell carcinoma, a basal cell carcinoma, an adenocarcinoma, a renal cell carcinoma, a hepatoma, a cervical cancer or tumor, a uterine cancer or tumor, a testicular cancer or tumor, a lung carcinoma, a small cell lung carcinoma, a bladder carcinoma, a glioma, an astrocytoma, a medulloblastoma, a melanoma, or a neuroblastoma. In some embodiments, the solid tumor comprises an adrenocortical tumor (adenoma and carcinoma), a carcinoma, a colorectal carcinoma, a Desmoid tumor, a Desmoplastic small round cell tumor, a germ cell tumor, a hepatoblastoma, a hepatocellular carcinoma, an osteosarcoma. In some embodiments, the solid tumor comprises an ovarian cancer or tumor for example but not limited to a high grade serous ovarian carcinoma (HGSG) or a colon carcinoma.

In some embodiments, methods disclosed herein treat an ovarian cancer or tumor for example but not limited to a high grade serous ovarian carcinoma (HGSG). In some embodiments, methods disclosed herein treat a colon carcinoma. In some embodiments, methods disclosed herein treat an ovarian cancer or tumor, or a colon carcinoma.

### Combination Uses

In some embodiments, antibodies disclosed herein may be used in combination therapies. Combination therapies may be used in methods of treating cancer, diseases, or conditions. In some embodiments, combination therapies are used for treating cancers. In some embodiments, combination therapies are used for treating diseases or conditions other than cancer. In some embodiments, cancers comprise solid cancers as described above. In some embodiments, diseases and conditions include aging, stroke, cardiovascular or liver disease. In some embodiments, combination therapies include Docetaxel, carboplatin, and or bevacizumab.

Various embodiments and aspects of the antibodies and uses thereof as delineated hereinabove and as claimed in the claims section below find experimental support in the following examples.

### Example 1

### Materials and Methods

### Cell Culture, Lentivirus-Assisted Overexpression, Knockdown, And CRISPR-Assisted Knockout

HEK 293T cells were obtained from ATCC, OVCAR4, and OVCAR8 cells were provided by Karuppaiyah Selvendiran (OSU), obtained from ATCC. In brief, OVCAR4, OVCAR8 and HEK 293T cells were cultured as suggested by the ATCC in Dulbecco's modified Eagle's medium (DMEM) supplemented with 10% (v/v) heat-inactivated fetal bovine serum (FBS) (Gibco) and 1% (v/v) penicillin/streptomycin (Thermo Fisher Scientific) and incubated at 37° C. in a humidified atmosphere of 95% air and 5% C02. Cells were tested for mycoplasma contamination. Cell lines were authenticated by the commercial vendor, authentication techniques can be found on the vendor website. Exosome inhibition was performed using Amiloride hydrochloride (Merck) and GW4869 (Merck), DNMT3A inhibition was performed using decitabine (Santa Cruz Biotechnology), and cytokine release inhibition using Brefeldin A (Thermo Fisher Scientific). Stromal cells were isolated from fresh patient surgery material as described previously. For extraction of the stromal cells, approximately 2 g of the ovarian cancer patient surgery sample was used as starting material for tissue digestion. After manual removal of fatty tissue, the remaining tissue was minced into 3-4 mm pieces and placed in a 1 μL sterile glass bottle containing DMEM/F12 medium supplemented with collagenase Type IV (200 U/ml) and hyaluronidase (100 U/ml), reaching three times the tissue volume. The bottle was incubated on a plate shaker at 37° C. overnight. The digested tissue was sequentially filtered through mesh sizes of 500 μm, 250 μm, 100 μm, 40 μm, and 20 μm to enrich for stromal cell fractions, as previously described. The stromal-enriched fractions were collected from the flow-through after passing through the 20 μm strainer and subsequently centrifuged. The resulting pellet was resuspended in DMEM medium to support stromal cell growth. Lentiviral production was performed under BL2/BSL-2 conditions. All lentiviruses were produced by transfecting 1×10⁶ HEK293 T cells with lentiviral plasmids using Lipofectamine® 3000 per manufacturer instructions. Cell supernatants containing lentiviruses were collected 72 h post-transfection, filtered through a 0.45 m filter and concentrated by ultracentrifugation. The lentiviral particles were tittered using p24 ELISA (Cell BioLabs) per the manufacturer's protocol. Target cells were infected with 10⁹ TU/ml of lentiviral particles containing 5 g/ml of polybrene for 24 h.

For knockout (KO) experiments, the gRNAs targeting Tu-Stroma 5′UTR and Intron 1 were cloned in pLentiCRISPR_v2 vectors (target 5′UTR: 5′-GCTTTGTACCAACTCATTGG-3′ (SEQ ID NO:207); target Intron 1: 5′-ACTCGCCAGAGTTCCACAAC-3′ (SEQ ID NO:208)). For DDX3X KO, the gRNAs targeting DDX3X Exon 1 and Intron 1 were cloned in pLentiCRISPR_v2 vectors (target Exon 1: 5′-CACGGATCTAATATACCCGC-3′ (SEQ ID NO:332); target Intron 1: 5′-TCCCGGCTAACATACCACAC-3′ (SEQ ID NO:333)). After producing lentiviral particles, OVCAR8 cells were infected with gRNAs targeting only Tu-Stroma, only DDX3X, or targeting Tu-Stroma and DDX3X and were selected with puromycin. Then cells were single-cell sorted in 96-well plates and the DNA of each colony was extracted to confirm the KO by PCR using the following primers for Tu-Stroma (F) 5′-GGCTGGGAGGTCTTACTCCT-3′ (SEQ ID NO:334) and (R) 5′-CTGACACTAATCGGGGGCAC-3′ (SEQ ID NO:335) and for DDX3X (F) 5′-AGGGTTTTAGCGGAGAGCAC-3′ (SEQ ID NO:336) and (R) 5′-TCTGTGCTGCTGGTAGGTTG-3′ (SEQ ID NO:337). We generated lentiviral particles to double KO Rab27a and Rab27b by cloning in pLentiCRISPR_v2 vectors the sequences for Rab27a 5′-CACCCAGTATTCATACCCACTCCG-3′ (Sense) (SEQ ID NO:338) and 5′-AAACCGGAGTGGGTATGAATACTG-3′ (Antisense) (SEQ ID NO:339), the sequences targeting Rab27b 5′-CACCGCACTCGCAGTCCTGACGGGGCAGGG-3′ (Sense) (SEQ ID NO:340) and 5′-AAACCCCCTGCCCGTCAGGACTGCGAGTG-3′ (Antisense) (SEQ ID NO:341) as described by Broner et a1⁵⁰. The double KO was confirmed by RT-qPCR using primers for Rab27a 5′-GCTTTGGGAGACTCTGGTGTA-3′ (F) (SEQ ID NO:342) and 5′-TCAATGCCCACTGTTGTGATAA-3′ (R) (SEQ ID NO:343) and for Rab27b 5′-TAGACTTTCGGGAAAAACGTGTG-3′ (F) (SEQ ID NO:344) and 5′-AGAAGCTCTGTTGACTGGTGA-3′ (R) (SEQ ID NO:345).

For knockdown (KD) experiments, 3 shRNAs against Tu-Stroma were cloned in pLKO.1-puro vectors by Genscript (target 1: 5′-ATCTCTGCAGTTACAATTATT-3′ (SEQ ID NO:211); target 2: 5′-ACCTGCAAATTCTACACATTA-3′ (SEQ ID NO:212); target 3: 5′-CAGCGGCCATGGAAAGAACTT-3′ (SEQ ID NO:213)). Additionally, we purchased from Merk shRNAs against the following genes: hFwe (target 1: 5′-TCTGGCCTCTTCAACTGCATCACCATCCA-3′ (SEQ ID NO:346); target 2: 5′-TGAATGCCTTCATCTTGTTGCTGTGTGAG-3′ (SEQ ID NO:347); target 3: 5′-CGCTCCTGGCAGAAGGCTGTCTTCTACTG-3′ (SEQ ID NO:348); target 4: 5′-GGCAGCAGGCGGATGAGGAGAAGCTCGCG-3′ (SEQ ID NO:349); hSTAT3 (target 1: 5′-GCAAAGAATCACATGCCACTT-3′ (SEQ ID NO:214); target 2: 5′-GCACAATCTACGAAGAATCAA-3′ (SEQ ID NO:215)); hDDX3X (target 1: 5′-CGGAGTGATTACGATGGCATT-3′ (SEQ ID NO:216); target 2: 5′-CGTAGAATAGTCGAACAAGAT-3′ (SEQ ID NO:217)); hDNMT3A (target: 5′-CCACCAGAAGAAGAGAAGAAT-3′ (SEQ ID NO:218)); hG9A (target: 5′-CGAGAGAGTTCATGGCTCTTT-3′ (SEQ ID NO:219)); hGLP (target: 5′-GGATTCAGATGTCACCTTAAA-3′ (SEQ ID NO:220)); hHPI (target: 5′-TAACAAGAGGAAATCCAATTT-3′ (SEQ ID NO:221)); and hSRSF3 (target: 5′-TGGAACTGTCGAATGGTGAAA-3′ (SEQ ID NO:222)). For Tu-Stroma silencing, a Poly-A sequence was inserted in LINC01914 Exon 1 and cloned in the pUC57 plasmid (Genscript). A gRNA targeting the LINC01914 (5′-GTTACTACTGTTGTGCCCGT-3′ (SEQ ID NO:350)) was cloned in an eSpCas9-2A-GFP vector (Genscript). Then, cells were transfected with both plasmids and selected for GFP and puromycin. The insertion of Poly-A was confirmed by PCR using the primers 5′-GAAGCTGACCATTGACCATTGTCTTG-3′ (F) (SEQ ID NO:351) and 5′-CAGGAATCTCATCCAGCTGCTC-3′ (SEQ ID NO:352).

For overexpression experiments, Tu-Stroma cDNA was cloned into pLenti-GIII-EFla vector. For luciferase assay experiments, we cloned on pGL4-puro vector the following sequences: hFwe 4k promoter; hFwe STAT3 DBS 1 (5′-TCGCTTCCCGGCCCCAGCCC-3′ (SEQ ID NO:223)); hFwe STAT3 DBS 2 (5′-TCACTGGGCCAGGAAGTGG-3′ (SEQ ID NO:224)); hFwe STAT3 DBS 3 (5′-CCTCAGTCCTGGAAGGGA-3′ (SEQ ID NO:225)); hFwe STAT3 DBS 4 (5′-GCGCTTCTGGGAACATTCC-3′ (SEQ ID NO:226)); and STAT3 c-FOS binding site (5′-GAGCAGTTCCCGTCAATCCCT-3′ (SEQ ID NO:227)). Tu-Stroma cDNA and all sequences for luciferase assay experiments were cloned by Genscript. For in vivo experiments, mice were injected with 10” TU/ml of lentiviral particles as described by Madan et al. CRISPR-off experiment was performed as described previously by Pan et al. Stromal cells were transfected in 24-well plates using Lipofectamine® 3000 and Opti-MEM™ I Reduced Serum Medium (Thermo Fisher Scientific), as per manufacturer instructions. We used 300 ng of plasmid encoding CRISPR-off, dCas9-KRAB, or dCas9-D3A-3 μL and 150 ng of plasmids encoding sgRNAs for hFwe Exon 3. As a second approach, we performed transfection with plasmid encoding CRISPR-off, dCas9-DNMT3A, or dCas9-DNMT3B, and plasmids encoding sgRNAs for hFwe Exon 3. After 48 hours, cells were selected, and hFwe Exon 3 methylation was confirmed by methylation-specific PCR (protocol described below) and by running the PCR products in agarose gel electrophoresis. As sgRNA, we used the following sequence targeting hFwe Exon 3 5′-CTTCATCTTGTTGCTGTGTG-3′ (SEQ ID NO:353).

### Patient Samples

The normal ovarian and high-grade serous carcinoma (HGSC) patient samples (both cancer and stroma) were obtained in the form of formalin-fixed paraffin-embedded (FFPE), fresh surgery, and blood samples from Dr Michael Idowu, Dr David Chelmow, Dr Katherine P Klein, Virginia Commonwealth University (VCU) Department of Pathology, VCU Department of ObGyn, Cancer Informatics and Data Analytics core at VCU Massey Comprehensive Cancer Center, VCU, Richmond, VA, USA (protocol numbers: HM20028457 & HM20021072); Dr Andrew K Godwin from Department of Pathology and Laboratory Medicine, University of Kansas Medical Center, Kansas City, KS, USA provided the samples banked by the Biospecimen Repository Core Facility University of Kansas Medical Center, under a longstanding IRB approved protocol (protocol number: HSC #5929); Kirsten D. Mertz, MD PhD, University Hospital Basel, Institute of Medical Genetics and Pathology, University of Basel, Schonbeinstrasse 40, CH-4031 Basel, Switzerland (protocol number: 2023-02225); Prof. Dr. med. Alexandar Tzankov, Head Histopathology and Autopsy, Pathology, Head of the Hematopathology Research Group, University Hospital Basel, Institute of Medical Genetics and Pathology, Schoenbeinstrasse 40, CH-4031, Basel, Switzerland (protocol number: EKNZ 2014-252); PD Dr. rer. nat. Mark P. K0hnel and Dr Danny Jonigk, Universitatsklinikum Aachen, Anstalt des offentlichen Rechts (AoR), Pauwelsstral3e 30,52074 Aachen, Germany (protocol number: EK24-221); Dr Kenneth P Nephew, Indiana University School of Medicine-Bloomington, Indiana University, Bloomington, IN, USA; Indiana University Simon Comprehensive Cancer Center, Indiana University School of Medicine, Indianapolis, IN, USA (protocol number: 1807389306); Dr Antonio Lopez-Beltran Pathology Department, at Champalimaud Foundation, Lisbon, Portugal and Department of Pathology, Cordoba University Medical School, 14071 Cordoba, Spain (protocol number—CETICO Acta 322 Ref 3800 and Ref N°211/17); Dr Maximilian Ackermann, (Institute of Pathology, University Clinics of RWTH University, Aachen, Germany, Institute of Pathology and Molecular Pathology, Helios University Clinic Wuppertal, University of Witten/Herdecke, Witten, Germany, Institute of Anatomy, University Medical Center of the Johannes Gutenberg-University, Mainz, GermanyI, Institute of Pathology, Hannover Medical School, Hannover, Germany, (protocol number: no. 6921_BO_K_2021 and no. 2702-2015). Dr Ronny Drapkin from Penn Ovarian Cancer Research Center, Perelman School of Medicine, University of Pennsylvania, Philadelphia, PA, USA provided the samples banked by the OCRC Tumor BioTrust Collection, and Penn Medicine Biobank (PMBB), Philadelphia, PA 19104, USA, under a longstanding IRB approved protocol (protocol number: 702679); Dr Denise Connolly from the Fox Chase Cancer Center (FCCC), Philadelphia, PA, USA, provided the samples banked by the Biosample Repository Facility, under a longstanding IRB approved protocol; Dr Christopher Moskaluk from Department of Pathology, University of Virginia, Charlottesville, VA, 22908, USA, provided the samples banked by the Biorepository and Tissue Research Facility (BTRF), under a longstanding IRB approved protocol (protocol number-300015); All tissue and blood samples used were de-identified and stored with no patient information. All samples were collected with the informed consent of donors and collected as per the Institutional Review Board (IRB)-approved protocols for the collection, banking, and distribution of biospecimens and any associated clinical data. Participants diagnosed with high-grade serous carcinoma of the ovary, fallopian tube or primary peritoneum who underwent debulking surgery followed by chemotherapy were included. Tumor histology and cellularity were confirmed by the pathologists. Tumor and stromal tissue regions were demarked and captured using the laser microdissection system Zeiss PALM MicroBeam IV (ZEISS).

### Immunohistochemistry and Immunofluorescence

Paraffin-embedded normal ovarian tissue, high-grade serous carcinoma (HGSC) tumor patient samples, and patient-derived (PDX) and cancer cell mice tumor xenografts were fixed in 4% buffered formalin. Serial sections of the specimens were cut with 5 m in thickness with a Leica RM2235 microtome (Leica Biosystems) and stained with H&E, hFwe and STAT3 antibodies. Briefly, samples were deparaffinized in xylol and rehydrated in a graded series of ethanol. Antigen retrieval was performed in 10 mM EDTA buffer pH 9 for 20 min at 96-98° C. Samples were allowed to cool at room temperature for 20 min, and blocked in 1% peroxide solution for 10 min, followed by washing with TBS+0.5% Triton X-100. Slides were blocked in 3% bovine serum albumin (BSA, Sigma-Aldrich), and incubated with anti-hFwe-N-term (Genscript Abl; 1:500), anti-hFwe-Win (Genscript Ab4; 1:500), anti-STAT3 (Abcam #ab31370; 1:200), anti-pSTAT3 Y705 (Abcam #ab76315; 1:500), and anti-caspase 3 (Abcam #ab32351; 1:100) primary antibodies in blocking solution overnight at 4° C. Sections treated with hFwe antibodies were incubated in anti-mouse IgG HRP-conjugated secondary antibody (1:1,000, Thermo Fisher Scientific #31430), while sections treated with STAT3 antibodies were incubated in anti-rabbit IgG HRP-conjugated secondary antibody (1:5,000, Thermo Fisher Scientific #31460) for 1 h at 25° C. Finally, sections were developed with 3,3′-diaminobenzidine (DAB, Vector Labs) for 10 min to visualize the color of the reaction. For immunofluorescence, HGSOC patient-derived WT cancer cells and WT fibroblasts were cultured for 2 days in a four-well chamber slide system (LAB-TEK, Thermo Fisher Scientific) overnight. Additionally, HGSOC patient-derived hFwe KD cancer and hFwe KD fibroblasts were co-cultured in the same conditions. Cells were fixed with 4% formaldehyde and permeabilized in 0.5% Triton X-100 in PBS for 10 min. Fixed cells were washed three times in PBS and blocked with 5% BSA in PBS for 45 min. After washing three times with PBS, co-cultures were co-stained for Alexa Fluor® 594 rabbit anti-Cytokeratin-19 (Abcam, #ab203443; 1:200); Alexa Fluor® 647 rabbit anti-Vimentin (Abcam, #ab194719; 1:100) and mouse anti-Flower-N(1:2000, Ab, Genscript)⁴, or mouse anti-Flower-Win (1:2000, Ab4, Genscript)⁴ for overnight at 4° C. The slides were then washed three times with PBS. The following day, samples were washed three times with PBS and incubated with goat anti-mouse IgG AlexaFluor 488 (1:1,000, Thermo Fisher Scientific). The stained cells were mounted with PBS and analyzed using Vectra PhenoImager HT with the HT version 2.0 software (Akoya Bioscience) and QuPath version 0.4.4.

### Multiple Immunohistochemistry

Multiplex immunohistochemistry (mIHC) was performed in ImmunoHistology Core Lab of the Earle A. Chiles Research Institute (Portland, Oregon) per our standard protocol. Five μm thin sections of FFPE human ovarian cancer samples were cut on a Leica RM2235 microtome (Leica Biosystems). Tissue sections were deparaffinized in xylene and rehydrated in graded alcohol. Tris-EDTA (10 mM Tris Base, 1 mM EDTA, pH9.0) and 0.1M Sodium Citrate pH6.0 were used for retrieving antigens and antibody stripping in a microwave oven, respectively. 3% H₂O₂ was used for blocking endogenous peroxidase. Tissue sections were blocked with blocking/antibody diluent (ARD1001EA, Akoya Biosciences) for 10 min at RT before antibody incubation. Tissue sections were incubated with primary antibody in a shaking staining tray. Anti-pSTAT3 Y705 (1:800, D3A7, Cell Signaling #9145), anti-Vimentin (1:2000, D21H3, Cell Signaling #5741), anti-Flower-N(1:2000, Ab, Genscript), anti-Flower-Win (1:2000, Ab4, Genscript), were incubated for 30, 20, 20, 20, 20, and 15 min respectively at RT. After briefly washing in 1X TBS, tissue sections were incubated with secondary antibody MACH2 Rb HRP-Polymer (RHRP520H, Biocare Medical) or MACH2 M HRP-Polymer (MHRP520H, Biocare Medical) for 10 min. Followed by a brief wash, tissue sections were incubated with Opa1690 (1:200, Akoya Bioscience #FP1497001KT), Opa1570 (1:400, Akoya Bioscience #FP1488001KT), Opa1620 (1:200, Akoya Bioscience #FP1495001KT), Opa1520 (1:200, Akoya Bioscience #FP1487001KT), and Opa1780 (1:25, Akoya Bioscience #FP1501001KT), respectively. Counterstaining was performed with DAPI (Akoya Biosciences,1 drop of DAPI to 0.5 ml 1X TBS) for 5 min at RT. After a quick wash, slides were mounted with Prolong Diamond Antifade Mountant (p36970, Thermofisher). Imaging was performed on a PhenoCycler™-Fusion system (Akoya Bioscience).

Antibody optimization and validation were performed by following recently described. Conventional single biomarker chromogenic staining was performed on serial tissue sections. Deparaffinization, rehydration, antigen retrieval, and blocking were performed as described above. Primary antibody incubation was performed overnight at 4° C. After washing with 1X TBS, tissue sections were incubated with HRP-conjugated secondary antibody for 30 min at RT. Followed by a brief wash, color development was performed with DAB (SK-4105, Vector) incubation for 3 min at RT. Counterstaining with hematoxylin (3801562, Leica) was done for 45 seconds followed by rinsing and bluing in flowing tap water for 2 min. Slides were mounted with cytoseal 60 (8310-4, ThermoFisher) after dehydration and clear. Imaging was performed on a PhenoCycler™-Fusion system.

### Methylation-Specific PCR and Methylight Assay

For methylation-specific PCR (MSP) and MethyLight Assay, we used bisulfite-treated and untreated DNA extracted from HGSC normal, tumor, and stromal tissues and followed the instructions of the EpiTect MethyLight PCR Kit+ROX Vial (Qiagen). For each of the hFwe exons, we designed a methylation-specific primers (Exon 1 MSF: 5′-GACGGTGGCACCATGAG-3′ (SEQ ID NO:77) and MSR 5′-CAGACAGGCGACACAGC-3′ (SEQ ID NO:78); Exon 2 MSF: 5′-GATCTCTGGCCTCTTCAACTG-3′ (SEQ ID NO:87) and MSR: 5′-GCCATGCATTACTCACATCATC-3′ (SEQ ID NO:88); Exon 3 MSF: 5‘-GAAGGCTTI’CTTCTACTGCGG-3 (SEQ ID NO:354) and MSR: 5′-CTGAATACACTGCCCACCCT-3′ (SEQ ID NO:355); Exon 4 MSF: 5′-GTCGTTCCCATCGTCATCAG-3′(SEQ ID NO:107) and MSR: 5′-CAGAGCAGAGAGTCCGTACA-3′ (SEQ ID NO:108); Exon 5 MSF: 5′-AGACTGAGGCTGGTTCCTT-3′ (SEQ ID NO:117) and MSR: 5′-TGCAGGCATTCTGATTTCCC-3′ (SEQ ID NO:118); Exon 6 MSF: 5′-GGCGATGCGATCTCCTATG-3′ (SEQ ID NO:125) and MSR: 5′-CACTTGGACCCACACAGAG-3′ (SEQ ID NO:126)), an unrnethylation-specific forward primer (Exon 1 UMSF: 5′-GATGGTGGTATTATGAG-3′ (SEQ ID NO:356) and UMSR: 5′-TAGATAGGTGATATAGT-3′ (SEQ ID NO:357); Exon 2 UMSF: 5′-GATTTTTGGTTTTTTCAATTG-3′ (SEQ ID NO:358) and UMSR: 5′-GTTATGTATTATTTATATTATT-3′ (SEQ ID NO:359); Exon 3 UMSF: 5′-GAAGGTTGTTTTTTATTGTGG-3′ (SEQ ID NO:360) and UMSR: 5′-CACTACCCACCCTCACCTAAC-3 (SEQ ID NO:361); Exon 4 UMSF: 5′-GTTGTTTTTATTGTTATTAG-3′ (SEQ ID NO:362) and UMSR: 5′-TAGAGTAGAGAGTTTGTATA-3′ (SEQ ID NO:363); Exon 5 UMSF: 5′-AGATTGAGGTTGGTTTTTT-3′ (SEQ ID NO:364) and UMSR: 5′-TGTAGGTATTTTGATTTTTT-3′ (SEQ ID NO:365); Exon 6 UMSF: 5′-GGCGATGCGATCTCCTATG-3′ (SEQ ID NO:125) and UMSR: 5′-CACTTGGACCCACACAGAG-3′ (SEQ ID NO:126)), a methylation-specific Tagmnan probe (Exon I MST:/56-FAM/GCGTCCGCCAGCTCTGCGCCGCCCGCGCA(SEQ ID NO:366)/36-TAMSp/; Exon 2 MST:/56-FAM/CATCCACCCTCTGAACATTGCGGCCGGC(SEQ ID NO:367)/36-TAMSp/; Exon 3 MST:/56-FAM/CAACCCCTCACCCGCAGTAGAAGACAGCC(SEQ ID NO:368)/36-TAMSp/; Exon 4 MST: /56-FAM/CTGACCACGCTGCTGGGCAACGCCATCG(SEQ ID NO:369)/36-TAMSp/; Exon 5 MST:/56-FAM/CTGGGCCATTCTCAGAGGGGACAAGACAGG(SEQ ID NO:370)/36-TAMSp/; Exon 6 MST:/56-FAM/GCAGGCGGATGAGGAGAAGCTCGCGGAGA(SEQ ID NO:371)/36-TAMSp/), and an unmethylation-specific Tagman probe (Exon I UMST: /5SUN/GTGTTTGTTAGTTTTGTGTTGTTTGTGTA(SEQ ID NO:372)/36-TAMSp/; Exon 2 UMST:/5SUN/TATTTATTTTTTGAATATTGTGGTTGGT(SEQ ID NO:373)/36-TAMSp/; Exon 3 UMST:/5SUN/CAACCCCTCACCCACAATAAAAAACAACC(SEQ ID NO:374)/36-TAMSp/; Exon 4 UMST:/5SUN/TTGATTATGTTGTTGGGTAATGTTATTG(SEQ ID NO:375)/36-TAMSp/; Exon 5 UMST:/56-5SUN/TTGGGTTATTTTTAGAGGGGATAAGATAGG(SEQ ID NO:376)/36-TAMSp/; Exon 6 UMST:/56-5SUN/GTAGGTGGATGAGGAGAAGTTTGTGGAGA(SEQ ID NO:377)/36-TAMSp/). Due to the small size of the methylated region, we could only test the specific methylation of Exon 3 by combining the methylation-specific forward primer and the unmethyIation-specific reverse primer during PCR. For MSP, we used bisulfite-treated and untreated DNA with the following primer combinations: MSF with MSR, UM SF with UMSR, and MSF with UMSR. For Methylight Assay, we used bisulfite-treated DNA with UMSF, UMSR, MST, and UMST. We performed PCR in a BIO-RAD C1000 Touch PCR Thermal Cycler w/CFX96 Real Time Optics Module (BIO-RAD) with an initial PCR activation step of 5 min at 95° C., followed by 40 cycles of 15s at 95° C. and 60s at 60° C. As controls, we used a no-template control sample, and EpiTect PCR Control DNA (Qiagen). For MSP, we confirmed the amplification of the PCR products by performing 1% agarose gel electrophoresis and revealing the gel using the ChemiDoc Touch System (BIO-RAD). For MethyLight, we calculated the PMR of each sample using the formula PMR=100×2^{-ΔΔCt}, where ΔΔCt=[ΔCt of sample—ΔCt of methylated DNA control]. The final products were confirmed by DNA sequencing. PCR data was analyzed using the CFX manager v2.3 software.

### Bisulfite Sequencing Analysis

Genomic DNA was extracted using a QIAamp DNA Mini kit (Qiagen, Valencia, CA, USA) and treated with sodium bisulfite using an EpiJET Bisulfite Conversion Kit (Thermo Scientific™) according to the manufacturer's instructions. During the modification, the unmethylated cytosines of the genomic DNA were converted to uracil, but the methylated cytosines remained unchanged. Bisulfite-treated DNA was stored at −80C until needed. 50ng of each sample was used for bisulfite hot-start PCR in 25 μl volume reactions containing 1 mM primers (Forward primer-TGAATGTTTTTATTTTGTTGTTGTGT (SEQ ID NO:378) and Reverse primer-CACCTAACTTTTAAATCCTATTTTATCAAT (SEQ ID NO:379)), and a 25 master mix containing Q5® High-Fidelity DNA Polymerase (NEB). PCR was performed in an Eppendorf Mastercycler® X40—PCR Thermocycler (Eppendorf) with the following program: Initial denaturation at 98C for 30 sec, followed by 35 cycles of (10 s at 98C,30 sec annealing at 60C and 30 sec elongation at 72C), followed by a final 2-min extension step at 72C). All PCR reactions were checked on a 2.0% agarose gel to ensure successful amplification of size and specificity before proceeding with sequencing. The amplification product of the expected size was cut from the gel and purified by QIA quick gel extraction kit (Qiagen). PCR products were diluted to 2 ng/ml and subjected to direct sequencing.

### Exosome Isolation

We isolated exosomes from blood and from media supplemented with 10% exosome-depleted fetal bovine serum as previously described. Briefly, blood and media collected from cell cultures were centrifuged at 500 g for 10 min to remove cells in suspension. Then, the supernatant was centrifuged at 20000 g for 20 min and exosomes were harvested after centrifugation at 100000 g for 70 min. Exosome pellet was resuspended in 15 mL of PBS. At this stage, 14.5 mL of exosome suspension were slowly added to 4 mL of sucrose solution and centrifuge at 100000 g for 70 min. The floating exosome fraction was collected, diluted in PBS, and centrifuged at 100000g overnight. The exome pellet was resuspended in 200 μL of PBS. Finally, exosomes were characterized using NanoSight and performing western blot with antibodies for CD9 (1:1,000, Cell Signaling Technology #13174) and TSG101 (1:1,000, Abcam #ab125011) (positive controls) and Calnexin (1:1,000, Abcam #ab133615; negative control). After capture and release of the vesicles from the microfluidics device, they are processed downstream for validation of our technology by different techniques. We used the NanoSight NS300 NTA with the Nanosight NTA version 3.0 software (Malvern Panalytical Ltd.) to perform NTA and ISF to compare and quantify the concentration and the size of the isolated vesicles across different samples from different stages of the disease, and control. Plots of scattered light spots by the vesicles and their speed of motion provide the data that facilitate the determination of total particle count and size distribution.

### Atomic Force Microscopy

AFM imaging was conducted to assess the morphology and size of exosomes. Exosomes were incubated on freshly cleaved mica for 10 min. The samples were dried using a gentle stream of nitrogen. Samples in phosphate-buffered saline (PBS) were imaged using AC40 Biolever mini probe (Bruker AFM Probes) with an Asylum Infinity Biosystem—“MFP-3D Infinity Bio” (Oxford Instruments) with the AFM Software Version 16. A minimum of 5 distinct points on each surface were probed, with approximately 15 force curves recorded at each point. Data acquisition was performed at a frequency of 0.5 Hz in the tapping (AC) mode with a scanning area of 2 m. To quantify the size, three particles were selected, and the size was quantified using ImageJ.

### Nanoparticle Tracking Analysis

The hydrodynamic diameter of exosomes was analyzed using nanoparticle tracking analysis (NTA). An NS300 NTA instrument with the Nanosight NTA version 3.0 software (Malvern Panalytical Ltd., Worcestershire, UK) was used, and data were collected using a 532 nm laser. Three 45 s videos were obtained and analyzed using the Nanosight 3.0 software. During analysis, the camera level was set to 14, and the detection threshold was set at 5.

### Cytoplasmatic and Nuclear Extraction and Fractioning

We performed cytoplasmic and nuclear extraction and fractioning from normal, HGSC and stromal cells as previously described. Briefly, cells were washed twice with ice-cold PBS, and lysed with ice-cold lysis buffer (1× PBS, 0.1% NP40, 10 mM Ribonucleoside Vanadyl Complex and protease inhibitor). Cells were centrifuged for 10 min at 16,000 g at 4° C. The supernatant containing the cytoplasmic fraction was transferred to a new tube and store. The pellets containing the nuclear fraction were washed with ice-cold PBS, briefly centrifuged, and stored.

RNA Isolation and Quantitative PCR (qPCR)

Total RNA from cell lines, and patient samples were isolated using the PureLink RNA Mini kit and RNeasy FFPE Kit, respectively, as described previously. RNA from cytoplasmic and nuclear fractions from normal, HGSC and stroma cells was isolated as described previously. Briefly, 500 μL of TRI reagent (MRC) were added to nuclei suspension. Then, 100 μL of chloroform were added to the mixture, incubated at room temperature for 5 min, centrifuged at 4° C. at 16,000 g for 15 min and the upper aqueous layer was transferred to a new tube. At this stage, 3.5x sample volumes of RLT buffer (Qiagen) were added to cytoplasmic and nuclear fractions. Followed by the addition of 2.5× volumes of 100% ethanol. Finally, RNA was isolated using RNeasy Kit (Qiagen) as per manufacturer's instructions, and RNA was eluted in 50 μL RNase/DNase-free H₂O. Nascent RNA was extracted from normal, tumor, and stromal tissues based on the protocol developed by Reiner and Neugebauer. Briefly, normal, tumor, and stromal tissues were digested to obtain normal, tumor, and stromal cells, which centrifuged and resuspended in 250 μL cell lysis buffer (10 mM Tris-HCL, 0.05% NP-40, 150 mM NaCl, 25 M a-amanitin, 40 U/ml SUPERase.IN, and proteinase inhibitors). After 5 min incubation on ice, sucrose buffer (cell lysis buffer with 24% w/v sucrose) was added to the cell lysates, followed by 10 min centrifugation at 360g, 4° C. Then nuclear pellets were rinsed with ice-cold PBS/1 mM EDTA and resuspended in 100 μl of nuclear resuspension buffer (20 mM Tris-HCl pH 8.0, 75 mM NaCl, 0.5 mM EDTA, 0.85 mM DTT, 50% glycerol, 25 M a-amanitin, 40 U/ml SUPERase.IN, and proteinase inhibitors). Then, the cell nuclei were lysed with 100 μl nuclear lysis buffer (20 mM HEPES pH 7.5, 1 mM DTT, 7.5 mM MgCl2, 0.2 mM EDTA, 0.3 M NaCl, 1M urea, 1% NP-40, 25 M a-amanitin, 40 U/ml SUPERase.IN, and proteinase inhibitors), incubated for 3 min on ice and centrifuged for 2 min at 12,000g, 4° C. Then, chromatin pellets were resuspended with 100 μl PBS and 400 μl Trizol and incubated at 50° C. for 10 min with constant shaking at 180g. To RNA extraction, we added 60 μl of chloroform to the samples, incubated them for 2 min at room temperature, centrifuged for 15 min at 12,000g, 4° C., and transferred the clear upper aqueous phase to a new tube. Then, the RNA was extracted following the RNeasy Mini Kit protocol (Qiagen), as described above. After RNA extraction, polyA tails were depleted from RNA using the magnetic beads from the DynaBeads mRNA Direct Micro Purification kit. Then, the samples were cleaned up with the Clean and Concentrator-5 kit and eluted in 30 μl of nuclease-free water. We used the RiboMinus™ Eukaryote Kit v2 on polyA-depleted samples to remove any ribosomal RNA. 10 ng of total RNA was reverse-transcribed to cDNA using Superscript Vilo cDNA synthesis kit. Real-time PCR (qPCR) was performed with PowerUp SYBR Green master mix (Thermo Fisher Scientific) using the BIO-RAD C1000 Touch PCR Thermal Cycler w/CFX96 Real Time Optics Module (BIO-RAD), and data was analyzed with the CFX manager v2.3 software. The reaction conditions established an initial denaturation step at 95° C. for 2 min, followed by 40 cycles of 95° C. for 15 s and 60° C. for 60 s. All samples were run in triplicate, and the relative gene expression analysis was performed with the comparative Ct method (ΔΔCt), normalized to the expression of the housekeeping gene GAPDH, as described previously.

The following primers were used: GAPDH: 5′-GGATGCAGGGATGATGTTC-3′ (F) (SEQ ID NO:1) and 5′-TGCACCACCAACTGCTTAG-3′ (R) (SEQ ID NO:2); hFwe-Win: 5′-GCCTTCATCTTGTTGCTGTG-3′ (F) (SEQ ID NO:3) and 5′-CATCCCGCAGTAGAAGACAG-3′ (R) (SEQ ID NO:4); hFwe-Lose: 5′-GCGTGTGGATGATGATGG-3′ (F) (SEQ ID NO:5) and 5′-AGCAGAGAGTCCGTACA GCA-3′ (R) (SEQ ID NO:6); LINC01914 (Tu-Stroma): 5′-GCGGCCATGGAAAGAACTT-3′ (F) (SEQ ID NO:7) and 5′-GAAATGGGTCAGCAGGGCTT-3′ (R) (SEQ ID NO:8); LOC105370401: 5′-CACACCAGGCTACTGAAAGA-3′ (F) (SEQ ID NO:9) and 5′-CACCTCTCCTCCTGAGTTTATG-3′ (R) (SEQ ID NO:10); LOCI01927402: 5′-GCCGCCAAACTGACAAATC-3′ (F) (SEQ ID NO:11) and 5′-GACCACCTTCTCCATCCATTAG-3′ (R) (SEQ ID NO:12); LINC02136: 5′-AGACTACTACGATGGTGCAAAG-3′ (F) (SEQ ID NO:13) and 5′-CCAATATCTGGAACTGGCATCTA-3′ (R) (SEQ ID NO:14); DGCR5: 5′-CTCCCTTCTCTACGCAGTGTCA-3′ (F) (SEQ ID NO:15) and 5′-AGACGTCCATGAGCACCTCGAA-3′ (R) (SEQ ID NO:16); LOC105376993: 5′-CAAGGCCAAAGAGCTAGTAAGT-3′ (F) (SEQ ID NO:17) and 5′-TCCATGCTCTCTCCTGTTTATTC-3′ (R) (SEQ ID NO:18); LOC105371768: 5′-GTTTCCTGCTGTTGCTTGTG-3′ (F) (SEQ ID NO:19) and 5′-CTGGATCTCCTGCTAACCATTC-3′ (R) (SEQ ID NO:20); LOC105373180: 5′-GGAGGAAACTGTCATGGGATAAG-3′ (F) (SEQ ID NO:21) and 5′-CGTGCGTGTGTTTGTGTATG-3′ (R) (SEQ ID NO:22); LOC105373323: 5′-TGGGTTTCCTGTTCCATGAG-3′ (F) (SEQ ID NO:23) and 5′-CTCGGGTTTGGACCGTATTT-3′ (R) (SEQ ID NO:24); LOC105372647: 5′-CCTCAGACTAGAACAGCAACAC-3′ (F) (SEQ ID NO:25) and 5′-AAGTGGAGACAAGGGTAGGA-3′ (R) (SEQ ID NO:26); PSAPI: 5′-TGACAAACCCAGAGGCAATC-3′ (F) (SEQ ID NO:27) and 5′-GTGACAGAGTGAGAGTCCATAATC-3′ (R) (SEQ ID NO:28); ADGRA]: 5′-GCGGCATCAATCGCACCAAGTA-3′ (F) (SEQ ID NO:29) and 5′-GGTCACTCCTATCCACAGCATG-3′ (R) (SEQ ID NO:30); TK2: 5′-GGATCTTGAGGAACATGGACGTG-3′ (F) (SEQ ID NO:31) and 5′-CAGCGGAATGACCTTCTCCTCT-3′ (R) (SEQ ID NO:32); SELE: 5′-TCAAGGGCAGTGGACACAGCAA (F) (SEQ ID NO:33) and 5′-GGAAACTGCCAGAAGCACTAGG-3′ (R) (SEQ ID NO:34); STAT3: 5′-TGGAACAGATGCTCACTGCG-3′ (F) (SEQ ID NO:35) and 5′-CTCCATCGCTGACAAAAGCC-3′ (R) (SEQ ID NO:36); DNMT3A: 5′-CCTGGGAGGAAGCGCAAG-3′ (F) (SEQ ID NO:37) and 5′-TGGATGGGGACTTGGAGATCA-3′ (R) (SEQ ID NO:38); HP]: 5′-ATATCGCTCGGGGCTTTGAG-3′ (F) (SEQ ID NO:39) and 5′-CCAGGTCAGCTTCATCTGTGT-3′ (R) (SEQ ID NO:40); SRSF3: 5′-TCCTCCACCTCGTCGCAGAT-3′ (F) (SEQ ID NO:41) and 5′-CGGGACGGCTTGTGATTTCT-3′ (R) (SEQ ID NO:42); GLP: 5′-CTACGCACTCTCCTTCTCTGCT-3′ (F) (SEQ ID NO:43) and 5′-CGGACAATGCTCGCAGGATGAA-3′ (R) (SEQ ID NO:44); G9a: 5′-GGTGAACAACCACCTGGAGGTA-3′ (F) (SEQ ID NO:45) and 5′-AGGCTGACCATCTCCAAGTTCC-3′ (R) (SEQ ID NO:46); PKDCC: 5′-TTAAGGAGATGGTGCTGCT-3′ (F) (SEQ ID NO:47) and 5′-ATGGTGGTCAGGGTGTCT-3′ (R) (SEQ ID NO:48); ADAMTS]3: 5′-GCTCACCAACCTCAACATC-3′ (F) (SEQ ID NO:49) and 5′-TCAGAATGACCATCTTCACC-3′ (R) (SEQ ID NO:50); SLC2A6: 5′-CGGTGTACGTGTCTGAGATT-3′ (F) (SEQ ID NO:51) and 5′-ATCCGAACACTGCCATGAG-3′ (R) (SEQ ID NO:52); EFTUD2 (SNU114): 5′-CACTCAGCCTCTCACAGAAC-3′ (F) (SEQ ID NO:53) and 5′-CAGGTAATGTCTGCTCCATC-3′ (R) (SEQ ID NO:54); SART](SNU66): 5′-GAAACCCTTGGAGGTTAATG-3′ (F) (SEQ ID NO:55) and 5′-TATCTTCCCCAGCTTTTGGT-3′(R) (SEQ ID NO:56); SF3A2 (SF3A66): 5′-ACAAAGTGACCAAGCAGAGA-3′ (F) (SEQ ID NO:57) and 5′-TCAGGGTAGTCAATCTGGAA-3′ (R) (SEQ ID NO:58); SNRNP200 (BRR2): 5′-GAGGAGGAAGGTGATGAAGA-3′ (F) (SEQ ID NO:59) and 5′-CTACGAGATTAGCCGAGAGG-3′ (R) (SEQ ID NO:60); SNRNP40 (U5SNRNP): 5′-CAGATGGCAGTATGCTTTTC-3′ (F) (SEQ ID NO:61) and 5′-ACCCTCTCACCTGTTTCACT-3′ (R) (SEQ ID NO:62); PRPF8 (PRP8): 5′-CATGCTCAACCTTCTCATTC-3′ (F) (SEQ ID NO:63) and 5′-TTCTTTCTTTCCTTGGTGGT-3′ (R) (SEQ ID NO:64); PRPF19: 5′-TCAAAGTTGCTCACCCAATC-3′ (F) (SEQ ID NO:65) and 5′-ACTGCATCCCACTCATCCT-3′ (R) (SEQ ID NO:66); PRPF3: 5′-TTGAGGAGGTGGAAGAAGAG-3′ (F) (SEQ ID NO:67) and 5′-GTCTTTGGCTGAGGTGTAGG-3′ (R) (SEQ ID NO:68); RNU2-1 (U2SNRNP): 5′-CGTCCTCTATCCGAGGACAATA-3′ (F) (SEQ ID NO:69) and 5′-GTACTGCAATACCAGGTCGATG-3′ (R) (SEQ ID NO:70); RNU6-1: 5′-GCTTCGGCAGCACATATACTA-3′ (F) (SEQ ID NO:71) and 5′-CGAATTTGCGTGTCATCCTTG (R) (SEQ ID NO:72); SNW1 (SKIP): 5′-TTGCTCTCGGTGTTCCTAATC-3′ (F) (SEQ ID NO:73) and 5′-TCTTCTCCACCTGCAAATCC-3′ (R) (SEQ ID NO:74); DHX15: 5′-TGAGATGGATGTGATGTTGG-3′ (F) (SEQ ID NO:75) and 5′-CAGGAGGGGATCATTCATAG-3′ (R) (SEQ ID NO:76); DDX3X 5′-ACTATGCCTCCAAAGGGTGTCC-3′ (F) (SEQ ID NO:173) and 5′-AGAGCCAACTCTTCCTACAGCC-3′ (R) (SEQ ID NO:174); hFwe Exon 2-4: 5′-GATCTCTGGCCTCTTCAACTG-3′ (F) (SEQ ID NO:87) and 5′-CAGAGCAGAGAGTCCGTACA-3′ (R) (SEQ ID NO:108); hFwe Exon 3: 5′-GCTGCCAGTTCATCGAGTT-3′ (F) (SEQ ID NO:97) and 5′-TCACCCGCAGTAGAAGACA -3′ (R) (SEQ ID NO:98).

### Chromatin Immunoprecipitation (ChIP)

ChIP experiments were performed on cultured cells and patient samples as described previously. Cells were fixed with 1% formaldehyde for 10 min at 22° C. and then quenched with 0.125 M glycine (Sigma). The cells were scraped in cold PBS and collected by centrifugation and rinsed in cold PBS. The cell pellets were re-suspended in swelling buffer (10 mM potassium acetate, 15 mM magnesium acetate, 0.1 M Tris; pH 7.6, 0.5 mM phenylmethylsulfonyl fluoride, and 100 ng of leupeptin and aprotinin/ml), incubated on ice for 20 min, and then dounce-homogenized. Chromatin was collected from pooled normal ovarian and HGSC tumor and adjacent stromal tissue (n=8) by micro-centrifugation and then resuspended in sonication buffer (1% sodium dodecyl sulfate, 10 mM EDTA, 50 mM Tris-HCl; pH 8.1, 0.5 mM phenylmethylsulfonyl fluoride, and 100 ng of leupeptin and aprotinin/ml) and incubated on ice for 10 min. Chromatin was sonicated using an ultrasonics sonicator to achieve an average length of approximately 1000 bps. 20% of total chromatin was saved as total input chromatin. Samples were immunoprecipitated overnight at 4° C. with antibody-conjugated beads or no antibody-bound beads as control. Cross-links were reversed by the addition of NaCl to a final concentration of 200 mM, and RNA was removed by the addition of 10 μg of RNase A per sample for 4-5 h at 65° C. The samples were then precipitated at 20° C. overnight by the addition of 2.5 volumes of ethanol and then pelleted by microcentrifugation. The samples were re-suspended in 100 μl of Tris-EDTA; pH 7.5, 25 μl of 5X proteinase K buffer (1.25% sodium dodecyl sulfate, 50 mM Tris; pH 7.5, and 25 mM EDTA), and 1.5 μl of proteinase K (Boehringer Mannheim, Stuttgart, Germany) and incubated at 45° C. for 2 h. Samples were extracted with phenol-chloroform-isoamyl alcohol (25:24:1) followed by extraction with chloroform-isoamyl alcohol and then precipitated with 1/10 volume of 3 M NaOAc (pH 5.3), 5 μg of glycogen, and 2.5 volumes of ethanol. The pellets were collected by micro-centrifugation, resuspended in 30 μl of water, and analyzed by PCR and qPCR. ChIP-grade antibodies used included: IgG (Abcam #ab172730, 1:100), Actin (Cell Signaling Technology #4970, 1:100), anti-5-methycytosine (5-mC) (Abcam #ab214727, 1:100); anti-DNMT3a (Abcam #ab2850, 1:100); hnRNPA2B1 (Abcam #ab31645, 1:100); hnRNPG (Novus Biologicals #NBP1-79904, 1:100); YTHDCl (Cell Signaling Technology #77422, 1:100); METTL3 (Cell Signaling Technology #86132, 1:100); METTL14 (Novus Biologicals #NBP1-81392,1:100); WTAP (Cell Signaling Technology #56501, 1:100); KIAA-1429 (Thermo Fisher Scientific #25712-1-AP, 1:100); RBM15 (Cell Signaling Technology #25261, 1:100); Spi-1 (Santa Cruz Biotechnology #sc-352, 1:100); TCERG1 (Novus Biologicals #NBP2-20584, 1:100); BRM (Santa Cruz Biotechnology sc-166579, 1:100); H3K9Me3 (Abcam #ab8898, 1:100); H3K4Me1 (Abcam #ab8895, 1:100); H3K4Me3 (Abcam #ab8580, 1:100); H3K27me3 (Abcam #ab6002, 1:100); H3K36Me3 (Abcam #ab9050, 1:100); Hyperacetylation-H3K27Ac (Abcam #ab4729, 1:100); CTCF (Abcam #abl28873, 1:100); MeCP2 (Abcam #abl95393, 1:100); HP1 (Santa Cruz Biotechnology sc-#515341, 1:100); PTB (Thermo Fisher Scientific #32-4800, 1:100); PSIP1 (Thermo Fisher Scientific #MA5-14821, 1:100); MRG15 (Abcam #abl83663, 1:100); SRSF3 (Thermo Fisher Scientific #33-9400, 1:100); RBP2 (Santa Cruz Biotechnology sc-#365993, 1:100); SETl (Abcam #ab70378, 1:100); MLL (Cell Signaling Technology #61295, 1:100); GLP (Santa Cruz Biotechnology #sc-80603, 1:100); SMYD2 (Santa Cruz Biotechnology #sc-393827, 1:100); NSD2 (Santa Cruz Biotechnology #sc-365627, 1:100); ASH1 (Thermo Fisher Scientific #A301-749A, 1:100); SUV39 h1 (Santa Cruz Biotechnology #sc-23961, 1:100); G9a (Santa Cruz Biotechnology #sc-515726, 1:100); SET7/9 (Santa Cruz Biotechnology #sc-390823, 1:100); RIZ (Santa Cruz Biotechnology #sc-130256, 1:100); EZH1 (Thermo Fisher Scientific #PA5-40850, 1:100); EZH2 (Thermo Fisher Scientific #49-1043, 1:100); DOT1 μL (Thermo Fisher Scientific #MA5-24294), 1:100; RNA Pol II (Santa Cruz Biotechnology #sc-55492, 1:100) STAT3 (Abcam #ab31370, 1:100); pSTAT3 Y705 (Abcam #ab76315, 1:100).

Following primers were used: Exon 1: 5′-GACGGTGGCACCATGAG-3′ (F) (SEQ ID NO:77) and 5′-CAGACAGGCGACACAGC-3′ (R) (SEQ ID NO:78), −100 bp Exon 1: 5′-TATGCTCCCTCTCCCACAAG-3′ (F) (SEQ ID NO:79) and 5′-CATGGTGCCACCGTCAG-3′ (R) (SEQ ID NO:80), −200 bp Exon 1: 5′-CCTAGTCGCGGCATGAG-3′ (F) (SEQ ID NO:81) and 5′-AGGGAGCATATTAGCATAGGG-3′ (R) (SEQ ID NO:82); +100 bp Exon 1: 5′-GACGGTGGCACCATGAGCGTG-3′ (F) (SEQ ID NO:83) and 5′-ACCCCCGACCCCCAGCGGGTC-3′ (R) (SEQ ID NO:84); +200 bp Exon 1: 5′-GACCCGCTGGGGGTCGG-3′ (F) (SEQ ID NO:85) and 5′-TCCCAGAAGCGCCAGCCCAG-3′ (R) (SEQ ID NO:86); Exon 2: 5′-GATCTCTGGCCTCTTCAACTG-3′ (F) (SEQ ID NO:87) and 5′-GCCATGCATTACTCACATCATC-3′ (R) (SEQ ID NO:88); −100 bp Exon 2: 5′-ACTCTCGTCCTTTCCAGTCA-3′ (F) (SEQ ID NO:89) and 5′-AAGAGACACGGGCAGGA-3′ (R) (SEQ IDNO:90); −200 bp Exon 2: 5′-CAAGGCACTGCACCTCTC-3′ (F) (SEQ IDNO:91) and 5′-CTCAGCACACAGTTCTCACA-3′ (R) (SEQ ID NO:92); +100 bp Exon 2: 5′-TAATGCATGGCCGTCCCA-3′ (F) (SEQ ID NO:93) and 5′-CCCTGCCACTCCTCTGT-3′ (R) (SEQ ID NO:94); +200 bp Exon 2: 5′-GTTGCCGTGGTTGCCAT-3′ (F) (SEQ IDNO:95) and 5′-AAGAAGCCGCATGCTGTG-3′ (R) (SEQ ID NO:96); Exon 3: 5′-GCTGCCAGTTCATCGAGTT-3′ (F) (SEQ ID NO:97) and 5′-TCACCCGCAGTAGAAGACA-3′ (R) (SEQ ID NO:98); −100 bp Exon 3: 5′-GGCATTCCTTATGGCACTGG-3′ (F) (SEQ ID NO:99) and 5′-TCATGCTGCAGGGACCA-3′ (R) (SEQ ID NO:100); −200 bp Exon 3: 5′-GGGAGTGCTGGAGTCTTC-3′ (F) (SEQ ID NO:101) and 5′-CCTCATCCCTCCTCCTCT-3′ (R) (SEQ ID NO:102); +100 bp Exon 3: 5′-GGTCCCGTGACACAGTT-3′ (F) (SEQ ID NO:103) and 5′-GAATACACTGCCCACCCT-3′ (R) (SEQ ID NO:104); +200 bp Exon 3: 5′-CAGTTCCTCTCTGTGGAAGTGTAA-3′ (F) (SEQ ID NO:105) and 5′-CTGGGCGCAGAACCACT-3′ (SEQ ID NO:106); Exon 4: 5′-GTCGTTCCCATCGTCATCAG-3′ (F) (SEQ ID NO:107) and 5′-CAGAGCAGAGAGTCCGTACA-3′ (R) (SEQ ID NO:108); -100 bp Exon 4: 5′-CTGGGTGTCTAGGAAGGA-3′ (F) (SEQ ID NO:109) and 5′-GGGAAAGCAGAGGTCAAGG-3′ (R) (SEQ ID NO:110); −200 bp Exon 4: 5′-GCTTTGGATCCTATTCCCAGAA-3′ (F) (SEQ ID NO:111) and 5′-AGACACAGCTTCAGAAAGTCC-3′ (R) (SEQ ID NO:112); +100 bp Exon 4: 5′-CTCTCTGCTCTGGGCAA-3′ (F) (SEQ ID NO:113) and 5′-CTCAGATCCTCCTCACTGAA-3′ (R) (SEQ ID NO:114); +200 bp Exon 4: 5′-CAGAGGTCCCAGTAACTCATTG-3′ (F) (SEQ ID NO:115) and 5′-CCACGCAGAAGTGTTGTAAGT-3′ (R) (SEQ ID NO:116); Exon 5: 5′-AGACTGAGGCTGGTTCCTT-3′ (F) (SEQ ID NO:117) and 5′-TGCAGGCATTCTGATTTCCC-3′ (R) (SEQ ID NO:118); −100 bp Exon 5: 5′-GGCCTGCAGCAAGGATAG-3′ (F) (SEQ ID NO: 119) and 5′-GGGTGGCAACTGAGTACAAT-3′ (R) (SEQ ID NO: 120); −200 bp Exon 5: 5′-CAGAGGTCCCAGTAACTCATTG-3′ (F) (SEQ ID NO:121) and 5′-CCACGCAGAAGTGTTGTAAGT-3′ (R) (SEQ ID NO:122); +100 bp Exon 5: 5′-AGGTCACACCTGGGTCA-3′ (F) (SEQ ID NO:123) and 5′-AGAGAGAGGCAGCGTCA-3′ 30 (R) (SEQ ID NO:124); Exon 6: 5′-GGCGATGCGATCTCCTATG-3′ (F) (SEQ ID NO:125) and 5′-CACTTGGACCCACACAGAG-3′ (R) (SEQ ID NO:126); −100 bp Exon 6: 5′-GGTCACAGGGCAGGAAC-3′ (F) (SEQ ID NO:127) and 5′-AGAGAGAGGCAGCGTCA-3′
(R) (SEQ ID NO:128); +100 bp Exon 6: 5′-AGTGAGGCCTGGACTGT-3′ (F) (SEQ ID NO:129) and 5′-TGGAGAAAGGAGCAGAGGA-3′ (R) (SEQ ID NO:130); +200 bp Exon 6: 5′-CTTAAGCCAGGAGCCACT-3′ (F) (SEQ ID NO:131) and 5′-TGGGTAGTGTCCACACAG-3′ (R) (SEQ IDNO:132); STAT3 BS1: 5′-GTGTATGTGTCCCAGTGCA-3′ (F) (SEQ IDNO:380) and 5′-CCACCTGCCTGTACCTCC-3′ (R) (SEQ ID NO:381); STAT3 BS2: 5′-GGAACCAGAGCCCAGAACAT-3′ (F) (SEQ ID NO:382) and 5′-GCACTGTGGTCAATATTGGGG-3′ (R) (SEQ ID NO:383); STAT3 BS3: 5′-GATGGAGTTCAGCGAGGG-3′ (F) (SEQ ID NO:384) and 5′-GCCAGACACGGAACAAATGT-3′ (R) (SEQ ID NO:385); STAT3 BS4: 5′-GTTGCCATGGTTACCCGC-3′ (F) (SEQ ID NO:386) and 5′-GGAGGGCTCAGTCACGTG-3′ (R) (SEQ ID NO:387).

The samples were run on the QIAxcel Advanced (Qiagen, Hilden, Germany) using QIAxcel DNA High-Resolution Kit (Qiagen) on the 0M500 method (sample injection voltage of 5 kV and a separation voltage of 5 kV), with a sample injection time of 15 seconds. During the run we used the QX DNA Size Marker 25 −500 bp v2.0 and the corresponding QX Alignment Marker 15 bp/600 bp (Qiagen). For the analysis of the results, QIAxcel ScreenGel version 1.6 software was used. The analysis of samples is performed using a two-step approach. First, peaks are detected in the raw data. In a second step, the peak sizes and peak concentrations are determined by mapping the detected peaks to the peaks of the reference size marker.

### Bioinformatic Analysis

The prediction of putative long non-coding RNAs (LncRNA) was obtained by downloading LNCipedia Version 5.2 FASTA database. The sequences of the LncRNAs were compared with hFwe Exon 3 genomic sequence using BLAST from NCBI. Phylogenetic Codon Substitution Frequency (PhyloCSF) score of was calculated using PhyloCSF score software develop by Lin et al. Coding Potential Assessment Tool (CPAT) was calculated following the guidelines published by Wang et al. which are publicly available. Tu-Stroma secondary structure was predicted using RNAfold Server software, accordingly with the algorithm developed by Kerpedjiev et al. Alu sequence analysis was performed using RepeatMasker Web Server as described previously. To predict potential binding site of DDX3X, we ran RBPsuite against RNA sequence of LINC01914. We used the sequence with top binding score to design the PCR primer. To obtain DNA conservation score, we used the Phastcon score.

Poly-A Tail and 5′ Cap Structure ofLncRNA

Poly-A fractioning of RNA was performed as described previously. Briefly, RNA was extracted from OVCAR8 cells and incubated with biotinylated oligo dT probes at room temperature with constant rotation for 15 min. After the incubation, MyOne Streptavidin C1 Magnetic Dynabeads were added to the mixture and incubated for 15 min with constant rotation. Then, the mixture was placed on a magnetic rack, the supernatant containing the poly A-RNA fraction was transferred to a new tube and kept on ice. The magnetic beads, containing the polyA+RNA fraction, were washed three times and eluted. Finally, we performed RT-PCR using Tu-Stroma primers as described in the later section. The presence of 5′cap structure on Tu-Stroma was accessed using FirstChoice RLM-RACE Kit (Ambion) as per manufacturer instructions.

Briefly, total RNA extracted from OVCAR8 cells was treated with Calf Intestine Alkaline Phosphatase (CIP) to remove free 5′P. Further, the cap structure was removed with Tobacco Acid Pyrophosphatase (TAP) treatment, and an RNA adapter oligonucleotide was added to the RNA. Finally, RNA was reversed transcribed and analyzed by PCR using the BIO-RAD C1000 Touch PCR Thermal Cycler w/CFX96 Real Time Optics Module (BIO-RAD).

### RNA Immunoprecipitation (RIP)

RIP assay was performed following the procedure described previously. Briefly, 4×10⁷ cells from normal tissue, HGSC cancer cells and HGSC stroma cells, or isolated exomes were crosslinked with 1% formaldehyde for 10 min and harvested in 10 mL of PBS. Glycine was added to a 330 mM final concentration to stop crosslinking reaction. Cells were briefly centrifuged and re-suspended in 500 μL of RIP lysis buffer (50 mM HEPES pH7.5, 1 mM EDTA, 1% triton) and sonicated 10 times with 30 sec ON and 30 sec OFF cycles. Samples were diluted in 500 μL of dilution buffer (50 mM MgCl2, 10 mM CaCl₂)) and pre-cleared with protein G beads for 2 hrs at 4° C. and followed by addition of 10 μg of antibodies for IgG (Abcam #ab172730, 1:100), Actin (Cell Signaling Technology #4970, 1:100), AGO2 (Abcam #ab186733, 1:100), DARS (Santa Cruz Biotechnology #sc-393275, 1:100), ELAC2 (Abcam #ab205948, 1:100), EPRS (Abcam #abA303-957A, 1:100), GNB2L1 (Cell Signaling Technology #5432, 1:100), IARS (Abcam #ab2296431:100), NCL (Abcam #abl366491:100), RARS (Abcam #ab2410851:100), RPS18 (Thermo Fisher Scientific #PAS-882111:100), RPS3 (Abcam #abl406881:100), RUVBL1 (Thermo Fisher Scientific #PA5-292781:100), TUFM (Thermo Fisher Scientific #A9861, 1:100), HNRNPA2B1 (Abcam #ab31645, 1:100), HNRNPH1 (Abcam #ab5832, 1:100), EEF1A1 (Thermo Fisher Scientific #11402-1-AP, 1:100), HNRNPK (Abcam #ab39975, 1:100), HNRNPM (Thermo Fisher Scientific #PA5-30247, 1:100), MOCS3 (Thermo Fisher Scientific #A13417-200, 1:100), SNW1 (Thermo Fisher Scientific #PA5-118274, 1:100), DDX3X (Santa Cruz Biotechnology #sc-81247, 1:100), EEF2 (Abcam #ab75748, 1:100), HNRNPD (Abcam #ab61193, 1:100), HNRNPU (Santa Cruz Biotechnology #sc-32315, 1:100), HNRNPUL1 (Abcam #ab180952, 1:100), NSUN2 (Cell Signaling Technology #44056, 1:100), WDR1 (Cell Signaling Technology #173574, 1:100), HSPA8 (Cell Signaling Technology #8444, 1:100), MVP (Abcam #ab97311, 1:100), PCBP1 (Novus Biologicals #NBP1-52114, 1:100). Then, 20 μl of protein G beads were added and left for another 1 h at 4° C. Beads were washed once with 1 ml binding buffer (50 mM HEPES/0.5% triton/25 mM MgCl2/5 mM CaCl₂)/20 mM EDTA), once with FA500 (50 mM HEPES/500 mM NaCL/1 mM EDTA/1% triton/0.1% Na deoxycholate), once with LiCl buffer (10 mM Tris/250 mM LiCl/1% triton/0.5% Na deoxycholate/1 mM EDTA) and once with TES (10 mM Tris/10 mM NaCL/1 mM EDTA). Immunoprecipitants were eluted with 75 μl RIP elution buffer (100 mM Tris pH 7.8/10 mM EDTA/1% SDS). NaCl was adjusted to 200 mM and the samples were treated with 20 μg of proteinase K for 1 h at 42° C. and 1 h at 65° C. RNA was extracted with TRIzol solution (Invitrogen), and DNA was digested with DNase (RNase free) to eliminate any DNA contamination. Finally, RNA was reverse transcribed to complementary DNA with Superscript Vilo cDNA synthesis kit (Thermo Fisher) per the manufacturer's instructions and quantitative PCR (qPCR) for was performed with PowerUp SYBR Green master mix (Thermo Fisher) using BIO-RAD C1000 Touch PCR Thermal Cycler w/CFX96 Real Time Optics Module (BIO-RAD) and the PCR data was analyzed using the CFX manager v2.3 software.

The primers used in the study include: SOCS1: 5′-CCTCCTCTTCCTCCTCCTC-3′ (F) (SEQ ID NO:133) and 5′-AACGGAATGTGCGGAAGT-3′ (R) (SEQ ID NO:134); LINC01914 (Tu-Stroma): 5′-GCGGCCATGGAAAGAACTT-3′ (F) (SEQ ID NO:135) and 5′-GAAATGGGTCAGCAGGGCTT-3′ (R) (SEQ ID NO:136); DARS: 5′-GCCTGAGGCAGAAGGAGAAGAG-3′ (F) (SEQ ID NO:137) and 5′-ATGGCAGATGCCAGACTGGAGA-3′ (R) (SEQ ID NO:138); ELAC2: 5′-CCAGCATCTGTGCTTGTGGACA-3′ (F) (SEQ ID NO:139) and 5′-CTGCGAAGGTTGTGAACTGAGG-3′ (R) (SEQ ID NO:140); EPRS: 5′-TTGACCCAGTGGCTCCACGATA-3′ (F) (SEQ ID NO:141) and 5′-CACAGGCTTCAAGCCAACCTCA-3′ (R) (SEQ ID NO:142); GNB2L1: 5′-GCCATACCAAGGATGTGCTGAG-3′ (F) (SEQ ID NO:143) and 5′-CACAAGACACCCACTCTGAGTG-3′ (R) (SEQ ID NO:144); IARS: 5′-CCTCTTTGGACAACCGCCTTTC-3′ (F) (SEQ ID NO:145) and 5′-GGCATCAGCACCATACTTCTGG-3′ (R) (SEQ ID NO:146); NCL: 5′-GCCTGTCAAAGAAGCACCTGGA-3′ (F) (SEQ ID NO:147) and 5′-GAAAGCCGTAGTCGGTTCTGTG-3′ (R) (SEQ ID NO:148); RARS: 5′-GAAACAGTGCGCCTCATGGATC-3′ (F) (SEQ ID NO:149) and 5′-AGCCATACGCAACGGATGTCTG-3′(R) (SEQ ID NO:150); RPS18: 5′-GCAGAATCCACGCCAGTACAAG-3′ (F) (SEQ ID NO:151) and 5′-GCTTGTTGTCCAGACCATTGGC-3′ (R) (SEQ ID NO:152); RPS3: 5′-GCTGAAGATGGCTACTCTGGAG-3′ (F) (SEQ ID NO:153) and 5′-ACAGCAGTCAGTTCCCGAATCC-3′ (R) (SEQ ID NO:154); RUVBLI: 5′-GAAGACAGAGGTGCTGATGGAG-3′ (F) (SEQ ID NO:155) and 5′-CTCTGTCTCACACGGAGTTAGC-3′ (R) (SEQ ID NO:156); TUFM: 5′-GCTACTGGATGCTGTGGACACT-3′ (F) (SEQ ID NO:157) and 5′-CACGCTCTAGTGTACCTGTCAC-3′ (R) (SEQ ID NO:158); HNRNPA2B1: 5′-CAGCAACCTTCTAACTACGGTCC-3′ (F) (SEQ ID NO:159) and 5′-CACTGCCTCCTGGACCATAGTT-3′ (R) (SEQ ID NO:160); HNRNPHI: 5′-TCCAGAGCACAACAGGACACTG-3′ (F) (SEQ ID NO:161) and 5′-GCTTCACCAGTTACTCTGCCATC-3′ (R) (SEQ ID NO:162); EEFIA]: 5′-GATGGCAATGCCAGTGGAACCA-3′ (F) (SEQ ID NO:163) and 5′-GAGAACACCAGTCTCCACTCGG-3′ (R) (SEQ ID NO:164); HNRNPK: 5′-GCAGATGGCTTATGAACCACAGG-3′ (F) (SEQ ID NO:165) and 5′-AATCCGCTGACCACCTTTGCCA-3′ (R) (SEQ ID NO:166); HNRNPM: 5′-CATGGGTCGATTTGGATCTGGG-3′ (F) (SEQ ID NO:167) and 5′-TCAATGCCAGGACCCATCCTCT-3′ (R) (SEQ ID NO:168); MOCS3: 5′-GCATTTCCGCTCTATTCGGCTG-3′ (F) (SEQ ID NO:169) and 5′-GAGCGGCATTTATCAGTGGCTG-3′ (R) (SEQ ID NO:170); SNWL: 5′-CTGCTGATGGAAGAGGACTACAG-3′ (F) (SEQ ID NO:171) and 5′-GGCACGCATTTCCACAGCTTCA-3′ (R) (SEQ ID NO: 172); DDX3X: 5′-ACTATGCCTCCAAAGGGTGTCC-3′ (F) (SEQ ID NO:173) and 5′-AGAGCCAACTCTTCCTACAGCC-3′ (R) (SEQ ID NO:174); EEF2: 5′-CCTCTACCTGAAGCCAATCCAG-3′ (F) (SEQ ID NO: 175) and 5′-CCGTCTTCACCAGGAACTGGTC-3′(R) (SEQ ID NO:176); HNRNPD: 5′-GCCAAGGTTACGGTGGTTATGG-3′ (F) (SEQ ID NO:177) and 5′-TGATGACCACCTCGCCTGGATA-3′ (R) (SEQ ID NO:178); HNRNPU. 5′-GAGATTGCTGCCCGAAAGAAGC-3′ (F) (SEQ ID NO:179) and 5′-TTCGCTGGAAGCCTGCAAACAG-3′ (R) (SEQ ID NO:180); HNRNPULI: 5′-GCAGAGAACGATGTGATTGGCTG-3′ (F) (SEQ ID NO:181) and 5′-CAAGGCTTCCTTCTGGATTCGG-3′ (R) (SEQ ID NO:182); NSUN2: 5′-ACCTGGCTCAAAGACCACACAG-3′ (F) (SEQ ID NO: 183) and 5′-TGGCTTGATGGACGAGCAGGTA-3′ (R) (SEQ ID NO:184); WDRI: 5′-AGAAGGACCACCTGCTCAGTGT-3′ (F) (SEQ ID NO: 185) and 5′-ATGCACCGTCAGACACTGGATC-3′ (R) (SEQ ID NO:186); HSPA8: 5′-TCCTACCAAGCAGACACAGACC-3′ (F) (SEQ ID NO:187) and 5′-CAGGAGGTATGCCTGTGAGTTC-3′ (R) (SEQ ID NO:188); MVP 5′-ATCGAAACGGCGGATCATGCCA-3′ (F) (SEQ ID NO: 189) and 5′-GGCATCACCTACAAAGTCTGGC-3′ (R) (SEQ ID NO:190); PCBP1: 5′-GGACAACACACCATTTCTCCGC-3′ (F) (SEQ ID NO:191) and 5′-AGCCTTTCACCTCTGGAGAGCT-3′ (R) (SEQ ID NO:192).

### Chromatin Oligo Precipitation (ChOP)

ChOP protocol was performed as described previously by Mondal et al. Briefly, biotin-labeled antisense and sense DNA probes against full length Tu-Stroma and MALAT1 (negative control) were designed using online probe designer. We used 4 biotin sense and anti-sense probes spanning the whole transcript of Tu-Stroma, and 4 biotin anti-sense probes spanning the whole transcript of MALAT1. As an additional negative control, we used a probe against GFP RNA as described previously. In this experiment we used samples from normal HGSC tumor and stromal tissues, primary HGSC cancer and stromal cells, and nuclei extracted from stromal tissues. Samples were crosslinked with 1% glutaraldehyde and chromatin was sonicated with 10 cycles of 30s ON and 30s OFF to obtain chromatin fragment of 100-500 base pairs. Hybridization was carried out either with 100 pmol of Tu-Stroma and MALAT1 probes (pooled 4 biotin probes) or with 100 pmol control GFP probe at 37° C. for 4 hr. Then, biotin probes were captured with MyOne Streptavidin C1 Magnetic Dynabeads (Life technologies). For elution of the RNA and the associated DNA from the captured streptavidin-magnetic we followed the protocol described by Chu et al. The eluted RNA fraction was converted to cDNA using Superscript Vilo cDNA synthesis kit (Thermo Fisher) per the manufacturer's instructions and Tu-Stroma enrichment was analyzed by qPCR performed with PowerUp SYBR Green master mix (Thermo Fisher) and the Tu-Stroma primers described previously on the qPCR section using BIO-RAD C1000 Touch PCR Thermal Cycler w/CFX96 Real Time Optics Module (BIO-RAD) and analyzing the PCR data in the CFX manager v2.3 software. Similarly, we performed PCR to analyze the eluted DNA pulled-down with Tu-Stroma probes in different regions of the hFwe genome to observe where Tu-Stroma was binding. The primers used in the PCR of pulled-down DNA were described previously on the ChIP section.

The sequences of the probes used in this experiment are as follows: GFP anti-sense 5′-CGTATGTTGCATCACCTTCA-BioTEG-3′ (SEQ ID NO:193); Tu-Stroma sense p1 5′-CGGAGTGGATAAAGCATATT-BioTEG-3′ (SEQ ID NO:194); Tu-Stroma sense p2 5′-GCTAAATTGGTTGGTGGAT-BioTEG-3′ (SEQ ID NO:195); Tu-Stroma sense p3 5′-ACTTCCTTCTGCAGTTGGAG-BioTEG-3′ (SEQ ID NO:196); Tu-Stroma sense p4 5′-ACCTAAGATGTGGACTTAAC-BioTEG-3′ (SEQ ID NO:197); Tu-Stroma anti-sense p1 5′-AATATGCTTTATCCACTCCG-BioTEG-3′ (SEQ ID NO:198); Tu-Stroma anti-sense p2 5′-ATCCACCAACCAATTTAGC-BioTEG-3′ (SEQ ID NO:199); Tu-Stroma anti-sense p3 5′-CTCCAACTGCAGAAGGAAGT-BioTEG-3′ (SEQ ID NO:200); Tu-Stroma anti-sense p4 5′-GTTAAGTCCACATCTTAGGT-BioTEG-3′ (SEQ ID NO:201); MALAT1 anti-sense p1 5′-GCGAGGCGTATTTATAGACG-BioTEG-3′ (SEQ ID NO:202); MALAT1 anti-sense p2 5′-TCATCTCAACCTCCGTCATG-BioTEG-3′ (SEQ ID NO:203); MALAT1 anti-sense p3 5′-TTAGCTTTTTGTTTCCTAGC-BioTEG-3′ (SEQ ID NO:204); MALAT1 anti-sense p4 5′-TACTTCCGTTACGAAAGTCC-BioTEG-3′ (SEQ ID NO:205).

In vitro RNA-DNA Capture

We performed PCR on the DNA extracted of HGSC stromal cells with the primer sets from all hFwe genomic regions used in ChIP. PCR reactions were performed either with unmodified dATP and dGTP or with modified 7-deaza-dATP and 7-deaza-dGTP. We synthetized Tu-Stroma single stranded RNA sequence using MEGAscript T7 Transcription Kit (Invitrogen) as per manufacturer instructions. Briefly, we used our Tu-Stroma_pCDH-CMV-MCS-EF1-GFP plasmid to obtain the DNA template of Tu-Stroma needed for RNA synthesis. Then, DNA template was added to RNA synthesis mixture containing biotin-16-UTP, and was incubated at 37° C. for 12 hr. The remaining DNA template was digested with DnaseI, and RNA was eluted in 30 μl of RNase and DNase free water. To pull-down in vitro DNA-RNA adduct, we prepared two 15 μl reaction in 1× Exonuclease I reaction buffer containing 150 fmol of unmodified or 7-deaza modified PCR-fragments and 15 units of Exo I. The reactions were incubated at 37° C. for 30 min. In parallel, we prepared a 5 μl reaction containing 1.5 μl of 10× buffer (100 mM Tris-HCl, pH 7.5, 200 mM KCl, 100 mM MgCl2, 0.5% Tween 20, ribonuclease inhibitors) and 1 pmol of biotin-labelled synthetic RNA. We added to this reaction 10 μl of DNA digested with Exonuclease I and incubated for 40 min at room temperature with gentle shaking. After incubation, we added 10 μl of MyOne Streptavidin C1 Magnetic Dynabeads and incubated for 40 minutes at room temperature with shanking. Then, the mixture was washed with washing buffer 1 (150 mM KCl, 10 mM Tris-HCl, pH 7.5, 5 mM MgCl2, 0.5% NP-40, ribonuclease inhibitors) followed by wash with washing buffer 2 (15 mM KCl, 10 mM Tris-HCl, pH 7.5, 5 mM MgCl2, ribonuclease inhibitors) in a magnetic rack. After the washing steps, we added 100 μl of TE buffer containing RNase A to the magnetic beads, and incubated them for 30 min at 37° C. with shaking. Then, the magnetic beads were briefly centrifuged, and the supernatants were collected after placing the tubes on a magnetic rack. Finally, the RNA-associated DNA was purified and subjected to PCR, together with the remaining 5 μl of DNA digested with Exonuclease I, which served as input. The RNA-associated DNA was normalized to the DNA input.

### UV-Assisted Triplex Capture Assay

UV-Assisted Triplex Capture Assay was performed as described by Postepska-Igielska et al. with modifications. In resume, 5′-6C-psoralen- and 3′-biotin-modified Tu-Stroma oligonucleotides were purchased from Eurogentec (5′-6C-Psoralen-Tu-Stroma-BIOTEG-3′: 5′-6C-Psoralen-CCTTCTGCAGTTGGAGACAGCCTTCTGTGTGG-3′ (SEQ ID NO:206)). HGSC stromal cells were transfected with the modified Tu-Stroma using Lipofectamine™ RNAiMAX as per manufacturer instructions. 24 h after transfection, HGSC stroma cells were harvested and re-suspended with cold 175 μL of NES1 solution (50 mM Tris HCl pH 8.0; 140 mM NaCl; 1.5 mM MgCl2; 0.5% NP-40; supplied with RNase inhibitor). After an incubation of 5 min on ice. HGSC stroma cells were centrifuged at 4° C. for 3 minutes at 300 g and the supernatant was discarded. The pellet containing nuclei was re-suspended in NES2 solution (50 mM Tris HCl pH 8.0; 500 mM NaCl; 1.5 mM MgCl2; 0.5% NP-40; supplied with RNase inhibitor), incubated for 5 min on ice and irradiated with UVA for 10 min (365 nm). Then, 2 mg of genomic DNA or chromatin lysate from HGSC stroma cells were incubated with streptavidin coupled Dynabeads M280 (Invitrogen).

Finally, DNA was purified, analyzed by qPCR, and normalized to input DNA. Primers used covered the hFwe genomic sequence and were described previously on ChIP section.

### Electrophoretic Mobility Shift Assay (EMSA)

EMSA was performed as described previously by Mondal et al. Double-stranded oligonucleotides of hFwe Exon 3 end-labeled with T4 polynucleotide kinase in the presence of [y-32P] ATP were used. We synthetized Tu-Stroma sRNAs as previously described in the in-vitro RNA-DNA capture section. Then, we heated Tu-Stroma at 70° C. for 5 minutes followed by 5 minutes incubation of ice to eliminate RNA secondary structures. To promote the binding between RNA and hFwe Exon 3 labeled dsDNA we used a 10 uL reaction mixture containing, by addition order, nuclease-free water, hFwe Exon 3 labeled dsDNA oligonucleotides (0.4 μmol), 1 μl 10× DNA-RNA adduct forming buffer (100 mM Tris pH 7.5, 250 mM NaCl and 100 mM MgCl2), 1 μl Yeast tRNA (1 mg/ml stock) 2.0 M of RNA oligonucleotides and incubated for 2 hours at 25° C.

In control assay, reaction was treated with 5 units of RNase H (Invitrogen) for 20 min at 30° C. We monitored DNA-RNA adduct formation on 20% polyacrylamide TBE gel (Life Technologies) in 1X TBE buffer supplemented with 8 mM MgCl2 at room temperature for 90 minutes at 200 volts.

### Circular Dichroism Spectroscopy (CDS)

CDS was performed as described by Mondal et al. with modifications. Briefly, we purchased from IDT Tu-Stroma ssRNA and hFwe Exon 3 dsDNA. We record CDS on a Jasco J-720 spectropolarimeter (Jasco) on cylindrical cells with a path length of 0.5 mm and using the Spectra Manager version 2.8. software. The CDS of 2.2 M Tu-Stroma ssRNA and 2.2 M hFwe Exon 3 dsDNA were recorded individually and mixed in a 1:1 ratio in 1×DNA-RNA adduct forming buffer (10 mM Tris pH 7.5, 25 mM NaCl and 10 mM MgCl2). Individual RNA and dsDNAs were equilibrated approximately 1 h at 30° C., while the mixed sample was equilibrated for 4 h at 30° C. or immediately measured. The measurements were performed as described previously.

### ELISA

ELISA was performed as described previously. Briefly, the wells of PVC microtiter plates were coated with antibodies for DARS, ELAC2, EPRS, GNB2L1, IARS, NCL, RARS, RLP12, RPS18, RPS3, RUVBL1, TUFM, HNRNPA2B1, HNRNPH1, EEF1A1, HNRNPK, HNRNPM, HSP90A1B, MOCS3, SNW1, DDX3X, EEF2, HNRNPD, HNRNPU, HNRNPUL1, NSUN2, WDR1, HSPA8, MVP, PCBP1 at a concentration of 1-10 g/ml in carbonate/bicarbonate buffer (pH 7.4). Plates were covered and incubated overnight at 4° C. After incubation, the coating solution on the plates was washed twice with 200 μl PBS. Further, the wells were blocked with 200 μL blocking buffer, 5% nonfat dry milk/PBS, and incubated overnight at 4° C. Next, exosomes extracted from blood of HGSC patients and normal non-cancer individuals were added to the wells and incubated from 90 min at 37° C. Wells were further incubated with 100 μL (0.5 μg/100 p1) of diluted detection antibodies for the above indicated antibodies at room temperature for 2 h, and washed 4 times with PBS. After the washing step, we added 100 μL of secondary antibody conjugated with horseradish peroxidase (HRP), covered the plates, and incubated them for 2 h at room temperature. Finally, the plates were washed four times with PBS and O.D was measured using SPARK Tecan Plate Reader (Tecan).

### Putative Transcription-Factor-Binding-Site (TFBS) Analysis

TFBSs specific to STAT3 in hFwe promoter and in FOS promoter were analyzed using MatInspector (Genomatix, Munich, Germany) with MatBase matrix library 8.0. MatInspector, following previously described details regarding the weight matrices used to identify potential TFBSs.

### Luciferase Assay

To perform luciferase experiments, approximately 5×10⁴ cells per well were seeded in 24-well plates 24 h prior to transfection. Cells were transfected with reporter plasmids for control STAT3 DBS (STAT3 c-FOS binding site), hFwe 4k promote, and hFwe STAT3 DBS 1-4 (1.0-1.5 mg/well) using Lipofectamine® 3000 as per the manufacturer's protocol. Renilla luciferase expression plasmid with empty vector control (0.5 g) and β-galactosidase construct (0.2 μg) were co-transfected as internal controls. After 48 hours of transfection, cells were starved for 6 hours in OPTI-MEM and incubated overnight, either in DMEM serum free medium (control group) or in DMEM serum free medium with 100 ng/mL of IL6 (STAT3 activation group). After the desired incubation, the cells were washed in cold PBS three times and lysed with 200 μl of the lysis buffer by a freeze-thaw cycle, and lysates were collected by centrifugation at 12,000 g for 2 min in a bench-top centrifuge. 20 μl of the supernatant was used for the assay of luciferase activity using a kit (Promega) as previously described. Each transfected well was assayed using SPARK Tecan Plate Reader (Tecan) in triplicates, and the firefly luciferase activity was normalized to p-galactosidase activity. All luciferase constructs were cloned into PGL4 vector.

### Antibody Specifications and Dilutions

We used the following antibodies and dilutions: anti-hFwe-N-term (Genscript Abl; 1:500 for RIC), anti-hFwe-Win (Genscript Ab4; 1:500 for RIC), anti-mouse IgG HRP (1:1000, Thermo Fisher Scientific #31430 for western blot); anti-rabbit IgG HRP (1:5000, Thermo Fisher Scientific #31460 for western blot), IgG (Abcam #ab172730, 1:100 for ChIP and RIP), Actin (Cell Signaling Technology #4970, 1:100 for ChIP and RIP), anti-5-methycytosine (5-mC) (Abcam #ab214727, 1:100 for ChIP); anti-DNMT3a (Abcam #ab2850, 1:100 for ChIP); hnRNPA2B1 (Abcam #ab31645, 1:100 for ChIP and RIP); hnRNPG (Novus Biologicals #NBP1-79904, 1:100 for ChIP); YTHDC1 (Cell Signaling Technology #77422, 1:100 for ChIP); METTL3 (Cell Signaling Technology #86132, 1:100 for ChIP); METTL14 (Novus Biologicals #NBP1-81392,1:100 for ChIP); WTAP (Cell Signaling Technology #56501, 1:100 for ChIP); KIAA-1429 (Thermo Fisher Scientific #25712-1-AP, 1:100 for ChIP); RBM15 (Cell Signaling Technology #25261, 1:100 for ChIP); Spi-1 (Santa Cruz Biotechnology #sc-352, 1:100 for ChIP); TCERG1 (Novus Biologicals #NBP2-20584, 1:100 for ChIP); BRM (Santa Cruz Biotechnology sc-166579, 1:100 for ChIP); H3K9Me3 (Abcam #ab8898, 1:100 for ChIP); H3K4Me1 (Abcam #ab8895, 1:100 for ChIP); H3K4Me3 (Abcam #ab8580, 1:100 for ChIP); H3K27me3 (Abcam #ab6002, 1:100 for ChIP); H3K36Me3 (Abcam #ab9050, 1:100 for ChIP); Hyperacetylation-H3K27Ac (Abcam #ab4729, 1:100 for ChIP); CTCF (Abcam #ab128873, 1:100 for ChIP); MeCP2 (Abcam #ab195393, 1:100 for ChIP); HP1 (Santa Cruz Biotechnology sc-#515341, 1:100 for ChIP); PTB (Thermo Fisher Scientific #32-4800, 1:100 for ChIP); PSIP1 (Thermo Fisher Scientific #MA5-14821, 1:100 for ChIP); MRG15 (Abcam #ab183663, 1:100 for ChIP); SRSF3 (Thermo Fisher Scientific #33-9400, 1:100 for ChIP); RBP2 (Santa Cruz Biotechnology sc-#365993, 1:100 for ChIP); SETl (Abcam #ab70378, 1:100 for ChIP); MLL (Cell Signaling Technology #61295, 1:100 for ChIP); GLP (Santa Cruz Biotechnology #sc-80603, 1:100 for ChIP); SMYD2 (Santa Cruz Biotechnology #sc-393827, 1:100 for ChIP); NSD2 (Santa Cruz Biotechnology #sc-365627, 1:100 for ChIP); ASH1 (Thermo Fisher Scientific #A301-749A, 1:100 for ChIP); SUV39 h1 (Santa Cruz Biotechnology #sc-23961, 1:100 for ChIP); G9a (Santa Cruz Biotechnology #sc-515726, 1:100 for ChIP); SET7/9 (Santa Cruz Biotechnology #sc-390823, 1:100 for ChIP); RIZ (Santa Cruz Biotechnology #sc-130256, 1:100 for ChIP); EZH1 (Thermo Fisher Scientific #PA5-40850, 1:100 for ChIP); EZH2 (Thermo Fisher Scientific #49-1043, 1:100 for ChIP); DOT1 μL (Thermo Fisher Scientific #MA5-24294, 1:100 for ChIP); RNA Pol II (Santa Cruz Biotechnology #sc-55492, 1:100 for ChIP) STAT3 (Abcam #ab31370, 1:100 for ChIP, 1:200 for IHC, and 1:1000 for western blot); pSTAT3 Y705 (Abcam #ab76315, 1:100 for ChIP, 1:500 for IHC, and 1:1000 for western blot), AGO2 (Abcam #abl86733, 1:100 for RIP), DARS (Santa Cruz Biotechnology #sc-393275, 1:100 for RIP), ELAC2 (Abcam #ab205948, 1:100 for RIP), EPRS (Abcam #abA303-957A, 1:100 for RIP), GNB2L1 (Cell Signaling Technology #5432, 1:100 for RIP), IARS (Abcam #ab2296431:100 for RIP), NCL (Abcam #ab1366491:100 for RIP), RARS (Abcam #ab2410851:100 for RIP), RPS18 (Thermo Fisher Scientific #PAS-882111:100 for RIP), RPS3 (Abcam #ab1406881:100 for RIP), RUVBL1 (Thermo Fisher Scientific #PA5-292781:100 for RIP), TUFM (Thermo Fisher Scientific #A9861, 1:100 for RIP), HNRNPH1 (Abcam #ab5832, 1:100 for RIP), EEF1A1 (Thermo Fisher Scientific #11402-1-AP, 1:100 for RIP), HNRNPK (Abcam #ab39975, 1:100 for RIP), HNRNPM (Thermo Fisher Scientific #PA5-30247, 1:100 for RIP), MOCS3 (Thermo Fisher Scientific #A13417-200, 1:100 for RIP), SNW1 (Thermo Fisher Scientific #PA5-118274, 1:100 for RIP), DDX3X (Santa Cruz Biotechnology #sc-81247, 1:100 for RIP and 1:1000 for western blot), EEF2 (Abcam #ab75748, 1:100 for RIP), HNRNPD (Abcam #ab61193, 1:100 for RIP), HNRNPU (Santa Cruz Biotechnology #sc-32315, 1:100 for RIP), HNRNPUL1 (Abcam #ab180952, 1:100 for RIP), NSUN2 (Cell Signaling Technology #44056, 1:100 for RIP), WDR1 (Cell Signaling Technology #173574, 1:100 for RIP), HSPA8 (Cell Signaling Technology #8444, 1:100 for RIP), MVP (Abcam #ab97311, 1:100), PCBP1 (Novus Biologicals #NBP1-52114, 1:100 for RIP), TSG101 (Abcam #abl25011, 1:1000 for western blot), CD9 (Abcam #ab263019, 1:1,000 for western blot), Calnexin (Abcam #abl33615, 1:1,000 for western blot), and GAPDH (Abcam #ab8245, 1:1,000 for western blot), humanized anti-Flower/hFwe/CACFD1 antibody-intravenous injection of the mAb (10 mg/kg, for hFwe monoclonal therapy experiments).

### Western Blot

Protein extracts from cells, human tissue and exosomes were obtained using RIPA buffer (Thermo Fisher Scientific) supplemented with phosphatase and protease inhibitors (Thermo Fisher Scientific), following manufacturer instructions. Briefly, samples were incubated on ice with constant agitation for 5 minutes, followed by centrifugation at 14000 g for 15 minutes. Then, 10 g of protein extracts were prepared with NuPAGE™ LDS Sample Buffer (Invitrogen) and NuPAGE™ Sample Reducing Agent (Invitrogen), and boiled at 70° C. for 10 min. The protein samples were separated on NuPAGE™ 4 to 12%, Bis-Tris (Invitrogen) gels and transferred to nitrocellulose membranes (Invitrogen) using the iBlot system (Invitrogen). After blocking in 5% BSA in TBST (Tris-buffered saline with Tween 20), the membrane was probed with primary antibody against TSG101 (Abcam #abl25011, 1:1,000), CD9 (Abcam #ab263019, 1:1,000), Calnexin (Abcam #abl33615, 1:1,000), STAT3 (Abcam #ab31370; 1:1000), anti-pSTAT3^{Y705}(Abcam #ab76315; 1:1000), DDX3X (Santa Cruz Biotechnology #sc-81247, 1:1000), and GAPDH (Abcam #ab8245, 1:1,000), overnight at 4° C. Membranes were further washed thrice with TBST for 10 min, incubated with appropriate HRP-conjugated-secondary antibodies⁵⁶. Then, membranes were washed as described in the previous step and incubated with Pierce™ ECL Western Blotting Substrate (Thermo Fisher) for 1 minute. Finally, protein expression was detected by chemiluminescence using a C-DiGit (LI-COR). 5′ and 3′ RACE

The 5′ and 3′ RACE experiments were conducted using the SMARTer RACE 5′/3′ Kit (Clontech, #634859), as described previously. Briefly, the total RNA of normal, tumor and stroma cells were extracted using the RNeasy Mini Kit (QIAGEN, #74104) according to the manufacturer's instruction. First-strand cDNA was synthesized using 5-RACE Tu stroma specific primer (TSP-1: 5′-GGCTTCCCTCTCTGCAGA-3′ (SEQ ID NO:388) and 3-RACE Tu stroma specific primer (TSP-2: 5′ TTCTGTGTGGGCTGAAGGAC-3′ (SEQ ID NO:389)) and SMARTer II A oligonucleotide as described in the manufacturer's manual. The touchdown nested PCR program (95° C. for 10 min; 5 PCR cycles of 94° C. for 30 s, 72° C. for 3 min; 5 PCR cycles of 94° C. for 30 s, 70° C. for 30 s,72° C. for 3 min; 25 PCR cycles of 94° C. for 30 s, 68° C. for 30 s,72° C. for 3 min;72° C. for 10 min) was used to amplify cDNA ends. The PCR product was purified from 2% agarose gel with the NucleoSpin Gel and PCR Clean-Up Kit (supplied with the SMARTer 25 RACE 5′/3′ Kit) and the primer sequences used for PCR are listed above. The PCR products were detected by 2% agarose gel electrophoresis and sequenced.

### Tu-Stroma Barcoded Synthesis and Experiments

We extracted total RNA from OVCAR8 cells using the PureLink RNA Mini kit. Then, 10 ng of total RNA was reverse-transcribed to cDNA using a Superscript Vilo cDNA synthesis kit. Tu-Stroma was barcoded at the 5′ and 3′ ends by performing PCR DreamTaq Green PCR Master Mix (Applied Biosystems) with the primers 5′Barcode 5′-GGCAAGCTTAATGATACGGCGACCACCGAGATCTACACTCTGGATGAGATTCCTGAT TTC-3′ (F) (SEQ ID NO:390) and 3′Barcode 5′-TCGATGTCGACTCGAGTCTTATGACTCT-3′ (R) (SEQ ID NO:391). We ran the PCR product and purified it with a QIAquick Gel Extraction Kit (Qiagen), following the manufacturer's instructions. Then, we linearized pLenti-EFla-empty with PmeI to create blunt-ends. The linearized vector was treated with terminal transferase (NEB) to add the nucleotide thymine at 5′ and 3′ ends. Finally, we performed TA cloning by mixing the barcoded Tu-Stroma PCR product with the linearized vector treated with terminal transferase using T4 DNA ligase. OVCAR8 Tu-Stroma KO, OVCAR8 Rab27a and Rab27b double KO, OVCAR8 Rab27a, Rab27b, and Tu-Stroma triple KO, and stromal cells were transduced with lentivirus containing the barcoded Tu-Stroma and cultured either in monocultures or cu-cultures. Finally, we analyzed the expression of endogenous Tu-Stroma expression and barcoded Tu-Stroma expression from monocultures and from stromal cells upon co-culture using primers specific for the endogenous Tu-Stroma 5′-GCGGCCATGGAAAGAACTT-3′ (F) (SEQ ID NO:7) and 5′-GAAATGGGTCAGCAGGGCTT-3′ (R) (SEQ ID NO:392), and specific for the barcoded Tu-Stroma 5′-GGCAAGCTTAATGATACGGCGACCACC-3′ (F) (SEQ ID NO:393) and 5′-TCGATGTCGACTCGAGTCTTATGACTCT-3′ (R) (SEQ ID NO:394). The PCR products were run in 1% agarose gels to confirm the amplification and band size of the amplicons using the ChemiDoc Touch System (BIO-RAD).

### CLIP Assay

OVCAR8 cells were UV-crosslinked at 150 mJ/cm² at 365 nm using a Stratalinker, as described previously. Cells were lysed in lysis buffer (50 mM Tris-HCL, pH 7.4, 100 mM NaCl, 1% NP-40, 0.1% SDS, 0.5% sodium deoxycholate, with protease inhibitors and transferred to 1.5 mL microtubes. Lysate was partially digested by 2 U/μL RNaseTl/A for 15 min at 22° C. for CLIP. RNA was immunoprecipitated with DDX3X antibody and protein A/G beads for overnight 25 at 4° C. After washed for 4 times, RNA was phosphorylated by T4 PNK and ligated RNA between 3′ and 5′ ends by RNA T4 ligase. SDS-PAGE loading buffer was added, and the mixture was incubated at 70° C. for 10 min. After running the SDS-PAGE gel, the RNA-protein complexes were transferred from gel to a nitrocellulose membrane using a wet transfer apparatus (30 V for 1 h). The membrane with target protein was cut up, and the targeted membrane piece was incubated with Proteinase K for de-crosslink. After de-crosslink, RNA was reverse transcribed into cDNA and subjected to real-time qPCR analysis using BIO-RAD C1000 Touch PCR Thermal Cycler w/CFX96 Real Time Optics Module (BIO-RAD) and the CFX manager v2.3 software. Five volumes of TRIzol were added to each sample, and the RNA extraction and the cDNA generation were performed according to the protocol mentioned in methodology. To perform CLIP assay, we used the primers 3′Linker: 5′-OH-GUGUCAGUCACUUCCAGCGG-3′ (SEQ ID NO:395) and 5′Linker: 5′-OH-AGGGAGGACGAUGCGG-OH-3′ (SEQ ID NO:396).

### Fluorescence In-Situ Hybridization

In situ hybridization was performed using the ViewRNA™ ISH Tissue Assay Kit (Thermo Fisher #QVC0001) following the manufacturer's protocol. Slides from high grade ovarian cancer tissue were baked at 60° C. for 60 min and deparaffinized in xylene twice for 10 min. Next, slides were incubated twice in 100% ethanol for 5 min and rehydrate with descending series of alcohol. To allow target accessibility slides were incubated in 1× pretreatment solution at 90° C. for 10 min, washed in ddH₂O, and treated with protease solution (1:100 in 1× PBS) at 40° C. for 20 min. After fixation in 4% buffered paraformaldehyde for 4 min at room temperature, slides were washed in PBS. Target probe sets (1:40 in Probe Set Diluent QT) designed against Tu-stroma were added to the slides and incubated at 40° C. for 2 h. Next, slides were washed in wash buffer, and signal amplification was achieved via a series of sequential hybridization steps. PreAmplifier Mix QT was added to the slides and incubated at 40° C. for 25 min. After washing the slides in wash buffer, Amplifier Mix QT was added to the slides followed by incubation at 40° C. for 25 min. Again, slides were washed in wash buffer and Label Probe-Alexa Fluor 647, according to target probe type was added to slides and incubated at 40° C. for 25 min. Afterwards, slides were washed in wash buffer, fast blue substrate was added, and the reaction took place at RT in the dark for 30 min. Slides were successively washed in wash buffer, PBS and ddH₂O. Counterstaining was done for 3 min using DAPI. Finally, slides were rinsed in PBS, mounted with ProLong™ glass antifade mountant (Thermo Fisher #P36980). Tissue slides were scanned and digitalized using Zeiss LSM-800 confocal microscopy and analyzed using Zeiss Zen 3.8 software.

### Live-Cell Imaging

We performed various live-cell imaging experiments using Leica TCS SP8 confocal laser microscopy (Leica Microsystems), a Nikon Al HD25 confocal microscope (Nikon), and a Zeiss LSM-800 with Airy Scan Confocal Microscope (Zeiss). Images were analyzed using Zeiss Zen 3.8 software and ImageJ/Fiji version 2.3.0/1.53e.

### Flow Cytometry

GFP-positive HGSC cancer cells and GFP-negative HGSC stromal cells were co-cultured in a 1:1 ratio in a 10 mm culture dish. Cells were harvested, washed twice with PBS, and suspended in FACS buffer (PBS, 1 mM EDTA, 25 mM HEPES pH 7, 2% FBS). HGSC cancer and stromal cells were sorted by GFP expression in a BD FACSAria™ Fusion cell sorter and analyzed in the BD FACSDiva™ version 8.0.2 software. Cells were first analyzed by FSC/SSC gates to define a homogeneous population, and FSC-H/FSC-A gates were used to sort singlets exclusively.

Then, GFP-positive HGSC cancer and GFP-negative HGSC stromal cells were sorted based on the analysis of GFP using the SSC/GFP gate. Finally, we analyzed the GFP expression of sorted cells to determine the purity of GFP-positive and GFP-negative populations.

### Survival Analysis

Cox's proportional hazard model was used to estimate the hazard ratio of the relative expression of hFwe-Lose or Tu-Stroma, adjusted for age and stage. Age was included as a continuous variable on a spline basis with two degrees of freedom to allow for some non-linear relationship between age and the log hazard. Stage was included as a categorical variable. The test cohort comprised only patients with stages III and IV. The validation cohort comprised patients of all 4 stages, but only 7 with stage 14 with stage II, which were combined. The relationship between log 2 relative expression and the log hazard was assumed linear.

### Invasion in 3D-Hydrogel Spheroids

Tumor spheroids were prepared from mCherry-labeled WT or Tu-Stroma-KO OVCAR8 cells, and tumor stromal cells (2:1 ratio). 3D Spheroids were prepared using modified hanging-drop method, in which drops of cell suspension are held hanging from the bottom of an inverted tissue-culture plate until cells agglomerate spontaneously at the lower part of the drop due to gravity. Cells were deposited in 25 μl droplets on the inner side of a 20 mm dish and incubated for 72 h at 37° C. when the plate is facing upside down to allow for spheroid formation. For assessment of cell invasion, 3D spheroids (total of 3,000 cells/spheroid) were embedded in Matrigel (Corning), seeded in a 96-well plate, and incubated with complete medium. 3D spheroid invasion was visualized after 72 h and 96 h with an EVOS FL Auto cell imaging system (ThermoFisher Scientific, Massachusetts, USA).

### Animal Housing Conditions

The mice were kept in a controlled environment with a 12-hour light/dark cycle, ensuring a consistent lighting pattern. The humidity levels in the room were maintained between 40 and 60 percent within the temperature range of 20 to 24° C. to provide optimal living conditions. Polycarbonate cages were utilized to house the mice, chosen for their durability and suitability in laboratory settings. These cages were cleaned regularly to maintain strict hygiene standards, preventing any potential contamination or adverse effects on the mice's health. Standard laboratory bedding was used, and water and food were provided ad libitum, ensuring the mice had continuous access to a balanced diet. The diet consisted of typical laboratory chow, formulated to meet the nutritional needs of the mice and support their overall health during the experiment. Items such as nesting materials and shelters were added to the cages, allowing the mice to engage in natural behaviors like burrowing and nest building. Throughout the study, temperature, humidity, and other aspects of the living environment were closely monitored to ensure stability. Regular checks were performed to confirm that these conditions remained within the specified ranges, minimizing any potential influence on the experimental results.

### Tumor Cell Growth in Patient-Derived Scaffolds

HGSC samples were used to generate PDSs as previously described by Landberg et al. Tumors were washed with a detergent mixture containing 3 mM sodium azide (G-biosciences), 5 mM Ethylenediaminetetraacetic acid (EDTA; Invitrogen), 3.5 mM sodium dodecyl sulfate (SDS; Applichem) and 0.4 mM phenylmethylsulfonyl fluoride (PMSF; Sigma Merck) for 6 h. Then, a second wash was performed with a second detergent mixture composed by 3 mM sodium azide, 5 mM EDTA and 0.4 mM PMSF for 6 h. After washing, tumors were rinsed with distilled water for 72 h and Phosphate Buffered Saline (Merk) for following 24 h. Finally, we sterilized PDS with 0.1% Peracetic acid (Merck) in distilled water for 1 h followed by 1% Antibiotic-Antimycotic (Gibco) in PBS for 24 h. The resulting PDSs were section into 3×3×2 mm pieces and placed in 24-well plates. Then, 6×10⁵ mix of patient-derived stromal cells and either OVCAR8 WT-GFP or OVCAR8 Tu-Stroma KO-GFP cells were added to PDSs in a ratio 9:1. After 24 h incubation, recellularized PDSs were transferred to a new 24-well plate to prevent cells from growing in monolayer. Finally, PDSs were incubated for 9 days and imaged with a ZEISS light-sheet Z.1.

### Light-Sheet Microscopy

The acquisition of the organoid images was done with a ZEISS light-sheet Z.1. Samples were attached to the ZEISS commercial holder by using the UV curated glue named “Bondic” while the ZEISS chamber was filled with PBS. We used 5x/0.1 illuminating lenses and an EC Plan-Neofluar 5x/0.16 lens for detection. To collect the GFP signal from the cells of interest, we used a double pumped solid state (DPSS) 488 nm laser as excitation light while the emitted light was collected by using a band-pass (BP) emission filter 505-545 nm. Images were recorded with a PCO Scientific CMOS. In order to ensure that the entire information was collected from every sample, each organoid was acquired from two different angles, 180 degrees apart, and with a redundancy in Z of at least 30%. Scaling was kept equal at every experiment with pixel size in X and Y of 2.53 μm and a Z step of 6.14 μm. After acquisition, the multi angular images were fused by using the software Arivis Vision4D version 4.0.

### Image Visualization and Analysis

Visualization, figures, movies, and analysis were done with the software IMARIS version 9.3.1. For better understanding of the content of each image, the single channel (GFP) files are presented with a double look-up-table (LUT) where cells are labeled in green and the scaffold and desmoplastic architecture of the tumor is in magenta. The different colors were assigned accordingly to a difference in threshold between the foreground and the background of the signal. We established the threshold of separation between the two structures by measuring the signal intensity across 10-20 randomly chosen cells in each organoid and comparing it with the signal measured in their surroundings. The limit of threshold used was always within the 2.5% of the entire signal.

Regarding the analysis, cells were automatically counted in 3D by using the plug-in “spots” of IMARIS version 9.3.1 and human eye verified. Again, the threshold used was the same as described above. In order to facilitate the automatic counting verification and handling of the data, cell counting was dome in boxes of 506 μm² (200×200 pixels) each of the including both frontal and rear surface of the organoid. The total amount of data for WT samples was n=11, while for KO samples was n=15.

### Orthotopic HGSC Implantation and Treatment

All animal studies were performed with the approval from the Virginia Commonwealth University and Ohio University Institutional Animal Care and Use Committee (IACUC). Surgeries were performed on female NOD.Cg-Prkdc^{scid}Il2rg^{tmJWjl}/SzJ (NSG) mice at 15-20 weeks of age. Mice were anesthetized with isoflurane and oriented with ventral side down. OVCAR8 cells were administered either orthotopically or via intraperitoneal injection. For orthotopic implantation, a vertical incision was made near the spine, between the costal margin and femur. A similar incision was made to open the parietal peritoneum. The ovarian fat pad was isolated from the peritoneum. A cell suspension with OVCAR8 cells (KO or WT) was injected into the ovary using an insulin syringe. After appropriate closure with suture and staples, the mice were observed for 50 days or until mice reached a protocol endpoint. For the Flower monoclonal antibody, the mice received a 10 mg/kg intravenous injection (three injections of 10 uL each). As per the guidelines, we have used a treatment regimen of Docetaxel/carboplatin/bevacizumab+maintenance bevacizumab. The administration included Docetaxel at a dose of 75 mg/m² IV (adjusted for the total body surface area of a mouse model, which is 0.007 m², resulting in the dose of 0.75 mg/kg) followed by Carboplatin AUC 6 IV day 1 (which is equivalent to 75 mg/kg in mouse), and Bevacizumab at 15 mg/kg intravenously. Metastatic lesions were counted microscopically after the experimental endpoint.

### In Vivo Bioluminescence Imaging and Ex Vivo MR Imaging

For in vivo imaging, we used the Living Image version 4.8.2 software and the Xenogen IVIS system-200 (Cambridge Scientific). After initializing and ensuring that the correct temperature was reached, we selected the Luminescent Imaging Mode on the control panel and activated the isoflurane anesthesia for both the chamber and IVIS system. Mice were anesthetized with 2.5-4.5% isoflurane and 1 L/min oxygen, adjusted to 1.5-2.5% for maintenance post-anesthesia. D-luciferin was administered at a dosage of 150 mg/kg via intraperitoneal injection, and mice were transferred to the IVIS system approximately 5 minutes post-luciferin injection, positioned on the temperature-controlled platform with noses in the cone. Imaging settings were configured with Medium Binning, and exposure times (20 seconds) were set. Ex vivo MRI scans of mouse ovarian tumors were conducted at The Molecular Imaging Center, University of Bergen, using a MRS*DRYMAG 7.01′ preclinical Powerscan PET/MR scanner (Philips MR Solutions). T2 weighted MR images were acquired with the following parameters: TR 3000 ms, TE 45 ms, 25 30 averages, slice thickness 0.5 mm, matrix size 256×256, field of view 14×14 mm, scan time 40 mins.

### Flower Monoclonal Antibody Development

Two peptides, peptide 1(CSGGAPGASASSAPPAQEEG (SEQ ID NO:251)) and peptide 2 (CGGAPGASASSAPPAQEEGMTWWYR (SEQ ID NO:252)) from CACFD1 were chosen based on the specificity and immunogenicity, synthesized and conjugated to KLH. The underlined sequences are identical in both the peptides. Four female Balb/c mice aged 8-10 weeks were immunized with 80 g of (primary dose) and with 40 g (booster dose) of KLH conjugated peptides with Complete Freund's adjuvant followed by 05 boosters with Incomplete Freund's adjuvant. The mouse with highest titer in ELISA was sacrificed and the spleen was collected under aseptic condition. After washing the spleen with sterile PBS, splenocytes were isolated. The splenocytes were washed with PBS and then counted. The splenocytes were fused with myeloma cells by using PEG-1500. After fusion, the cells were plated in 96 well plates in the presence of HAT and were incubated in the CO2 incubator at 37° C. with 5% CO2. After 2 weeks of incubation, indirect ELISA was performed with the supernatants of 96 well plates against the peptides coated at 200 ng/well. Peroxidase conjugated goat anti-mouse IgG secondary antibody (ABEOMICS, Inc. San Diego, CA) was used at 1:5000 dilution, and the absorbance was measured at 450 nm. The variable regions of the mouse hFwe monoclonal antibody were amplified by polymerase chain reaction of cDNA derived from hybridoma cells. The amplicons were sequenced and sub-cloned into expression plasmids. The recombinant antibody was expressed in CHO cells and purified on the Protein A resin. The reliability of the PCR-based sequencing results was confirmed by testing the recombinant antibody for binding to the Flower peptides. The variable region sequences of the mouse hFwe monoclonal antibody were analyzed via structural modeling, and 5 humanized variants of the heavy chain variable region (VH) and the light chain variable region (VL) were designed for construction and expression of 25 humanized antibodies (combination of 5 designed heavy chains and 5 designed light chains) for ELISA with the parental chimeric antibody as the control. The expression levels of the humanized and chimeric antibodies were also assessed to ensure that antibody humanization affects neither the activity nor yield of the hFwe antibody. Based on ELISA and western blot results, the clones were selected for further single-cell cloning processes by limiting dilution and were plated into 96-well culture plate. Immune sera were used as positive controls. Six subclones were tested separately against Peptide-1 and peptide-2 coated at 200 ng/well by ELISA. Since there are amino acid identity between two peptides, all the selected subclones were reacted against both peptides. The sub-clone with strongest reactivity was selected. CHO-K1 cells were cultured in a 6-well plate and were transfected with plasmid containing humanized mAb. After 48 h post transfection, cell media was replaced with medium supplemented with 800 g/mL G-418. To screen for high-producing clones, the transfected single cell was transferred into 96-well plates with G-418 containing medium. Only the single cells per well were selected for the subsequent measurements. Following dilution, the cells were incubated for 2-3 weeks. The level of mAb expression in the cell culture supernatants was determined by indirect ELISA. The CHO cells stably expressing the monoclonal expression sequence were expanded and subjected to large-scale culture in a spinner flask (1L) for 2 weeks in 4% IMDM with continuous stirring. The harvested culture supernatant was affinity purified by Protein G column (Cytiva) according to manufacturer's protocol. In brief, the Protein G column was equilibrated with PBS (pH 7.2). The culture supernatant was passed through the column at the rate of 1 ml per minute and the flow through was loaded again to allow maximum binding. The column was washed with PBS and the bound antibody was eluted with glycine buffer (pH 2.7). After dialysing against PBS (pH 7.2) the antibody was estimated by measuring the absorption at 280 nm in a Spectrophotometer (Bio-rad).

While the above provides the particulars for antibodies 1A-1B, 2A-2B, 3A-3B, and 4A-4B, it should be noted that antibody heavy chains VH1-VH6 and light chains VL1-VL6 were produced, humanized, and analyzed as described above.

### Statistics and Reproducibility

FIG. 1C-This experiment was repeated independently 4 times with similar results. FIG. 1F-This experiment was repeated independently 3 times with similar results. FIG. 1H-This experiment was repeated independently 5 times with similar results. FIG. 2A-This experiment was repeated independently 4 times with similar results. FIGS. 2D, 2H, 2I, 2J, and 2M. These experiments were repeated independently 3 times with similar results. FIGS. 3B and 5B. These experiments were repeated independently 4 times with similar results. FIGS. 25B and 25C. These experiments were repeated independently 5 times with similar results. FIGS. 26A and 26C. These experiments were repeated independently 3 times with similar results. FIGS. 26D, 27H, 27I, 27J, 27K, and 27L. These experiments were repeated independently 4 times with similar results. FIGS. 29B, 30A, 30B, 30D and 30E. These experiments were repeated independently 3 times with similar results. FIG. 31A. This experiment was repeated independently 4 times with similar results. FIGS. 7A and 8A. These experiments were repeated independently 4 times with similar results. FIGS. 9B and 9E. These experiments were repeated independently 5 times with similar results. FIGS. 11C and 11F. These experiments were repeated independently 3 times with similar results. FIGS. 16C, 16D, and 17D. These experiments were repeated independently 5 times with similar results. FIGS. 19B, 19C, 19D, 19E, and 19F. These experiments were repeated independently 4 times with similar results.

### Statistical Analysis and Data Visualization

Data were analyzed using R 4.1 1 and Python 3.7.4 using scikit-learn. If not stated otherwise, values are expressed as means±standard error (SE) of independent experiments. Samples sizes were selected as large as possible within practical and financial limits of feasibility. For the analysis of patient samples, there were 2 groups, noncancer, and cancer patients; this further randomization was not relevant. All statistical tests are 2-sided. Appropriate linear models were fit to estimate mean differences and slopes using Im function from the stats (version 3.6.2) package. Concentration and intensity values were log-transformed before analyses. Reasonability of the model assumptions were checked using residual diagnostic plots. Estimates were considered to be significantly different from zero at P<0.05. The principle component analysis was performed in R using the prcomp function from the stats (version 3.6.2) package. Principle components 1 and 2 were used for default visualization. The diffusion matrix and pseudotime were calculated using destiny (version 3.8.0) package with standard parameters. The diffusion components 1 and 2 were used for default visualization. The significance of the model, defined by the pseudotime analysis, was estimated as the sum of Type I and Type II errors (p-values) in the student's t-tests between hFwe-Win and hFwe-Lose isoforms expressions for respective steps. The survival analysis was done using the coxph function from the survival (version 3.2-13) package.

### Example 2

### Tumor Cells Gain Competitive Advantage by Actively Reducing The Cellular Fitness of Microenvironment Cells

Flower Exon 3 Methylation in Stromal Cells is Associated with Flower-Lose Expression

The hFwe gene locus (Flower, CACFD1, chr-9q34.2) is comprised of 6 exons, which are alternatively spliced to generate four protein isoforms (hFwe1—hFwe4) (FIGS. 1A & 1B). hFwe isoforms 1 & 3 function as Flower-Lose proteins, whereas hFwe2 & hFwe4 isoforms function as Flower-Win proteins. Exon 3 inclusion or exclusion determines the generation of Flower-Win or Flower-Lose isoforms, respectively (FIGS. 1A & 1B). Multiplex and standard IHC staining with hFwe N-terminus Ab (stains both Win and Lose isoforms) (FIGS. 1C, 7, and 8), and Flower mRNA studies show high hFwe-Win expression in HGSC tissue and loss of Exon 3 with increased hFwe-Lose expression in the stromal tissue (FIGS. 9A and 9B). DNA methylation is known to regulate exon inclusion and exclusion during splicing. As such, we performed experiments to determine if methylation plays a role in Exon 3 exclusion in stromal tissue. DNA was extracted from normal ovarian, HGSC stromal, and tumor tissue samples and subjected to bisulfite treatment, followed by methylation-specific PCR, sequencing of the Exon 3 loci, and Methylight Assay. Results demonstrate the presence of methylated cytosines in the stromal DNA (FIGS. 9C-9F). ChIP using anti-DNA methylation and DNAMT3A Abs on Exon 3 region in stroma shows high chromatin occupancy for DNA methylation and DNMT3A binding and both showed a significant correlation with hFwe-Lose expression in the stromal tissue (FIGS. 23A-23C). We performed a ChIP-based screening in the stromal tissue and the results show a high chromatin association of proteins involved in epigenetic modifications such as H3K9Me3, HP1, SRSF3, GLP, and G9a (FIG. 23D). We compared RNA expression levels of multiple spliceosome components in tumor and stromal tissue, which demonstrated no changes (FIG. 10A). Exon 3 of the hFwe gene shows a pattern similar to the methylation-dependent exons with a high G+C content of the first 100 nucleotides downstream at the 5′ splice site (FIG. 10B). We analyzed the conservation of Exons 2, 3, and 4 of the hFwe gene in 100 vertebrates and found significant conservation peaks in Exon 3 (FIG. 10C). Overall, the alternative Exon 3 of the hFwe gene is conserved, has high downstream GC content, and contains intermediate to strong splice sites.

Exon 3 Exclusion in the Stroma is Associated with DNA Methylation and Epigenetic Changes

We decided to test whether DNA methylation of Exon 3 is associated with its exclusion in the HGSC stromal tissue. The binding pattern of G9a, GLP, H3K9Me3, HP1, SRSF3, RNA-Pol II, and DNA methylation status was observed on all six exons in the normal, HGSC (cancerous), and stromal tissue (FIG. 24A). G9a and GLP showed high association specifically and exclusively on the Exon 3 body of the stromal tissue. A similar pattern was observed for H3K9Me3, HP1 and SRSF3 binding, suggesting a role of site-specific epigenetic modification in regulation of splicing at the Exon 3 body (FIG. 9A). We used Methylight to evaluate methylation patterns and results show that only Exon 3 has a high methylation level specifically in HGSC stromal tissue (FIG. 24B). This suggests that the basic design of the splicing pattern is to always include Exon 3, and a site-specific epigenetic intervention on the Exon 3 body alters this natural pattern resulting in Exon 3 exclusion in the stroma. This is consistent with the near inversion of the binding pattern of H3K9Me3 and HP1 enrichment at the Exon 3 body in stroma compared to RNA polymerase II on the heatmap (FIGS. 11A-11C, 24A). A quantified map summary of the association of the epigenetic modifiers with particular emphasis on the unusual enrichment pattern of the proteins on Exon 3 in stromal tissue is shown (FIGS. 11D-11E). Since SRSF3 association is a critical factor, 5 we revalidated the qChIP analysis with standard ChIP (FIG. 11F). We analyzed the DNA methylation and SRSF3 enrichment levels on the Exon 3 body and both showed a significant correlation (p=1.4×10⁻⁶) (FIG. 11G). A model based on these findings is presented in FIG. 1D, which shows Exon 3 inclusion in the HGSC tissue. In the adjacent stromal tissue, the Exon 3 body is hypermethylated with high association of G9a, GLP, H3K9Me3, HP1, and SRSF3 resulting in Exon 3 exclusion, thus generating the hFwe-Lose isoform (FIG. 1D).

### Linc01914 Regulates DNA Methylation and Epigenetic Cascade at Flower Exon 3

The next question we addressed is how Exon 3 is hypermethylated. We performed a screen observing all expressed sequences as BLAST targets and mapped them based on the site of association and the expression of these targets in the stromal tissue of HGSC samples (FIG. 12A). The screen reveals a LncRNA (LINC01914) with a 21 bp region that has 90% complementarity and repeat palindrome sequences in the middle of the Exon 3 body.

We performed analysis to determine if LINC01914 specifically alters the expression of the Flower gene or if it impacts the adjacent gene locus in Cis (chromosome 2) or Trans (chromosome 9). LINC01914 shRNA-mediated knockdown did not affect the expression of its nearby genes on chromosome 2 and the genes adjacent to the Flower gene locus (FIGS. 12B, 13A-13B), thus excluding the possibility that LINC01914 acts by influencing its nearby genes in Cis. We characterized LINC01914 gene (chromosome 2p21); RNA-sequencing reveals that it has two splice variants (FIGS. 1E, 12B, 14A-14B), one with four and another (function variant) with three exons (Exon 2 excluded; FIG. 14C—SEQ ID NO: 399). The gene has an active promoter region with significant H3K27Ac and H3K4Me3 binding, and DNase cluster analysis reveals active exon sites. The gene is also conserved in mammals and other vertebrates (FIGS. 12B, 14B-14D). LncRNA has a 5′-cap and is polyadenylated (FIG. 1E). 5′ and 3′ end RACE (rapid amplification of cDNA ends) was performed to identify the synthesized ends of this lncRNA (FIG. 1F). RT-PCR amplification using end-sequence primers showed a consistent length of 490 bp of LINC01914. Phylo CSF score and CAPT coding probability analysis reveal the non-coding protein nature of LINC01914 gene locus (FIG. 15A). ALU elements are absent and LINC01914 does not bind to AGO2 protein (component of the microRNA-containing RISC complex) (FIGS. 15B-15C). The predicted structure of LINC01914 is presented with the potential hFwe Exon 3 interacting region (FIG. 15D). Significant correlations between SRSF3 binding on Exon 3 of the stromal tissue and LINC01914 expression (FIGS. 15E-15F), the expression of LINC01914 and hFwe-Lose (FIGS. 15G, 1511), and the expression of LINC01914 and DNA methylation (FIG. 151) were observed. ChIP and MethyLight Assay demonstrate consistent Exon 3 methylation pattern in the stromal tissue (FIGS. 15J and 16A).

Overexpression of LINC01914 significantly increased DNA methylation, DNMT3A, G9a, GLP, H3K9Me3, HP1, and SRSF3 association with Exon 3 (FIGS. 1G, 16B, 16D). RT-PCR and qPCR of the hFwe-Win, hFwe-Lose, and Exon 3 transcripts demonstrates amplification of the Lose isoform in the LINC01914 overexpressed stromal cells (FIGS. 1H, 9B, and 16C).

To validate the critical role of DNA methylation in activating hFwe alternative splicing, we performed CRISPR-off targeting hFwe at Exon 3 in stromal cells as described previously. CRISPR-OFF induced the methylation efficiency of hFwe at Exon 3 as confirmed by bisulfite treatment followed by PCR (FIGS. 25A-25B). We observed that co-culture of WT cells shows binding of LINC01914 to hFwe at Exon 3 (top lane, 1^{st} plot), high DNA methylation (top lane, 2^{nd} plot), recruitment of splicing machinery (top lane, heatmap), low expression of hFwe-Win, and high expression of hFwe-Lose (top lane, 3^{rd} plot) in stromal cells. LINC01914 KO in OVCAR8 cells lead to no observation of binding between this lncRNA and hFwe Exon 3 in stromal cells. Co-culture between OVCAR8 LINC01914 KO cells and hFwe Exon 3 CRISPR-off stromal cells showed no binding between LINC01914 and hFwe Exon 3 in stromal cells (bottom lane, 1^{st} plot). These results suggest that DNA methylation mediated by LINC01914 is a critical step for the alternative splicing of hFwe isoforms in stromal cells, which results in the expression of hFwe-Lose isoforms (FIG. 25C).

To further demonstrate the functional role of LINC01914 in hFwe-Lose expression, we performed a series of knockout (KO), Poly-A knock-in, and rescue experiments utilizing WT and mutant (MT) LINC01914 (FIGS. 16E and 26A). A model explaining these experiments is shown in FIG. 26B. RT-PCR analysis from these 3 co-cultures demonstrates the ability of exogenous WT LINC01914 to increase or rescue the expression of the Flower Lose isoform in the presence of both LINC01914 KO and Poly-A knock-in (FIG. 2A). Bisulfite treatment and MSP shows the presence of methylation in the KO and Poly-A knock-in experiments with the addition of WT LINC01914 (FIG. 26C). ChOP analysis further confirms these findings with increased relative expression of hFwe-Lose in the stromal cells of the rescue groups (FIG. 2B).

Stromal cells in conditional media and in co-culture with tumor cells were subjected to treatment with Brefeldin A (cytokine inhibitor). The results demonstrate that cytokines released from tumor cells are not responsible for Exon 3 splicing in stromal cells as there is no difference in hFwe-Lose generation in the presence of Brefeldin A (FIG. 26D). From this, we conclude that LINC01914 is the most upstream regulatory event that controls hFwe-Lose expression by promoting DNA methylation-induced alternative splicing at Exon 3.

### Linc01914 Binds at Exon 3 of the Flower Gene

We explored the possibility of a physical interaction between LINC01914 and Exon 3. We fine-mapped LINC01914 and Exon 3 association using the ChOP method (FIG. 26E). The ChOP pull-down using LINC01914 anti-sense oligos detected specific enrichment of LINC01914-RNA on the Exon 3 body (FIGS. 17A-17B). CHOP shows that LINC01914 associates exclusively with the Exon 3 body of the stromal tissue (FIG. 17C). A model representing the interaction site and the GA-enriched motif sequences in hFwe Exon 3 body and LINC01914 is shown. The complementarity and the mirror image sequences within the GA-enriched motifs are depicted (FIG. 2C).

The primary cancer cells were transfected with blank-GFP and co-cultured with primary stromal cells, and these cells were sorted after 8 days (FIGS. 17D-17E). ChOP shows that LINC01914 exclusively associates with the Exon 3 body in stromal cells (FIG. 27A). To examine the potential of LINC01914 to form DNA:RNA complexes in vitro, we incubated DNA fragments of all six hFwe exons and their 200 bp 5′ and 3′ flanking regions with biotinylated LINC01914 and monitored RNA-associated DNA. The association of LINC01914 was compromised when the PCR fragment was generated in the presence of 7-deaza-purine nucleotides (FIGS. 27B & 27C). A similar binding specificity was observed after incubation of biotinylated LINC01914 with isolated nuclei of stromal tissue from HGSC patients (FIG. 27D). We investigated if LINC01914 binds at the Exon 3 body in vivo, as FIG. 27E shows that only Exon 3 was exclusively associated with ectopic LINC01914. In FIG. 27F, the primary stromal cells were transfected with biotinylated oligoribonucleotides that carry a psoralen moiety at the end. This allows fixation of nucleic acid interactions by photo-activation, without cross-linking proteins bound to RNA or DNA. When photo-activation was removed, minimal amounts of Exon 3 DNA were associated with psoralen modified oligoribonucleotides, suggesting that the interaction of LINC01914 with the Exon 3 body is direct and not mediated by other auxiliary proteins.

We performed circular dichroism (CD) spectroscopy to investigate the binding ability of LINC1914 with Exon 3 (FIG. 27G). We monitored the formation of this DNA-RNA adduct via electrophoretic mobility shift assays (EMSA). This experiment demonstrates full-length (490 bp) LINC01914 interacting with the double-stranded oligonucleotide comprising full-length Exon 3 (126 bp), forming a low-mobility complex (super shift) (FIG. 2D). An EMSA utilizing the precise 21 bp site of interaction between LINC01914 and Exon 3 as shown in FIG. 2C was performed (FIG. 27H). Additional EMSA experiments were performed demonstrating the competitive binding of LINC01914 to Exon 3 in the stromal cells (FIG. 27I). Addition of increasing concentrations of unlabeled LINC01914 leads to decreased signal of the full length, labeled LINC01914-Exon 3 complex (FIG. 27J). Addition of an RNA transcript containing the first 245 bp of LINC1914 (including Exon 3 binding site) leads to formation of a DNA-RNA complex (FIG. 27K). A similar EMSA was performed with an RNA transcript containing the last 245 bp of LINC01914 (without the Exon 3 binding region) (FIG. 27L). From this, we identify the binding region of Exon 3 with LINC01914 (FIG. 15D) and conclude that the mechanism of action of LINC01914 involves forming a DNA-RNA adduct (likely a triplex structure) with Exon 3 of the hFwe gene locus in the stromal tissue.

### Ddx3Xbindsand Transports Tu-Stroma Via Exosomes to the Stromal Tissue.

The next question we addressed was how LINC01914 expression is regulated. ChIP data shows that in normal ovarian tissue and HGSC stromal tissue, the LINC01914 promoter is present in a repressed status, with high binding of H3K27Me3 and poor association of H3K27Ac and H3K36Me3. The LINC01914 promoter was present in an active state in the HGSC cancer tissue (FIGS. 28A and 28B). This data suggests that, most likely, LINC01914 RNA is not synthesized in either normal ovarian tissue or the HGSC stromal tissue; however, the mRNA expression of LINC01914 was found to be high in the HGSC stromal tissue. The LINC01914 nascent RNA expression data shows that LINC01914 was found to be synthesized exclusively in the HGSC cancer tissue (FIG. 2E & FIG. 28C).

To test if LINC01914 is exported, we isolated exosomes from the blood samples of HGSC patients (FIG. 2F). RT-qPCR analysis shows LINC01914 was exclusively present in the exosomal fractions of the HGSC patients. To further test this hypothesis, we generated a LINC01914 barcoded at the 5′ and 3′ ends and used it in co-culture experiments as shown in FIGS. 2G, 2H & 28D. These results show that tumor cells transfer this lncRNA to stromal cells since the barcoded LINC01914 was exclusively synthesized by OVCAR8 cells but present in stromal cells after co-culture.

The Rab27 subfamily which regulates exocytosis of multivesicular endosomes and control different steps of the exosome secretion pathway, consists of two isoforms Rab27a and Rab27b. To investigate if cancer cells were shipping LINC01914 to stromal cells via exosomes, we repeated this experiment using stromal cells co-cultured with OVCAR8 Rab27a b double KO cells (FIG. 2H bottom gel and FIG. 28D bottom plot). Controls demonstrating high-quality exosome isolation are presented in FIGS. 29A-29C. RT-PCR analysis using LINC01914 end sequences identified by RACE (FIG. 1H) shows LINC01914 was present in its full length (490 bp) in exosomal fractions of the HGSC patients (FIG. 21). To further study the role of exosomes in this molecular pathway, we generated CRISPR-assisted Rab27a and Rab27b double knockout (KO) OVCAR8 cells. The poor expression of Rab27a and Rab27b in WT versus KO OVCAR8 cells is demonstrated by qPCR (FIG. 29D). The size and morphology of exosomes extracted from WT OVCAR8 and Rab27a and Rab27b double KO OVCAR8 cells were evaluated using nanoparticle tracking analysis (NTA) and atomic force microscopy (AFM) (FIGS. 2J, 29E-29F). RT-qPCR analysis shows that LINC01914 is poorly expressed in its cytoplasmic, nuclear, or exosomal fractions in normal ovarian tissue (FIG. 29G). Fluorescent in-situ hybridization (FISH) was performed using HGSC samples stained for LINC01914 (FIG. 29H). Overall, this data suggests that LINC01914 is synthesized in the HGSC cancer tissue and transported via exosomes to the surrounding HGSC stromal tissue. Because of this, we have renamed LINC01914 as Tu-Stroma (coming from the tumor and going to the stroma).

Next, we identified the transport mechanisms active in HGSC cancer tissue that load Tu-Stroma into the exosomal fraction. We used information from a LC-MS/MS-based screen, which identified 30 RNA binding proteins (RBPs) capable of interacting and packaging cell-RNA and cell-exosomal shuttle RNA (esRNA). We performed ELISA-based quantitative analysis to determine the relative expression of 30 proteins extracted from the exosomes isolated from the blood of HGSC cancer patients and non-HGSC individuals. A ratio of the expression of these proteins between HGSC and normal exosomes reveals a high presence of an RBP, DEAD-Box Helicase 3 X-Linked (DDX3X) (FIG. 2K). DDX3X immunoprecipitation from the exosomal fractions shows that Tu-Stroma binds to DDX3X exclusively in the exosomes from HGSC tumors (FIGS. 29I-29J). Western blotting and RT-qPCR show high presence of DDX3X in HGSC tumor samples (FIG. 29K). We performed RT-qPCR analysis to study DDX3X expression in the laser-captured tumor and stromal tissue, and its expression was extremely low in HGSC stromal tissue (FIG. 29L).

To observe the binding of DDX3X with Tu-Stroma, we performed a cross-linking immunoprecipitation (CLIP) assay, a modification of previously performed DDX3X CLIP seq. Results point towards a 44 bp region in the middle of Tu-Stroma that binds to DDX3X (model showing binding site presented in FIGS. 2M & 18A-18C).

The physiological role of DDX33X and its exosome-mediated transfer from cancer to stromal cells in the expression of hFwe-Win and hFwe-Lose isoforms was observed in the HGSC stromal cells (FIGS. 2L, 17D, 17E, and 30A). Upon addition of exosome inhibitors, the increase in total hFwe mRNA and hFwe-Lose fraction were reversed. DDX3X knockdown in GFP-positive HGSC cancer cells were co-cultured with HGSC stromal cells. In this experiment, we observed an increase in expression of total hFwe mRNA in the stromal cells, albeit lower than in the control experiment, and loss of the hFwe-Lose isoform.

To understand the role of DDX3X in the exosomal packaging of Tu-Stroma, we isolated exosomal fractions from a large culture of primary HGSC cells on day 0, day 2, day 4, day 6, and day 8. Immunoprecipitation of DDX3X in these exosomal fractions reveals higher DDX3X release with increased time. In the same exosomal fractions, we also observed a progressive increase in the Tu-Stroma expression (FIG. 30B). We then evaluated primary HGSC cancer cells with DDX3X knockdown (shRNA-mediated) and noted that the exosomal fractions of these cells show diminished presence of Tu-Stroma between day 0 to day 8 (FIG. 30C). We observed the impact of exosome inhibitors and DDX3X knockdown in nuclear and cytoplasmic localization of Tu-Stroma in HGSC cancer cells (FIG. 30D). Live-cell imaging of tumor and stroma co-culture with RFP-tagged DDX3X shows the movement of DDX3X from tumor cells to the surrounding stroma (FIGS. 2N, 30E).

### Cancer-Released Tu-Stroma Reduces Stromal Cell Fitness

Next, we decided to study this epistatic cascade. We performed three experiments: a) genetic manipulation of HGSC cancer cells before co-culture, b) genetic and chemical interference in the process during co-culture, and c) genetic manipulation of HGSC stromal cells before co-culture. We used five sets of molecular experiments to read out individual epistatic steps involved in this molecular mechanism. These molecular experiments were a) ChOP on Exon 3 region of HGSC stromal cells to identify the association of Tu-Stroma at this gene locus, b) MethyLight Assay to evaluate level of DNA methylation on this region, c) a series of ChIP experiments, d) RT-qPCR-based analysis of hFwe-Lose isoform, hFwe-Win isoform, and Exon 3 expression in HGSC stromal cells, and e) RT-PCR based validation of Exon 3 cassette deletion. Results of these experiments are shown in each row of FIG. 31A. We applied a Pseudotime algorithm (FIG. 31B) to our ChOP-ChIP-RT-qPCR data to determine the sequence of epigenetic events at the Exon 3 hFwe locus. The value of pseudotime increased among the nodes and formed a continuous curve in the plane of the first and second diffusion components (Tu-Stroma->DNMT3A->G9a->GLP->HP1->SRSF3).

### Stat3 Transcriptionallyregulates Hfwe Promoter

STAT3 mRNA expression analysis shows high expression in HGSC cancer and stromal tissue (FIG. 19A). Western blotting and IHC with STAT3 and pSTAT3^{Y705} antibodies show similar results (FIGS. 19B-19F). We investigated the potential role of STAT3 in regulating the hFwe gene locus. Luciferase assay in OVCAR8 cells shows STAT3-dependent regulation of hFwe 4kb promoter (FIG. 20A). We used the MatInspecter module of Genomatix and identified four STAT3 binding sites the hFwe gene promoter (FIG. 20B), which was confirmed by ChIP and luciferase assay (FIGS. 20C-20F, 21A-21B). Functional data with STAT3 shRNA is shown in (FIG. 21C), which demonstrates that STAT3 does not differentiate between the alternate splicing modes responsible for generating hFwe-Win or hFwe-Lose isoforms.

### Tu-Stroma Release in Tme Regulates Cancer Growth, Metastasis, and Host Survival

We embedded the 3D-spheroids in Matrigel and utilized the capacity of OVCAR8 WT and Tu-Stroma KO cells to invade the surrounding Matrigel (FIG. 21D). Tu-Stroma KO significantly reduces OVCAR8 invasiveness, which is observed by fewer invaginations (FIGS. 21E-21F). Next, we utilized patient-derived 3D scaffolds based on surgically extracted and de-cellularized patient tissue. These scaffolds were re-cellularized with 90% primary stromal cells derived from surgical specimens and 10% GFP labeled WT or Tu-Stroma KO OVCAR8 cells (FIG. 3A). Light-sheet microscopy shows that Tu-Stroma KO decreases the growth of OVCAR8 cells in patient-derived scaffolds (FIG. 3B). Finally, we used an orthotopic HGSC mice model to evaluate the influence of Tu-Stroma on tumor growth. MRI images of ovaries extracted 50 days after injection demonstrate smaller tumors in mice injected with Tu-Stroma KO cells (FIG. 32A). RT-qPCR analysis shows that hFwe-Win expression is high in both tumor and stromal tissue of Tu-Stroma KO OVCAR8 xenografts (FIG. 32B).

We evaluated the efficacy of standard of care chemotherapy for HGSC in the setting of Tu-Stroma and DDX3X knockout (KO) HGSC lines. Based on the latest NCCN guidelines, we used IV docetaxel (75 mg/m²) and carboplatin (75 mg/kg) as our primary treatment. IVIS imaging of a representative mouse from each group of the PDX1 line is shown (FIG. 3C). Knockdown of either Tu-Stroma or DDX3X substantially reduces tumor progression, and addition of chemotherapy completely abolishes the tumor signal on IVIS. IHC with staining for the active form of caspase-3 is shown (FIG. 3C). Tumor harvest of every mouse carrying orthotopic xenografts of PDX2 demonstrate a similar result (FIG. 3D).

Knockdown of Tu-Stroma, DDX3X, or both leads to significant attenuation of the growth 15 curves (FIG. 3E). The number of metastatic or carcinomatosis lesions are noted for both PDX1 and PDX2 (FIG. 3F). Each column of the heatmap represents a cohort in the same order as the IVIS images in FIG. 3C. Kaplan Meier analysis of both PDX1 and PDX2 demonstrates a similar pattern, with chemotherapy and PDX knockdown improving survival (FIG. 32C). The group with Tu-Stroma/DDX3X knockdown and chemotherapy (group color coding noted in FIG. 3F) had the highest survival, with 80% of the cohort remaining at the end of the experiment. A similar set of experiments was performed using the OVCAR4 and OVCAR8 cells lines (FIGS. 22A-22C, 32C-32D). These results demonstrate that Tu-Stroma and DDX3X play a significant role in tumor initiation and propagation.

We performed a knockout and rescue experiment with Tu-Stroma and DDX3X. As demonstrated by IVIS (FIG. 4A), knockout of either Tu-Stroma or DDX3X (rows 2 and 3) slows tumor growth compared to WT injection (row 1). However, addition of exogenous Tu-Stroma (row 4) or DDX3X (row 5) “rescues” the WT phenotype and promotes tumor growth in the subsequent weeks. Average tumor volume measurements over time corroborate these findings and demonstrate a continued exponential growth rate with the exogenous treatment (FIG. 4B). Rescue with Tu-Stroma or DDX3X also increases the metastatic potential of the tumor (FIG. 4C).

We evaluated the role of the individual Flower isoforms in tumor progression. For both OVCAR4 and OVCAR8, four different lines were generated by knocking out hFwe expression and performing a knock-in with each of the four isoforms. Mice (n=5 in each group) received orthotopic injections of each of the KO lines (hFwe^{KO}-hFwe1^{+/+}, hFwe^{KO}-hFwe2^{+/+}, hFwe^{KO}-hFwe3^{+/+}, hFwe^{KO}-hFwe4^{+/+}), with either no subsequent treatment or vehicle treatment. In addition to these four groups, mice received hFwe^{KO}-hFwe1 2 3 4^{+/+} lines that also had Tu-Stroma KO or DDX3X KO. Representative tumors from each group are shown (FIGS. 4D and 22D). Overall, these findings demonstrate that key components in the Flower cell competition pathway play an active role in promoting tumor growth, metastasis, and survival.

### Flower Monoclonal Antibody Therapy Attenuates Cancer Growth and Metastasis

We developed a humanized anti-Flower (hFwe) monoclonal antibody (mAb); the site of interaction and its specificity are shown in FIGS. 5A and 5B. Live-cell imaging of a cell-competition assay shows the efficiency of hFwe mAb to block competitive apoptotic elimination of hFwe-Lose expressing cells (GFP) in presence of hFwe-Win expressing cells (RFP) (row 3 of FIGS. 5C & 22E-22G).

In patient-derived 3D scaffolds, GFP-labeled OVCAR8 (shown as white spots) cells were co-cultured with unlabeled stromal cells (gray regions) (FIGS. 5D and 5E). In the left two panels, tumor and stromal cells are co-cultured in the scaffold with no subsequent treatment, resulting in tumor proliferation throughout the scaffold. In contrast, addition of hFwe mAb (right two panels of FIG. 5E), attenuated the spread of tumor cells. Patient-derived HGSC cells (PDX1 and PDX2) were infected with blank luciferase vector and were orthotopically injected into mice ovaries. A subset of mice received an intravenous injection of the mAb (10 mg/kg) at regular intervals (FIG. 5F). Chemotherapy agents included docetaxel, carboplatin, and bevacizumab. As demonstrated by IVIS imaging, there was substantial tumor spread in the untreated, vehicle, and IgG-treated groups, which was significantly curtailed by hFwe mAb administration (FIG. 5G). Addition of Docetaxel/Carboplatin and Bevacizumab decreased tumor proliferation compared to the control groups. Treatment with Docetaxel/Carboplatin ±Bevacizumab and hFwe mAb further decreased tumor size (columns 7 and 9 of FIG. 5G).

Tumor volume was tracked over time with 95% confidence intervals shown (FIG. 5H). WT, vehicle, and IgG treated tumors (first column) demonstrate a high growth rate that is mitigated with chemotherapy treatment (second column). Treatment with hFwe mAb alone or in combination with chemotherapy regimen substantially attenuate the growth curve with reversal in tumor size (third column). The number of metastatic or carcinomatosis lesions were noted at various locations (FIG. 5I). Each column of the heatmap represents a cohort in the same order as the IVIS images in FIG. 5G. Groups with chemotherapy treatment had fewer lesions compared to the control, vehicle, and IgG groups. Cohorts with combination of hFwe mAb treatment and standard chemotherapy had the fewest lesions. Kaplan Meier analysis of both PDX cohorts demonstrates a similar pattern, with chemotherapy and hFwe mAb improving survival (FIG. 5J). The group with Docetaxel, Carboplatin, Bevacizumab, and hFwe mAb (group color coding noted in FIG. 5I) had the highest survival, with 80% of the cohort remaining at the end of the experiment. A similar experiment was performed using the OVCAR4 and OVCAR8 cell lines (FIGS. 6A, 22H-22J). The findings from these experiments demonstrate that hFwe mAb can reduce tumor growth, attenuate metastatic spread, and improve overall survival.

Next, we determined the antibody's ability to condition the microenvironment by prophylactically treating a set of mice with the antibody as shown (FIG. 6B). After two weeks, the mice received an intraperitoneal injection of 2000 Luciferase labelled HGSC (PDX1 and OVCAR8) cells. The mice continued to receive regular hFwe mAb treatments till the end of the experiment. At 45 days, IVIS imaging was performed, which demonstrated a remarkable attenuation in cancer growth and propagation in the mAb-treated cohort (fifth column) compared to the untreated (column 2), vehicle (column 3), and IgG-treated (column 4) groups (FIG. 6C).

Kaplan-Meier analysis reveals that mice receiving hFwe mAb treatment had improved overall survival (FIG. 6D). Results show that the antibody not only effectively reduces tumor growth and spread in the setting of active disease but can also act as a prophylactic agent by altering the tumor microenvironment to prevent the growth of tumors in the future.

### Flower-Lose and Tu-Stroma Expression in Tme Prognosticates Patient Survival

We analyzed the relationship between the relative expressions of hFwe-Lose and Tu-Stroma and survival in 99 HGSC patient samples (testing set). These patients showed a large variability in survival (less than 3 years and more than 8 years). In this cohort, the correlation between the relative expression and survival was analyzed using Cox proportional hazard models adjusting for age (continuous) and tumor stage (categorical). The estimated hazard ratios were 6.2 for hFwe-Lose and 4.8 for Tu-Stroma, indicating that higher expression of these genes is associated with lower survival. To validate these findings, we acquired an independent and randomly collected cohort of 296 patient samples. These samples were pooled under the validation set. In this cohort, the estimated hazard ratios were 2.0 for both hFwe-Lose and Tu-Stroma (both p-values <1×10⁻¹⁰). The results are summarized in (Table 1), and the Kaplan-Meier curves are shown in FIG. 6E.

TABLE 1

| | Test cohort (n = 99, 72 events) | | | Validation cohort (n = 296, 121 events) | | |
|---|---|---|---|---|---|---|
| | Hazard ratio | χ² value | p-value | Hazard ratio | χ² value | p-value |
| Flower-Loose | 6.2 (3.4, 11.1) | 55.7 | 8.46 · 10⁻¹⁴ | 2.0 (1.6, 2.4) | 54.0 | 1.99 · 10⁻¹³ |
| Tu-Stroma | 4.8 (3.1, 7.6) | 71.2 | <2.2 · 10⁻¹⁶ | 2.0 (1.6, 2.4) | 50.5 | 1.20 · 10⁻¹² |

Survival from HGSC patients was analyzed in proportional hazard models to estimate the association of hFwe and Tu-Stroma expression on survival. The table shows the results for the test cohort and for the validation cohort. Hazard ratios (estimate and 95% confidence interval) are for log 2 relative expression and are adjusted for age and stage. β-values are from type-II likelihood ratio tests based on the x statistic with 1 degree of freedom. The estimated hazard ratio refers to a doubling of the relative expression of the respective gene. The values above 1.0 indicate a higher hazard or a worse survival with increasing expression.

### DISCUSSION

Here, we studied the splicing regulation of the human Flower gene in the stromal tissue of high grade serous ovarian carcinoma (HGSC), which is the most aggressive ovarian cancer with less than 28% 5-year survival. We found that Exon 3 inclusion or exclusion in the final Flower mRNA transcript determines the generation of Flower-Win or Flower-Lose isoforms, respectively. We further found that HGSC tumor cells gain a competitive advantage by using exosomes to secrete a long non-coding RNA. Upon reaching stromal cells, this lncRNA manipulates the standard splicing, forcing the microenvironment cells to express Flower-Lose isoforms, and therefore inducing low fitness status. We named this lncRNA Tu-Stroma because it is exclusively synthesized by the Tumor and delivered to the Stroma via exosomes. Tu-Stroma is only functional in the stromal cells because it is actively packaged into the exosomes by binding to a tumor specific protein, DDX3X. Because of this interaction, Tu-Stroma is unable to influence splicing towards the generation of Flower-Lose isoforms in the cancer cells. In the stromal cells, however, Tu-Stroma establishes a DNA-RNA adduct with Exon 3 of the Flower gene and induces DNA methylation, histone methylation, HP1 binding and recruitment of splicing factor SRSF3, which results in Exon 3 exclusion and generation of Flower-Lose.

To our knowledge, this is the first time that a lncRNA has been shown to regulate cell fitness by controlling splicing in trans (from chromosome 2 to chromosome 9), in a non-cell autonomous manner (from cancer cells to stromal cells).

Additionally, we have studied the dynamics of tumor and stromal interactions in the setting of Tu-stroma in in-vitro and in-vivo models, including a patient derived scaffold containing tumor cells mixed with patient stroma cells. Overall, these studies have demonstrated the impact of Tu-stroma attenuation on tumor cell invasion, proliferation, and metastasis.

Next, we demonstrate the clinical relevance of this discovery. Patients with elevated expression of Tu-Stroma and hFwe-Lose had lower median survival compared to those with lower expression. This was further validated with Cox proportional hazard models, which demonstrate that the expression of both Flower-Lose and Tu-Stroma correlate significantly with patient survival and are predictors for survival.

Finally, we harnessed the therapeutic potential of the Flower pathway by developing a monoclonal antibody against Flower. Survival experiments in mice demonstrated successful attenuation of both primary tumor growth and metastases and improved survival in mAb-treated mice compared to control and vehicle cohorts. Furthermore, we demonstrated the ability of the antibody to create a favorable, less competitive tumor microenvironment that prevents the spread of future cancers.

In summary, we investigated the mechanism for solid tumor growth in a stromal environment with reduced fitness. In principle, high expression of Lose could be a pre-existing phenotype of the host tissue. For example, we have recently shown that Flower-Lose levels correlate with old age and several diseases including diabetes and obesity. Here, we show that reduced microenvironment fitness is a tumor-induced effect and that cancer cells are programmed to create an environment designed to win competitive interactions.

### Example 3

### Anti-Calcium Channel Flower Homologs Antibodies

As discussed above, the human Fwe locus generates four isoforms: two hFweWin and two hFwe^{Lose}. Two Flower isoforms (hFwe2 and hFwe4) behave as Flower-Win proteins, whereas the other isoforms (hFwe1 and hFwe3) behave as Flower-Lose proteins.

The amino acid sequence of hFwe1 is shown below (accession number NM_017586.5):

(SEQ ID NO: 231)
MSSSGGAPGASASSAPPAQEEGMTWWYRWLCRLSGVLGAVSCAISG
LFNCITIHPLNIAAGVWMMMAVVPIVISLTLTTLLGNAIAFATGVL
YGLSALGKKAQTEAGSFAAQHPREPGPFSEGTRQAFATPAVVSGEI
RMPAGAMRSPMPGSSSRGSRRMRRSSRRPWRGSCEGLGAPPSLSPL
LALCGSK

The amino acid sequence of hFwe2 is shown below (accession number NM_001135775.4):

(SEQ ID NO: 232)
MSSSGGAPGASASSAPPAQEEGMTWWYRWLCRLSGVLGAVSCAISG
LFNCITIHPLNIAAGVWMIMNAFILLLCEAPFCCQFIEFANTVAEK
VDRLRSWQKAVFYCGMAVVPIVISLTLTTLLGNAIAFATGVLYGLS
ALGKKAQTEAGSFAAQHPREPGPFSEGTRQAFATPAVVSGEIRMPA
GAMRSPMPGSSSRGSRRMRRSSRRPWRGSCEGLGAPPSLSPLLALC
GSK

The amino acid sequence of hFwe3 is shown below (accession number NM_001242369.2):

(SEQ ID NO: 233)
MSSSGGAPGASASSAPPAQEEGMTWWYRWLCRLSGVLGAVSCAISG
LFNCITIHPLNIAAGVWMMMAVVPIVISLTLTTLLGNAIAFATGVL
YGLSALGKKGDAISYARIQQQRQQADEEKLAETLEGEL

The amino acid sequence of hFwe4 is shown below (accession number NM_001242370.2):

(SEQ ID NO: 234)
MSSSGGAPGASASSAPPAQEEGMTWWYRWLCRLSGVLGAVSCAISG
LFNCITIHPLNIAAGVWMIMNAFILLLCEAPFCCQFIEFANTVAEK
VDRLRSWQKAVFYCGMAVVPIVISLTLTTLLGNAIAFATGVLYGLS
ALGKKGDAISYARIQQQRQQADEEKLAETLEGEL

In one embodiment, the present disclosure provides anti-calcium channel flower homologs antibodies having the following amino acid sequences. ANTIBODY JA

Antibody 1a binds to the N-terminus of the calcium channel flower homolog proteins. The target region is present on hFwe1, hFwe2, hFwe3 and hFwe4.

The heavy chain CDRs (HCDR1, HCDR2, HCDR3) and light chain CDRs (LCDR1, LCDR2, LCDR3) of antibody 1a are shown below:

| | | |
|---|---|---|
| HCDR1 | GNSAVTYE | SEQ ID NO: 266 |
| HCDR2 | VPPLLTNYSSLL | SEQ ID NO: 267 |
| HCDR3 | VQTSGELV | SEQ ID NO: 268 |
| LCDR1 | QLYKPFTQ | SEQ ID NO: 269 |
| LCDR2 | CSKSANIA | SEQ ID NO: 270 |
| LCDR3 | SNSNQGS | SEQ ID NO: 271 |

VH of antibody 1a:
(SEQ ID NO: 235)
GRVLFYSDSAVVKFMGSPNTLKWENWPLYATKRGPSSVVGAG
TAQVMHSALRYASSGNSAVTYEGESSNYGTSMFSPSKKKPST
FCLRNYSYTESTKYCYYCGDRGTMVPPLLTNYSSLLGSPFVW
ARTIVSDPWTQQSDWGTYIALNESNSAASWPDLLYQSQSAQE
TVQTSGELVWVKAGPSADPQGDVFSGTPTVQGGAVVSKTLLA
DSKCSTTVRPEIGARVVLNTHEVIAPTGRLSTTRPERAEGML
LNDQLVSPQVSEPFMVTVQSYSV
VL of antibody 1a:
(SEQ ID NO: 236)
STFTVELSGQKLNEPPSSVYIATNIVCVTQSKDLLSDFWNVD
EDVFCDAQLYKPFTQTEDYSNEGVTSERGPSSMPTDEPTYMI
KRQSYACSKSANIAWSGDDKLSVRHSGLVATGKVQKTRTYFL
NPREVGLAYTSPSLGGPFVTEGSESNSNQGSSSSNMDQSIKW
STYTYSNCLSSEHWSKIGQSYAIATYDSKQTFPVTFSSMIHA
LCLQTKKYLR

Antibody 1a binds to a target region having the amino acid sequence CSGGAPGASASSAPPAQEEG (SEQ ID NO:251). ANTIBODY 1B

Antibody 1b binds to the N-terminus of the calcium channel flower homolog proteins. The target region is present on hFwe1, hFwe2, hFwe3 and hFwe4.

| | | |
|---|---|---|
| HCDR1 | LGTDTVSTT | SEQ ID NO: 272 |
| HCDR2 | VGLTWCASSP | SEQ ID NO: 273 |
| HCDR3 | LTRVPTT | SEQ ID NO: 274 |
| LCDR1 | QALLPSAS | SEQ ID NO: 275 |
| LCDR2 | ALNATVIASA | SEQ ID NO: 276 |
| LCDR3 | SQDNLPG | SEQ ID NO: 277 |

VH of antibody 1b:
(SEQ ID NO: 237)
SGGGVVWKPSLVEGYQGLVVSPLVVAGTLVAKSDEPPNYLSSYSTLGTD
TVSTTVYARVKLKAVDTFVDTESWSDGVGLTWCASSPYDISVWFHQTFK
HGGQQIGEHCSYGTYIPGTLGGQQAATQSQGISKWATSGSTAKVVASGS
PLTRVPTTKSGFSSNTSVYAKFPPAARSPKVNADKLSASVVFTSQQGKR
CTCYVTTQVVPRSLAGRPDDENLVGSRLFK
VL of antibody 1b:
(SEQ ID NO: 238)
YCCTGQGGTLYQKEYVCGWSKSITCKFREDNSSIQRAFSQALLPSASPA
DSESDTKAEPDQKSSHGSFEDVEESTSTRIQVLHSTLTTGQLRAGGFKA
LNATVIASAEPERWVSSLLVQQGKPPAYATELDRVPVADVCGRLDVFHR
SQDNLPGFRTRYQQVRWLYSERSTYGQYAKESSVTLKVGGFVVVITIRN
TSPSLSATSNSYFKNPPPT

Antibody 1b binds to a target region having the amino acid sequence CGGAPGASASSAPPAQEEGMTWWYR (SEQ ID NO:252).

### Antibody 2A

Antibody 2a binds to a region encoded by exon 3 of the calcium channel flower homolog proteins. The target region is present on hFwe2 and hFwe4.

The heavy chain CDRs (HCDR1, HCDR2, HCDR3) and light chain CDRs (LCDR1, LCDR2, LCDR3) of antibody 2a are shown below:

| | | |
|---|---|---|
| HCDR1 | APEGQKKGS | SEQ ID NO: 278 |
| HCDR2 | CRVRVSSNK | SEQ ID NO: 279 |
| HCDR3 | VWLWFSPQI | SEQ ID NO: 280 |
| LCDR1 | WSKRVEVAGSFT | SEQ ID NO: 281 |
| LCDR2 | QPSP | SEQ ID NO: 282 |
| LCDR3 | NGNQKRLRSRDVSTS | SEQ ID NO: 283 |

VH of antibody 2a:
(SEQ ID NO: 239)
GRVLDSCKVGVSVGSSAAVTSPTSSYRQVTMGSKSNXSTSQAXLTSGRM
FPSTAPEGQKKGSVYLXSTSATQSADKRPNPTKKPSSNVYKPGGGECRV
RVSSNKVCSTFIFEVYXQPKELNSCXFIWVATQAEISSMSGVDWGYPAV
MLACAQLGEWVAATVFTQNHTDXLSVIQKMCEYVWLWFSPQICDHTLSV
TLVYFIGVPRKQISYGGLSPTSNVVASGGLTLTD
VL of antibody 2a:
(SEQ ID NO: 240)
GIVAPVGIIPSYKQVYGVNGWGVALESWSKRVEVAGSFTHPGLVKSPVS
AYQLACLHSRSTLESEELRGSGTQYPTATLFYGDLSRTDDILADKSQPS
PFRKSGGTPTYNFISILRFSTDSVQEFSGSSQCQRALKLWAEDEATEKK
SSYSQLPSYAKPLFTQNGNQKRLRSRDVSTSVNTSSHSGCEQSESRLCT
CKTGQDVFAALTVQAYYTPE

Antibody 2a binds to a target region having the amino acid sequence EKVDRLRSW (SEQ ID NO:253).

### Antibody 2B

Antibody 2b binds to a region encoded by exon 3 of the calcium channel flower homolog proteins. The target region is present on hFwe2 and hFwe4.

The heavy chain CDRs (HCDR1, HCDR2, HCDR3) and light chain CDRs (LCDR1, LCDR2, LCDR3) of antibody 2b are shown below:

| | | |
|---|---|---|
| HCDR1 | SSHSALGDAQNTLPKPWYFY | SEQ ID NO: 284 |
| HCDR2 | ELPCQYASKLASKWPT | SEQ ID NO: 285 |
| HCDR3 | PSQTKCVAKVHSSL | SEQ ID NO: 286 |
| LCDR1 | RKATDTLVAVVC | SEQ ID NO: 287 |
| LCDR2 | KYEHKHCF | SEQ ID NO: 288 |
| LCDR3 | TQPPIKKQSYGFSS | SEQ ID NO: 289 |

VH of antibody 2b:
(SEQ ID NO: 241)
GRVLYEDVKSCRNSGSGTVTSQYATTSVTFQPAVTPGSTSSHSALGDAQ
NTLPKPWYFTDVKSVLGKTTDQGRLASGMLSVSDEIELPCQYASKLASK
WPTGMVYSVGFELEVTIKIMSSGTGAAWVISYSALMTGSLPTAVEFTDY
ESSVLNSLPVQASGAGSPPAINWTHYDAANRGPSQTKCVAKVHSSLYVA
QKVYVTTKVPAKCVDDPTSLWYNTS
VL of antibody 2b:
(SEQ ID NO: 242)
KTLVLYRSLTGNSINTCGEGPILQSIYLWTKTGDFYSMYSQNYARKATD
TLVAVVCGPPAKLLQKRSEVSLVLQMGQGATTQASDTPGISKYEHKHCF
LGSDYNWGPDSDTVTWLSKTANFMGRRNEETQTTQPPIKKQSYGFSSNL
VTDPSSTYAFETSEVPEYPSTYDASSSVDNKCKTSASSQSSNNVGLEQK
ITWNERVNCTNSKFTDGDSFLLSI
Antibody 2b binds to a target region having the
amino acid sequence
(SEQ ID NO: 254)
NTVAEKVDRLRSWQKAVF.

### Antibody 3A

Antibody 3a binds to a region encoded by exon 5 of the calcium channel flower homolog proteins. The target region is present on hFwe1 and hFwe2.

The heavy chain CDRs (HCDR1, HCDR2, HCDR3) and light chain CDRs (LCDR1, LCDR2, LCDR3) of antibody 3a are shown below:

| | | |
|---|---|---|
| HCDR1 | GFSPTNSLMC | SEQ ID NO: 290 |
| HCDR2 | VSKELTGQTAYV | SEQ ID NO: 291 |
| HCDR3 | VAKFIYTPPG | SEQ ID NO: 292 |
| LCDR1 | SPSVIVGQTVVAG | SEQ ID NO: 293 |
| LCDR2 | PWTELNITGS | SEQ ID NO: 294 |
| LCDR3 | VSTNTGL | SEQ ID NO: 295 |

VH of antibody 3a:
(SEQ ID NO: 243)
HNFKKSMLWSKPNTSDYDTVPSAILVDVVMTGASYVSTQQGSVTAARR
TGEAGSCLHGFSPTNSLMCVDSVVVKSTRTKSEIGGAGCSDGAPDAYK
WVSKELTGQTAYVGTTIWTSTWTSKGQYGPMYRITKGQGALNGCSDVA
KFIYTPPGYFSVSVRSAVLSGLVRPLLDDGNTSESQLAKLNAESTKYL
NLSAIVICGPFINVSGQSWGKQYVVAFQQGPPSVYSS
VL of antibody 3a:
(SEQ ID NO: 244)
SPGARSETVNQKKLSHQEAASCICLLQGGNSFSAYSGETPWSPSVIVG
QTVVAGSKPFGKDTVSVSSGTPPQPNLANSVLSSSARQEPSPALPWTE
LNITGSVRKSSGSHTTPCTKSPYLLETQPQVSPLGLCEDNGDCTKYGA
VSTNTGLADGATYIDPGSKIVQNSEKAQGTYLPYAAWEAVQWPSTKSY
DRFVHCSAGQKTGVYDSFLTLVTAYSA

Antibody 3a binds to a target region having the amino acid sequence KKAQTEAGSFAAQHPREPGPFSEGTRQAFATPAV (SEQ ID NO:255).

### Antibody 3B

Antibody 3b binds to a region encoded by exon 5 of the calcium channel flower homolog proteins. The target region is present on hFwe1 and hFwe2.

The heavy chain CDRs (HCDR1, HCDR2, HCDR3) and light chain CDRs (LCDR1, LCDR2, LCDR3) of antibody 3b are shown below:

| | | |
|---|---|---|
| HCDR1 | VVANIGNSPQENSKGST | SEQ ID NO: 296 |
| HCDR2 | SSPWSFCLVPPFI | SEQ ID NO: 297 |
| HCDR3 | SLWTDVH | SEQ ID NO: 298 |
| LCDR1 | SDPGYCKLSLAM | SEQ ID NO: 299 |
| LCDR2 | RERRTVKFK | SEQ ID NO: 300 |
| LCDR3 | QGTEGYDRSYFSYI | SEQ ID NO: 301 |

VH of antibody 3b:
(SEQ ID NO: 245)
GRVLSNMTSPYVYNPSDKSWLLVTPSYAVTLSADAGTPVVANIGNSPQE
NSKGSTTTTLSISSRADYAVSSQLTDQDAEVLLWTGAQVVGAETTEKAP
YGAVVSSPWSFCLVPPFIGGLHYQLFGTKWRYSTSASKVAILHDRTQLT
SSYSSYGGQFASTDPANSTDWKCVGGYKSPQSLWTDVHQAVTETESSTK
NRIVPAEKMVACLYVEQRTSPLSTCPL
VL of antibody 3b:
(SEQ ID NO: 246)
AVDPLSTYHSYRIGCGGTIYSDDPSINSGGGSVLKSSSLSDPGYCKLSL
AMFKVLTAVQLEEDFQVVASLAVSAWRECGITLKPNNKSSTWYTTSCSQ
FKNVAFGEIGSSPSIRERRTVKFKEMPYSMDYVVNGNQEMSRRTTDLPY
LLTLTYNQSDSTTQETSSQPQGTEGYDRSYFSYISVNQRISWKASHNHD
WNSQVASTDYEPRLQSKTKTCKF

Antibody 3b binds to a target region having the amino acid sequence EAGSFAAQHPREPGPFSEGTRQAFAT (SEQ ID NO:256).

### Antibody 4A

Antibody 4a binds to C-terminus proximal region of the calcium channel flower homolog proteins. The target region is present on hFwe1, hFwe2, hFwe3 and hFwe4.

The heavy chain CDRs (HCDR1, HCDR2, HCDR3) and light chain CDRs (LCDR1, LCDR2, LCDR3) of antibody 4a are shown below:

| | | |
|---|---|---|
| HCDR1 | PLFVSMAHSYDKKSS | SEQ ID NO: 302 |
| HCDR2 | WSYNFVNSSL | SEQ ID NO: 303 |
| HCDR3 | YKGTPSGSSSQF | SEQ ID NO: 304 |
| LCDR1 | ESSPVLWQNSTTVG | SEQ ID NO: 305 |
| LCDR2 | TLKTTVWVNALSCT | SEQ ID NO: 306 |
| LCDR3 | RYQQNVQPVP | SEQ ID NO: 307 |

VH of antibody 4a:
(SEQ ID NO: 247)
DGETNPSSTKLKLVLTSTGQLNVSPSSVSPHPLFVSMAHSYDKKSSSAS
RNVVFVSGYSGYYLDWSSQKSGVYVSGYGYKSRGYTVQSKTDNAASPKL
ADAYSLPLCFMGGCTGGTKSTVQLRWSYNFVNSSLYSDDFQAATTFRRD
NCAVAVTYKGTPSGSSSQFGGPEETIVNPVHLDGGWVTKLRLNSPPTKE
YFLGWALDMVDTIVKTGIGVSCSQEGFWNGKVDLT
VL of antibody 4a:
(SEQ ID NO: 248)
EARDTEATLQPDAYCNDTVSVGLGGAVAGHSLESSPVLWQNSTTVGKKV
KNWKFVTNTASQYAPISNPGKTWSGPSKYDAYFVTLKTTVWVNALSCTS
SESTVSNASQQGYGSEESARTTLTSYPYESDSPYSYTLGGDKVKSPSKN
TAIIIDRHILPAQDVLQAVAGGIRDRYQQNVQPVPQHGHNALSAFVVPP
KEVDGPPPFCSVAC

Antibody 4a binds to a target region having the amino acid sequence GKKGDAISYARIQQQRQQADEEKLAETL (SEQ ID NO:257).

### Antibody 4B

Antibody 4b binds to C-terminus distal region of the calcium channel flower homolog proteins. The target region is present on hFwe1 and hFwe2.

The heavy chain CDRs (HCDR1, HCDR2, HCDR3) and light chain CDRs (LCDR1, LCDR2, LCDR3) of antibody 4a are shown below:

| | | |
|---|---|---|
| HCDR1 | LSYKCAEKYTCGVIT | SEQ ID NO: 308 |
| HCDR2 | ATLYYGVRKL | SEQ ID NO: 309 |
| HCDR3 | NSDSHTVTLAIMP | SEQ ID NO: 310 |
| LCDR1 | CYPGLQVIXSTGVYE | SEQ ID NO: 311 |
| LCDR2 | QYWISVAIWATSY | SEQ ID NO: 312 |
| LCDR3 | APKCKVLGGFLVPSLVGLQ | SEQ ID NO: 313 |

VH of antibody 4b:
(SEQ ID NO: 249)
ELCWGMSPTVLRPSSQYSKPDADARKFGCVYLLSYKCAEKYTCGVITQW
WTDAVSSLNLGGKSVYAPYTRTMVQNGLLKTTSSVTPSTSSGESWTSVT
PATLYYGVRKLLLVVTPKTELQVAAGHLSSPSFVNSDSHTVTLAIMPPQ
SPKKLDVDTKADFTFSSLSSSGQTSVDSVVVRGVLPSSRGALSGTSXCN
LVLKDVGSTRTDSILPVWITTFSPDDFGNST
VL of antibody 4b:
(SEQ ID NO: 250)
QSYVKPQTTTAWPWPLTTPSKGDNFDGHSGSPVSGSLCYPGLQVIXSTG
VYELDAAAAFFTTSETRSPNSFWGSPAAPQPAVSSKASTSGACVTSGES
NQYWISVAIWATSYGASKKEPAQGDTKSESARYTPSIYQSSATKDGQEK
QGRKPNESNCAGYSAPKCKVLGGFLVPSLVGLQTDIVLTECLVHPSSEV
VTKSASLLNLDNEPNTLSSVV

Antibody 4b binds to a target region having the amino acid sequence SGEIRMPAGAMRSPMPGSSSRGSRRMRRSSRRPWRGSCEGLGAPPS (SEQ ID NO:258).

The binding affinity of the eight hFwe antibodies (Antibody 1a, Antibody 1b, Antibody 2a, Antibody 2b, Antibody 3a, Antibody 3b, Antibody 4a, Antibody 4b) was tested by indirect ELISA for their respective peptides. Briefly, the supernatants of each antibody were added to 96-well plates coated with the respective peptide of each antibody at 200ng/well. Immune sera were used as a positive control in place of primary Ab. Peroxidase conjugated goat anti-mouse IgG secondary antibody (ABEOMICS, Inc. San Diego, CA) was used at 1:5000 dilution, and the absorbance was measured at 450 nm. Samples were measured in triplicate. We observed that the eight antibodies show an average of 2.5-fold higher affinity than the blank samples (uncoated wells) and a similar affinity to the one observed by the immune sera (FIG. 33).

FIG. 34 shows schematics and sequences where the eight antibodies disclosed herein (antibody 1a, antibody 1b, antibody 2a, antibody 2b, antibody 3a, antibody 3b, antibody 4a, antibody 4b) bind to the Flower proteins hFwe1, hFwe2, hFwe3, and hFwe4. Antibody 1a binds to a target region having the amino acid sequence CSGGAPGASASSAPPAQEEG (SEQ ID NO:251). Antibody 1b binds to a target region having the amino acid sequence CGGAPGASASSAPPAQEEGMTWWYR (SEQ ID NO:252). Antibody 2a binds to a target region having the amino acid sequence EKVDRLRSW (SEQ ID NO:253). Antibody 2b binds to a target region having the amino acid sequence NTVAEKVDRLRSWQKAVF (SEQ ID NO:254). Antibody 3a binds to a target region having the amino acid sequence KKAQTEAGSFAAQHPREPGPFSEGTRQAFATPAV (SEQ ID NO:255). Antibody 3b binds to a target region having the amino acid sequence EAGSFAAQHPREPGPFSEGTRQAFAT (SEQ ID NO:256). Antibody 4a binds to a target region having the amino acid sequence GKKGDAISYARIQQQRQQADEEKLAETL (SEQ ID NO:257). Antibody 4b binds to a target region having the amino acid sequence SGEIRMPAGAMRSPMPGSSSRGSRRMR RSSRRPWRGSCEGLGAPPS (SEQ ID NO:258).

### Combinations of Six Vh and Six Vl Sequences

Disclosed below are six humanized VH sequences (VH1, VH2, VH3, VH4, VH5, VH6) and six humanized VL sequences (VL1, VL2, VL3, VL4, VL5, VL6), produced as described above. Any one of these six VH sequences can pair with any one of these six VL sequences to form an antibody that binds to calcium channel flower homolog proteins, i.e., there are thirty six different combinations of VH and VL. All these VH/VL pairs bind to the same epitope and essentially have the same binding affinities (FIG. 35). The ELISA binding assays were performed as follows. Wells of 96-well microtiter plates were coated with purified recombinant peptides 200 ng/well at 4° C. overnight. Plates were blocked with 2% non-fat dry milk and 2% normal goat serum in DPBS containing 0.05% Tween-20 (DPBS-T) for 1 h. For mAb screening assays, purified anti-flower antibody(hFwe) were diluted 1:20 in blocking buffer, added to the wells and incubated for 1 h at ambient temperature, and washed four times with DPBS-T. Goat anti-human IgG conjugated with horseradish peroxidase (HRP) diluted in DPBS-T (1:5000) was added to the wells and incubated for 1 h at RT. After washing the plates four times with DPBS-T, 100 μL of the 3,3′,5,5′-tetramethylbenzidine (TMB) substrate was added to each well. The reaction was terminated by adding 50 μL of 2 N H₂SO₄ and the absorbance was measured at 450 nm. Collected data were analyzed using Gen5 IVD software and were expressed upon background subtraction as A₄₅₀-A₀.

These VH and VL sequences all have the same HCDR1, HCDR2, HCDR3 and LCDR1, LCDR2, LCDR3 sequences as shown below:

| | | |
|---|---|---|
| HCDR1 | GFNIKDSI | SEQ ID NO: 326 |
| HCDR2 | IDPEDGDT | SEQ ID NO: 327 |
| HCDR3 | SKSYYGPDF | SEQ ID NO: 328 |
| LCDR1 | QSIVHSNGNTY | SEQ ID NO: 329 |
| LCDR2 | RVS | SEQ ID NO: 330 |
| LCDR3 | FQGTHVPRT | SEQ ID NO: 331 |

Since the HCDR1, HCDR2, HCDR3 and LCDR1, LCDR2, LCDR3 sequences are the same for the VH and VL sequences presented as VH1-VH6 and VL1-VL6, respectively, any pairing of VH1-VH6 and VL1-VL6 provides an anti-CACFD1 binding fragment binding to the same epitope. An antibody comprising any pairing of VH1-VH6 and VL1-VL6 provides an anti-CACFD1 antibody binding to the same epitope.

Sequence of VH1:
(SEQ ID NO: 314)
EVQLQQSGAELVRPGASVKLSCTASGFNIKDSIMHWVKQRPEQGLEWIG
WIDPEDGDTEYAPKFQDKATITADTSSNTAYLQLSSLTSEDTAVYYCSK
SYYGPDFWGQGTTLTVSS
Sequence of VH2:
(SEQ ID NO: 316)
EVQLVQSGAEVKKPGATVKISCKVSGFNIKDSIMHWVQQAPGKGLEWMG
WIDPEDGDTEYAPKFQDRVTITADTSTDTAYMELSSLRSEDTAVYYCSK
SYYGPDFWGQGTTVTVSS
Sequence of VH3:
(SEQ ID NO: 318)
EVQLVQSGAEVKKPGATVKISCKVSGFNIKDSIMHWVQQAPGKGLEWMG
WIDPEDGDTEYAPKFQDRATITADTSTNTAYMELSSLRSEDTAVYYCSK
SYYGPDFWGQGTTVTVSS
Sequence of VH4:
(SEQ ID NO: 320)
EVQLVQSGAEVKKPGATVKISCKASGFNIKDSIMHWVQQAPGKGLEWIG
WIDPEDGDTEYAPKFQDRATITADTSTNTAYMELSSLRSEDTAVYYCSK
SYYGPDFWGQGTTVTVSS
Sequence of VH5:
(SEQ ID NO: 322)
EVQLVQSGAEVKKPGATVKISCKASGFNIKDSIMHWVQQAPGKGLEWIG
WIDPEDGDTEYAEKFQDRATITADTSTNTAYMELSSLRSEDTAVYYCSK
SYYGPDFWGQGTTVTVSS
Sequence of VH6:
(SEQ ID NO: 324)
EVQLVQSGAEVKKPGASVKVSCKASGFNIKDSIMHWVRQAPGQGLEWIG
WIDPEDGDTEYAQKFQGRATITADTSINTAYMELSRLRSDDTAVYYCSK
SYYGPDFWGQGTTVTVSS
Sequence of VL1:
(SEQ ID NO: 315)
DVVMTQTPLSLPVSLGDQASISCRSSQSIVHSNGNTYLFWYLQKPGQSP
KLLIYRVSNRVSGVPDRFSGSGSGTDFTLKISRVEAEDVGVYYCFQGTH
VPRTFGGGTKLEIR
Sequence of VL2:
(SEQ ID NO: 317)
DVVMTQSPLSLPVTLGQPASISCRSSQSIVHSNGNTYLFWFQQRPGQSP
RRLIYRVSNRVSGVPDRFSGSGSGTDFTLKISRVEAEDVGVYYCFQGTH
VPRTFGGGTKVEIK
Sequence of VL3:
(SEQ ID NO: 319)
DVVMTQSPLSLPVTLGQPASISCRSSQSIVHSNGNTYLFWFQQRPGQSP
RLLIYRVSNRVSGVPDRFSGSGSGTDFTLKISRVEAEDVGVYYCFQGTH
VPRTFGGGTKVEIK
Sequence of VL4:
(SEQ ID NO: 321)
DVVMTQSPLSLPVTLGQPASISCRSSQSIVHSNGNTYLFWYQQRPGQSP
RLLIYRVSNRVSGVPDRFSGSGSGTDFTLKISRVEAEDVGVYYCFQGTH
VPRTFGGGTKVEIK
Sequence of VL5:
(SEQ ID NO: 323)
DIVMTQSPLSLPVTPGEPASISCRSSQSIVHSNGNTYLFWYLQKPGQSP
QLLIYRVSNRVSGVPDRFSGSGSGTDFTLKISRVEAEDVGVYYCFQGTH
VPRTFGGGTKVEIK
Sequence of VL6:
(SEQ ID NO: 325)
DVVMTQSPLSLPVTPGEPASISCRSSQSIVHSNGNTYLFWYLQKPGQSP
QLLIYRVSNRVSGVPDRFSGSGSGTDFTLKISRVEAEDVGVYYCFQGTH
VPRTFGGGTKVEIK

While certain features of the binding agents and uses thereof have been illustrated and described herein, many modifications, substitutions, changes, and equivalents will now occur to those of ordinary skill in the art. It is, therefore, to be understood that the appended claims are intended to cover all such modifications and changes as fall within the true spirit of these compositions of matter and methods of use.

What is claimed is:

1. An isolated antibody that binds to calcium channel flower homolog protein, said antibody comprising three heavy chain complementarity determining regions (HCDR1, HCDR2, HCDR3) and three light chain CDRs (LCDR1, LCDR2, LCDR3), wherein
(a) the HCDR1, HCDR2, and HCDR3 have the sequences of SEQ ID NOs:266-268 respectively, and the LCDR1, LCDR2, and LCDR3 have the sequences of SEQ ID NOs:269-271 respectively; or
(b) the HCDR1, HCDR2, and HCDR3 have the sequences of SEQ ID NOs:326-328 respectively, and the LCDR1, LCDR2, and LCDR3 have the sequences of SEQ ID NOs:329-331 respectively; or
(c) the HCDR1, HCDR2, and HCDR3 have the sequences of SEQ ID NOs:272-274 respectively, and the LCDR1, LCDR2, and LCDR3 have the sequences of SEQ ID NOs:275-277 respectively; or
(d) the HCDR1, HCDR2, and HCDR3 have the sequences of SEQ ID NOs:278-280 respectively, and the LCDR1, LCDR2, and LCDR3 have the sequences of SEQ ID NOs:281-283 respectively; or
(e) the HCDR1, HCDR2, and HCDR3 have the sequences of SEQ ID NOs:284-286 respectively, and the LCDR1, LCDR2, and LCDR3 have the sequences of SEQ ID NOs:287-289 respectively; or
(f) the HCDR1, HCDR2, and HCDR3 have the sequences of SEQ ID NOs:290-292 respectively, and the LCDR1, LCDR2, and LCDR3 have the sequences of SEQ ID NOs:293-295 respectively; or
(g) the HCDR1, HCDR2, and HCDR3 have the sequences of SEQ ID NOs:296-298 respectively, and the LCDR1, LCDR2, and LCDR3 have the sequences of SEQ ID NOs:299-301 respectively; or
(h) the HCDR1, HCDR2, and HCDR3 have the sequences of SEQ ID NOs:302-304 respectively, and the LCDR1, LCDR2, and LCDR3 have the sequences of SEQ ID NOs:305-307 respectively; or
(i) the HCDR1, HCDR2, and HCDR3 have the sequences of SEQ ID NOs:308-310 respectively, and the LCDR1, LCDR2, and LCDR3 have the sequences of SEQ ID NOs:311-313, respectively.

2. The isolated antibody of claim 1, wherein the antibody comprises (a) a heavy chain variable region having an amino acid sequence at least 80% identical to SEQ ID NO:235, and a light chain variable region having an amino acid sequence at least 80% identical to SEQ ID NO:236; or
(b) a heavy chain variable region having an amino acid sequence at least 80% identical to SEQ ID NO:237, and a light chain variable region having an amino acid sequence at least 80% identical to SEQ ID NO:238; or
(c) a heavy chain variable region having an amino acid sequence at least 80% identical to SEQ ID NO:239, and a light chain variable region having an amino acid sequence at least 80% identical to SEQ ID NO:240; or
(d) a heavy chain variable region having an amino acid sequence at least 80% identical to SEQ ID NO:241, and a light chain variable region having an amino acid sequence at least 80% identical to SEQ ID NO:242; or
(e) a heavy chain variable region having an amino acid sequence at least 80% identical to SEQ ID NO:243, and a light chain variable region having an amino acid sequence at least 80% identical to SEQ ID NO:244; or
(f) a heavy chain variable region having an amino acid sequence at least 80% identical to SEQ ID NO:245, and a light chain variable region having an amino acid sequence at least 80% identical to SEQ ID NO:246; or
(g) a heavy chain variable region having an amino acid sequence at least 80% identical to SEQ ID NO:247, and a light chain variable region having an amino acid sequence at least 80% identical to SEQ ID NO:248; or
(h) a heavy chain variable region having an amino acid sequence at least 80% identical to SEQ ID NO:249, and a light chain variable region having an amino acid sequence at least 80% identical to SEQ ID NO:25; or
(i) a heavy chain variable region having an amino acid sequence at least 80% identical to one of SEQ ID NOs:314, 316, 318, 320, 322, or 324, and a light chain variable region having an amino acid sequence at least 80% identical to one of SEQ ID NOs:315, 317, 319, 321, 323, or 325.

3. The isolated antibody of claim 1, wherein the antibody comprises an IgG, IgA, IgM, IgE, IgD, a Fv, a scFv, a Fab, a F(ab′)2, a minibody, a diabody, a triabody, a multibody, a bispecific antibody, or a multi-specific antibody, wherein said IgG comprises an IgG1, IgG2, IgG3, or IgG4.

4. A pharmaceutical composition comprising the isolated antibody of claim 1 and a pharmaceutically acceptable carrier.

5. A method of increasing cellular fitness of a population of cells, or preventing apoptosis of a population of cells, or a combination thereof, comprising contacting said population of cells with an antibody that binds to calcium channel flower homolog protein, said antibody comprising three heavy chain complementarity determining regions (HCDR1, HCDR2, HCDR3) and three light chain CDRs (LCDR1, LCDR2, LCDR3), wherein
(a) the HCDR1, HCDR2, and HCDR3 have the sequences of SEQ ID NOs:266-268 respectively, and the LCDR1, LCDR2, and LCDR3 have the sequences of SEQ ID NOs:269-271 respectively; or
(b) the HCDR1, HCDR2, and HCDR3 have the sequences of SEQ ID NOs:326-328 respectively, and the LCDR1, LCDR2, and LCDR3 have the sequences of SEQ ID NOs:329-331 respectively; or
(c) the HCDR1, HCDR2, and HCDR3 have the sequences of SEQ ID NOs:272-274 respectively, and the LCDR1, LCDR2, and LCDR3 have the sequences of SEQ ID NOs:275-277 respectively; or
(d) the HCDR1, HCDR2, and HCDR3 have the sequences of SEQ ID NOs:278-280 respectively, and the LCDR1, LCDR2, and LCDR3 have the sequences of SEQ ID NOs:281-283 respectively; or
(e) the HCDR1, HCDR2, and HCDR3 have the sequences of SEQ ID NOs:284-286 respectively, and the LCDR1, LCDR2, and LCDR3 have the sequences of SEQ ID NOs:287-289 respectively; or
(f) the HCDR1, HCDR2, and HCDR3 have the sequences of SEQ ID NOs:290-292 respectively, and the LCDR1, LCDR2, and LCDR3 have the sequences of SEQ ID NOs:293-295 respectively; or
(g) the HCDR1, HCDR2, and HCDR3 have the sequences of SEQ ID NOs:296-298 respectively, and the LCDR1, LCDR2, and LCDR3 have the sequences of SEQ ID NOs:299-301 respectively; or
(h) the HCDR1, HCDR2, and HCDR3 have the sequences of SEQ ID NOs:302-304 respectively, and the LCDR1, LCDR2, and LCDR3 have the sequences of SEQ ID NOs:305-307 respectively; or
(i) the HCDR1, HCDR2, and HCDR3 have the sequences of SEQ ID NOs:308-310 respectively, and the LCDR1, LCDR2, and LCDR3 have the sequences of SEQ ID NOs:311-313 respectively, thereby increasing the cellular fitness of said population of cells.

6. The method of claim 5, wherein said antibody comprises (a) a heavy chain variable region having an amino acid sequence at least 80% identical to SEQ ID NO:235, and a light chain variable region having an amino acid sequence at least 80% identical to SEQ ID NO:236; or
(b) a heavy chain variable region having an amino acid sequence at least 80% identical to SEQ ID NO:237, and a light chain variable region having an amino acid sequence at least 80% identical to SEQ ID NO:238; or
(c) a heavy chain variable region having an amino acid sequence at least 80% identical to SEQ ID NO:239, and a light chain variable region having an amino acid sequence at least 80% identical to SEQ ID NO:240; or
(d) a heavy chain variable region having an amino acid sequence at least 80% identical to SEQ ID NO:241, and a light chain variable region having an amino acid sequence at least 80% identical to SEQ ID NO:242; or
(e) a heavy chain variable region having an amino acid sequence at least 80% identical to SEQ ID NO:243, and a light chain variable region having an amino acid sequence at least 80% identical to SEQ ID NO:244; or
(f) a heavy chain variable region having an amino acid sequence at least 80% identical to SEQ ID NO:245, and a light chain variable region having an amino acid sequence at least 80% identical to SEQ ID NO:246; or
(g) a heavy chain variable region having an amino acid sequence at least 80% identical to SEQ ID NO:247, and a light chain variable region having an amino acid sequence at least 80% identical to SEQ ID NO:248; or
(h) a heavy chain variable region having an amino acid sequence at least 80% identical to SEQ ID NO:249, and a light chain variable region having an amino acid sequence at least 80% identical to SEQ ID NO:250; or
(i) a heavy chain variable region having an amino acid sequence at least 80% identical to one of SEQ ID NOs:314, 316, 318, 320, 322, or 324, and a light chain variable region having an amino acid sequence at least 80% identical to one of SEQ ID NOs:315, 317, 319, 321, 323, or 325.

7. The method of claim 5, wherein the antibody comprises an IgG, IgA, IgM, IgE, IgD, a Fv, a scFv, a Fab, a F(ab′)2, a minibody, a diabody, a triabody, a multibody, a bispecific antibody, or a multi-specific antibody, wherein said IgG comprises an IgG1, IgG2, IgG3, or IgG4.

8. A method of treating a cancer, or a disease or condition in a subject in need thereof, comprising administering to said subject an antibody that binds to calcium channel flower homolog protein, said antibody comprising three heavy chain complementarity determining regions (HCDR1, HCDR2, HCDR3) and three light chain CDRs (LCDR1, LCDR2, LCDR3), wherein
(a) the HCDR1, HCDR2, and HCDR3 have the sequences of SEQ ID NOs:266-268 respectively, and the LCDR1, LCDR2, and LCDR3 have the sequences of SEQ ID NOs:269-271 respectively; or
(b) the HCDR1, HCDR2, and HCDR3 have the sequences of SEQ ID NOs:326-328 respectively, and the LCDR1, LCDR2, and LCDR3 have the sequences of SEQ ID NOs:329-331 respectively; or
(c) the HCDR1, HCDR2, and HCDR3 have the sequences of SEQ ID NOs:272-274 respectively, and the LCDR1, LCDR2, and LCDR3 have the sequences of SEQ ID NOs:275-277 respectively; or
(d) the HCDR1, HCDR2, and HCDR3 have the sequences of SEQ ID NOs:278-280 respectively, and the LCDR1, LCDR2, and LCDR3 have the sequences of SEQ ID NOs:281-283 respectively; or
(e) the HCDR1, HCDR2, and HCDR3 have the sequences of SEQ ID NOs:284-286 respectively, and the LCDR1, LCDR2, and LCDR3 have the sequences of SEQ ID NOs:287-289 respectively; or
(f) the HCDR1, HCDR2, and HCDR3 have the sequences of SEQ ID NOs:290-292 respectively, and the LCDR1, LCDR2, and LCDR3 have the sequences of SEQ ID NOs:293-295 respectively; or
(g) the HCDR1, HCDR2, and HCDR3 have the sequences of SEQ ID NOs:296-298 respectively, and the LCDR1, LCDR2, and LCDR3 have the sequences of SEQ ID NOs:299-301 respectively; or
(h) the HCDR1, HCDR2, and HCDR3 have the sequences of SEQ ID NOs:302-304 respectively, and the LCDR1, LCDR2, and LCDR3 have the sequences of SEQ ID NOs:305-307 respectively; or
(i) the HCDR1, HCDR2, and HCDR3 have the sequences of SEQ ID NOs:308-310 respectively, and the LCDR1, LCDR2, and LCDR3 have the sequences of SEQ ID NOs:311-313 respectively, thereby increasing the cellular fitness of said population of cells, thereby treating said cancer, disease or condition in said subject.

9. The method of claim 8, wherein said antibody comprises (a) a heavy chain variable region having an amino acid sequence at least 80% identical to SEQ ID NO:235, and a light chain variable region having an amino acid sequence at least 80% identical to SEQ ID NO:236; or
(b) a heavy chain variable region having an amino acid sequence at least 80% identical to SEQ ID NO:237, and a light chain variable region having an amino acid sequence at least 80% identical to SEQ ID NO:238; or
(c) a heavy chain variable region having an amino acid sequence at least 80% identical to SEQ ID NO:239, and a light chain variable region having an amino acid sequence at least 80% identical to SEQ ID NO:240; or
(d) a heavy chain variable region having an amino acid sequence at least 80% identical to SEQ ID NO:241, and a light chain variable region having an amino acid sequence at least 80% identical to SEQ ID NO:242; or
(e) a heavy chain variable region having an amino acid sequence at least 80% identical to SEQ ID NO:243, and a light chain variable region having an amino acid sequence at least 80% identical to SEQ ID NO:244; or
(f) a heavy chain variable region having an amino acid sequence at least 80% identical to SEQ ID NO:245, and a light chain variable region having an amino acid sequence at least 80% identical to SEQ ID NO:246; or
(g) a heavy chain variable region having an amino acid sequence at least 80% identical to SEQ ID NO:247, and a light chain variable region having an amino acid sequence at least 80% identical to SEQ ID NO:248; or
(h) a heavy chain variable region having an amino acid sequence at least 80% identical to SEQ ID NO:249, and a light chain variable region having an amino acid sequence at least 80% identical to SEQ ID NO:250; or
(i) a heavy chain variable region having an amino acid sequence at least 80% identical to one of SEQ ID NOs:314, 316, 318, 320, 322, or 324, and a light chain variable region having an amino acid sequence at least 80% identical to one of SEQ ID NOs:315, 317, 319, 321, 323, or 325.

10. The method of claim 8, wherein the antibody comprises an IgG, IgA, IgM, IgE, IgD, a Fv, a scFv, a Fab, a F(ab′)2, a minibody, a diabody, a triabody, a multibody, a bispecific antibody, or a multi-specific antibody, wherein said IgG comprises an IgG1, IgG2, IgG3, or IgG4.

11. The method of claim 8, wherein said disease or condition comprises stroke, cardiovascular disease, neurodegenerative diseases, or aging.

12. The method of claim 8, wherein
(a) said antibody binds to a calcium channel flower homolog protein that behaves as Flower-Win protein; or
(b) said antibody binds to a calcium channel flower homolog protein that behaves as Flower-Lose protein; or
(c) said antibody binds to a calcium channel flower homolog protein that behaves as Flower-Win protein and binds to a calcium channel flower homolog protein that behaves as Flower-Lose protein.


SEQUENCE LISTING (400 sequences)

SEQ ID NO: 1 - DNA, 19 bp, synthetic construct
  source 1..19: mol_type = other DNA
ggatgcagggatgatgttc

SEQ ID NO: 2 - DNA, 19 bp, synthetic construct
  source 1..19: mol_type = other DNA
tgcaccaccaactgcttag

SEQ ID NO: 3 - DNA, 20 bp, synthetic construct
  source 1..20: mol_type = other DNA
gccttcatcttgttgctgtg

SEQ ID NO: 4 - DNA, 20 bp, synthetic construct
  source 1..20: mol_type = other DNA
catcccgcagtagaagacag

SEQ ID NO: 5 - DNA, 18 bp, synthetic construct
  source 1..18: mol_type = other DNA
gcgtgtggatgatgatgg

SEQ ID NO: 6 - DNA, 20 bp, synthetic construct
  source 1..20: mol_type = other DNA
agcagagagtccgtacagca

SEQ ID NO: 7 - DNA, 19 bp, synthetic construct
  source 1..19: mol_type = other DNA
gcggccatggaaagaactt

SEQ ID NO: 8 - DNA, 19 bp, synthetic construct
  source 1..19: mol_type = other DNA
aactgtgtcacgggaccct

SEQ ID NO: 9 - DNA, 20 bp, synthetic construct
  source 1..20: mol_type = other DNA
cacaccaggctactgaaaga

SEQ ID NO: 10 - DNA, 22 bp, synthetic construct
  source 1..22: mol_type = other DNA
cacctctcctcctgagtttatg

SEQ ID NO: 11 - DNA, 19 bp, synthetic construct
  source 1..19: mol_type = other DNA
gccgccaaactgacaaatc

SEQ ID NO: 12 - DNA, 22 bp, synthetic construct
  source 1..22: mol_type = other DNA
gaccaccttctccatccattag

SEQ ID NO: 13 - DNA, 22 bp, synthetic construct
  source 1..22: mol_type = other DNA
agactactacgatggtgcaaag

SEQ ID NO: 14 - DNA, 23 bp, synthetic construct
  source 1..23: mol_type = other DNA
ccaatatctggaactggcatcta

SEQ ID NO: 15 - DNA, 22 bp, synthetic construct
  source 1..22: mol_type = other DNA
ctcccttctctacgcagtgtca

SEQ ID NO: 16 - DNA, 22 bp, synthetic construct
  source 1..22: mol_type = other DNA
agacgtccatgagcacctcgaa

SEQ ID NO: 17 - DNA, 22 bp, synthetic construct
  source 1..22: mol_type = other DNA
caaggccaaagagctagtaagt

SEQ ID NO: 18 - DNA, 23 bp, synthetic construct
  source 1..23: mol_type = other DNA
tccatgctctctcctgtttattc

SEQ ID NO: 19 - DNA, 20 bp, synthetic construct
  source 1..20: mol_type = other DNA
gtttcctgctgttgcttgtg

SEQ ID NO: 20 - DNA, 22 bp, synthetic construct
  source 1..22: mol_type = other DNA
ctggatctcctgctaaccattc

SEQ ID NO: 21 - DNA, 23 bp, synthetic construct
  source 1..23: mol_type = other DNA
ggaggaaactgtcatgggataag

SEQ ID NO: 22 - DNA, 20 bp, synthetic construct
  source 1..20: mol_type = other DNA
cgtgcgtgtgtttgtgtatg

SEQ ID NO: 23 - DNA, 20 bp, synthetic construct
  source 1..20: mol_type = other DNA
tgggtttcctgttccatgag

SEQ ID NO: 24 - DNA, 20 bp, synthetic construct
  source 1..20: mol_type = other DNA
ctcgggtttggaccgtattt

SEQ ID NO: 25 - DNA, 22 bp, synthetic construct
  source 1..22: mol_type = other DNA
cctcagactagaacagcaacac

SEQ ID NO: 26 - DNA, 20 bp, synthetic construct
  source 1..20: mol_type = other DNA
aagtggagacaagggtagga

SEQ ID NO: 27 - DNA, 20 bp, synthetic construct
  source 1..20: mol_type = other DNA
tgacaaacccagaggcaatc

SEQ ID NO: 28 - DNA, 24 bp, synthetic construct
  source 1..24: mol_type = other DNA
gtgacagagtgagagtccataatc

SEQ ID NO: 29 - DNA, 22 bp, synthetic construct
  source 1..22: mol_type = other DNA
gcggcatcaatcgcaccaagta

SEQ ID NO: 30 - DNA, 22 bp, synthetic construct
  source 1..22: mol_type = other DNA
ggtcactcctatccacagcatg

SEQ ID NO: 31 - DNA, 23 bp, synthetic construct
  source 1..23: mol_type = other DNA
ggatcttgaggaacatggacgtg

SEQ ID NO: 32 - DNA, 22 bp, synthetic construct
  source 1..22: mol_type = other DNA
cagcggaatgaccttctcctct

SEQ ID NO: 33 - DNA, 22 bp, synthetic construct
  source 1..22: mol_type = other DNA
tcaagggcagtggacacagcaa

SEQ ID NO: 34 - DNA, 22 bp, synthetic construct
  source 1..22: mol_type = other DNA
ggaaactgccagaagcactagg

SEQ ID NO: 35 - DNA, 20 bp, synthetic construct
  source 1..20: mol_type = other DNA
tggaacagatgctcactgcg

SEQ ID NO: 36 - DNA, 20 bp, synthetic construct
  source 1..20: mol_type = other DNA
ctccatcgctgacaaaagcc

SEQ ID NO: 37 - DNA, 18 bp, synthetic construct
  source 1..18: mol_type = other DNA
cctgggaggaagcgcaag

SEQ ID NO: 38 - DNA, 21 bp, synthetic construct
  source 1..21: mol_type = other DNA
tggatggggacttggagatca

SEQ ID NO: 39 - DNA, 20 bp, synthetic construct
  source 1..20: mol_type = other DNA
atatcgctcggggctttgag

SEQ ID NO: 40 - DNA, 21 bp, synthetic construct
  source 1..21: mol_type = other DNA
ccaggtcagcttcatctgtgt

SEQ ID NO: 41 - DNA, 20 bp, synthetic construct
  source 1..20: mol_type = other DNA
tcctccacctcgtcgcagat

SEQ ID NO: 42 - DNA, 20 bp, synthetic construct
  source 1..20: mol_type = other DNA
cgggacggcttgtgatttct

SEQ ID NO: 43 - DNA, 22 bp, synthetic construct
  source 1..22: mol_type = other DNA
ctacgcactctccttctctgct

SEQ ID NO: 44 - DNA, 22 bp, synthetic construct
  source 1..22: mol_type = other DNA
cggacaatgctcgcaggatgaa

SEQ ID NO: 45 - DNA, 22 bp, synthetic construct
  source 1..22: mol_type = other DNA
ggtgaacaaccacctggaggta

SEQ ID NO: 46 - DNA, 22 bp, synthetic construct
  source 1..22: mol_type = other DNA
aggctgaccatctccaagttcc

SEQ ID NO: 47 - DNA, 19 bp, synthetic construct
  source 1..19: mol_type = other DNA
ttaaggagatggtgctgct

SEQ ID NO: 48 - DNA, 18 bp, synthetic construct
  source 1..18: mol_type = other DNA
atggtggtcagggtgtct

SEQ ID NO: 49 - DNA, 19 bp, synthetic construct
  source 1..19: mol_type = other DNA
gctcaccaacctcaacatc

SEQ ID NO: 50 - DNA, 20 bp, synthetic construct
  source 1..20: mol_type = other DNA
tcagaatgaccatcttcacc

SEQ ID NO: 51 - DNA, 20 bp, synthetic construct
  source 1..20: mol_type = other DNA
cggtgtacgtgtctgagatt

SEQ ID NO: 52 - DNA, 19 bp, synthetic construct
  source 1..19: mol_type = other DNA
atccgaacactgccatgag

SEQ ID NO: 53 - DNA, 20 bp, synthetic construct
  source 1..20: mol_type = other DNA
cactcagcctctcacagaac

SEQ ID NO: 54 - DNA, 20 bp, synthetic construct
  source 1..20: mol_type = other DNA
caggtaatgtctgctccatc

SEQ ID NO: 55 - DNA, 20 bp, synthetic construct
  source 1..20: mol_type = other DNA
gaaacccttggaggttaatg

SEQ ID NO: 56 - DNA, 20 bp, synthetic construct
  source 1..20: mol_type = other DNA
tatcttccccagcttttggt

SEQ ID NO: 57 - DNA, 20 bp, synthetic construct
  source 1..20: mol_type = other DNA
acaaagtgaccaagcagaga

SEQ ID NO: 58 - DNA, 20 bp, synthetic construct
  source 1..20: mol_type = other DNA
tcagggtagtcaatctggaa

SEQ ID NO: 59 - DNA, 20 bp, synthetic construct
  source 1..20: mol_type = other DNA
gaggaggaaggtgatgaaga

SEQ ID NO: 60 - DNA, 20 bp, synthetic construct
  source 1..20: mol_type = other DNA
ctacgagattagccgagagg

SEQ ID NO: 61 - DNA, 20 bp, synthetic construct
  source 1..20: mol_type = other DNA
cagatggcagtatgcttttc

SEQ ID NO: 62 - DNA, 20 bp, synthetic construct
  source 1..20: mol_type = other DNA
accctctcacctgtttcact

SEQ ID NO: 63 - DNA, 20 bp, synthetic construct
  source 1..20: mol_type = other DNA
catgctcaaccttctcattc

SEQ ID NO: 64 - DNA, 20 bp, synthetic construct
  source 1..20: mol_type = other DNA
ttctttctttccttggtggt

SEQ ID NO: 65 - DNA, 20 bp, synthetic construct
  source 1..20: mol_type = other DNA
tcaaagttgctcacccaatc

SEQ ID NO: 66 - DNA, 19 bp, synthetic construct
  source 1..19: mol_type = other DNA
actgcatcccactcatcct

SEQ ID NO: 67 - DNA, 20 bp, synthetic construct
  source 1..20: mol_type = other DNA
ttgaggaggtggaagaagag

SEQ ID NO: 68 - DNA, 20 bp, synthetic construct
  source 1..20: mol_type = other DNA
gtctttggctgaggtgtagg

SEQ ID NO: 69 - DNA, 22 bp, synthetic construct
  source 1..22: mol_type = other DNA
cgtcctctatccgaggacaata

SEQ ID NO: 70 - DNA, 22 bp, synthetic construct
  source 1..22: mol_type = other DNA
gtactgcaataccaggtcgatg

SEQ ID NO: 71 - DNA, 21 bp, synthetic construct
  source 1..21: mol_type = other DNA
gcttcggcagcacatatacta

SEQ ID NO: 72 - DNA, 21 bp, synthetic construct
  source 1..21: mol_type = other DNA
cgaatttgcgtgtcatccttg

SEQ ID NO: 73 - DNA, 21 bp, synthetic construct
  source 1..21: mol_type = other DNA
ttgctctcggtgttcctaatc

SEQ ID NO: 74 - DNA, 20 bp, synthetic construct
  source 1..20: mol_type = other DNA
tcttctccacctgcaaatcc

SEQ ID NO: 75 - DNA, 20 bp, synthetic construct
  source 1..20: mol_type = other DNA
tgagatggatgtgatgttgg

SEQ ID NO: 76 - DNA, 20 bp, synthetic construct
  source 1..20: mol_type = other DNA
caggaggggatcattcatag

SEQ ID NO: 77 - DNA, 17 bp, synthetic construct
  source 1..17: mol_type = other DNA
gacggtggcaccatgag

SEQ ID NO: 78 - DNA, 17 bp, synthetic construct
  source 1..17: mol_type = other DNA
cagacaggcgacacagc

SEQ ID NO: 79 - DNA, 20 bp, synthetic construct
  source 1..20: mol_type = other DNA
tatgctccctctcccacaag

SEQ ID NO: 80 - DNA, 17 bp, synthetic construct
  source 1..17: mol_type = other DNA
catggtgccaccgtcag

SEQ ID NO: 81 - DNA, 17 bp, synthetic construct
  source 1..17: mol_type = other DNA
cctagtcgcggcatgag

SEQ ID NO: 82 - DNA, 21 bp, synthetic construct
  source 1..21: mol_type = other DNA
agggagcatattagcataggg

SEQ ID NO: 83 - DNA, 21 bp, synthetic construct
  source 1..21: mol_type = other DNA
gacggtggcaccatgagcgtg

SEQ ID NO: 84 - DNA, 21 bp, synthetic construct
  source 1..21: mol_type = other DNA
acccccgacccccagcgggtc

SEQ ID NO: 85 - DNA, 17 bp, synthetic construct
  source 1..17: mol_type = other DNA
gacccgctgggggtcgg

SEQ ID NO: 86 - DNA, 20 bp, synthetic construct
  source 1..20: mol_type = other DNA
tcccagaagcgccagcccag

SEQ ID NO: 87 - DNA, 21 bp, synthetic construct
  source 1..21: mol_type = other DNA
gatctctggcctcttcaactg

SEQ ID NO: 88 - DNA, 22 bp, synthetic construct
  source 1..22: mol_type = other DNA
gccatgcattactcacatcatc

SEQ ID NO: 89 - DNA, 20 bp, synthetic construct
  source 1..20: mol_type = other DNA
actctcgtcctttccagtca

SEQ ID NO: 90 - DNA, 17 bp, synthetic construct
  source 1..17: mol_type = other DNA
aagagacacgggcagga

SEQ ID NO: 91 - DNA, 18 bp, synthetic construct
  source 1..18: mol_type = other DNA
caaggcactgcacctctc

SEQ ID NO: 92 - DNA, 20 bp, synthetic construct
  source 1..20: mol_type = other DNA
ctcagcacacagttctcaca

SEQ ID NO: 93 - DNA, 18 bp, synthetic construct
  source 1..18: mol_type = other DNA
taatgcatggccgtccca

SEQ ID NO: 94 - DNA, 17 bp, synthetic construct
  source 1..17: mol_type = other DNA
ccctgccactcctctgt

SEQ ID NO: 95 - DNA, 17 bp, synthetic construct
  source 1..17: mol_type = other DNA
gttgccgtggttgccat

SEQ ID NO: 96 - DNA, 18 bp, synthetic construct
  source 1..18: mol_type = other DNA
aagaagccgcatgctgtg

SEQ ID NO: 97 - DNA, 19 bp, synthetic construct
  source 1..19: mol_type = other DNA
gctgccagttcatcgagtt

SEQ ID NO: 98 - DNA, 19 bp, synthetic construct
  source 1..19: mol_type = other DNA
tcacccgcagtagaagaca

SEQ ID NO: 99 - DNA, 20 bp, synthetic construct
  source 1..20: mol_type = other DNA
ggcattccttatggcactgg

SEQ ID NO: 100 - DNA, 17 bp, synthetic construct
  source 1..17: mol_type = other DNA
tcatgctgcagggacca

SEQ ID NO: 101 - DNA, 18 bp, synthetic construct
  source 1..18: mol_type = other DNA
gggagtgctggagtcttc

SEQ ID NO: 102 - DNA, 18 bp, synthetic construct
  source 1..18: mol_type = other DNA
cctcatccctcctcctct

SEQ ID NO: 103 - DNA, 17 bp, synthetic construct
  source 1..17: mol_type = other DNA
ggtcccgtgacacagtt

SEQ ID NO: 104 - DNA, 18 bp, synthetic construct
  source 1..18: mol_type = other DNA
gaatacactgcccaccct

SEQ ID NO: 105 - DNA, 24 bp, synthetic construct
  source 1..24: mol_type = other DNA
cagttcctctctgtggaagtgtaa

SEQ ID NO: 106 - DNA, 17 bp, synthetic construct
  source 1..17: mol_type = other DNA
ctgggcgcagaaccact

SEQ ID NO: 107 - DNA, 20 bp, synthetic construct
  source 1..20: mol_type = other DNA
gtcgttcccatcgtcatcag

SEQ ID NO: 108 - DNA, 20 bp, synthetic construct
  source 1..20: mol_type = other DNA
cagagcagagagtccgtaca

SEQ ID NO: 109 - DNA, 18 bp, synthetic construct
  source 1..18: mol_type = other DNA
ctgggtgtctaggaagga

SEQ ID NO: 110 - DNA, 19 bp, synthetic construct
  source 1..19: mol_type = other DNA
gggaaagcagaggtcaagg

SEQ ID NO: 111 - DNA, 22 bp, synthetic construct
  source 1..22: mol_type = other DNA
gctttggatcctattcccagaa

SEQ ID NO: 112 - DNA, 21 bp, synthetic construct
  source 1..21: mol_type = other DNA
agacacagcttcagaaagtcc

SEQ ID NO: 113 - DNA, 17 bp, synthetic construct
  source 1..17: mol_type = other DNA
ctctctgctctgggcaa

SEQ ID NO: 114 - DNA, 20 bp, synthetic construct
  source 1..20: mol_type = other DNA
ctcagatcctcctcactgaa

SEQ ID NO: 115 - DNA, 22 bp, synthetic construct
  source 1..22: mol_type = other DNA
cagaggtcccagtaactcattg

SEQ ID NO: 116 - DNA, 21 bp, synthetic construct
  source 1..21: mol_type = other DNA
ccacgcagaagtgttgtaagt

SEQ ID NO: 117 - DNA, 19 bp, synthetic construct
  source 1..19: mol_type = other DNA
agactgaggctggttcctt

SEQ ID NO: 118 - DNA, 20 bp, synthetic construct
  source 1..20: mol_type = other DNA
tgcaggcattctgatttccc

SEQ ID NO: 119 - DNA, 18 bp, synthetic construct
  source 1..18: mol_type = other DNA
ggcctgcagcaaggatag

SEQ ID NO: 120 - DNA, 20 bp, synthetic construct
  source 1..20: mol_type = other DNA
gggtggcaactgagtacaat

SEQ ID NO: 121 - DNA, 22 bp, synthetic construct
  source 1..22: mol_type = other DNA
cagaggtcccagtaactcattg

SEQ ID NO: 122 - DNA, 21 bp, synthetic construct
  source 1..21: mol_type = other DNA
ccacgcagaagtgttgtaagt

SEQ ID NO: 123 - DNA, 17 bp, synthetic construct
  source 1..17: mol_type = other DNA
aggtcacacctgggtca

SEQ ID NO: 124 - DNA, 17 bp, synthetic construct
  source 1..17: mol_type = other DNA
agagagaggcagcgtca

SEQ ID NO: 125 - DNA, 19 bp, synthetic construct
  source 1..19: mol_type = other DNA
ggcgatgcgatctcctatg

SEQ ID NO: 126 - DNA, 19 bp, synthetic construct
  source 1..19: mol_type = other DNA
cacttggacccacacagag

SEQ ID NO: 127 - DNA, 17 bp, synthetic construct
  source 1..17: mol_type = other DNA
ggtcacagggcaggaac

SEQ ID NO: 128 - DNA, 17 bp, synthetic construct
  source 1..17: mol_type = other DNA
agagagaggcagcgtca

SEQ ID NO: 129 - DNA, 17 bp, synthetic construct
  source 1..17: mol_type = other DNA
agtgaggcctggactgt

SEQ ID NO: 130 - DNA, 19 bp, synthetic construct
  source 1..19: mol_type = other DNA
tggagaaaggagcagagga

SEQ ID NO: 131 - DNA, 18 bp, synthetic construct
  source 1..18: mol_type = other DNA
cttaagccaggagccact

SEQ ID NO: 132 - DNA, 18 bp, synthetic construct
  source 1..18: mol_type = other DNA
tgggtagtgtccacacag

SEQ ID NO: 133 - DNA, 19 bp, synthetic construct
  source 1..19: mol_type = other DNA
cctcctcttcctcctcctc

SEQ ID NO: 134 - DNA, 18 bp, synthetic construct
  source 1..18: mol_type = other DNA
aacggaatgtgcggaagt

SEQ ID NO: 135 - DNA, 19 bp, synthetic construct
  source 1..19: mol_type = other DNA
gcggccatggaaagaactt

SEQ ID NO: 136 - DNA, 19 bp, synthetic construct
  source 1..19: mol_type = other DNA
aactgtgtcacgggaccct

SEQ ID NO: 137 - DNA, 22 bp, synthetic construct
  source 1..22: mol_type = other DNA
gcctgaggcagaaggagaagag

SEQ ID NO: 138 - DNA, 22 bp, synthetic construct
  source 1..22: mol_type = other DNA
atggcagatgccagactggaga

SEQ ID NO: 139 - DNA, 22 bp, synthetic construct
  source 1..22: mol_type = other DNA
ccagcatctgtgcttgtggaca

SEQ ID NO: 140 - DNA, 22 bp, synthetic construct
  source 1..22: mol_type = other DNA
ctgcgaaggttgtgaactgagg

SEQ ID NO: 141 - DNA, 22 bp, synthetic construct
  source 1..22: mol_type = other DNA
ttgacccagtggctccacgata

SEQ ID NO: 142 - DNA, 22 bp, synthetic construct
  source 1..22: mol_type = other DNA
cacaggcttcaagccaacctca

SEQ ID NO: 143 - DNA, 22 bp, synthetic construct
  source 1..22: mol_type = other DNA
gccataccaaggatgtgctgag

SEQ ID NO: 144 - DNA, 22 bp, synthetic construct
  source 1..22: mol_type = other DNA
cacaagacacccactctgagtg

SEQ ID NO: 145 - DNA, 22 bp, synthetic construct
  source 1..22: mol_type = other DNA
cctctttggacaaccgcctttc

SEQ ID NO: 146 - DNA, 22 bp, synthetic construct
  source 1..22: mol_type = other DNA
ggcatcagcaccatacttctgg

SEQ ID NO: 147 - DNA, 22 bp, synthetic construct
  source 1..22: mol_type = other DNA
gcctgtcaaagaagcacctgga

SEQ ID NO: 148 - DNA, 22 bp, synthetic construct
  source 1..22: mol_type = other DNA
gaaagccgtagtcggttctgtg

SEQ ID NO: 149 - DNA, 22 bp, synthetic construct
  source 1..22: mol_type = other DNA
gaaacagtgcgcctcatggatc

SEQ ID NO: 150 - DNA, 22 bp, synthetic construct
  source 1..22: mol_type = other DNA
agccatacgcaacggatgtctg

SEQ ID NO: 151 - DNA, 22 bp, synthetic construct
  source 1..22: mol_type = other DNA
gcagaatccacgccagtacaag

SEQ ID NO: 152 - DNA, 22 bp, synthetic construct
  source 1..22: mol_type = other DNA
gcttgttgtccagaccattggc

SEQ ID NO: 153 - DNA, 22 bp, synthetic construct
  source 1..22: mol_type = other DNA
gctgaagatggctactctggag

SEQ ID NO: 154 - DNA, 22 bp, synthetic construct
  source 1..22: mol_type = other DNA
acagcagtcagttcccgaatcc

SEQ ID NO: 155 - DNA, 22 bp, synthetic construct
  source 1..22: mol_type = other DNA
gaagacagaggtgctgatggag

SEQ ID NO: 156 - DNA, 22 bp, synthetic construct
  source 1..22: mol_type = other DNA
ctctgtctcacacggagttagc

SEQ ID NO: 157 - DNA, 22 bp, synthetic construct
  source 1..22: mol_type = other DNA
gctactggatgctgtggacact

SEQ ID NO: 158 - DNA, 22 bp, synthetic construct
  source 1..22: mol_type = other DNA
cacgctctagtgtacctgtcac

SEQ ID NO: 159 - DNA, 23 bp, synthetic construct
  source 1..23: mol_type = other DNA
cagcaaccttctaactacggtcc

SEQ ID NO: 160 - DNA, 22 bp, synthetic construct
  source 1..22: mol_type = other DNA
cactgcctcctggaccatagtt

SEQ ID NO: 161 - DNA, 22 bp, synthetic construct
  source 1..22: mol_type = other DNA
tccagagcacaacaggacactg

SEQ ID NO: 162 - DNA, 23 bp, synthetic construct
  source 1..23: mol_type = other DNA
gcttcaccagttactctgccatc

SEQ ID NO: 163 - DNA, 22 bp, synthetic construct
  source 1..22: mol_type = other DNA
gatggcaatgccagtggaacca

SEQ ID NO: 164 - DNA, 22 bp, synthetic construct
  source 1..22: mol_type = other DNA
gagaacaccagtctccactcgg

SEQ ID NO: 165 - DNA, 23 bp, synthetic construct
  source 1..23: mol_type = other DNA
gcagatggcttatgaaccacagg

SEQ ID NO: 166 - DNA, 22 bp, synthetic construct
  source 1..22: mol_type = other DNA
aatccgctgaccacctttgcca

SEQ ID NO: 167 - DNA, 22 bp, synthetic construct
  source 1..22: mol_type = other DNA
catgggtcgatttggatctggg

SEQ ID NO: 168 - DNA, 22 bp, synthetic construct
  source 1..22: mol_type = other DNA
tcaatgccaggacccatcctct

SEQ ID NO: 169 - DNA, 22 bp, synthetic construct
  source 1..22: mol_type = other DNA
gcatttccgctctattcggctg

SEQ ID NO: 170 - DNA, 22 bp, synthetic construct
  source 1..22: mol_type = other DNA
gagcggcatttatcagtggctg

SEQ ID NO: 171 - DNA, 23 bp, synthetic construct
  source 1..23: mol_type = other DNA
ctgctgatggaagaggactacag

SEQ ID NO: 172 - DNA, 22 bp, synthetic construct
  source 1..22: mol_type = other DNA
ggcacgcatttccacagcttca

SEQ ID NO: 173 - DNA, 22 bp, synthetic construct
  source 1..22: mol_type = other DNA
actatgcctccaaagggtgtcc

SEQ ID NO: 174 - DNA, 22 bp, synthetic construct
  source 1..22: mol_type = other DNA
agagccaactcttcctacagcc

SEQ ID NO: 175 - DNA, 22 bp, synthetic construct
  source 1..22: mol_type = other DNA
cctctacctgaagccaatccag

SEQ ID NO: 176 - DNA, 22 bp, synthetic construct
  source 1..22: mol_type = other DNA
ccgtcttcaccaggaactggtc

SEQ ID NO: 177 - DNA, 22 bp, synthetic construct
  source 1..22: mol_type = other DNA
gccaaggttacggtggttatgg

SEQ ID NO: 178 - DNA, 22 bp, synthetic construct
  source 1..22: mol_type = other DNA
tgatgaccacctcgcctggata

SEQ ID NO: 179 - DNA, 22 bp, synthetic construct
  source 1..22: mol_type = other DNA
gagattgctgcccgaaagaagc

SEQ ID NO: 180 - DNA, 22 bp, synthetic construct
  source 1..22: mol_type = other DNA
ttcgctggaagcctgcaaacag

SEQ ID NO: 181 - DNA, 23 bp, synthetic construct
  source 1..23: mol_type = other DNA
gcagagaacgatgtgattggctg

SEQ ID NO: 182 - DNA, 22 bp, synthetic construct
  source 1..22: mol_type = other DNA
caaggcttccttctggattcgg

SEQ ID NO: 183 - DNA, 22 bp, synthetic construct
  source 1..22: mol_type = other DNA
acctggctcaaagaccacacag

SEQ ID NO: 184 - DNA, 22 bp, synthetic construct
  source 1..22: mol_type = other DNA
tggcttgatggacgagcaggta

SEQ ID NO: 185 - DNA, 22 bp, synthetic construct
  source 1..22: mol_type = other DNA
agaaggaccacctgctcagtgt

SEQ ID NO: 186 - DNA, 22 bp, synthetic construct
  source 1..22: mol_type = other DNA
atgcaccgtcagacactggatc

SEQ ID NO: 187 - DNA, 22 bp, synthetic construct
  source 1..22: mol_type = other DNA
tcctaccaagcagacacagacc

SEQ ID NO: 188 - DNA, 22 bp, synthetic construct
  source 1..22: mol_type = other DNA
caggaggtatgcctgtgagttc

SEQ ID NO: 189 - DNA, 22 bp, synthetic construct
  source 1..22: mol_type = other DNA
atcgaaacggcggatcatgcca

SEQ ID NO: 190 - DNA, 22 bp, synthetic construct
  source 1..22: mol_type = other DNA
ggcatcacctacaaagtctggc

SEQ ID NO: 191 - DNA, 22 bp, synthetic construct
  source 1..22: mol_type = other DNA
ggacaacacaccatttctccgc

SEQ ID NO: 192 - DNA, 22 bp, synthetic construct
  source 1..22: mol_type = other DNA
agcctttcacctctggagagct

SEQ ID NO: 193 - DNA, 20 bp, synthetic construct
  source 1..20: mol_type = other DNA
  modified_base 20: mod_base = OTHER; note = Biotin-Triethylene glycol modified nucleotide
cgtatgttgcatcaccttca

SEQ ID NO: 194 - DNA, 20 bp, synthetic construct
  source 1..20: mol_type = other DNA
  modified_base 20: mod_base = OTHER; note = Biotin-Triethylene glycol modified nucleotide
cggagtggataaagcatatt

SEQ ID NO: 195 - DNA, 19 bp, synthetic construct
  source 1..19: mol_type = other DNA
  modified_base 19: mod_base = OTHER; note = Biotin-Triethylene glycol modified nucleotide
gctaaattggttggtggat

SEQ ID NO: 196 - DNA, 20 bp, synthetic construct
  source 1..20: mol_type = other DNA
  modified_base 20: mod_base = OTHER; note = Biotin-Triethylene glycol modified nucleotide
acttccttctgcagttggag

SEQ ID NO: 197 - DNA, 20 bp, synthetic construct
  source 1..20: mol_type = other DNA
  modified_base 20: mod_base = OTHER; note = Biotin-Triethylene glycol modified nucleotide
acctaagatgtggacttaac

SEQ ID NO: 198 - DNA, 20 bp, synthetic construct
  source 1..20: mol_type = other DNA
  modified_base 20: mod_base = OTHER; note = Biotin-Triethylene glycol modified nucleotide
aatatgctttatccactccg

SEQ ID NO: 199 - DNA, 19 bp, synthetic construct
  source 1..19: mol_type = other DNA
  modified_base 19: mod_base = OTHER; note = Biotin-Triethylene glycol modified nucleotide
atccaccaaccaatttagc

SEQ ID NO: 200 - DNA, 20 bp, synthetic construct
  source 1..20: mol_type = other DNA
  modified_base 20: mod_base = OTHER; note = Biotin-Triethylene glycol modified nucleotide
ctccaactgcagaaggaagt

SEQ ID NO: 201 - DNA, 20 bp, synthetic construct
  source 1..20: mol_type = other DNA
  modified_base 20: mod_base = OTHER; note = Biotin-Triethylene glycol modified nucleotide
gttaagtccacatcttaggt

SEQ ID NO: 202 - DNA, 20 bp, synthetic construct
  source 1..20: mol_type = other DNA
  modified_base 20: mod_base = OTHER; note = Biotin-Triethylene glycol modified nucleotide
gcgaggcgtatttatagacg

SEQ ID NO: 203 - DNA, 20 bp, synthetic construct
  source 1..20: mol_type = other DNA
  modified_base 20: mod_base = OTHER; note = Biotin-Triethylene glycol modified nucleotide
tcatctcaacctccgtcatg

SEQ ID NO: 204 - DNA, 20 bp, synthetic construct
  source 1..20: mol_type = other DNA
  modified_base 20: mod_base = OTHER; note = Biotin-Triethylene glycol modified nucleotide
ttagctttttgtttcctagc

SEQ ID NO: 205 - DNA, 20 bp, synthetic construct
  source 1..20: mol_type = other DNA
  modified_base 20: mod_base = OTHER; note = Biotin-Triethylene glycol modified nucleotide
tacttccgttacgaaagtcc

SEQ ID NO: 206 - DNA, 32 bp, synthetic construct
  source 1..32: mol_type = other DNA
  modified_base 1: mod_base = OTHER; note = 5'-6C-Psoralen modified nucleotide
ccttctgcagttggagacagccttctgtgtgg

SEQ ID NO: 207 - DNA, 20 bp, synthetic construct
  source 1..20: mol_type = other DNA
gctttgtaccaactcattgg

SEQ ID NO: 208 - DNA, 20 bp, synthetic construct
  source 1..20: mol_type = other DNA
actcgccagagttccacaac

SEQ ID NO: 209 - DNA, 20 bp, synthetic construct
  source 1..20: mol_type = other DNA
ggctgggaggtcttactcct

SEQ ID NO: 210 - DNA, 20 bp, synthetic construct
  source 1..20: mol_type = other DNA
ctgacactaatcgggggcac

SEQ ID NO: 211 - DNA, 21 bp, synthetic construct
  source 1..21: mol_type = other DNA
atctctgcagttacaattatt

SEQ ID NO: 212 - DNA, 21 bp, synthetic construct
  source 1..21: mol_type = other DNA
acctgcaaattctacacatta

SEQ ID NO: 213 - DNA, 21 bp, synthetic construct
  source 1..21: mol_type = other DNA
cagcggccatggaaagaactt

SEQ ID NO: 214 - DNA, 21 bp, synthetic construct
  source 1..21: mol_type = other DNA
gcaaagaatcacatgccactt

SEQ ID NO: 215 - DNA, 21 bp, synthetic construct
  source 1..21: mol_type = other DNA
gcacaatctacgaagaatcaa

SEQ ID NO: 216 - DNA, 21 bp, synthetic construct
  source 1..21: mol_type = other DNA
cggagtgattacgatggcatt

SEQ ID NO: 217 - DNA, 21 bp, synthetic construct
  source 1..21: mol_type = other DNA
cgtagaatagtcgaacaagat

SEQ ID NO: 218 - DNA, 21 bp, synthetic construct
  source 1..21: mol_type = other DNA
ccaccagaagaagagaagaat

SEQ ID NO: 219 - DNA, 21 bp, synthetic construct
  source 1..21: mol_type = other DNA
cgagagagttcatggctcttt

SEQ ID NO: 220 - DNA, 21 bp, synthetic construct
  source 1..21: mol_type = other DNA
ggattcagatgtcaccttaaa

SEQ ID NO: 221 - DNA, 21 bp, synthetic construct
  source 1..21: mol_type = other DNA
taacaagaggaaatccaattt

SEQ ID NO: 222 - DNA, 21 bp, synthetic construct
  source 1..21: mol_type = other DNA
tggaactgtcgaatggtgaaa

SEQ ID NO: 223 - DNA, 20 bp, synthetic construct
  source 1..20: mol_type = other DNA
tcgcttcccggccccagccc

SEQ ID NO: 224 - DNA, 19 bp, synthetic construct
  source 1..19: mol_type = other DNA
tcactgggccaggaagtgg

SEQ ID NO: 225 - DNA, 18 bp, synthetic construct
  source 1..18: mol_type = other DNA
cctcagtcctggaaggga

SEQ ID NO: 226 - DNA, 19 bp, synthetic construct
  source 1..19: mol_type = other DNA
gcgcttctgggaacattcc

SEQ ID NO: 227 - DNA, 21 bp, synthetic construct
  source 1..21: mol_type = other DNA
gagcagttcccgtcaatccct

SEQ ID NO: 228 - protein, 20 aa, synthetic construct
CSGGAPGASASSAPPAQEEG

SEQ ID NO: 229 - protein, 25 aa, synthetic construct
CGGAPGASASSAPPAQEEGMTWWYR

SEQ ID NO: 230 - protein, 18 aa, synthetic construct
GGAPGASASSAPPAQEEG

SEQ ID NO: 231 - protein, 191 aa, Homo sapiens
MSSSGGAPGASASSAPPAQEEGMTWWYRWLCRLSGVLGAVSCAISGLFNCITIHPLNIAAGVWMMMAVVPIVISLTLTTLLGNAIAFATGVLYGLSALGKKAQTEAGSFAAQHPREPGPFSEGTRQAFATPAVVSGEIRMPAGAMRSPMPGSSSRGSRRMRRSSRRPWRGSCEGLGAPPSLSPLLALCGSK

SEQ ID NO: 232 - protein, 233 aa, Homo sapiens
MSSSGGAPGASASSAPPAQEEGMTWWYRWLCRLSGVLGAVSCAISGLFNCITIHPLNIAAGVWMIMNAFILLLCEAPFCCQFIEFANTVAEKVDRLRSWQKAVFYCGMAVVPIVISLTLTTLLGNAIAFATGVLYGLSALGKKAQTEAGSFAAQHPREPGPFSEGTRQAFATPAVVSGEIRMPAGAMRSPMPGSSSRGSRRMRRSSRRPWRGSCEGLGAPPSLSPLLALCGSK

SEQ ID NO: 233 - protein, 130 aa, Homo sapiens
MSSSGGAPGASASSAPPAQEEGMTWWYRWLCRLSGVLGAVSCAISGLFNCITIHPLNIAAGVWMMMAVVPIVISLTLTTLLGNAIAFATGVLYGLSALGKKGDAISYARIQQQRQQADEEKLAETLEGEL

SEQ ID NO: 234 - protein, 172 aa, Homo sapiens
MSSSGGAPGASASSAPPAQEEGMTWWYRWLCRLSGVLGAVSCAISGLFNCITIHPLNIAAGVWMIMNAFILLLCEAPFCCQFIEFANTVAEKVDRLRSWQKAVFYCGMAVVPIVISLTLTTLLGNAIAFATGVLYGLSALGKKGDAISYARIQQQRQQADEEKLAETLEGEL

SEQ ID NO: 235 - protein, 275 aa, synthetic construct
GRVLFYSDSAVVKFMGSPNTLKWENWPLYATKRGPSSVVGAGTAQVMHSALRYASSGNSAVTYEGESSNYGTSMFSPSKKKPSTFCLRNYSYTESTKYCYYCGDRGTMVPPLLTNYSSLLGSPFVWARTIVSDPWTQQSDWGTYIALNESNSAASWPDLLYQSQSAQETVQTSGELVWVKAGPSADPQGDVFSGTPTVQGGAVVSKTLLADSKCSTTVRPEIGARVVLNTHEVIAPTGRLSTTRPERAEGMLLNDQLVSPQVSEPFMVTVQSYSV

SEQ ID NO: 236 - protein, 220 aa, synthetic construct
STFTVELSGQKLNEPPSSVYIATNIVCVTQSKDLLSDFWNVDEDVFCDAQLYKPFTQTEDYSNEGVTSERGPSSMPTDEPTYMIKRQSYACSKSANIAWSGDDKLSVRHSGLVATGKVQKTRTYFLNPREVGLAYTSPSLGGPFVTEGSESNSNQGSSSSNMDQSIKWSTYTYSNCLSSEHWSKIGQSYAIATYDSKQTFPVTFSSMIHALCLQTKKYLR

SEQ ID NO: 237 - protein, 226 aa, synthetic construct
SGGGVVWKPSLVEGYQGLVVSPLVVAGTLVAKSDEPPNYLSSYSTLGTDTVSTTVYARVKLKAVDTFVDTESWSDGVGLTWCASSPYDISVWFHQTFKHGGQQIGEHCSYGTYIPGTLGGQQAATQSQGISKWATSGSTAKVVASGSPLTRVPTTKSGFSSNTSVYAKFPPAARSPKVNADKLSASVVFTSQQGKRCTCYVTTQVVPRSLAGRPDDENLVGSRLFK

SEQ ID NO: 238 - protein, 215 aa, synthetic construct
YCCTGQGGTLYQKEYVCGWSKSITCKFREDNSSIQRAFSQALLPSASPADSESDTKAEPDQKSSHGSFEDVEESTSTRIQVLHSTLTTGQLRAGGFKALNATVIASAEPERWVSSLLVQQGKPPAYATELDRVPVADVCGRLDVFHRSQDNLPGFRTRYQQVRWLYSERSTYGQYAKESSVTLKVGGFVVVITIRNTSPSLSATSNSYFKNPPPT

SEQ ID NO: 239 - protein, 230 aa, synthetic construct
  VARIANT 37: note = Any amino acid
  VARIANT 43: note = Any amino acid
  VARIANT 66: note = Any amino acid
  VARIANT 115: note = Any amino acid
  VARIANT 124: note = Any amino acid
  VARIANT 170: note = Any amino acid
GRVLDSCKVGVSVGSSAAVTSPTSSYRQVTMGSKSNXSTSQAXLTSGRMFPSTAPEGQKKGSVYLXSTSATQSADKRPNPTKKPSSNVYKPGGGECRVRVSSNKVCSTFIFEVYXQPKELNSCXFIWVATQAEISSMSGVDWGYPAVMLACAQLGEWVAATVFTQNHTDXLSVIQKMCEYVWLWFSPQICDHTLSVTLVYFIGVPRKQISYGGLSPTSNVVASGGLTLTD

SEQ ID NO: 240 - protein, 216 aa, synthetic construct
GIVAPVGIIPSYKQVYGVNGWGVALESWSKRVEVAGSFTHPGLVKSPVSAYQLACLHSRSTLESEELRGSGTQYPTATLFYGDLSRTDDILADKSQPSPFRKSGGTPTYNFISILRFSTDSVQEFSGSSQCQRALKLWAEDEATEKKSSYSQLPSYAKPLFTQNGNQKRLRSRDVSTSVNTSSHSGCEQSESRLCTCKTGQDVFAALTVQAYYTPE

SEQ ID NO: 241 - protein, 221 aa, synthetic construct
GRVLYEDVKSCRNSGSGTVTSQYATTSVTFQPAVTPGSTSSHSALGDAQNTLPKPWYFTDVKSVLGKTTDQGRLASGMLSVSDEIELPCQYASKLASKWPTGMVYSVGFELEVTIKIMSSGTGAAWVISYSALMTGSLPTAVEFTDYESSVLNSLPVQASGAGSPPAINWTHYDAANRGPSQTKCVAKVHSSLYVAQKVYVTTKVPAKCVDDPTSLWYNTS

SEQ ID NO: 242 - protein, 220 aa, synthetic construct
KTLVLYRSLTGNSINTCGEGPILQSIYLWTKTGDFYSMYSQNYARKATDTLVAVVCGPPAKLLQKRSEVSLVLQMGQGATTQASDTPGISKYEHKHCFLGSDYNWGPDSDTVTWLSKTANFMGRRNEETQTTQPPIKKQSYGFSSNLVTDPSSTYAFETSEVPEYPSTYDASSSVDNKCKTSASSQSSNNVGLEQKITWNERVNCTNSKFTDGDSFLLSI

SEQ ID NO: 243 - protein, 229 aa, synthetic construct
HNFKKSMLWSKPNTSDYDTVPSAILVDVVMTGASYVSTQQGSVTAARRTGEAGSCLHGFSPTNSLMCVDSVVVKSTRTKSEIGGAGCSDGAPDAYKWVSKELTGQTAYVGTTIWTSTWTSKGQYGPMYRITKGQGALNGCSDVAKFIYTPPGYFSVSVRSAVLSGLVRPLLDDGNTSESQLAKLNAESTKYLNLSAIVICGPFINVSGQSWGKQYVVAFQQGPPSVYSS

SEQ ID NO: 244 - protein, 219 aa, synthetic construct
SPGARSETVNQKKLSHQEAASCICLLQGGNSFSAYSGETPWSPSVIVGQTVVAGSKPFGKDTVSVSSGTPPQPNLANSVLSSSARQEPSPALPWTELNITGSVRKSSGSHTTPCTKSPYLLETQPQVSPLGLCEDNGDCTKYGAVSTNTGLADGATYIDPGSKIVQNSEKAQGTYLPYAAWEAVQWPSTKSYDRFVHCSAGQKTGVYDSFLTLVTAYSA

SEQ ID NO: 245 - protein, 223 aa, synthetic construct
GRVLSNMTSPYVYNPSDKSWLLVTPSYAVTLSADAGTPVVANIGNSPQENSKGSTTTTLSISSRADYAVSSQLTDQDAEVLLWTGAQVVGAETTEKAPYGAVVSSPWSFCLVPPFIGGLHYQLFGTKWRYSTSASKVAILHDRTQLTSSYSSYGGQFASTDPANSTDWKCVGGYKSPQSLWTDVHQAVTETESSTKNRIVPAEKMVACLYVEQRTSPLSTCPL

SEQ ID NO: 246 - protein, 219 aa, synthetic construct
AVDPLSTYHSYRIGCGGTIYSDDPSINSGGGSVLKSSSLSDPGYCKLSLAMFKVLTAVQLEEDFQVVASLAVSAWRECGITLKPNNKSSTWYTTSCSQFKNVAFGEIGSSPSIRERRTVKFKEMPYSMDYVVNGNQEMSRRTTDLPYLLTLTYNQSDSTTQETSSQPQGTEGYDRSYFSYISVNQRISWKASHNHDWNSQVASTDYEPRLQSKTKTCKF

SEQ ID NO: 247 - protein, 231 aa, synthetic construct
DGETNPSSTKLKLVLTSTGQLNVSPSSVSPHPLFVSMAHSYDKKSSSASRNVVFVSGYSGYYLDWSSQKSGVYVSGYGYKSRGYTVQSKTDNAASPKLADAYSLPLCFMGGCTGGTKSTVQLRWSYNFVNSSLYSDDFQAATTFRRDNCAVAVTYKGTPSGSSSQFGGPEETIVNPVHLDGGWVTKLRLNSPPTKEYFLGWALDMVDTIVKTGIGVSCSQEGFWNGKVDLT

SEQ ID NO: 248 - protein, 210 aa, synthetic construct
EARDTEATLQPDAYCNDTVSVGLGGAVAGHSLESSPVLWQNSTTVGKKVKNWKFVTNTASQYAPISNPGKTWSGPSKYDAYFVTLKTTVWVNALSCTSSESTVSNASQQGYGSEESARTTLTSYPYESDSPYSYTLGGDKVKSPSKNTAIIIDRHILPAQDVLQAVAGGIRDRYQQNVQPVPQHGHNALSAFVVPPKEVDGPPPFCSVAC

SEQ ID NO: 249 - protein, 227 aa, synthetic construct
  VARIANT 194: note = Any amino acid
ELCWGMSPTVLRPSSQYSKPDADARKFGCVYLLSYKCAEKYTCGVITQWWTDAVSSLNLGGKSVYAPYTRTMVQNGLLKTTSSVTPSTSSGESWTSVTPATLYYGVRKLLLVVTPKTELQVAAGHLSSPSFVNSDSHTVTLAIMPPQSPKKLDVDTKADFTFSSLSSSGQTSVDSVVVRGVLPSSRGALSGTSXCNLVLKDVGSTRTDSILPVWITTFSPDDFGNST

SEQ ID NO: 250 - protein, 217 aa, synthetic construct
  VARIANT 46: note = Any amino acid
QSYVKPQTTTAWPWPLTTPSKGDNFDGHSGSPVSGSLCYPGLQVIXSTGVYELDAAAAFFTTSETRSPNSFWGSPAAPQPAVSSKASTSGACVTSGESNQYWISVAIWATSYGASKKEPAQGDTKSESARYTPSIYQSSATKDGQEKQGRKPNESNCAGYSAPKCKVLGGFLVPSLVGLQTDIVLTECLVHPSSEVVTKSASLLNLDNEPNTLSSVV

SEQ ID NO: 251 - protein, 20 aa, synthetic construct
CSGGAPGASASSAPPAQEEG

SEQ ID NO: 252 - protein, 25 aa, synthetic construct
CGGAPGASASSAPPAQEEGMTWWYR

SEQ ID NO: 253 - protein, 9 aa, synthetic construct
EKVDRLRSW

SEQ ID NO: 254 - protein, 18 aa, synthetic construct
NTVAEKVDRLRSWQKAVF

SEQ ID NO: 255 - protein, 34 aa, synthetic construct
KKAQTEAGSFAAQHPREPGPFSEGTRQAFATPAV

SEQ ID NO: 256 - protein, 26 aa, synthetic construct
EAGSFAAQHPREPGPFSEGTRQAFAT

SEQ ID NO: 257 - protein, 28 aa, synthetic construct
GKKGDAISYARIQQQRQQADEEKLAETL

SEQ ID NO: 258 - protein, 46 aa, synthetic construct
SGEIRMPAGAMRSPMPGSSSRGSRRMRRSSRRPWRGSCEGLGAPPS

SEQ ID NO: 259 - DNA, 21 bp, synthetic construct
  source 1..21: mol_type = other DNA
cagaaggctgtcttctactgc

SEQ ID NO: 260 - DNA, 21 bp, synthetic construct
  source 1..21: mol_type = other DNA
gcagtagaagacagccttctg

SEQ ID NO: 261 - RNA, 21 nt, synthetic construct
  source 1..21: mol_type = other RNA
cgtcaacctctgtcggaagac

SEQ ID NO: 262 - DNA, 19 bp, synthetic construct
  source 1..19: mol_type = other DNA
gcgcttctgggaacattcc

SEQ ID NO: 263 - DNA, 18 bp, synthetic construct
  source 1..18: mol_type = other DNA
cctcagtcctggaaggga

SEQ ID NO: 264 - DNA, 19 bp, synthetic construct
  source 1..19: mol_type = other DNA
tcactgggccaggaagtgg

SEQ ID NO: 265 - DNA, 19 bp, synthetic construct
  source 1..19: mol_type = other DNA
tcgcttcccggcccagccc

SEQ ID NO: 266 - protein, 8 aa, synthetic construct
GNSAVTYE

SEQ ID NO: 267 - protein, 12 aa, synthetic construct
VPPLLTNYSSLL

SEQ ID NO: 268 - protein, 8 aa, synthetic construct
VQTSGELV

SEQ ID NO: 269 - protein, 8 aa, synthetic construct
QLYKPFTQ

SEQ ID NO: 270 - protein, 8 aa, synthetic construct
CSKSANIA

SEQ ID NO: 271 - protein, 7 aa, synthetic construct
SNSNQGS

SEQ ID NO: 272 - protein, 9 aa, synthetic construct
LGTDTVSTT

SEQ ID NO: 273 - protein, 10 aa, synthetic construct
VGLTWCASSP

SEQ ID NO: 274 - protein, 7 aa, synthetic construct
LTRVPTT

SEQ ID NO: 275 - protein, 8 aa, synthetic construct
QALLPSAS

SEQ ID NO: 276 - protein, 10 aa, synthetic construct
ALNATVIASA

SEQ ID NO: 277 - protein, 7 aa, synthetic construct
SQDNLPG

SEQ ID NO: 278 - protein, 9 aa, synthetic construct
APEGQKKGS

SEQ ID NO: 279 - protein, 9 aa, synthetic construct
CRVRVSSNK

SEQ ID NO: 280 - protein, 9 aa, synthetic construct
VWLWFSPQI

SEQ ID NO: 281 - protein, 12 aa, synthetic construct
WSKRVEVAGSFT

SEQ ID NO: 282 - protein, 4 aa, synthetic construct
QPSP

SEQ ID NO: 283 - protein, 15 aa, synthetic construct
NGNQKRLRSRDVSTS

SEQ ID NO: 284 - protein, 20 aa, synthetic construct
SSHSALGDAQNTLPKPWYFY

SEQ ID NO: 285 - protein, 16 aa, synthetic construct
ELPCQYASKLASKWPT

SEQ ID NO: 286 - protein, 14 aa, synthetic construct
PSQTKCVAKVHSSL

SEQ ID NO: 287 - protein, 12 aa, synthetic construct
RKATDTLVAVVC

SEQ ID NO: 288 - protein, 8 aa, synthetic construct
KYEHKHCF

SEQ ID NO: 289 - protein, 14 aa, synthetic construct
TQPPIKKQSYGFSS

SEQ ID NO: 290 - protein, 10 aa, synthetic construct
GFSPTNSLMC

SEQ ID NO: 291 - protein, 12 aa, synthetic construct
VSKELTGQTAYV

SEQ ID NO: 292 - protein, 10 aa, synthetic construct
VAKFIYTPPG

SEQ ID NO: 293 - protein, 13 aa, synthetic construct
SPSVIVGQTVVAG

SEQ ID NO: 294 - protein, 10 aa, synthetic construct
PWTELNITGS

SEQ ID NO: 295 - protein, 7 aa, synthetic construct
VSTNTGL

SEQ ID NO: 296 - protein, 17 aa, synthetic construct
VVANIGNSPQENSKGST

SEQ ID NO: 297 - protein, 13 aa, synthetic construct
SSPWSFCLVPPFI

SEQ ID NO: 298 - protein, 7 aa, synthetic construct
SLWTDVH

SEQ ID NO: 299 - protein, 12 aa, synthetic construct
SDPGYCKLSLAM

SEQ ID NO: 300 - protein, 9 aa, synthetic construct
RERRTVKFK

SEQ ID NO: 301 - protein, 14 aa, synthetic construct
QGTEGYDRSYFSYI

SEQ ID NO: 302 - protein, 15 aa, synthetic construct
PLFVSMAHSYDKKSS

SEQ ID NO: 303 - protein, 10 aa, synthetic construct
WSYNFVNSSL

SEQ ID NO: 304 - protein, 12 aa, synthetic construct
YKGTPSGSSSQF

SEQ ID NO: 305 - protein, 14 aa, synthetic construct
ESSPVLWQNSTTVG

SEQ ID NO: 306 - protein, 14 aa, synthetic construct
TLKTTVWVNALSCT

SEQ ID NO: 307 - protein, 10 aa, synthetic construct
RYQQNVQPVP

SEQ ID NO: 308 - protein, 15 aa, synthetic construct
LSYKCAEKYTCGVIT

SEQ ID NO: 309 - protein, 10 aa, synthetic construct
ATLYYGVRKL

SEQ ID NO: 310 - protein, 13 aa, synthetic construct
NSDSHTVTLAIMP

SEQ ID NO: 311 - protein, 15 aa, synthetic construct
  VARIANT 9: note = Any amino acid
CYPGLQVIXSTGVYE

SEQ ID NO: 312 - protein, 13 aa, synthetic construct
QYWISVAIWATSY

SEQ ID NO: 313 - protein, 19 aa, synthetic construct
APKCKVLGGFLVPSLVGLQ

SEQ ID NO: 314 - protein, 116 aa, synthetic construct
EVQLQQSGAELVRPGASVKLSCTASGFNIKDSIMHWVKQRPEQGLEWIGWIDPEDGDTEYAPKFQDKATITADTSSNTAYLQLSSLTSEDTAVYYCSKSYYGPDFWGQGTTLTVSS

SEQ ID NO: 315 - protein, 112 aa, synthetic construct
DVVMTQTPLSLPVSLGDQASISCRSSQSIVHSNGNTYLFWYLQKPGQSPKLLIYRVSNRVSGVPDRFSGSGSGTDFTLKISRVEAEDVGVYYCFQGTHVPRTFGGGTKLEIR

SEQ ID NO: 316 - protein, 116 aa, synthetic construct
EVQLVQSGAEVKKPGATVKISCKVSGFNIKDSIMHWVQQAPGKGLEWMGWIDPEDGDTEYAPKFQDRVTITADTSTDTAYMELSSLRSEDTAVYYCSKSYYGPDFWGQGTTVTVSS

SEQ ID NO: 317 - protein, 112 aa, synthetic construct
DVVMTQSPLSLPVTLGQPASISCRSSQSIVHSNGNTYLFWFQQRPGQSPRRLIYRVSNRVSGVPDRFSGSGSGTDFTLKISRVEAEDVGVYYCFQGTHVPRTFGGGTKVEIK

SEQ ID NO: 318 - protein, 116 aa, synthetic construct
EVQLVQSGAEVKKPGATVKISCKVSGFNIKDSIMHWVQQAPGKGLEWMGWIDPEDGDTEYAPKFQDRATITADTSTNTAYMELSSLRSEDTAVYYCSKSYYGPDFWGQGTTVTVSS

SEQ ID NO: 319 - protein, 112 aa, synthetic construct
DVVMTQSPLSLPVTLGQPASISCRSSQSIVHSNGNTYLFWFQQRPGQSPRLLIYRVSNRVSGVPDRFSGSGSGTDFTLKISRVEAEDVGVYYCFQGTHVPRTFGGGTKVEIK

SEQ ID NO: 320 - protein, 116 aa, synthetic construct
EVQLVQSGAEVKKPGATVKISCKASGFNIKDSIMHWVQQAPGKGLEWIGWIDPEDGDTEYAPKFQDRATITADTSTNTAYMELSSLRSEDTAVYYCSKSYYGPDFWGQGTTVTVSS

SEQ ID NO: 321 - protein, 112 aa, synthetic construct
DVVMTQSPLSLPVTLGQPASISCRSSQSIVHSNGNTYLFWYQQRPGQSPRLLIYRVSNRVSGVPDRFSGSGSGTDFTLKISRVEAEDVGVYYCFQGTHVPRTFGGGTKVEIK

SEQ ID NO: 322 - protein, 116 aa, synthetic construct
EVQLVQSGAEVKKPGATVKISCKASGFNIKDSIMHWVQQAPGKGLEWIGWIDPEDGDTEYAEKFQDRATITADTSTNTAYMELSSLRSEDTAVYYCSKSYYGPDFWGQGTTVTVSS

SEQ ID NO: 323 - protein, 112 aa, synthetic construct
DIVMTQSPLSLPVTPGEPASISCRSSQSIVHSNGNTYLFWYLQKPGQSPQLLIYRVSNRVSGVPDRFSGSGSGTDFTLKISRVEAEDVGVYYCFQGTHVPRTFGGGTKVEIK

SEQ ID NO: 324 - protein, 116 aa, synthetic construct
EVQLVQSGAEVKKPGASVKVSCKASGFNIKDSIMHWVRQAPGQGLEWIGWIDPEDGDTEYAQKFQGRATITADTSINTAYMELSRLRSDDTAVYYCSKSYYGPDFWGQGTTVTVSS

SEQ ID NO: 325 - protein, 112 aa, synthetic construct
DVVMTQSPLSLPVTPGEPASISCRSSQSIVHSNGNTYLFWYLQKPGQSPQLLIYRVSNRVSGVPDRFSGSGSGTDFTLKISRVEAEDVGVYYCFQGTHVPRTFGGGTKVEIK

SEQ ID NO: 326 - protein, 8 aa, synthetic construct
GFNIKDSI

SEQ ID NO: 327 - protein, 8 aa, synthetic construct
IDPEDGDT

SEQ ID NO: 328 - protein, 9 aa, synthetic construct
SKSYYGPDF

SEQ ID NO: 329 - protein, 11 aa, synthetic construct
QSIVHSNGNTY

SEQ ID NO: 330 - skipped

SEQ ID NO: 331 - protein, 9 aa, synthetic construct
FQGTHVPRT

SEQ ID NO: 332 - DNA, 20 bp, synthetic construct
  source 1..20: mol_type = other DNA
cacggatctaatatacccgc

SEQ ID NO: 333 - DNA, 20 bp, synthetic construct
  source 1..20: mol_type = other DNA
tcccggctaacataccacac

SEQ ID NO: 334 - DNA, 20 bp, synthetic construct
  source 1..20: mol_type = other DNA
ggctgggaggtcttactcct

SEQ ID NO: 335 - DNA, 20 bp, synthetic construct
  source 1..20: mol_type = other DNA
ctgacactaatcgggggcac

SEQ ID NO: 336 - DNA, 20 bp, synthetic construct
  source 1..20: mol_type = other DNA
agggttttagcggagagcac

SEQ ID NO: 337 - DNA, 20 bp, synthetic construct
  source 1..20: mol_type = other DNA
tctgtgctgctggtaggttg

SEQ ID NO: 338 - DNA, 24 bp, synthetic construct
  source 1..24: mol_type = other DNA
cacccagtattcatacccactccg

SEQ ID NO: 339 - DNA, 24 bp, synthetic construct
  source 1..24: mol_type = other DNA
aaaccggagtgggtatgaatactg

SEQ ID NO: 340 - DNA, 30 bp, synthetic construct
  source 1..30: mol_type = other DNA
caccgcactcgcagtcctgacggggcaggg

SEQ ID NO: 341 - DNA, 29 bp, synthetic construct
  source 1..29: mol_type = other DNA
aaaccccctgcccgtcaggactgcgagtg

SEQ ID NO: 342 - DNA, 21 bp, synthetic construct
  source 1..21: mol_type = other DNA
gctttgggagactctggtgta

SEQ ID NO: 343 - DNA, 22 bp, synthetic construct
  source 1..22: mol_type = other DNA
tcaatgcccactgttgtgataa

SEQ ID NO: 344 - DNA, 23 bp, synthetic construct
  source 1..23: mol_type = other DNA
tagactttcgggaaaaacgtgtg

SEQ ID NO: 345 - DNA, 21 bp, synthetic construct
  source 1..21: mol_type = other DNA
agaagctctgttgactggtga

SEQ ID NO: 346 - DNA, 29 bp, synthetic construct
  source 1..29: mol_type = other DNA
tctggcctcttcaactgcatcaccatcca

SEQ ID NO: 347 - DNA, 29 bp, synthetic construct
  source 1..29: mol_type = other DNA
tgaatgccttcatcttgttgctgtgtgag

SEQ ID NO: 348 - DNA, 29 bp, synthetic construct
  source 1..29: mol_type = other DNA
cgctcctggcagaaggctgtcttctactg

SEQ ID NO: 349 - DNA, 29 bp, synthetic construct
  source 1..29: mol_type = other DNA
ggcagcaggcggatgaggagaagctcgcg

SEQ ID NO: 350 - DNA, 20 bp, synthetic construct
  source 1..20: mol_type = other DNA
gttactactgttgtgcccgt

SEQ ID NO: 351 - DNA, 26 bp, synthetic construct
  source 1..26: mol_type = other DNA
gaagctgaccattgaccattgtcttg

SEQ ID NO: 352 - DNA, 22 bp, synthetic construct
  source 1..22: mol_type = other DNA
caggaatctcatccagctgctc

SEQ ID NO: 353 - DNA, 20 bp, synthetic construct
  source 1..20: mol_type = other DNA
cttcatcttgttgctgtgtg

SEQ ID NO: 354 - DNA, 21 bp, synthetic construct
  source 1..21: mol_type = other DNA
gaaggctgtcttctactgcgg

SEQ ID NO: 355 - DNA, 20 bp, synthetic construct
  source 1..20: mol_type = other DNA
ctgaatacactgcccaccct

SEQ ID NO: 356 - DNA, 17 bp, synthetic construct
  source 1..17: mol_type = other DNA
gatggtggtattatgag

SEQ ID NO: 357 - DNA, 17 bp, synthetic construct
  source 1..17: mol_type = other DNA
tagataggtgatatagt

SEQ ID NO: 358 - DNA, 21 bp, synthetic construct
  source 1..21: mol_type = other DNA
gatttttggttttttcaattg

SEQ ID NO: 359 - DNA, 22 bp, synthetic construct
  source 1..22: mol_type = other DNA
gttatgtattatttatattatt

SEQ ID NO: 360 - DNA, 21 bp, synthetic construct
  source 1..21: mol_type = other DNA
gaaggttgttttttattgtgg

SEQ ID NO: 361 - DNA, 21 bp, synthetic construct
  source 1..21: mol_type = other DNA
cactacccaccctcacctaac

SEQ ID NO: 362 - DNA, 20 bp, synthetic construct
  source 1..20: mol_type = other DNA
gttgtttttattgttattag

SEQ ID NO: 363 - DNA, 20 bp, synthetic construct
  source 1..20: mol_type = other DNA
tagagtagagagtttgtata

SEQ ID NO: 364 - DNA, 19 bp, synthetic construct
  source 1..19: mol_type = other DNA
agattgaggttggtttttt

SEQ ID NO: 365 - DNA, 20 bp, synthetic construct
  source 1..20: mol_type = other DNA
tgtaggtattttgatttttt

SEQ ID NO: 366 - DNA, 29 bp, synthetic construct
  source 1..29: mol_type = other DNA
gcgtccgccagctctgcgccgcccgcgca

SEQ ID NO: 367 - DNA, 28 bp, synthetic construct
  source 1..28: mol_type = other DNA
catccaccctctgaacattgcggccggc

SEQ ID NO: 368 - DNA, 29 bp, synthetic construct
  source 1..29: mol_type = other DNA
caacccctcacccgcagtagaagacagcc

SEQ ID NO: 369 - DNA, 28 bp, synthetic construct
  source 1..28: mol_type = other DNA
ctgaccacgctgctgggcaacgccatcg

SEQ ID NO: 370 - DNA, 30 bp, synthetic construct
  source 1..30: mol_type = other DNA
ctgggccattctcagaggggacaagacagg

SEQ ID NO: 371 - DNA, 29 bp, synthetic construct
  source 1..29: mol_type = other DNA
gcaggcggatgaggagaagctcgcggaga

SEQ ID NO: 372 - DNA, 29 bp, synthetic construct
  source 1..29: mol_type = other DNA
gtgtttgttagttttgtgttgtttgtgta

SEQ ID NO: 373 - DNA, 28 bp, synthetic construct
  source 1..28: mol_type = other DNA
tatttattttttgaatattgtggttggt

SEQ ID NO: 374 - DNA, 29 bp, synthetic construct
  source 1..29: mol_type = other DNA
caacccctcacccacaataaaaaacaacc

SEQ ID NO: 375 - DNA, 28 bp, synthetic construct
  source 1..28: mol_type = other DNA
ttgattatgttgttgggtaatgttattg

SEQ ID NO: 376 - DNA, 30 bp, synthetic construct
  source 1..30: mol_type = other DNA
ttgggttatttttagaggggataagatagg

SEQ ID NO: 377 - DNA, 29 bp, synthetic construct
  source 1..29: mol_type = other DNA
gtaggtggatgaggagaagtttgtggaga

SEQ ID NO: 378 - DNA, 26 bp, synthetic construct
  source 1..26: mol_type = other DNA
tgaatgtttttattttgttgttgtgt

SEQ ID NO: 379 - DNA, 30 bp, synthetic construct
  source 1..30: mol_type = other DNA
cacctaacttttaaatcctattttatcaat

SEQ ID NO: 380 - DNA, 19 bp, synthetic construct
  source 1..19: mol_type = other DNA
gtgtatgtgtcccagtgca

SEQ ID NO: 381 - DNA, 18 bp, synthetic construct
  source 1..18: mol_type = other DNA
ccacctgcctgtacctcc

SEQ ID NO: 382 - DNA, 20 bp, synthetic construct
  source 1..20: mol_type = other DNA
ggaaccagagcccagaacat

SEQ ID NO: 383 - DNA, 21 bp, synthetic construct
  source 1..21: mol_type = other DNA
gcactgtggtcaatattgggg

SEQ ID NO: 384 - DNA, 18 bp, synthetic construct
  source 1..18: mol_type = other DNA
gatggagttcagcgaggg

SEQ ID NO: 385 - DNA, 20 bp, synthetic construct
  source 1..20: mol_type = other DNA
gccagacacggaacaaatgt

SEQ ID NO: 386 - DNA, 18 bp, synthetic construct
  source 1..18: mol_type = other DNA
gttgccatggttacccgc

SEQ ID NO: 387 - DNA, 18 bp, synthetic construct
  source 1..18: mol_type = other DNA
ggagggctcagtcacgtg

SEQ ID NO: 388 - DNA, 18 bp, synthetic construct
  source 1..18: mol_type = other DNA
ggcttccctctctgcaga

SEQ ID NO: 389 - DNA, 20 bp, synthetic construct
  source 1..20: mol_type = other DNA
ttctgtgtgggctgaaggac

SEQ ID NO: 390 - DNA, 60 bp, synthetic construct
  source 1..60: mol_type = other DNA
ggcaagcttaatgatacggcgaccaccgagatctacactctggatgagattcctgatttc

SEQ ID NO: 391 - DNA, 28 bp, synthetic construct
  source 1..28: mol_type = other DNA
tcgatgtcgactcgagtcttatgactct

SEQ ID NO: 392 - DNA, 20 bp, synthetic construct
  source 1..20: mol_type = other DNA
gaaatgggtcagcagggctt

SEQ ID NO: 393 - DNA, 27 bp, synthetic construct
  source 1..27: mol_type = other DNA
ggcaagcttaatgatacggcgaccacc

SEQ ID NO: 394 - DNA, 28 bp, synthetic construct
  source 1..28: mol_type = other DNA
tcgatgtcgactcgagtcttatgactct

SEQ ID NO: 395 - RNA, 20 nt, synthetic construct
  source 1..20: mol_type = other RNA
gtgtcagtcacttccagcgg

SEQ ID NO: 396 - RNA, 16 nt, synthetic construct
  source 1..16: mol_type = other RNA
agggaggacgatgcgg

SEQ ID NO: 397 - RNA, 490 nt, synthetic construct
  source 1..490: mol_type = other RNA
ttatgactctattttgggtgtaatttccatgaaattatttatgaatctttttttccctaaggggtggccataaatatcatctttaatgcatcttcattagtagctttcatcttctgatgggcctgcatcttttgttggaagttggatgttgttggtagaaatgttaagtccacatcttaggtgtacggagtttgtataaaatatgaactctaatgtgtagaatttgcaggtcgctgaagtgtcacagtctgctgaaatgggtcagcagggcttccctctctgcagagccctggagtctcttccaagtagaaaatcacactctgtccttcagcccacacagaaggctgtctccaactgcagaaggaagttctttccatggccgctggagcaccctccccacttgtgggcaccatggttcaagtctgccccagtgatcctctcctgtataaacctccaggtacagattatggaaatcaggaatctcatccag

SEQ ID NO: 398 - protein, 42 aa, Homo sapiens
MNAFILLLCEAPFCCQFIEFANTVAEKVDRLRSWQKAVFYCG

SEQ ID NO: 399 - DNA, 490 bp, Homo sapiens
  source 1..490: mol_type = genomic DNA
ctggatgagattcctgatttccataatctgtacctggaggtttatacaggagaggatcactggggcagacttgaaccatggtgcccacaagtggggagggtgctccagcggccatggaaagaacttccttctgcagttggagacagccttctgtgtgggctgaaggacagagtgtgattttctacttggaagagactccagggctctgcagagagggaagccctgctgacccatttcagcagactgtgacacttcagcgacctgcaaattctacacattagagttcatattttatacaaactccgtacacctaagatgtggacttaacatttctaccaacaacatccaacttccaacaaaagatgcaggcccatcagaagatgaaagctactaatgaagatgcattaaagatgatatttatggccaccccttagggaaaaaaagattcataaataatttcatggaaattacacccaaaatagagtcataa

SEQ ID NO: 400 - DNA, 53 bp, Homo sapiens
  source 1..53: mol_type = genomic DNA
agagccctggagtctcttccaagtagaaaatcacactctgtccttcagcctga